(19) United States
(12) Patent Application Publication
(10) Pub. No.: US 2022/0296517 A1
(43) Pub. Date: Sep. 22, 2022

(54) COMPOSITIONS AND METHODS FOR ENHANCED DELIVERY OF AGENTS

(71) Applicant: Moderna TX, Inc., Cambridge, MA (US)

(72) Inventors: Kerry Benenato, Sudbury, MA (US); Staci Sabnis, Medford, MA (US); Edward Hennessy, Westwood, MA (US); Kristine Burke, South Boston, MA (US); Matthew Theisen, Wakefield, MA (US); Jaclyn Milton, Somerville, MA (US); Timothy Salerno, Somerville, MA (US); Stephen Hoge, Brookline, MA (US)

(21) Appl. No.: 17/632,938

(22) PCT Filed: Aug. 6, 2020
(86) PCT No.: PCT/US2020/045213

§ 371 Date: Feb. 4, 2022

Related U.S. Application Data
(60) Provisional application No. 62/884,133, filed on Aug. 7, 2019.

(51) Int. Cl. A61K 9/127; A61K 48/00; A61K 31/7105; A61K 31/711; A61K 47/28; A61K 47/24; A61K 47/18

(52) U.S. Cl. CPC A61K 9/1272; A61K 48/0008; A61K 31/7105; A61K 31/711; A61K 47/28; A61K 47/24; A61K 47/18

(57) ABSTRACT

The disclosure features target cell delivery lipid nanoparticle (LNP) compositions that allow for enhanced delivery of agents, e.g., nucleic acids, such as therapeutic and/or prophylactic RNAs, to target cells, in particular liver cells and/or splenic cells. The LNPs comprise an effective amount of a target cell delivery potentiating lipid such that delivery of an agent by a target cell target cell delivery LNP is enhanced as compared to an LNP lacking the target cell delivery potentiating agent. Methods of using the target cell target cell delivery LNPs for delivery of agents, e.g., nucleic acid delivery, for protein expression, and for modulating target cell activity are also disclosed.

### CROSS-REFERENCE TO RELATED APPLICATIONS

This application claims priority to U.S. Provisional Application 62/884,133 filed on Aug. 7, 2019, the entire contents of which is hereby incorporated by reference.

### SEQUENCE LISTING

The instant application contains a Sequence Listing which has been submitted electronically in ASCII format and is hereby incorporated by reference in its entirety. Said ASCII copy, created on Jul. 21, 2020, is named M2180-7000WO_SL.txt and is 12,612 bytes in size.

### BACKGROUND OF THE DISCLOSURE

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutics and/or prophylactics such as nucleic acids to cells.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more: (1) “cationic” and/or amino (ionizable) lipids, (2) phospholipids and/or polyunsaturated lipids (helper lipids), (3) structural lipids (e.g., sterols), and/or (4) lipids containing polyethylene glycol (PEG lipids). Optimally, lipid nanoparticle compositions contain each of 1) an amino (ionizable) lipid, 2) a phospholipid, 3) a structural lipid or blend thereof, 4) a PEG lipid and 5) an agent. Cationic and/or ionizable lipids include, for example, amine-containing lipids that can be readily protonated. Though a variety of such lipid-containing nanoparticle compositions have been demonstrated, effective delivery vehicles for reaching desired cell populations while maintaining safety, and efficacy, are still lacking.

### SUMMARY OF THE DISCLOSURE

In some aspects, by using a target cell target cell delivery LNP, delivery to a target cell is enhanced in vitro, while in other aspects, delivery to a target cell is enhanced in vivo. When administered in vivo, in one embodiment, target cell target cell delivery LNPs demonstrate enhanced delivery of agents to the liver and spleen when compared to reference LNPs. In some aspects, the target cell, e.g., a liver cell (e.g., a hepatocyte) or splenic cell, is contacted with the LNP in vitro. In some aspects, the target cell is contacted with the LNP in vivo by administering the LNP to a subject, e.g., a human subject. In one embodiment, the subject is one that would benefit from modulation of protein expression of a target protein, e.g., in a target cell. In some aspects, the LNP is administered intravenously. In some aspects, the LNP is administered intramuscularly. In some aspects, the LNP is administered by a route selected from the group consisting of subcutaneously, intranodally and intratumorally.

In one embodiment, the agent may comprise or consist of a nucleic acid molecule. In some aspects, the nucleic acid molecule is selected from the group consisting of RNA, mRNA, RNAi, dsRNA, siRNA, antisense RNA, ribozyme, CRISPR/Cas9, ssDNA and DNA. In some aspects, the nucleic acid molecule is RNA selected from the group consisting of a shortmer, an antagomir, an antisense, a ribozyme, a small interfering RNA (siRNA), an asymmetrical interfering RNA (aiRNA), a microRNA (miRNA or miR), a Dicer-substrate RNA (dsRNA), a small hairpin RNA (shRNA), a messenger RNA (mRNA), and mixtures thereof. In some embodiments, the nucleic acid molecule is an siRNA molecule. In some embodiments, the nucleic acid molecule is a miR. In some embodiments, the nucleic acid molecule is an antagomir. In some aspects, the nucleic acid molecule is DNA. In some aspects, the nucleic acid molecule is mRNA.

Accordingly, in one aspect the invention features a target cell delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP results in one, two, three or all of:

(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

In an embodiment the target cell is a liver cell, e.g., a hepatocyte. In an embodiment, the target cell is a hepatocyte.

In an embodiment, the target cell delivery LNP, results in expression and/or activity of payload in greater than 30%, 40%, 50%, 55%, 60%, 65%, 70%, 75% or more total liver cells. In an embodiment, the target cell delivery LNP, results in expression and/or activity of payload in about 30-75%, 40-75%, 50-75%, 55-75%, 60-75%, 65-75%, 70-75%, 30-70%, 30-65%, 30-60%, 30-55%, 30-50%, or 30-40% total liver cells, e.g., as measured by an assay of Example 6. In an embodiment, the target cell delivery LNP, results in expression and/or activity of payload in about 30%, 35%, 40%, 45%, 50%, 51%, 52%, 53%, 54%, 555, 56%, 57%, 58%, 59%, 60%, 61%, 62%, 63%, 64% 65%, 66%, 67%, 68%, 69%, or 70% of total liver cells. In an embodiment, the target cell delivery LNP, results in expression and/or activity of payload in about 60% of total liver cells.

In an embodiment, the target cell delivery LNP, results in enhanced payload level (e.g., expression) in liver cells, e.g., hepatocytes, relative to a reference LNP. In an embodiment, the target cell delivery LNP, results in about 1.5-fold, 2-fold, 3-fold, 4-fold, 5-fold, 6-fold increase in liver cell expression, e.g., hepatocyte expression, relative to a reference LNP. In an embodiment, the target cell delivery LNP, results in about 3-fold increase in liver cell expression, e.g., hepatocyte expression, relative to a reference LNP.

In an embodiment, the target cell delivery LNP has an increased efficiency of cytosolic delivery, e.g., as compared to a reference LNP, e.g., as described herein.

In an embodiment, the target cell delivery LNP results in one, two or all of:

a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;

b) greater half-life (t ½) in the liver relative to plasma, e.g., a t ½ that is at least 1-, 1.1-1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or

c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.

In an embodiment, the target cell delivery LNP has an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced endosomal escape; or
8) slower lipid metabolism in the liver relative to the spleen, e.g., at least 10%, 20%, 30%, 40%, 50%, 60%, 70%, 80%, 90% or more lipid remaining in the liver 24 hours post-administration.

In another aspect, the invention features a method of enhancing a payload level (e.g., payload expression) in a subject, comprising:

administering to the subject a delivery lipid nanoparticle (LNP) described herein, in an amount sufficient to enhance the payload level in the subject.

In an embodiment, the target cell is a liver cell, e.g., a hepatocyte. In an embodiment, the target cell is a hepatocyte.

In an aspect, the invention features a method of enhancing a payload level (e.g., payload expression) in a subject. The method comprising:

administering to the subject a target cell delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP is administered in an amount sufficient to result in one, two, three or all of:

(a) enhanced payload level in a target cell, organ, cellular compartment, or fluid compartment, e.g., the liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP; or

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

In an embodiment the target cell is a liver cell, e.g., a hepatocyte. In an embodiment, the target cell is a hepatocyte.

In an aspect, the invention features a method of treating or ameliorating a symptom of a disorder or disease, e.g., a rare disease, in a subject. The method comprising:

administering to the subject a target cell delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP is administered in an amount sufficient to result in one, two, three or all of:

(a) enhanced payload level in a target cell, organ, cellular compartment, or fluid compartment, e.g., the liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP; or

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP,

thereby treating or ameliorating a symptom of the disorder or disease.

In an embodiment, the target cell is a liver cell, e.g., a hepatocyte. In an embodiment, the target cell is a hepatocyte.

In an embodiment of any of the methods disclosed herein, the target cell delivery LNP, results in expression and/or activity of payload in greater than 30%, 40%, 50%, 55%, 60%, 65%, 70%, 75% or more total liver cells. In an embodiment, the target cell delivery LNP, results in expression and/or activity of payload in about 30-75%, 40-75%, 50-75%, 55-75%, 60-75%, 65-75%, 70-75%, 30-70%, 30-65%, 30-60%, 30-55%, 30-50%, or 30-40% total liver cells, e.g., as measured by an assay of Example 6. In an embodiment, the target cell delivery LNP, results in expression and/or activity of payload in about 30%, 35%, 40%, 45%, 50%, 51%, 52%, 53%, 54%, 555, 56%, 57%, 58%, 59%, 60%, 61%, 62%, 63%, 64% 65%, 66%, 67%, 68%, 69%, or 70% of total liver cells. In an embodiment, the target cell delivery LNP, results in expression and/or activity of payload in about 60% of total liver cells.

In an embodiment of any of the methods disclosed herein, the target cell delivery LNP, results in enhanced payload level (e.g., expression) in liver cells, e.g., hepatocytes, relative to a reference LNP. In an embodiment, the target cell delivery LNP, results in about 1.5-fold, 2-fold, 3-fold, 4-fold, 5-fold, 6-fold increase in liver cell expression, e.g., hepatocyte expression, relative to a reference LNP. In an embodiment, the target cell delivery LNP, results in about 3-fold increase in liver cell expression, e.g., hepatocyte expression, relative to a reference LNP.

In an embodiment of any of the methods disclosed herein, the target cell delivery LNP has an increased efficiency of cytosolic delivery, e.g., as compared to a reference LNP, e.g., as described herein.

In an embodiment of any of the methods disclosed herein, the target cell delivery LNP is administered in an amount that results in one, two or all of:
a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;
b) greater half-life (t_{1/2}) in the liver relative to plasma, e.g., a t_{1/2} that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or
c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.

In an embodiment of any of the methods disclosed herein, the target cell delivery LNP is administered in an amount that results in an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced endosomal escape; or
8) slower lipid metabolism in the liver relative to the spleen, e.g., at least 10%, 20%, 30%, 40%, 50%, 60%, 70%, 80%, 90% or more lipid remaining in the liver 24 hours post-administration.

In some aspects, the method further comprises administering, concurrently or consecutively, a second LNP encapsulating the same or different nucleic acid molecule, wherein the second LNP lacks a target cell delivery potentiating lipid, e.g., comprises a different ionizable lipid. In other aspects, the method further comprises administering, concurrently or consecutively, a second LNP encapsulating a different nucleic acid molecule, wherein the second LNP comprises a target cell delivery potentiating lipid, e.g., comprises the same ionizable lipid.

In one embodiment of the LNPs or methods of the disclosure, the enhanced delivery is relative to a reference LNP, e.g., an LNP comprising a different ionizable lipid, e.g., as described herein. In another embodiment of the LNPs or methods of the disclosure, the enhanced delivery is relative to a suitable control.

In one embodiment of the LNPs or methods of the disclosure, the agent stimulates protein expression in the target cell, e.g., as described herein, e.g., a liver cell or a splenic cell. In another embodiment of the LNPs or methods of the disclosure, the agent inhibits protein expression in the target cell, e.g., as described herein, e.g., a liver cell or a splenic cell. In another embodiment of the LNPs or methods of the disclosure, the agent encodes a soluble protein that modulates target cell activity, e.g., liver cell or splenic cell activity. In another embodiment of the LNPs or methods of the disclosure, the agent encodes an intracellular protein that modulates target cell activity, e.g., liver cell or splenic cell activity. In another embodiment of the LNPs or methods of the disclosure, the agent encodes a transmembrane protein that modulates target cell activity, e.g., liver cell or splenic cell activity. In another embodiment of the LNPs or methods of the disclosure, the agent enhances target cell function, e.g., liver cell or splenic cell function. In another embodiment of the LNPs or methods of the disclosure, the agent inhibits target cell function, e.g., liver cell or splenic cell function.

In one embodiment of the LNPs or methods of the disclosure, the target cell is a liver cell, e.g., a hepatocyte, a hepatic stellate cell, a Kupffer cell, or a liver sinusoidal cell, or a combination thereof.

In one embodiment of the LNPs or methods of the disclosure, the target cell is a splenic cell, e.g., a non-immune splenic cell (e.g., a splenocyte).

In one embodiment of the LNPs or methods of the disclosure, the target cell is chosen from an ovarian cell, a lung cell, an intestinal cell, a heart cell, a skin cell, an eye cell or a brain cell, or a skeletal muscle cell.

In one embodiment of the LNPs or methods of the disclosure, the target cell is a non-immune cell.

In one embodiment of the LNPs or methods of the disclosure, the LNP comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In one embodiment, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In one embodiment, the phytosterol is a combination of Compound S-141, Compound S-140, Compound S-143 and Compound S-148. In one embodiment, the phytosterol comprises a sitosterol or a salt or an ester thereof. In one embodiment, the phytosterol comprises a stigmasterol or a salt or an ester thereof. In one embodiment, the phytosterol is beta-sitosterol

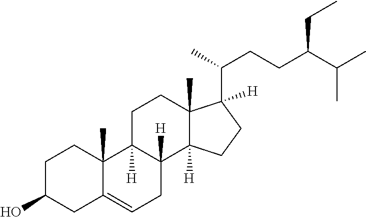

or a salt or an ester thereof.

In one embodiment of the LNPs or methods of the disclosures, the LNP comprises a phytosterol, or a salt or ester thereof, and cholesterol or a salt thereof.

In some embodiments, the target cell is a cell described herein (e.g., a liver cell or a splenic cell), and the phytosterol or a salt or ester thereof is selected from the group consisting of β-sitosterol, β-sitostanol, campesterol, and brassicasterol, and combinations thereof. In one embodiment, the phytosterol is β-sitosterol. In one embodiment, the phytosterol is β-sitostanol. In one embodiment, the phytosterol is campesterol. In one embodiment, the phytosterol is brassicasterol.

In some embodiments, the target cell is a cell described herein (e.g., a liver cell or a splenic cell), and the phytosterol or a salt or ester thereof is selected from the group consisting of β-sitosterol, and stigmasterol, and combinations thereof. In one embodiment, the phytosterol is β-sitosterol. In one embodiment, the phytosterol is stigmasterol.

In some embodiments of the LNPs or methods of the disclosure, the LNP comprises a sterol, or a salt or ester thereof, and cholesterol or a salt thereof, wherein the target cell is a cell described herein (e.g., a liver cell or a splenic cell), and the sterol or a salt or ester thereof is selected from the group consisting of β-sitosterol-d7, brassicasterol, Compound S-30, Compound S-31 and Compound S-32.

In one embodiment, the mol % cholesterol is between about 1% and 50% of the mol % of phytosterol present in the lipid nanoparticle. In one embodiment, the mol % cholesterol is between about 10% and 40% of the mol % of phytosterol present in the lipid nanoparticle. In one embodiment, the mol % cholesterol is between about 20% and 30% of the mol % of phytosterol present in the lipid nanoparticle. In one embodiment, the mol % cholesterol is about 30% of the mol % of phytosterol present in the lipid nanoparticle.

In one embodiment of the LNPs or methods of the disclosure, the ionizable lipid comprises a compound of any of Formulae (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIIa), (I VIIb-1), (I VIIb-2), (I VIIb-3), (I VIIb-4), (I VIIb-5), (I VIIc), (I VIId), (I VIII), (I VIIIa), (I VIIIb), (I VIIIc), (I VIIId), (I XI), (I XI-a), or (I XI-b), and/or comprises a compound selected from the group consisting of: Compound I-18, Compound I-48, Compound I-49, Compound I-50, Compound I-182, Compound I-184, Compound I-292, Compound I-301, Compound I-309, Compound I-317, Compound I-321, Compound I-326, Compound I-347, Compound I-348, Compound I-349, Compound I-350, and Compound I-352.

In one embodiment, the ionizable lipid comprises a compound selected from the group consisting of Compound X, Compound I-48, Compound I-49, Compound I-50, Compound I-182, Compound I-184, Compound I-292, Compound I-301, Compound I-309, Compound I-317, Compound I-321, Compound I-326, Compound I-347, Compound I-348, Compound I-349, Compound I-350, and Compound I-352. In one embodiment, the ionizable lipid comprises a compound selected from the group consisting of Compound I-182, Compound I-292, Compound I-301, Compound I-309, Compound I-317, Compound I-321, Compound I-326, Compound I-347, Compound I-348, Compound I-349, Compound I-350, and Compound I-352. In one embodiment, the ionizable lipid comprises a compound selected from the group consisting of Compound X, Compound I-48, Compound I-49, Compound I-50, and Compound I-184. In one embodiment, the ionizable lipid comprises a compound selected from the group consisting of Compound X, Compound I-49, Compound I-182, Compound I-184, Compound I-301, and Compound I-321. In one embodiment, the ionizable lipid comprises a compound selected from the group consisting of Compound I-301 and Compound I-49. In one embodiment, the ionizable lipid comprises Compound I-301. In one embodiment, the ionizable lipid comprises Compound I-49.

In some embodiments, the target cell is a cell described herein and the ionizable lipid comprises a compound selected from the group consisting of Compound I-301, and Compound I-49. In other embodiments, the target cell is a liver cell or a splenic cell, and the ionizable lipid comprises a compound selected from the group consisting of Compound I-301, and Compound I-49.

In any of the foregoing or related aspects, the ionizable lipid of the LNP of the disclosure comprises at least one compound selected from the group consisting of: Compound I-301, and Compound I-49. In one embodiment, the ionizable lipid comprises Compound I-301. In one embodiment, the ionizable lipid comprises Compound I-49.

In some embodiments, the ionizable lipid comprises an enantiomer, e.g., an (R)-enantiomer or an (S)-enantiomer of an amino lipid. In some embodiments, the ionizable lipid comprises a substantially pure enantiomer, e.g., at least 80%, 90%, 95%, 95%, 97%, 98%, 99% or 100% pure enantiomer. In some embodiments, the ionizable lipid comprises a substantially pure enantiomer of an amino lipid, e.g., at least 80%, 90%, 95%, 95%, 97%, 98%, 99% or 100% pure enantiomer. In some embodiments, the ionizable lipid comprises a substantially pure (R)-enantiomer of an amino lipid, e.g., at least 80%, 90%, 95%, 95%, 97%, 98%, 99% or 100% pure (R)-enantiomer. In some embodiments, the ionizable lipid comprises a substantially pure (S)-enantiomer of an amino lipid, e.g., at least 80%, 90%, 95%, 95%, 97%, 98%, 99% or 100% pure (S)-enantiomer.

In one embodiment, the ionizable lipid comprises a racemic mixture of an amino lipid, e.g., a mixture comprising a (R)-enantiomer and an (S)-enantiomer of an amino lipid. In one embodiment, the racemic mixture comprises about 1-99%, 5-99%, 10-99%, 15-99%, 20-99%, 25-99%, 30-99%, 35-99%, 40-99%, 45-99%, 50-99%, 55-99%, 60-99%, 65-99%, 70-99%, 75-99%, 80-99%, 85-99%, 90-99%, 95-99%, 1-95%, 1-90%, 1-85%, 1-80%, 1-75%, 1-70%, 1-65%, 1-60%, 1-55%, 1-50%, 1-45%, 1-40%, 1-35%, 1-30%, 1-25%, 1-20%, 1-15%, 1-10%, 1-5%, 1-10%, 10-20%, 20-30%, 30-40%, 40-50%, 50-60%, 60-70%, 70-805, 80-90%, or 90-99% of a (R)-enantiomer. In one embodiment, the racemic mixture comprises about 1-99%, 5-99%, 10-99%, 15-99%, 20-99%, 25-99%, 30-99%, 35-99%, 40-99%, 45-99% 50-99%, 55-99%, 60-99%, 65-99%, 70-99%, 75-99%, 80-99%, 85-99%, 90-99%, 95-99%, 1-95%, 1-90%, 1-85%, 1-80%, 1-75%, 1-70%, 1-65%, 1-60%, 1-55%, 1-50%, 1-45%, 1-40%, 1-35%, 1-30%, 1-25%, 1-20%, 1-15%, 1-10%, 1-5%, 1-10%, 10-20%, 20-30%, 30-40%, 40-50%, 50-60%, 60-70%, 70-805, 80-90%, or 90-99% of an (S)-enantiomer.

In one embodiment of the LNPs or methods of the disclosure, the non-cationic helper lipid or phospholipid comprises a compound selected from the group consisting of DSPC, DMPE, DOPC and Compound H-409. In one embodiment of the LNPs or methods of the disclosure, the non-cationic helper lipid or phospholipid comprises a compound selected from the group consisting of DSPC, DPPC, DMPE, DMPC, DOPC, Compound H-409, Compound H-418, Compound H-420, Compound H-421 and Compound H-422. In one embodiment, the phospholipid is DSPC. In one embodiment of the LNPs or methods of the disclosure, the non-cationic helper lipid or phospholipid comprises a compound selected from the group consisting of DPPC, DMPC, Compound H-418, Compound H-420, Compound H-421 and Compound H-422.

In one embodiment of the LNPs or methods of the disclosure, the target cell is a cell described herein and the non-cationic helper lipid or phospholipid comprises a compound selected from the group consisting of DSPC, DMPE, and Compound H-409. In one embodiment, the phospholipid is DSPC. In one embodiment, the phospholipid is DMPE. In one embodiment, the phospholipid is Compound H-409.

In one embodiment of the LNPs or methods of the disclosure, the target cell is a cell described herein and the non-cationic helper lipid or phospholipid comprises a compound selected from the group consisting of DOPC, DMPE, and Compound H-409. In one embodiment, the phospholipid is DSPC. In one embodiment, the phospholipid is DMPE. In one embodiment, the phospholipid is Compound H-409.

In one embodiment of the LNPs or methods of the disclosure, the LNP comprises a PEG-lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. In one embodiment, the PEG lipid is selected from the group consisting of Compound 428, Compound P-L16, Compound P-L17, Compound P-L18, Compound P-L19, Compound P-L1, and Compound P-L2. In one embodiment, the PEG lipid is selected from the group consisting of Compound P 415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23. Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In one embodiment, the PEG lipid is selected from the group consisting of Compound P-L3, Compound P-L4, Compound P-L6, Compound P-L8, Compound P-L9 and Compound P-L25.

In one embodiment of the LNPs or methods of the disclosure, the LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In one embodiment of the LNPs or methods of the disclosure, the LNP comprises about 41 mol % to about 50 mol % ionizable lipid and about 10 mol % to about 19 mol % non-cationic helper lipid or phospholipid. In one embodiment of the LNPs or methods of the disclosure, the LNP comprises about 50 mol % ionizable lipid and about 10 mol % non-cationic helper lipid or phospholipid. In one embodiment of the LNPs or methods of the disclosure, the LNP comprises 50 mol % ionizable lipid and 10 mol % non-cationic helper lipid or phospholipid.

In one embodiment of the LNPs or methods of the disclosure, the LNP comprises about 50 mol % Compound I-301 and about 10 mol % non-cationic helper lipid or phospholipid. In one embodiment of the LNPs or methods of the disclosure, the LNP comprises 50 mol % Compound I-301 and about 10 mol % non-cationic helper lipid or phospholipid. In one embodiment of the LNPs or methods of the disclosure, the LNP comprises about 50 mol % Compound I-301 and 10 mol % non-cationic helper lipid or phospholipid. In one embodiment of the LNPs or methods of the disclosure, the LNP comprises 50 mol % Compound I-301 and 10 mol % non-cationic helper lipid or phospholipid.

In one embodiment of the LNPs or methods of the disclosure, the LNP comprises about 50 mol % Compound I-49 and about 10 mol % non-cationic helper lipid or phospholipid. In one embodiment of the LNPs or methods of the disclosure, the LNP comprises 50 mol % Compound I-49 and about 10 mol % non-cationic helper lipid or phospholipid. In one embodiment of the LNPs or methods of the disclosure, the LNP comprises about 50 mol % Compound I-49 and 10 mol % non-cationic helper lipid or phospholipid. In one embodiment of the LNPs or methods of the disclosure, the LNP comprises 50 mol % Compound I-49 and 10 mol % non-cationic helper lipid or phospholipid.

In one embodiment of the LNPs or methods of the disclosure, the LNP comprises: (i) about 50 mol % ionizable lipid, wherein the ionizable lipid is a compound selected from the group consisting of Compound I-301, and Compound I-49;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

In some aspects, the disclosure provides a target cell delivery lipid nanoparticle (LNP) for use in a method of enhancing a payload level (e.g., payload expression) in a subject, wherein the LNP comprises:
(i) a sterol or other structural lipid;
(ii) an ionizable lipid; and
(iii) an agent for delivery to a target cell in the subject;

wherein one or more of (i) the sterol or other structural lipid and/or (ii) the ionizable lipid comprises a target cell delivery potentiating lipid in an amount effective to enhance the payload level in the subject or enhance delivery of the LNP to the target cell subject.

In an embodiment, the enhanced delivery is a characteristic of said LNP relative to a reference LNP. In an embodiment, the reference LNP lacks the target cell delivery potentiating lipid. In an embodiment, the reference LNP comprises an ionizable lipid having Formula I-XII.

In an embodiment the target cell is a liver cell, e.g., a hepatocyte. In an embodiment, the target cell is a hepatocyte.

In some aspects, the disclosure provides a target cell delivery lipid nanoparticle (LNP) for use in a method of enhancing a payload level (e.g., payload expression) in a subject, wherein the LNP comprises
(i) a sterol or other structural lipid;
(ii) an ionizable lipid; and
(iii) an agent for delivery to a target cell in the subject;

wherein the sterol or other structural lipid comprises a target cell delivery potentiating lipid in an amount effective to enhance the payload level in the subject or enhance delivery of the LNP to the target cell subject,

wherein the enhanced delivery is a characteristic of said LNP relative to a reference LNP.

In an embodiment, the reference LNP lacks the target cell delivery potentiating lipid. In an embodiment, the reference LNP comprises an ionizable lipid having Formula I-XII.

In an embodiment the target cell is a liver cell, e.g., a hepatocyte. In an embodiment, the target cell is a hepatocyte.

In some aspects, the disclosure provides a target cell delivery lipid nanoparticle (LNP) for use in a method of enhancing a payload level (e.g., payload expression) in a subject,

wherein the LNP comprises
(i) a sterol or other structural lipid;
(ii) an ionizable lipid; and
(iii) an agent for delivery to a target cell in the subject;

wherein the ionizable lipid comprises a target cell delivery potentiating lipid in an amount effective to enhance delivery of the LNP to a target cell (e.g., as described herein, e.g., a liver cell or splenic cell),

wherein the enhanced delivery is a characteristic of said LNP relative to a reference LNP.

In an embodiment, the reference LNP lacks the target cell delivery potentiating lipid. In an embodiment, the reference LNP comprises an ionizable lipid having Formula I-XII.

In an embodiment the target cell is a liver cell, e.g., a hepatocyte. In an embodiment, the target cell is a hepatocyte.

In any of the foregoing or related aspects, the sterol or other structural lipid is a phytosterol or cholesterol.

In any of the foregoing or related aspects, the target cell delivery potentiating lipid is preferentially taken up by a liver cell (e.g., a hepatocyte), a splenic cell, an ovarian cell, a lung cell, an intestinal cell, a heart cell, a skin cell, an eye cell or a brain cell, or a skeletal muscle cell compared to a reference LNP. In an embodiment the reference LNP lacks the target cell delivery potentiating lipid and/or is not preferentially taken up by a liver cell (e.g., a hepatocyte), a splenic cell, an ovarian cell, a lung cell, an intestinal cell, a heart cell, a skin cell, an eye cell or a brain cell, or a skeletal muscle cell.

In any of the foregoing or related aspects, the agent for delivery to a target cell described herein is a nucleic acid molecule. In some aspects, the agent stimulates expression of a protein of interest in the target cell. In some aspects, the agent for delivery to a target cell is a nucleic acid molecule encoding a protein of interest. In some aspects, the agent for delivery to a target cell is an mRNA encoding a protein of interest.

In any of the foregoing or related aspects, the expression of the protein of interest in the target cell is enhanced relative to a reference LNP lacking the target cell delivery potentiating lipid. In some aspects, the agent encodes a protein that modulates target cell activity.

In any of the foregoing or related aspects, the target cell is a liver cell, e.g., a hepatocyte, a hepatic stellate cell, a Kupffer cell, or a liver sinusoidal cell, or a combination thereof. In some aspects, the liver cell is a hepatocyte. In some aspects, the liver cell is a hepatic stellate cell. In some aspects, the liver cell is a Kupffer cell. In some aspects the liver cell is a liver sinusoidal cell.

In any of the foregoing or related aspects, the target cell is a splenic cell, e.g., a non-immune splenic cell (e.g., a splenocyte).

In any of the foregoing or related aspects, the target cell is chosen from an ovarian cell, a lung cell, an intestinal cell, a heart cell, a skin cell, an eye cell or a brain cell, or a skeletal muscle cell.

In any of the foregoing or related aspects, the target cell is not an immune cell.

In any of the foregoing or related aspects, the target cell delivery lipid nanoparticle (LNP) further comprises (iv) a non-cationic helper lipid or phospholipid, and/or (v) a PEG-lipid.

In some aspects, the target cell delivery lipid nanoparticle (LNP) further comprises a non cationic helper lipid or phospholipid. In some aspects, the target cell delivery LNP further comprise a PEG-lipid. In some aspects, the target cell delivery LNP further comprises a non-cationic helper lipid or phospholipid, and a PEG-lipid.

In some aspects, the disclosure provides an in vitro method of delivering an agent to a target cell (e.g., as described herein, e.g., a liver cell, e.g., a hepatocyte), the method comprising contacting the target cell with a target cell delivery LNP comprising a target cell delivery potentiating lipid. In some aspects of the in vitro method, the method results in modulation of activation or activity of the target cell.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

The disclosure relates to the following embodiments. Throughout this section, the term embodiment is abbreviated as ‘E’ followed by an ordinal. For example, E1 is equivalent to Embodiment 1.

E1. In an aspect, the invention features a target cell delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP results in one, two, three or all of:

(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

E2. The target cell delivery LNP of E1, wherein the target cell is a liver cell, e.g., a hepatocyte.
E3. The target cell delivery LNP of E1 or E2, which results in expression and/or activity of payload in greater than 30%, 40%, 50%, 55%, 60%, 65%, 70%, 75% or more total liver cells.
E4. The target cell delivery LNP of any one of the preceding embodiments, which results in expression and/or activity of payload in about 30-75%, 40-75%, 50-75%, 55-75%, 60-75%, 65-75%, 70-75%, 30-70%, 30-65%, 30-60%, 30-55%, 30-50%, or 30-40% total liver cells, e.g., as measured by an assay of Example 6.
E5. The target cell delivery LNP of any one of the preceding embodiments, which results in expression and/or activity of payload in about 30%, 35%, 40%, 45%, 50%, 51%, 52%, 53%, 54%, 555, 56%, 57%, 58%, 59%, 60%, 61%, 62%, 63%, 64% 65%, 66%, 67%, 68%, 69%, or 70% of total liver cells.
E6. The target cell delivery LNP of any one of the preceding embodiments, which results in expression and/or activity of payload in about 60% of total liver cells.
E7. The target cell delivery LNP of any one of the preceding embodiments, which results in enhanced payload level (e.g., expression) in liver cells, e.g., hepatocytes, relative to a reference LNP.
E8. The target cell delivery LNP of any one of the preceding embodiments, which results in about 1.5-fold, 2-fold, 3-fold, 4-fold, 5-fold, or 6-fold increase in liver cell expression, e.g., hepatocyte expression, relative to a reference LNP.
E9. The target cell delivery LNP of any one of the preceding embodiments, which results in 1.5-6 fold, 1.5-5 fold, 1.5-4 fold, 1.5-3 fold, 1.5-2 fold, 2-6 fold, 3-6 fold, 4-6 fold or 5-6 fold increase in liver cell expression, e.g., hepatocyte expression, relative to a reference LNP.
E10. The target cell delivery LNP of any one of the preceding embodiments, which results in about 3-fold increase in liver cell expression, e.g., hepatocyte expression, relative to a reference LNP.
E11. The target cell delivery LNP of any one of the preceding embodiments, which has an increased efficiency of cytosolic delivery, e.g., as compared to a reference LNP, e.g., as described herein.
E12. The target cell delivery LNP of any one of the preceding embodiments, which results in one, two or all of:
a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;
b) greater half-life (t_{1/2}) in the liver relative to plasma, e.g., a t_{1/2} that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or
c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.
   E13. The target cell delivery LNP of any one of the preceding embodiments, which has an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced expression and/or activity of payload in greater than 30%, 40%, 50%, 60%, 65%, 70%, 75% or more total liver cells; or
8) enhanced endosomal escape.
   E14. The target cell delivery LNP of any one of the preceding embodiments, which results in one, two, three or all of:
9) an increased response rate, e.g., a defined by at specified threshold of liver cell transfection;
10) at least 5%, 10%, 15%, 20%, 25%, 30%, 34%, 35%, 36%, 37%, 38%, 39%, 40% or more liver cell transfection;
11) an increased responder rate, e.g., a defined by at specified threshold of liver cell transfection; or
12) an increased response rate greater than a reference LNP, e.g., at least 1-fold, 1.5-fold, 2-fold, 2.5-fold, or 3-fold or greater response rate.
   E15. The target cell delivery LNP of any one of the preceding embodiments, wherein the target cell delivery LNP is formulated for systemic delivery.
   E16. The target cell delivery LNP of any one of the preceding embodiments, wherein the target cell delivery LNP is administered systemically, e.g., parenterally (e.g., intravenously, intramuscularly, subcutaneously, intrathecally, or intradermally) or enterally (e.g., orally, rectally or sublingually).
   E17. The target cell delivery LNP of any one of the preceding embodiments, which delivers the payload to a cell capable of protein synthesis and/or a cell having a high engulfing capacity.
   E18. The target cell delivery LNP of any one of the preceding embodiments, which delivers the payload to a liver cell, e.g., a hepatocyte, a hepatic stellate cell, a Kupffer cell, or a liver sinusoidal cell, or a combination thereof.
   E19. The target cell delivery LNP of any one of the preceding embodiments, which delivers the payload to a hepatocyte.
   E20. The target cell delivery LNP of any one of the preceding embodiments, which delivers the payload to a non-immune cell.
   E21. The target cell delivery LNP of any one of the preceding embodiments, which delivers the payload to a splenic cell, e.g., a non-immune splenic cell (e.g., a splenocyte).
   E22. The target cell delivery LNP of any one of the preceding embodiments, which delivers the payload to a cell chosen from an ovarian cell, a lung cell, an intestinal cell, a heart cell, a skin cell, an eye cell or a brain cell, or a skeletal muscle cell.
   E23. The target cell delivery LNP of any one of the preceding embodiments, wherein an intracellular concentration of the nucleic acid molecule in the target cell is enhanced.
   E24. The target cell delivery LNP of any one of the preceding embodiments, wherein uptake of the nucleic acid molecule by the target cell is enhanced.
   E25. The target cell delivery LNP of any one of the preceding embodiments, wherein an activity of the nucleic acid molecule in the target cell is enhanced.
   E26. The target cell delivery LNP of any one of the preceding embodiments, wherein expression of the nucleic acid molecule in the target cell is enhanced.
   E27. The target cell delivery LNP of any one of the preceding embodiments, wherein an activity of a protein encoded by the nucleic acid molecule in the target cell is enhanced.
   E28. The target cell delivery LNP of any one of the preceding embodiments, wherein expression of a protein encoded by the nucleic acid molecule in the target cell is enhanced.
   E29. The target cell delivery LNP of any one of the preceding embodiments, wherein delivery is enhanced in vivo.
   E30. The target cell delivery LNP of any one of the preceding embodiments, wherein the payload is a peptide, polypeptide, protein or a nucleic acid.
   E31. The target cell delivery LNP of any one of the preceding embodiments, wherein the payload is a nucleic acid molecule chosen from RNA, mRNA, dsRNA, siRNA, antisense RNA, ribozyme, CRISPR/Cas9, ssDNA and DNA.
   E32. The target cell delivery LNP of any one of the preceding embodiments, wherein the payload is chosen from a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), or a combination thereof.
   E33. The target cell delivery LNP of any one of the preceding embodiments, wherein the payload is an mRNA, a siRNA, a miR, or a CRISPR.
   E34. The target cell delivery LNP of any one of the preceding embodiments, wherein the payload is an mRNA.
   E35. The target cell delivery LNP of any one of the preceding embodiments, wherein the payload is an mRNA encoding a protein of interest other than an immune cell payload.
   E36. The target cell delivery LNP of any one of the preceding embodiments, wherein the payload is chosen from an mRNA encoding secreted protein, a membrane-bound protein, an intracellular protein, an antibody molecule or an enzyme.
   E37. The target cell delivery LNP of any one of the preceding embodiments, wherein the payload is an mRNA encoding an antibody molecule.
   E38. The target cell delivery LNP of any one of the preceding embodiments, wherein the payload is an mRNA encoding an enzyme.
   E39. The target cell delivery LNP of E38, wherein the enzyme is associated with a rare disease (e.g., a lysosomal storage disease).
   E40. The target cell delivery LNP of E38, wherein the enzyme is associated with a metabolic disorder (e.g., as described herein).
   E41. The target cell delivery LNP of E38 or E39, wherein the payload is an mRNA encoding a urea cycle enzyme.
   E42. The target cell delivery LNP of any one of the preceding embodiments, wherein the target cell delivery LNP can be administered at a lower dose compared to a reference LNP, e.g., as described herein.
   E43. The target cell delivery LNP of E42, wherein the target cell delivery LNP administered at a dose that is at least 10%, 20%, 30%, 40%, 50%, 60%, 70%, 80%, or 90% lower compared to the dose of a reference LNP.
   E44. The target cell delivery LNP of E42 or E43, wherein the target cell delivery LNP delivered at a lower dose results in similar or enhanced lipid and/or payload level in a target cell, organ or cellular compartment.
   E45. The target cell delivery LNP of any one of the preceding embodiments, wherein the target cell delivery LNP can be administered at a reduced frequency compared to a reference LNP, e.g., as described herein.
   E46. The target cell delivery LNP of E45, wherein the administration frequency of the target cell delivery LNP is at least 10%, 20%, 30%, 40%, 50%, 60%, 70%, 80%, or 90% lesser than the administration frequency of a reference LNP.
   E47. The target cell delivery LNP of E45 or E46, wherein the target cell delivery LNP delivered at a lesser frequency results in similar or enhanced lipid and/or payload level in a target cell, organ or cellular compartment.
   E48. In an aspect, the invention features a method of enhancing a payload level (e.g., payload expression) in a subject, comprising:

administering to the subject the delivery lipid nanoparticle (LNP) of any one of E1 to E47, in an amount sufficient to enhance the payload level in the subject.

E49. In an aspect, the invention features a method of enhancing a payload level (e.g., payload expression) in a subject, comprising:

administering to the subject a delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP is administered in an amount sufficient to result in one, two or all of:

(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

E50. In an aspect, the invention features a method of treating or ameliorating a symptom of a disorder or disease, e.g., a rare disease, in a subject, comprising:

administering to the subject a delivery lipid nanoparticle (LNP) comprising:

(i) an ionizable lipid, e.g., an amino lipid;

(ii) a sterol or other structural lipid;

(iii) a non-cationic helper lipid or phospholipid;

(iv) a payload; and

(v) optionally, a PEG-lipid,

wherein the target cell delivery LNP is administered in an amount sufficient to result in one, two, three or all of:

(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;

(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;

(c) expression and/or activity of payload in greater than 30%, 40%, 50%, 60%, 65%, 70%, 75% or more total liver cells, e.g., in about 60% of total liver cells; or

(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP, thereby treating or ameliorating a symptom of the disorder or disease.

E51. The method of E49 or E50, wherein the target cell is a liver cell, e.g., a hepatocyte. In an embodiment, the target cell is a hepatocyte.
E52. The method of any one of E49-E51, wherein the target cell delivery LNP, results in expression and/or activity of payload in greater than 30%, 40%, 50%, 55%, 60%, 65%, 70%, 75% or more total liver cells.
E53. The method of any one of E49-E52, wherein target cell delivery LNP, results in expression and/or activity of payload in about 30-75%, 40-75%, 50-75%, 55-75%, 60-75%, 65-75%, 70-75%, 30-70%, 30-65%, 30-60%, 30-55%, 30-50%, or 30-40% total liver cells, e.g., as measured by an assay of Example 6.
E54. The method of any one of E49-E53, wherein the target cell delivery LNP, results in expression and/or activity of payload in about 30%, 35%, 40%, 45%, 50%, 51%, 52%, 53%, 54%, 555, 56%, 57%, 58%, 59%, 60%, 61%, 62%, 63%, 64% 65%, 66%, 67%, 68%, 69%, or 70% of total liver cells.
E55. The method of any one of E49-E54, wherein the target cell delivery LNP, results in expression and/or activity of payload in about 60% of total liver cells.
E56. The method of any one of E49-E55, wherein the target cell delivery LNP, results in enhanced payload level (e.g., expression) in liver cells, e.g., hepatocytes, relative to a reference LNP.
E57. The method of any one of E49-E56, wherein the target cell delivery LNP, results in about 1.5-fold, 2-fold, 3-fold, 4-fold, 5-fold, 6-fold increase in liver cell expression, e.g., hepatocyte expression, relative to a reference LNP.
E58. The method of any one of E49-E57, wherein the target cell delivery LNP, results in about 3-fold increase in liver cell expression, e.g., hepatocyte expression, relative to a reference LNP.
E59. The method of any one of E49-E54, wherein the target cell delivery LNP has an increased efficiency of cytosolic delivery, e.g., as compared to a reference LNP, e.g., as described herein.
E60. The method of any one of E49-E59, wherein the target cell delivery LNP is administered in an amount that results in one, two or all of:
a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;
b) greater half-life (t_{1/2}) in the liver relative to plasma, e.g., a t_{1/2} that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or
c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.
   E61. The method of any one of E49-E60, wherein the target cell delivery LNP is administered in an amount that results in an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced expression and/or activity of payload in greater than 30%, 40%, 50%, 60%, 65%, 70%, 75% or more total liver cells; or
8) enhanced endosomal escape.
   E62. The method of any one of E49-E61, wherein the target cell delivery LNP is administered in an amount that results in one, two, three or all of:
1) an increased response rate, e.g., a defined by at specified threshold of liver cell transfection;
2) at least 5%, 10%, 15%, 20%, 25%, 30%, 34%, 35%, 36%, 37%, 38%, 39%, 40% or more liver cell transfection;
3) an increased responder rate, e.g., a defined by at specified threshold of liver cell transfection; or
4) an increased response rate greater than a reference LNP, e.g., at least 1-fold, 1.5-fold, 2-fold, 2.5-fold, or 3-fold or greater response rate.
   E63. The method of any one of E49-E62, wherein the target cell delivery LNP is formulated for systemic delivery.
   E64. The method of any one of E49-E63, wherein the target cell delivery LNP is administered systemically, e.g., parenterally (e.g., intravenously, intramuscularly, subcutaneously, intrathecally, or intradermally) or enterally (e.g., orally, rectally or sublingually).
   E65. The method of any one of E49-E64, wherein the target cell delivery LNP delivers the payload to a cell capable of protein synthesis and/or a cell having a high engulfing capacity.
   E66. The method of any one of E49-E65, wherein the target cell delivery LNP delivers the payload to a liver cell, e.g., a hepatocyte, a hepatic stellate cell, a Kupffer cell, or a liver sinusoidal cell, or a combination thereof.
   E67. The method of any one of E49-E66, wherein the target cell delivery LNP delivers the payload to a hepatocyte.
   E68. The method of any one of E49-E67, wherein the target cell delivery LNP delivers the payload to a splenic cell, e.g., a non-immune splenic cell (e.g., a splenocyte).
   E69. The method of any one of E49-E68, wherein the target cell delivery LNP delivers the payload to a cell chosen from an ovarian cell, a lung cell, an intestinal cell, a heart cell, a skin cell, an eye cell or a brain cell, or a skeletal muscle cell.
   E70. The method of any one of E49-E69, wherein the target cell delivery LNP delivers the payload to a non-immune cell.
   E71. The method of any one of E49-E69, wherein an intracellular concentration of the nucleic acid molecule in the target cell is enhanced.
   E72. The method of any one of E49-E71, wherein uptake of the nucleic acid molecule by the target cell is enhanced.
   E73. The method of any one of E49-E72, wherein an activity of the nucleic acid molecule in the target cell is enhanced.
   E74. The method of any one of E49-E73, wherein expression of the nucleic acid molecule in the target cell is enhanced.
   E75. The method of any one of E49-E74, wherein an activity of a protein encoded by the nucleic acid molecule in the target cell is enhanced.
   E76. The method of any one of E49-E75, wherein expression of a protein encoded by the nucleic acid molecule in the target cell is enhanced.
   E77. The method of any one of E49-E76, wherein delivery is enhanced in vivo.
   E78. The method of any one of E49-E76, wherein the payload is a peptide, polypeptide, protein or a nucleic acid.
   E79. The method of any one of E49-E78, wherein the is a nucleic acid molecule chosen from RNA, mRNA, dsRNA, siRNA, antisense RNA, ribozyme, CRISPR/Cas9, ssDNA and DNA.
   E80. The method of any one of E49-E79, wherein the payload is chosen from a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), or a combination thereof.
   E81. The method of any one of E49-E80, wherein the payload is an mRNA, a siRNA, a miR, or a CRISPR.
   E82. The method of any one of E49-E81, wherein the payload is an mRNA encoding a protein of interest other than an immune cell payload.
   E83. The method of any one of E49-E82, wherein the payload is chosen from an mRNA encoding secreted protein, a membrane-bound protein, an intracellular protein, an enzyme.
   E84. The method of any one of E49-E83, wherein the payload is an mRNA encoding an antibody molecule.
   E85. The method of any one of E49-E84, wherein the payload is an mRNA encoding an enzyme.
   E86. The method of any one of E49-E85, wherein the enzyme is associated with a rare disease (e.g., a lysosomal storage disease), or a metabolic disorder (e.g., as described herein).
   E87. The method of E86, wherein the payload is an mRNA encoding a urea cycle enzyme.
   E88. The method of E86, wherein the disease is a metabolic disorder.
   E89. The method of any one of E49-E88, wherein the target cell delivery LNP can be administered at a lower dose compared to a reference LNP, e.g., as described herein.
   E90. The method of any one of E49-E89, wherein the target cell delivery LNP administered at a dose that is at least 10%, 20%, 30%, 40%, 50%, 60%, 70%, 80%, or 90% lower compared to the dose of a reference LNP.
   E91. The method of E90, wherein the target cell delivery LNP delivered at a lower dose results in similar or enhanced lipid and/or payload level in a target cell, organ or cellular compartment.
   E92. The method of E90 or E91, wherein the target cell delivery LNP can be administered at a reduced frequency compared to a reference LNP, e.g., as described herein.
   E93. The method of E92, wherein the administration frequency of the target cell delivery LNP is at least 10%, 20%, 30%, 40%, 50%, 60%, 70%, 80%, or 90% lesser than the administration frequency of a reference LNP.
   E94. The method of E92 or E93, wherein the target cell delivery LNP delivered at a lesser frequency results in similar or enhanced lipid and/or payload level in a target cell, organ or cellular compartment.
   E95. The target cell delivery LNP or the method of any of the preceding embodiments, wherein the ionizable lipid comprises an amino lipid.
   E96. The target cell delivery LNP or the method of any of the preceding embodiments, wherein the ionizable lipid comprises a compound of any of Formulae (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), or (I VIIId).
   E97. The target cell delivery LNP or the method of any of the preceding embodiments, wherein the ionizable lipid comprises an amino lipid having a squaramide head group.
   E98. The target cell delivery LNP or the method of any of the preceding embodiments, wherein the ionizable lipid comprises a compound selected from the group consisting of Compound I-301, Compound (R)-I-301, Compound (S)-I-301, Compound I-49, Compound (R)-I-49, Compound (S)-I-49, Compound I-292, Compound I-309, Compound I-317, Compound I-326, Compound I-347, Compound I-348, Compound I-349, Compound I-350, and Compound I-352.
   E99. The target cell delivery LNP or the method of any of the preceding embodiments, wherein the ionizable lipid comprises a compound selected from Compound I-301 and Compound I-49.
   E100. The target cell delivery LNP or the method of any of the preceding embodiments, wherein the ionizable lipid comprises Compound I-301.
   E101. The target cell delivery LNP or the method of any of E1-E99, wherein the ionizable lipid comprises Compound I-49.
   E102. The target cell delivery LNP or the method of any of E1-E99, wherein the cell is a liver cell, e.g., a hepatocyte, and the ionizable lipid comprises a compound selected from the group consisting of Compound I-301 and Compound I-49.
   E103. The target cell delivery LNP or the method of any of E1-E99, wherein the cell is a splenic cell, e.g., a splenocyte, and the ionizable lipid comprises a compound selected from the group consisting of Compound I-301 and Compound I-49.
   E104. The target cell delivery LNP or the method of any of the preceding embodiments, wherein the ionizable lipid comprises is a racemic mixture of the amino lipid, e.g., a mixture comprising a (R)-enantiomer and an (S)-enantiomer of an amino lipid.
   E105. The target cell delivery LNP or the method of any of the preceding embodiments, wherein the ionizable lipid comprises an enantiomer, e.g., an (R)-enantiomer or an (S)-enantiomer of an amino lipid.
   E106. The target cell delivery LNP or the method of E105, wherein the ionizable lipid comprises a substantially pure (R) enantiomer of the amino lipid, e.g., at least 80%, 90%, 95%, 95%, 97%, 98% 99% or 100% pure enantiomer.
   E107. The target cell delivery LNP or the method of E105, wherein the ionizable lipid comprises a substantially pure (S) enantiomer of the amino lipid, e.g., at least 80%, 90%, 95%, 95%, 97%, 98% 99% or 100% pure enantiomer.
   E108. The target cell delivery LNP or the method of any of the preceding embodiments, wherein the reference LNP comprises an ionizable lipid having Formula I-XII.
   E109. The target cell delivery LNP or the method of E108, wherein the reference LNP does not comprises an ionizable lipid having a chiral center.
   E110. The target cell delivery LNP or the method of E108, wherein the reference LNP does not comprises an ionizable lipid comprising more than one branched alkyl chains.
   E111. The target cell delivery LNP or the method of E108, wherein the reference LNP does not comprises a cyclic-substituted amino lipid.
   E112. The target cell delivery LNP or the method of E108, wherein the reference LNP does not comprise a carbocyclic-substituted amino lipid.
   E113. The target cell delivery LNP or the method of E108, wherein the reference LNP does not comprise a cycloalkenyl-substituted amino lipid.
   E114. The target cell delivery LNP or the method of any of the preceding embodiments, wherein the target cell delivery LNP comprises an amino lipid having a chiral center.
   E115. The target cell delivery LNP or the method of any of the preceding embodiments, wherein the target cell delivery LNP comprises an amino lipid comprising more than one branched alkyl chains.
   E116. The target cell delivery LNP or the method of any of the preceding embodiments, wherein the target cell delivery LNP comprises a cyclic-substituted amino lipid.
   E117. The target cell delivery LNP or the method of any of E1-E114 or E116, wherein the target cell delivery LNP comprises a carbocyclic-substituted amino lipid.
   E118. The target cell delivery LNP or the method of any of E1-E114 or E116-E117, wherein the target cell delivery LNP comprises a cycloalkenyl-substituted amino lipid.
   E119. The target cell delivery LNP or the method of any of the preceding embodiments, wherein the target cell delivery LNP comprises a cyclobutenyl-substituted amino lipid.
   E120. The target cell delivery LNP or the method of any of the preceding embodiments, wherein the target cell delivery LNP comprises a cyclobutene-1,2-dione-substituted amino lipid.
   E121. The target cell delivery LNP or the method of any of the preceding embodiments, wherein the target cell delivery LNP comprises a squaramide-substituted amino lipid, e.g., an amino lipid comprising a squaramide group.
   E122. The target cell delivery LNP or the method of any of the preceding embodiments, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.
   E123. The target cell delivery LNP or the method of E122, wherein the cell is a liver cell, e.g., a hepatocyte, and the non-cationic helper lipid or phospholipid comprises a compound selected from the group consisting of DSPC, DMPE, and Compound H-409.
   E124. The target cell delivery LNP or the method of E122, wherein the phospholipid is DSPC.
   E125. The target cell delivery LNP or the method of E122, wherein the phospholipid is DMPE.
   E126. The target cell delivery LNP or the method of E122 wherein the phospholipid is Compound H-409.
   E127. The target cell delivery LNP or the method of any of the preceding embodiments, which comprises a PEG-lipid.
   E128. The target cell delivery LNP or the method of E127, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
   E129. The target cell delivery LNP or the method of E127, wherein the PEG lipid is selected from the group consisting of PEG-c-DOMG, PEG-DMG, PEG-DLPE, PEG-DMPE, PEG-DPPC and PEG-DSPE lipid.
   E130. The target cell delivery LNP or the method of E127, wherein the PEG-lipid is PEG-DMG.
   E131. The target cell delivery LNP or the method of any of E127-E130, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.
   E132. The target cell delivery LNP or the method of any of E127-E130, wherein the PEG lipid comprises a compound selected from the group consisting of Compound P-428, Compound PL-16, Compound PL-17, Compound PL-18, Compound PL-19, Compound PL-1, and Compound PL-2.
   E133. The target cell delivery LNP, or method of any one of the preceding embodiments, wherein the LNP comprises a molar ratio of (i) ionizable lipid: (iii) a non-cationic helper lipid or phospholipid, of about 50:10, 49:11, 48:12, 47:13, 46:14, 45:15, 44:16, 43:17, 42:18 or 41:19.
   E134. The target cell delivery LNP, or method of any one of the preceding embodiments, wherein the LNP comprises about 41 mol % to about 50 mol % of ionizable lipid and about 10 mol % to about 19 mol % of non-cationic helper lipid or phospholipid.
   E135. The target cell delivery LNP, or method of any one of the preceding embodiments, wherein the LNP comprises about 50 mol % of ionizable lipid and about 10 mol % of non-cationic helper lipid or phospholipid.
   E136. The target cell delivery LNP, or method of any one of the preceding embodiments, wherein the molar ratio of (i) ionizable lipid: (iii) a non-cationic helper lipid or phospholipid, is about 50:10.
   E137. The target cell delivery LNP, or method of any one of the preceding embodiments which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.
   E138. The target cell delivery LNP, or method of any one of the preceding embodiments, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.
   E139. The target cell delivery LNP, or method of any one of the preceding embodiments, which comprises about 50 mol % ionizable lipid, about 10 mol % non-cationic helper lipid or phospholipid, about 38.5 mol % sterol or other structural lipid, and about 1.5 mol % PEG lipid.
   E140. The target cell delivery LNP, or method of any one of the preceding embodiments, wherein the mol % sterol or other structural lipid is 18.5% phytosterol and the total mol % structural lipid is 38.5%.
   E141. The target cell delivery LNP, or method of any one of the preceding embodiments, wherein the mol % sterol or other structural lipid is 28.5% phytosterol and the total mol % structural lipid is 38.5%.
   E142. The delivery LNP, or method of any of the preceding embodiments, wherein the lipid nanoparticle comprises Compound I-301 as the ionizable lipid, DSPC as the phospholipid, cholesterol or a cholesterol/β-sitosterol blend as the structural lipid and Compound 428 as the PEG lipid.
   E143. The target cell delivery LNP, or method of any of the preceding embodiments, wherein the ionizable lipid:phospholipid:structural lipid:PEG lipid are in a ratio chosen from: (i) 50:10:38:2; (ii) 50:20:28:2; (iii) 40:20:38:2; or (iv) 40:30:28:2.
   E144. The target cell delivery LNP, or method of E143, wherein the structural lipid is entirely cholesterol at 38% or 28%.
   E145. The target cell delivery LNP, or method of E143, wherein the structural lipid is cholesterol/β-sitosterol at a total percentage of 38% or 28%, wherein the blend comprises: (i) 20% cholesterol and 18% β-sitosterol; (ii) 10% cholesterol and 18% β-sitosterol or (iii) 10% cholesterol and 28% β-sitosterol.
   E146. The target cell delivery LNP, or method of E143-E145, wherein the LNP comprises:

i) about 50 mol % ionizable lipid, wherein the ionizable lipid is a compound selected from the group consisting of Compound I-301, Compound I-321, Compound I-182 or Compound I-49;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

E147. A pharmaceutical composition comprising the delivery lipid nanoparticle of any of the preceding embodiments and a pharmaceutically acceptable carrier.
E148. A GMP-grade pharmaceutical composition comprising the delivery lipid nanoparticle of any of the preceding embodiments and a pharmaceutically acceptable carrier.
E149. The pharmaceutical composition of either of E147 or E148, which has greater than 95%, 96%, 97%, 98%, or 99% purity, e.g., at least 1%, 2%, 3%, 4%, 5%, or more contaminants removed.
E150. The pharmaceutical composition of any of E147-E149, which is in large scale, e.g., at least 20 g, 30 g, 40 g, 50 g, 100 g, 200 g, 300 g, 400 g or more.

### BRIEF DESCRIPTION OF THE DRAWINGS

FIG. 1 is a set of graphs showing the concentration of Compound 301 containing lipid in the liver, spleen or plasma on Day 1 (left) or Day 15 (right). Rats were dosed intravenously with an NPI-Luc mRNA-encapsulated LNP at 2 mg/kg and lipid levels were assessed at the indicated time points.

FIG. 2 is a set of graphs showing the NPI-luc mRNA expression in the liver, spleen or plasma on Day 1 (left) or Day 15 (right). Rats were dosed intravenously with an NPI-Luc mRNA-encapsulated LNP at 2 mg/kg and mRNA levels were assessed at the indicated time points.

FIG. 3 is a graph showing lipid metabolism of Compound 301, Compound 18 or Compound 50 containing LNPs in the liver and spleen of mice.

FIGS. 4A-4B show expression of NPI-Luc in animals dosed with NPI-Luc mRNA-encapsulated Compound 301 LNP or dosed with NPI-Luc mRNA-encapsulated Compound 18 LNP. FIG. 4A shows NPI-luc expression in the liver over total liver cells. FIG. 4B shows NPI-luc expression in the spleen over total spleen cells.

FIG. 5 shows the results of immunohistochemistry analysis of NPI-luc protein expression in liver samples from animals dosed with NPI-Luc mRNA-encapsulated Compound 301 LNP or dosed with NPI-Luc mRNA-encapsulated Compound 18 LNP.

FIG. 6 is a graph depicting NPI-Luc protein levels in liver samples from animals dosed with NPI-Luc mRNA-encapsulated Compound 301 LNP or dosed with NPI-Luc mRNA-encapsulated Compound 18 LNP. An ELISA from Meso Scale Discovery (MSD) was used to quantitate NPI-Luc protein expression.

FIGS. 7A-7B show human EPO protein concentration in the plasma of animals dosed with human EPO mRNA-encapsulated LNPs. FIG. 7A shows human EPO protein levels in animals dosed with human EPO mRNA-encapsulated Compound 18 containing LNP. FIG. 7B shows human EPO protein levels in animals dosed with Compound 301 containing LNP.

FIGS. 8A-8C show human EPO levels in the plasma of animals dosed with various LNP formulations as indicated. FIG. 8A shows human EPO levels in the plasma at 3 hours post-dosing. FIG. 8B shows human EPO levels in the plasma at 6 hours post-dosing. FIG. 8C shows human EPO levels in the plasma at 24 hours post-dosing.

FIG. 9 shows expression of human EPO levels over time in the plasma of animals dosed with various LNP formulations as indicated.

FIGS. 10A-10B show physical properties of the indicated formulations of Compound 301 containing LNPs. FIG. 10A shows the diameter of the LNPs. FIG. 10B shows the surface polarity of the LNPs.

FIG. 11 is a diagram depicting the optimal composition ratio of ionizable lipid:DSPC:cholesterol for in vivo expression.

### DETAILED DESCRIPTION

The present disclosure provides improved lipid-based compositions, specifically delivery lipid nanoparticles (LNPs), that comprise lipids and which exhibit increased delivery of an agent(s) to a target cell, e.g., a liver cell or a splenic cell, as compared to LNPs lacking target cell delivery potentiating lipids. In various aspects, the present disclosure provides improved LNPs comprising target cell delivery potentiating lipids, such LNPs comprising an agent(s) for delivery to a target cell or population of target cells, methods for enhancing delivery of an agent (e.g., a nucleic acid molecule) to a target cell or population of target cells, methods of delivering such LNPs to subjects that would benefit from modulation of target cell activity, and methods of treating such subjects. The present disclosure is based, at least in part, on the discovery that certain lipid components of an LNP, when present in the LNP, enhance association of LNPs with target cells and delivery of an agent into the target cells, e.g., as demonstrated by expression of nucleic acid molecules by target cells. Although the LNPs of the disclosure have demonstrated enhanced delivery to target cells (e.g., liver cells or splenic cells) by measuring increased expression of an mRNA in said target cells, the same approach can be demonstrated using knock down of (i.e., decrease of) existing expression, depending on the nucleic acid molecule delivered.

In addition, one of ordinary skill in the art will recognize that having demonstrated enhanced delivery to target cells such as liver cells and/or splenic cells in this model system using mRNA, other agents may now be delivered to target cells using the subject target cell target cell delivery LNPs. Such agents are known in the art and, in one embodiment, an agent comprises or consists of a nucleic acid molecule. In particular, certain potentially therapeutic nucleic acid molecules are known and, in some cases, proteins encoded by such nucleic acid molecules or the nucleic acid molecules themselves are currently being used therapeutically. In view of the advance provided by the subject target cell (e.g., liver cell or splenic cell) enhancing LNPs, improved therapies are possible. In some aspects, the agent is a nucleic acid molecule selected from the group consisting of mRNA, RNAi, dsRNA, siRNA, mirs, antagomirs, antisense RNA, ribozyrne, CRISPR/Cas9, ssDNA and DNA.

In a particular embodiment, a target cell target cell delivery LNP enhances delivery of an agent, (e.g., a nucleic acid molecule) to target cells, such as liver cells (e.g., a hepatocyte, a hepatic stellate cell, a Kupffer cell, or a liver sinusoidal cell, or a combination thereof) or splenic cells (e.g., splenocytes), relative to an LNP lacking a target cell delivery potentiating lipid, e.g. an LNP comprising an amino lipid of Formula I-XII. In one embodiment, it has been demonstrated that expression of an mRNA encoding a protein of interest is enhanced in a target cell when the mRNA is delivered by a target cell target cell delivery LNP that includes a target cell delivery potentiating lipid, relative to an LNP lacking the target cell delivery potentiating lipid, e.g. an LNP comprising an amino lipid of Formula I-XII. Delivery of an agent associated with (e.g., encapsulated in) target cell delivery enhancing LNPs to target cells (e.g., live cells or splenic cells) has been demonstrated in vitro and in vivo.

As demonstrated herein, target cell delivery enhancing LNPs have been shown to result in at least about 2-fold increased expression of proteins in target cells (e.g., liver cells or splenic cells). Delivery to target cells has also been demonstrated in vivo. In vivo delivery of an encapsulated mRNA was demonstrated to at least about 302% liver cells following a single intravenous injection of an LNP of the disclosure. Delivery of encapsulated mRNA to greater than 20% of splenic cells has also been demonstrated. The levels of delivery demonstrated herein using LNPs comprising target cell delivery potentiating lipids make in vivo therapy possible. The disclosure provides methods for modulation of a variety of proteins, including upregulation and downreguiation of protein expression and/or activity, in a wide variety of clinical situations, including cancer, infectious diseases, vaccination and autoimmune diseases.

The LNPs of the disclosure are particularly useful to target liver cells or splenic cells. LNPs can comprise nucleic acid molecules (e.g., mRNA) encoding proteins that are intracellular or secreted proteins.

While not intending to be bound by any particular mechanism or theory, the enhanced delivery of a nucleic acid molecule to target cells (e.g., liver cells or splenic cells) by the LNPs of the disclosure is believed to be due to the presence of an effective amount of a target cell delivery potentiating lipid, e.g., a cholesterol analog or an amino lipid or combination thereof, that, when present in an LNP, may function by enhancing cellular association and/or uptake, internalization, intracellular trafficking and/or processing, and/or endosomal escape and/or may enhance recognition by and/or binding to target cells, relative to an LNP lacking the target cell delivery potentiating lipid.

Accordingly, while not intending to be bound by any particular mechanism or theory, in one embodiment, a target cell delivery potentiating lipid of the disclosure is preferentially taken up by a liver cell, a splenic cell, an ovarian cell, a lung cell, an intestinal cell, a heart cell, a skin cell, an eye cell or a brain cell, or a skeletal muscle cell compared to a reference LNP. In an embodiment, the reference LNP lacks the target cell delivery potentiating lipid and/or is not preferentially taken up by a liver cell, a splenic cell, an ovarian cell, a lung cell, an intestinal cell, a heart cell, a skin cell, an eye cell or a brain cell, or a skeletal muscle cell.

The ability to effectively deliver agents (e.g., nucleic acid molecules including mRNA) to target cells is useful for modulating protein expression and/or activity in the target cells. Moreover, cell activity and/or function can be altered in cells to which the LNP is delivered or in cells which interact with and/or are influenced by such cells (e.g., in an autocrine or paracrine fashion).

Target cell target cell delivery LNPs are useful for delivery of, e.g., nucleic acid molecules which modulate the expression of naturally occurring or engineered molecules. In one embodiment, expression of a soluble/secreted protein is modulated (e.g., a naturally occurring soluble molecule or one that has been modified or engineered to promote improved function/half-life/and/or stability). In another embodiment, expression of an intracellular protein is modulated (e.g., a naturally occurring intracellular protein or an engineered or modified intracellular protein that possesses altered function). In another embodiment, the expression of a transmembrane protein is modulated (e.g., a naturally occurring soluble molecule or one that has been modified or engineered to possess altered function.

In one embodiment the nucleic acid molecule may encode a protein that is not naturally expressed in the target cell (e.g., a heterologous protein or a modified protein). In one embodiment, the nucleic acid molecule may encode or knock down a protein that is naturally expressed in the target cell.

For example, in some aspects, LNPs of the disclosure are useful to enhance delivery and expression in target cells of an mRNA encoding a soluble/secreted protein, a transmembrane protein, or an intracellular protein. Exemplary transmembrane proteins may impart a new binding specificity to a target cell. Exemplary intracellular molecules may modulate cell signaling or cell fate.

The disclosure also provides methods for use of multiple LNPs in combination for delivery of the same (e.g., in different LNPs) or different agents, e.g., nucleic acid molecules (e.g., in the same LNP or different LNPs (e.g., one that is a target cell delivery enhancing LNP and one that is not) to deliver nucleic acid molecules to target cells or to different cell populations.

### Target Cell Delivery LNPs

Target cell target cell delivery LNPs can be characterized in that they result in increased delivery of agents to target cells (e.g., liver cells or splenic cells) as compared to a reference LNP (e.g., an LNP lacking the target cell delivery potentiating lipid). In particular, in one embodiment, target cell target cell delivery LNPs result in an increase (e.g., a 2-fold or more increase) in the percentage of LNPs associated with target cells as compared to a reference LNP (e.g., an LNP comprising an amino lipid of Formula I XII). In another embodiment, target cell target cell delivery LNPs result in an increase (e.g., a 2-fold or more increase) in the percentage of target cells expressing the agent carried by the LNP (e.g., expressing the protein encoded by the mRNA associated with/encapsulated by the LNP) as compared to a reference LNP (e.g., an LNP comprising an amino lipid of Formula I XII). In another embodiment, target cell target cell delivery LNPs result in preferentially uptake by a liver cell, a splenic cell, an ovarian cell, a lung cell, an intestinal cell, a heart cell, a skin cell, an eye cell or a brain cell, or a skeletal muscle cell compared to a reference LNP. In an embodiment the reference LNP lacks the target cell delivery potentiating lipid and/or is not preferentially taken up by a liver cell, a splenic cell, an ovarian cell, a lung cell, an intestinal cell, a heart cell, a skin cell, an eye cell or a brain cell, or a skeletal muscle cell.

In another embodiment, target cell target cell delivery LNPs result in an increase in the delivery of an agent (e.g., a nucleic acid molecule) to target cells as compared to a reference LNP (e.g., an LNP comprising an amino lipid of Formula I XII). In one embodiment, target cell target cell delivery LNPs result in an increase in the delivery of a nucleic acid molecule agent to liver cells as compared to a reference LNP. In one embodiment, target cell target cell delivery LNPs result in an increase in the delivery of a nucleic acid molecule agent to hepatocytes as compared to a reference LNP. In one embodiment, target cell target cell delivery LNPs result in an increase in the delivery of a nucleic acid molecule agent to hepatic stellate cells as compared to a reference LNP. In one embodiment, target cell target cell delivery LNPs result in an increase in the delivery of a nucleic acid molecule agent to Kupffer cells as compared to a reference LNP. In one embodiment, target cell target cell delivery LNPs result in an increase in the delivery of a nucleic acid molecule agent to liver sinusoidal cells as compared to a reference LNP.

In one embodiment, when the nucleic acid molecule is an mRNA, an increase in the delivery of a nucleic acid agent to target cells can be measured by the ability of an LNP to effect at least about 2-fold greater expression of a protein molecule encoded by the mRNA in target cells, (e.g., liver cells or splenic cells) as compared to a reference LNP.

Target cell delivery LNPs comprise an (i) ionizable lipid; (ii) sterol or other structural lipid; (iii) a non-cationic helper lipid or phospholipid; a (iv) PEG lipid and (v) an agent (e.g., a nucleic acid molecule) encapsulated in and/or associated with the LNP, wherein one or more of (i) the ionizable lipid or (ii) the structural lipid or sterol in a target cell target cell delivery LNPs comprises an effective amount of a target cell delivery potentiating lipid.

In another embodiment, a target cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to a target cell, and

(v) optionally, a PEG-lipid

wherein one or more of (i) the ionizable lipid or (ii) the sterol or other structural lipid comprises a target cell delivery potentiating lipid in an amount effective to enhance delivery of the lipid nanoparticle to a target cell. In one embodiment, enhanced delivery is relative to a lipid nanoparticle lacking the target cell delivery potentiating lipid. In another embodiment, the enhanced delivery is relative to a suitable control, e.g., reference LNP.

In another embodiment, a target cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to a target cell, and

(v) optionally, a PEG-lipid

wherein one or more of (i) the ionizable lipid or (ii) the sterol or other structural lipid or (iii) the non-cationic helper lipid or phospholipid or (v) the PEG lipid is preferentially taken up by a target cell (e.g., a liver cell or a splenic cell), as compared to a reference LNP.

In another embodiment, a target cell delivery lipid nanoparticle of the disclosure comprises:

(i) an ionizable lipid;

(ii) a sterol or other structural lipid;

(iii) a non-cationic helper lipid or phospholipid;

(iv) an agent for delivery to a target cell, and

(v) optionally, a PEG-lipid

wherein one or more of (i) the ionizable lipid or (ii) the sterol or other structural lipid is preferentially taken up by a target cell (e.g., a liver cell or a splenic cell), as compared to a reference LNP.

### Lipid Content of LNPs

As set forth above, with respect to lipids, target cell delivery LNPs comprise an (i) ionizable lipid; (ii) sterol or other structural lipid; (iii) a non-cationic helper lipid or phospholipid; a (iv) PEG lipid, wherein one or more of (i) the ionizable lipid or (ii) the structural lipid or sterol in a target cell target cell delivery LNPs comprises an effective amount of a target cell delivery potentiating lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

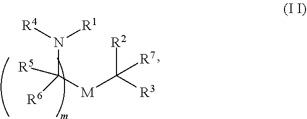

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected

from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III):

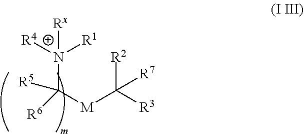

### or its N-oxide,

or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥOH, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I) includes those of Formula (IA):

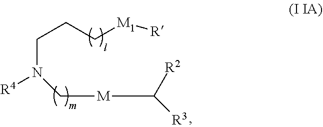

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

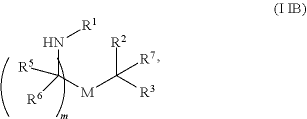

or its N-oxide, or a salt or isomer thereof in which all variables are as defined herein. For example, m is selected from 5, 6, 7, 8, and 9; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (II):

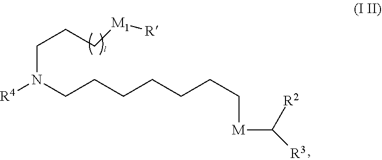

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (I VI):

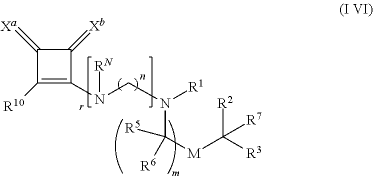

### or its N-oxide,

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

each R is independently selected from the group consisting of H, C₁₋₃ alkyl, and C₂₋₃ alkenyl;

R^{N} is H, or C₁₋₃ alkyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I;

X^{a} and X^{b} are each independently O or S;
R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;
m is selected from 5, 6, 7, 8, 9, 10, 11, 12, and 13;
n is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, 4, and 5.

In one embodiment, a subset of compounds of Formula (VI) includes those of Formula (VI-a):

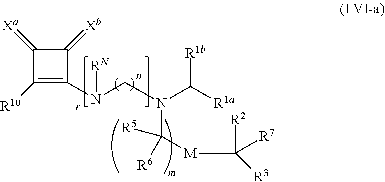

### or its N-oxide,

or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

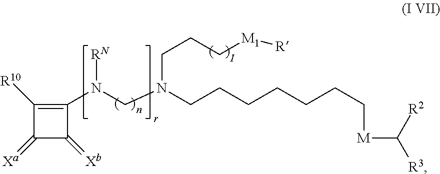

or its N-oxide, or a salt or isomer thereof, wherein

1 is selected from 1, 2, 3, 4, and 5;

M₁ is a bond or M′; and

R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

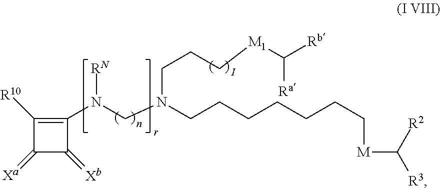

or its N-oxide, or a salt or isomer thereof, wherein

1 is selected from 1, 2, 3, 4, and 5;

M₁ is a bond or M′; and

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b}— is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

Another aspect of the disclosure relates to compounds of Formula (I XI):

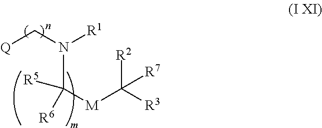

### or its N-oxide,

or a salt or isomer thereof, wherein

Q is selected from —OR, —OC(O)R, or —OC(O)N(R)₂;

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃₃ alkyl, C₂₋₃ alkenyl, and H;

each R is independently selected from the group consisting of H, C₁₋₃ alkyl, and C₂₋₃ alkenyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

m is selected from 5, 6, 7, 8, 9, 10, 11, 12, and 13; and

n is selected from 1, 2, 3, 4, 5, 6, 7, 8, 9, and 10.

In another embodiment, a subset of compounds of Formula (I XI) includes those of Formula (I XI-a):

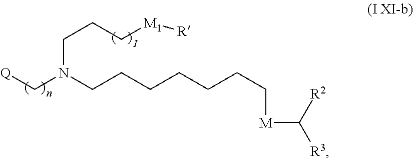

or its N-oxide, or a salt or isomer thereof, wherein

Q is —OR;

1 is selected from 1, 2, 3, 4, and 5;

M₁ is a bond or M′;

R² and R³ are independently selected from the group consisting of H, C₁₋₁₄ alkyl, and C₂₋₁₄ alkenyl; and

n is selected from 1, 2, and 3.

In another embodiment, a subset of compounds of Formula (I XI) includes those of Formula (I XI-b):

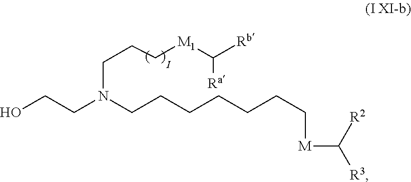

or its N-oxide, or a salt or isomer thereof, wherein:

1 is selected from 1, 2, 3, 4, and 5;

M₁ is a bond or M′;

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compound of any one of formula (I XI), (I XI-a), or (I XI-b) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, Q is —OR.

In some embodiments, n is 2.

In some embodiments, n is 3.

In some embodiments, n is 4.

In some embodiments, M₁ is absent.

In some embodiments, R is H.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen. In some embodiments, each of R⁵ and R⁶ is hydrogen.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In some embodiments, M₁ is M′. In some embodiments, M and M′ are each —C(O)O—. In some embodiments, 1 is 5. In some embodiments, Q is —OH. In some embodiments, n is 2. In some embodiments, each of R⁵ and R⁶ is hydrogen. In some embodiments, R′ is C₁₋₁₈ alkyl. In some embodiments, R′ is C₁₁ alkyl. In some embodiments, R² and R³ are independently C₃₋₁₄ alkyl. In some embodiments, R² and R³ are independently C₈ alkyl. In some embodiments, R⁷ is H. In some embodiments, R^{a′} is C₁₋₁₄ alkyl. In some embodiments, R^{a′} is C₈ alkyl. In some embodiments, R^{b′} is C₁₋₃ alkyl. In some embodiments, R^{b′} is C₂ alkyl.

In one embodiment, the compounds of Formula (I) are of Formula (IIa):

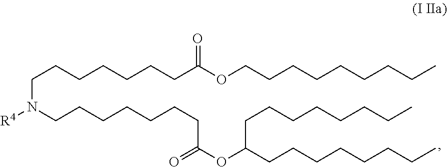

or their N-oxides, or salts or isomers thereof, wherein R⁴ is as described herein.

In another embodiment, the compounds of Formula (I) are of Formula (IIb):

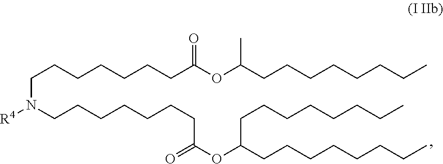

or their N-oxides, or salts or isomers thereof, wherein R⁴ is as described herein.

In another embodiment, the compounds of Formula (I) are of Formula (IIc) or (IIe):

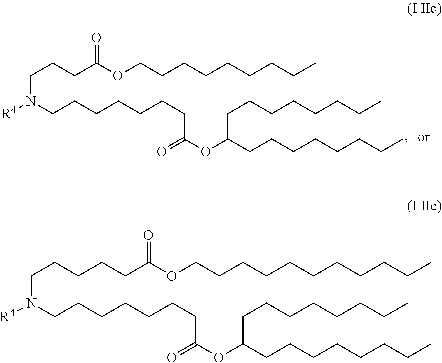

or their N-oxides, or salts or isomers thereof, wherein R⁴ is as described herein.

In another embodiment, the compounds of Formula (I) are of Formula (I IIh):

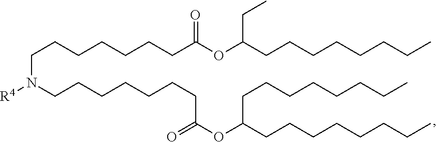

or their N-oxides, or salts or isomers thereof, wherein R⁴ is as described herein.

In another embodiment, the compounds of Formula (I) are of Formula (I IIj):

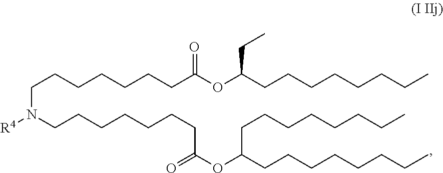

or their N-oxides, or salts or isomers thereof, wherein R⁴ is as described herein.

In another embodiment, the compounds of Formula (I) are of Formula (I IIk):

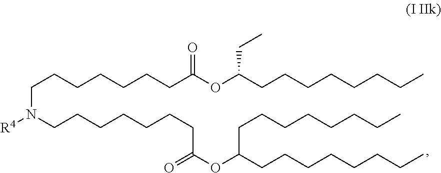

or their N-oxides, or salts or isomers thereof, wherein R⁴ is as described herein.

In another embodiment, the compounds of Formula (I I) are of Formula (I IIf):

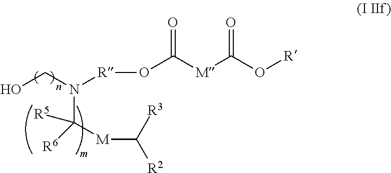

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

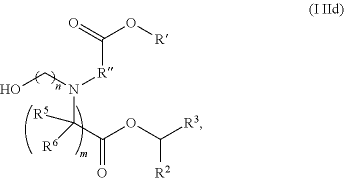

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

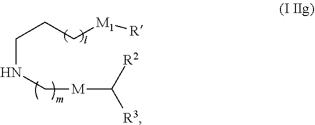

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

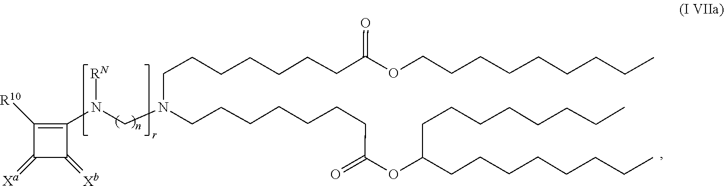

or its N-oxide, or a salt or isomer thereof.

In another embodiment, a subset of compounds of Formula (I VI) includes those of Formula (I VIIIa):

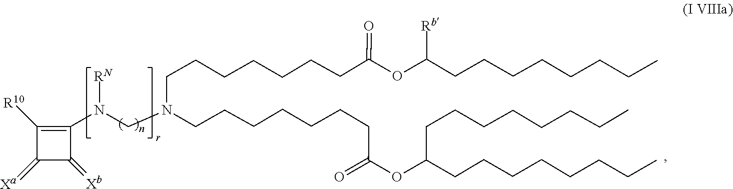

or its N-oxide, or a salt or isomer thereof.

In another embodiment, a subset of compounds of Formula (I VI) includes those of Formula (I VIIIb):

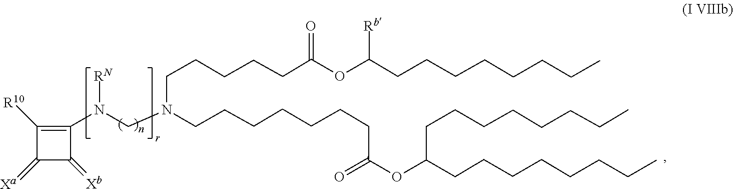

or its N-oxide, or a salt or isomer thereof.

In another embodiment, a subset of compounds of Formula (I VI) includes those of Formula (I VIIb-1):

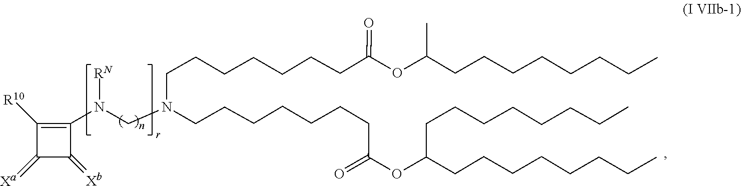

or its N-oxide, or a salt or isomer thereof.

In another embodiment, a subset of compounds of Formula (I VI) includes those of Formula (I VIIb-2):

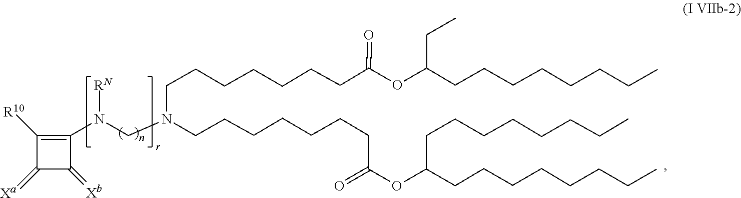

or its N-oxide, or a salt or isomer thereof.

In another embodiment, a subset of compounds of Formula (I VI) includes those of Formula (I VIIb-3):

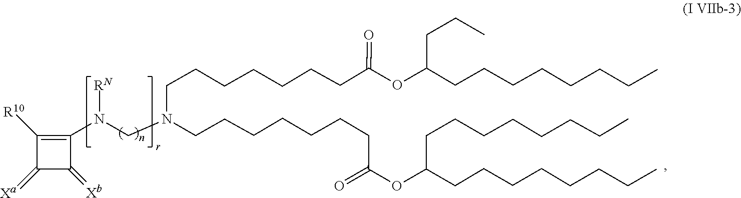

or its N-oxide, or a salt or isomer thereof. In another embodiment, a subset of compounds of Formula (VI) includes those of Formula (VIIc):

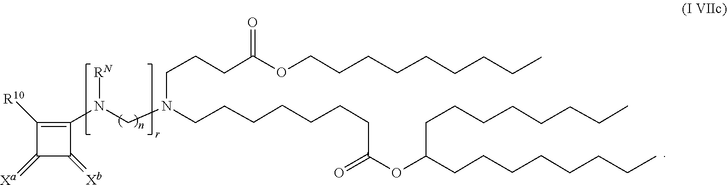

In another embodiment, a subset of compounds of Formula (I VI) includes those of Formula (VIId):

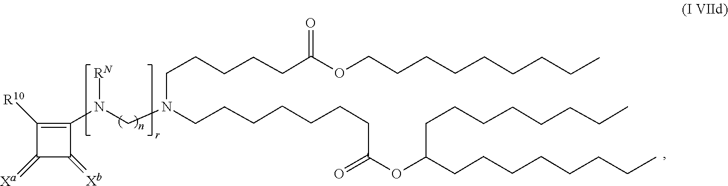

or its N-oxide, or a salt or isomer thereof.

In another embodiment, a subset of compounds of Formula (I VI) includes those of Formula (I VIIIc):

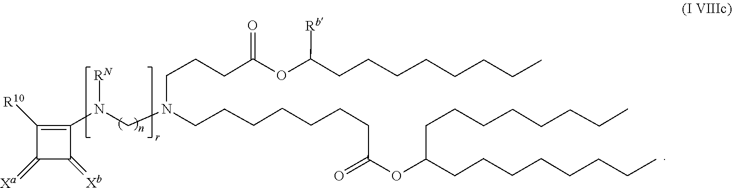

In another embodiment, a subset of compounds of Formula I VI) includes those of Formula (I VIIId):

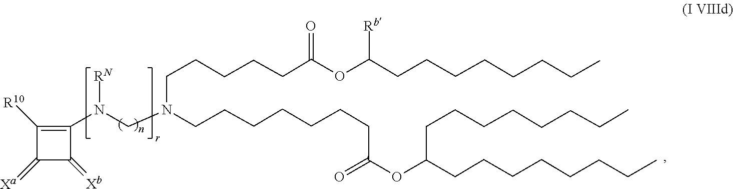

or its N-oxide, or a salt or isomer thereof.

In another embodiment, a subset of compounds of Formula (I VI) includes those of Formula (I VIIb-4):

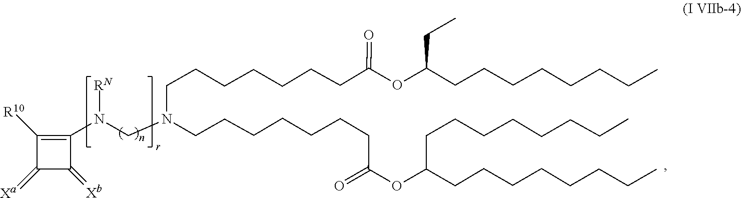

or its N-oxide, or a salt or isomer thereof.

In another embodiment, a subset of compounds of Formula (I VI) includes those of Formula (I VIIb-5):

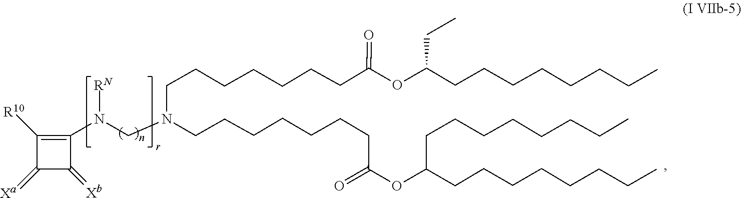

or its N-oxide, or a salt or isomer thereof.

The compounds of any one of formulae (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), (I VIIId), (I XI), (I XI-a), or (I XI-b) include one or more of the following features when applicable.

In some embodiments, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment, R⁴ is is —(CH₂)ₒC(R¹⁰)₂(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is C₁₋₃ alkyl or C₂₋₃ alkenyl. In another embodiment, each R¹⁰ is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is OH.

In another embodiment, R⁴ is —(CH₂)ₙQ, where Q is —OR, and n is selected from 1, 2, 3, 4, and 5. In a further embodiment, R⁴ is —(CH₂)ₙQ, where Q is —OR, in which R is H, and n is selected from 1, 2, and 3. For example, R⁴ is —(CH₂)₂OH.

In another embodiment, R⁴ is unsubstituted C₁₋₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆ dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

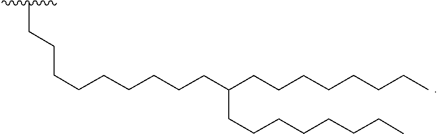 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

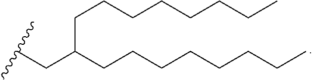 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

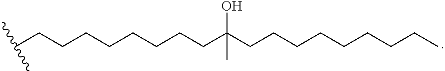 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

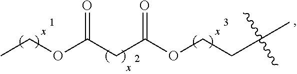

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is

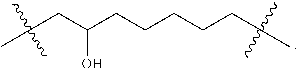 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl, each of which is either linear or branched.

In some embodiments, R′ is linear. In some embodiments R′ is branched.

In some embodiments, R′ is

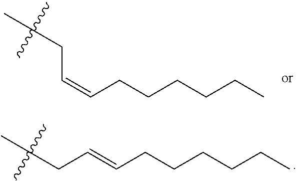

In some embodiments, R′ is

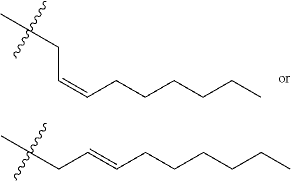

and M′ is —OC(O)—. In other embodiments, R′ is

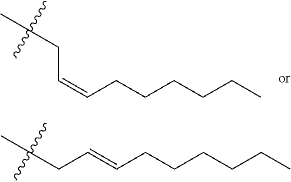

### and M′ is —C(O)O—.

In other embodiments, R′ is selected from C₁₁ alkyl and C₁₁ alkenyl. In other embodiments, R is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′

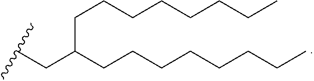 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl, or C(O)O-alkyl or OC(O)-alkyl such as C(O)OCH₃ or OC(O)CH₃). For example, R′ is

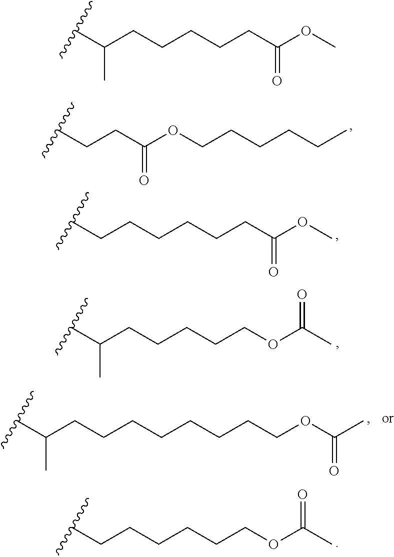

In certain embodiments, R′ is branched C₁₋₁₈ alkyl. For example, R′ is

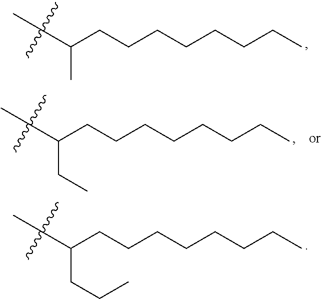

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, C₁₄ alkyl, or C₁₅ alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

In certain embodiments, Q is thiourea or an isostere thereof, e.g.,

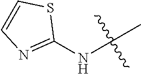 [SMILES: *Nc1nccs1]

or —NHC(═NR⁹)N(R)₂.

In certain embodiments, Q is —C(═NR⁹)N(R)₂. For example, when Q is —C(═NR⁹)N(R)₂, n is 4 or 5. For example, R⁹ is —S(O)₂N(R)₂.

In certain embodiments, Q is —C(═NR⁹)R or —C(O)N(R)OR, e.g., —CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyl)amino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O(CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

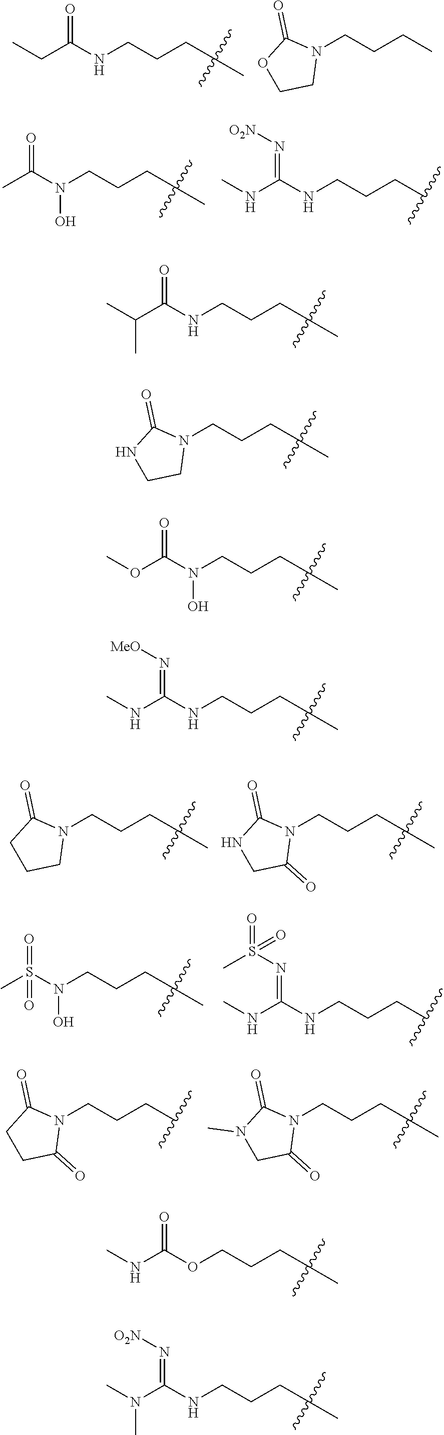

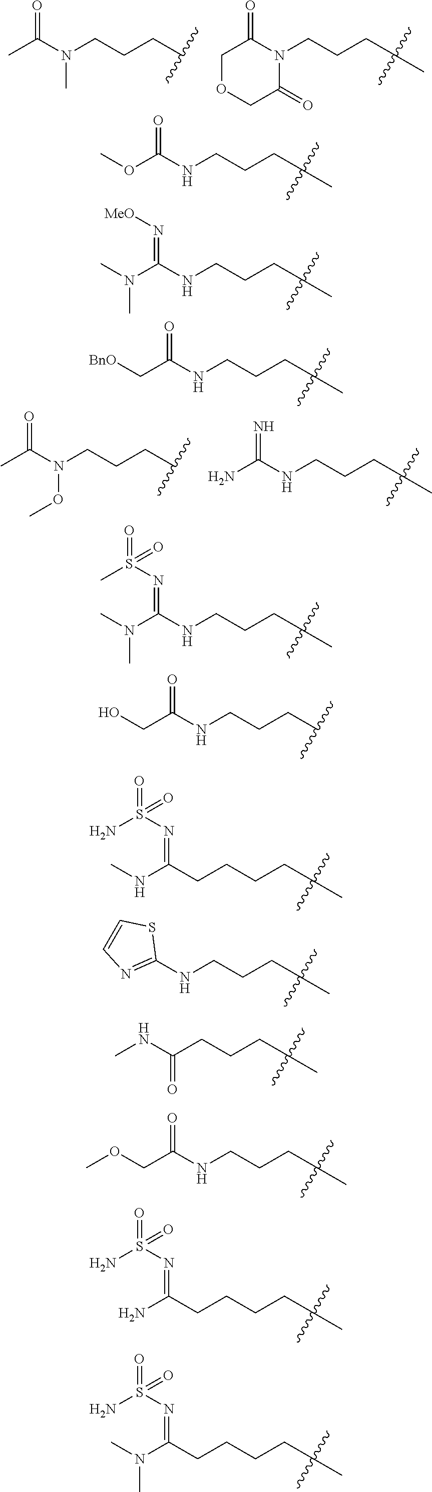

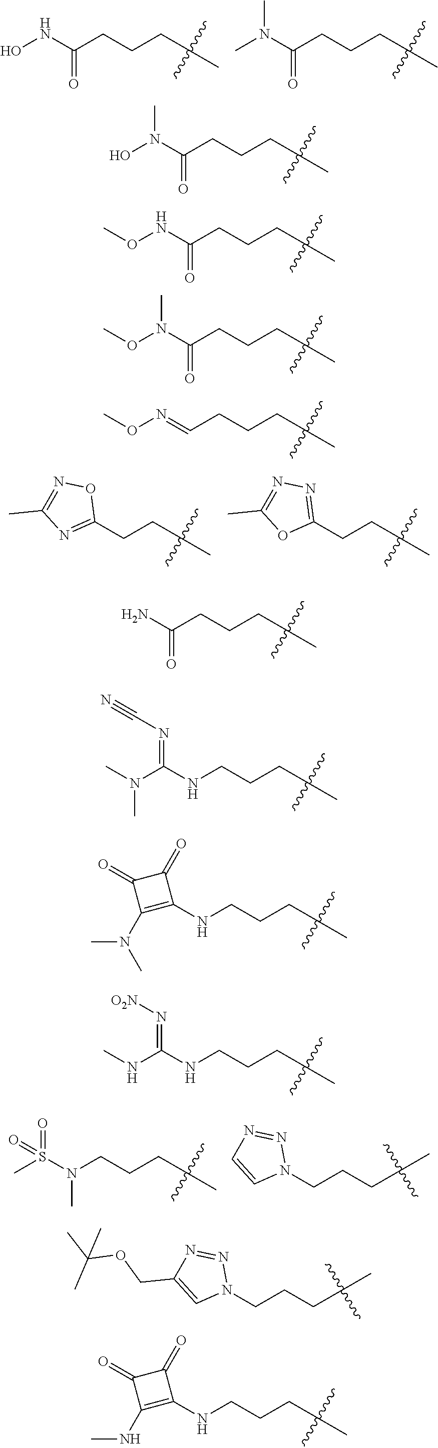

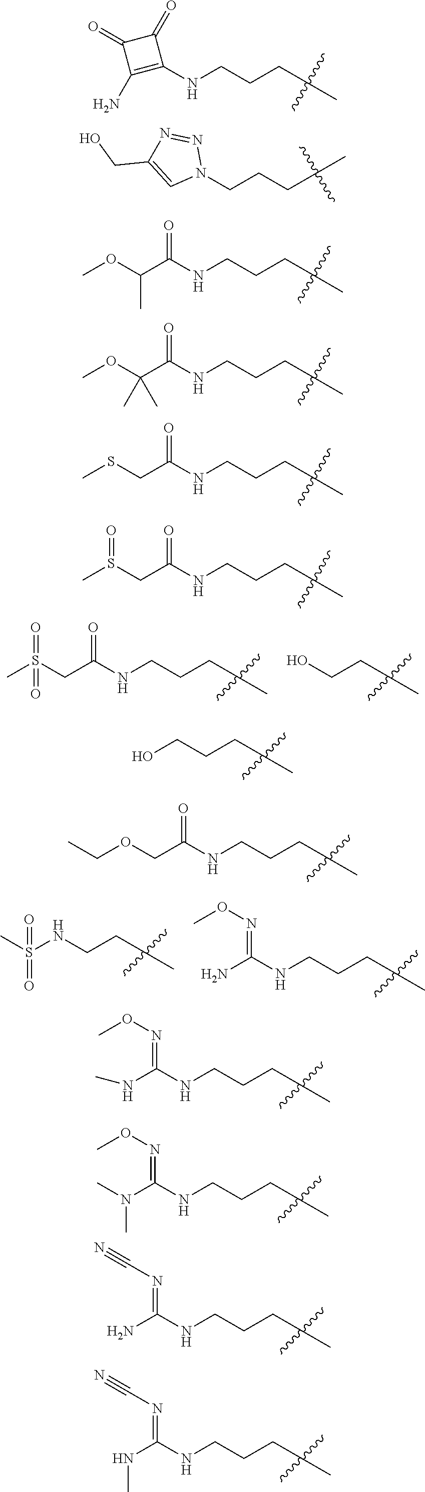

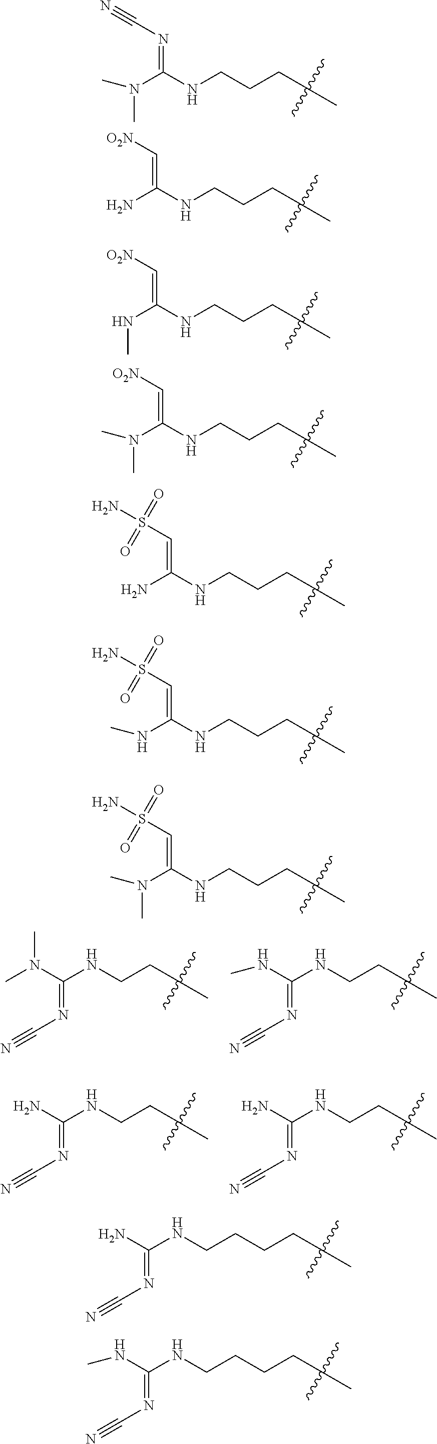

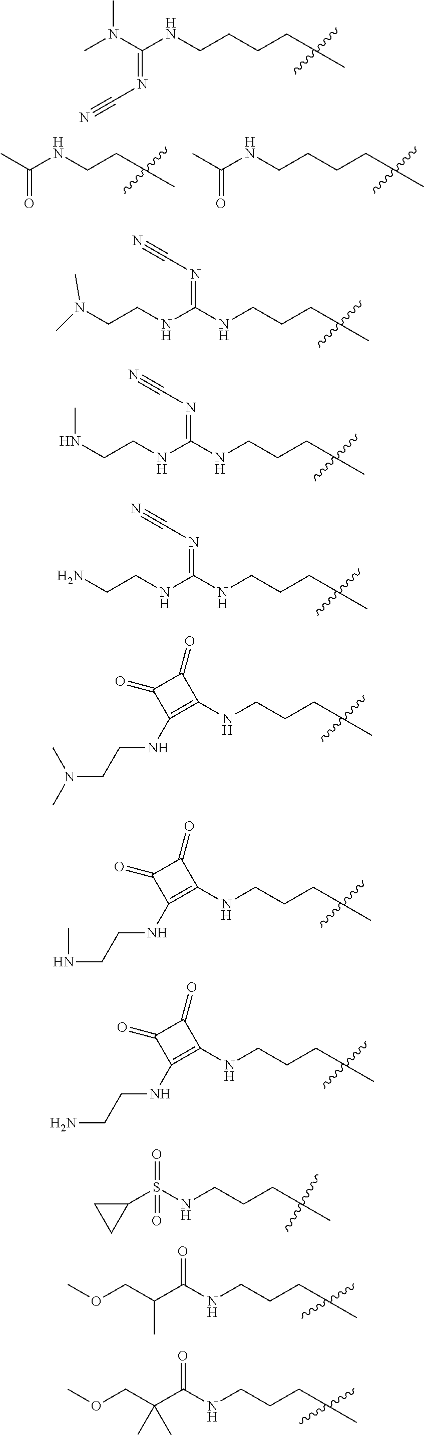

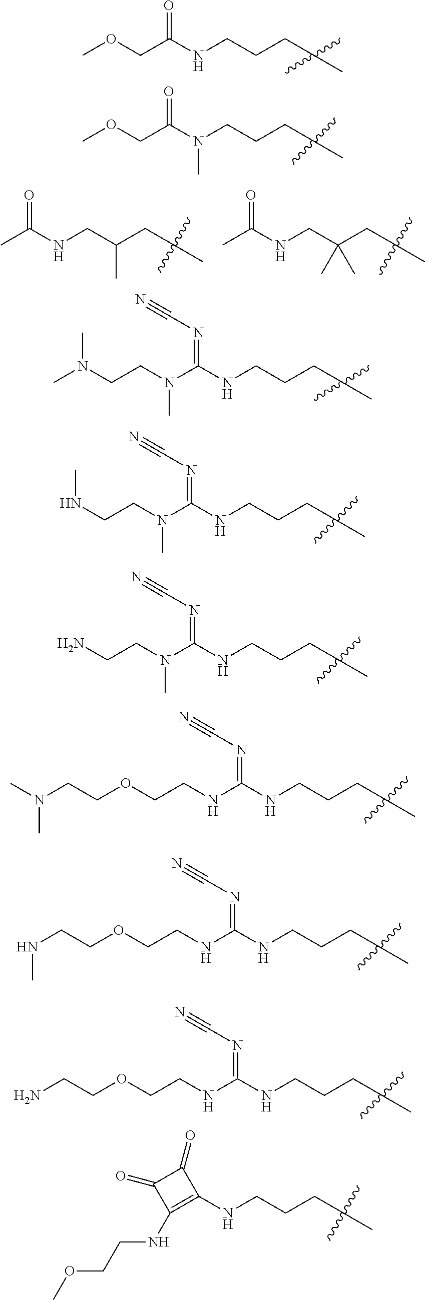

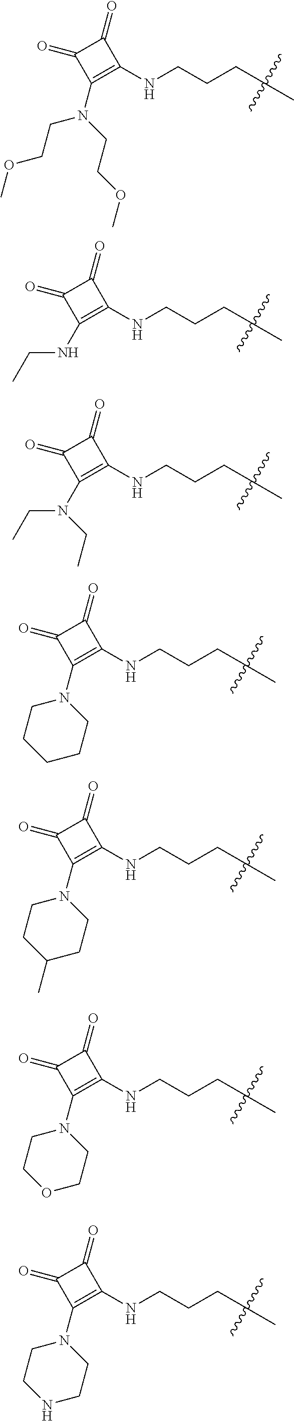

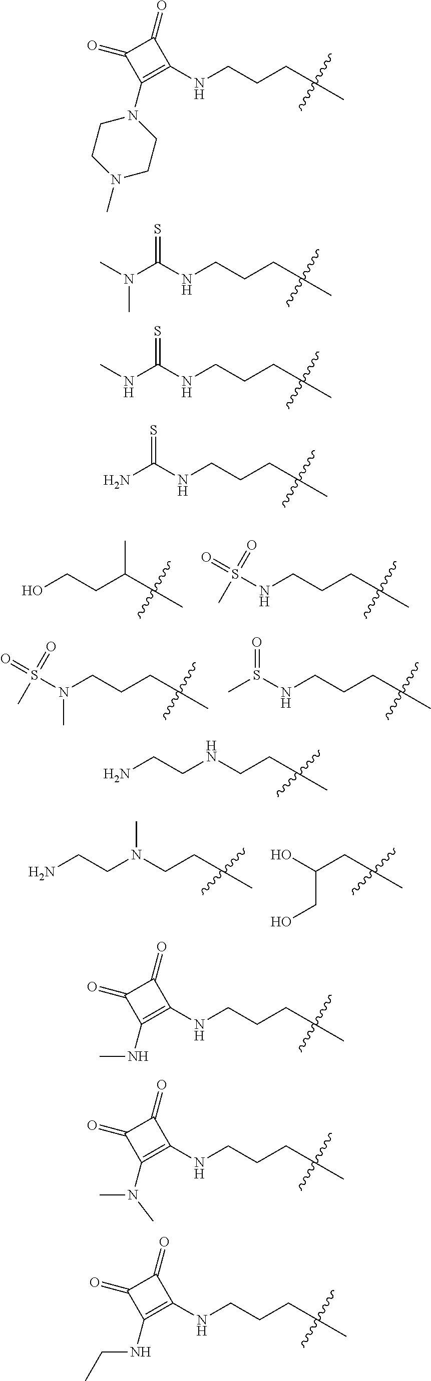

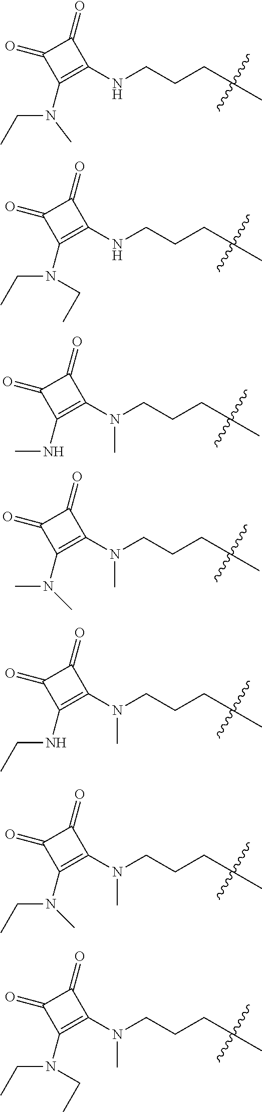

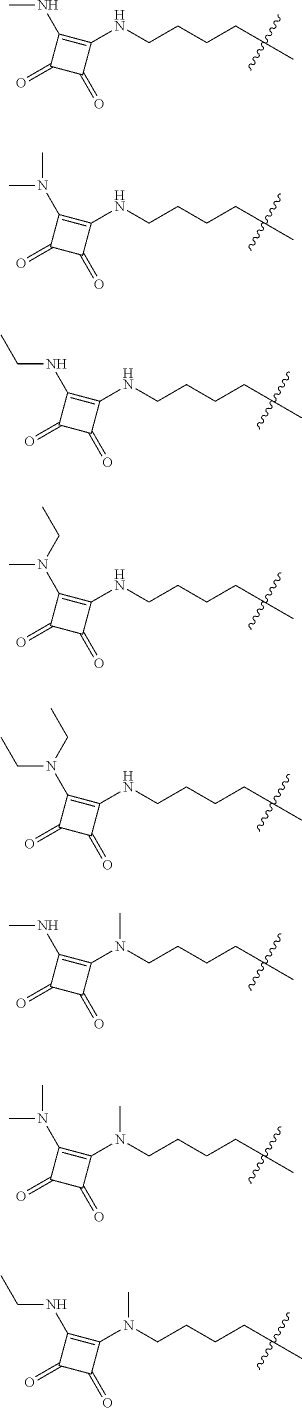

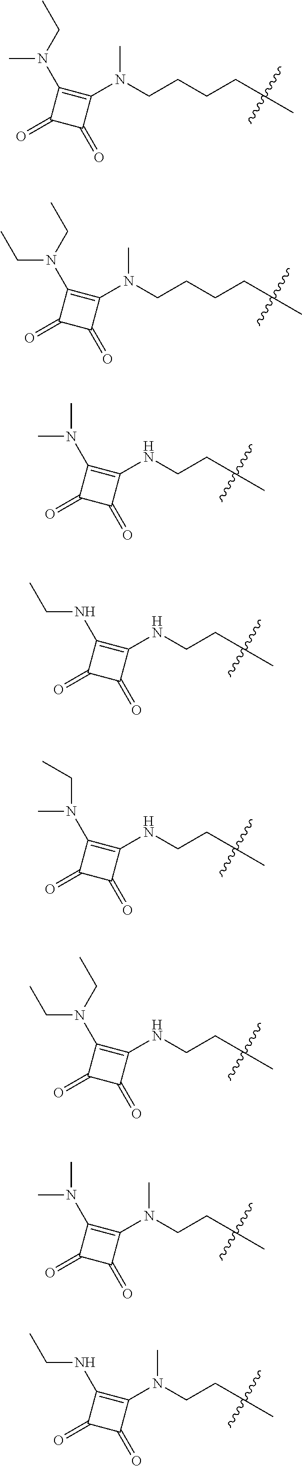

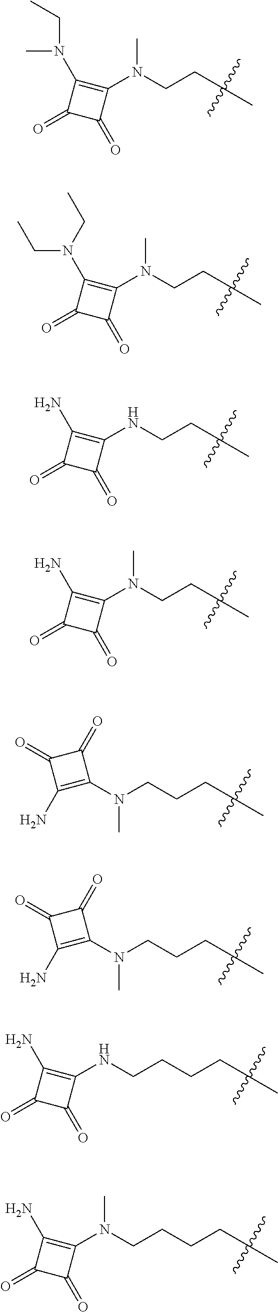

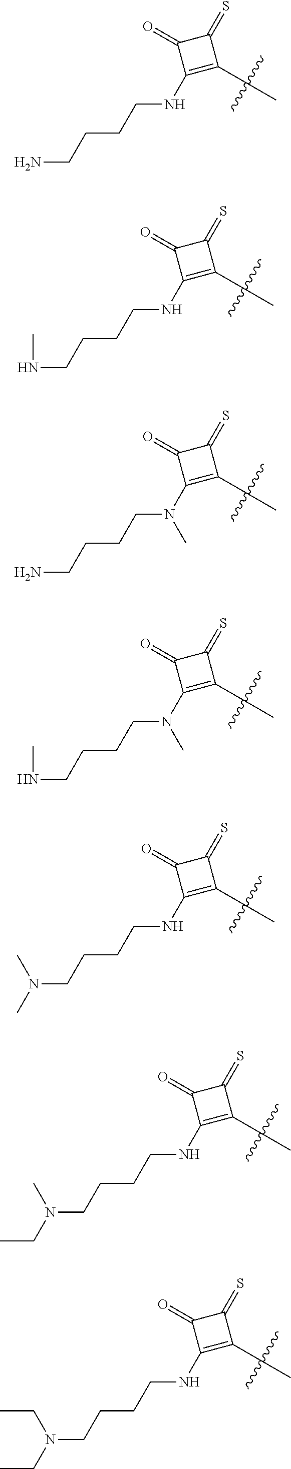

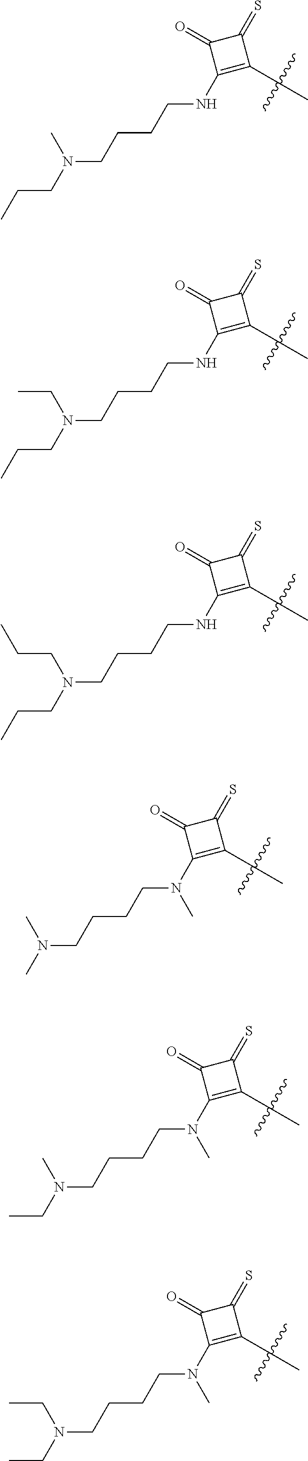 [SMILES: *c1c(NCCCCN(C)CCC)c(=O)c1=S ; *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S]

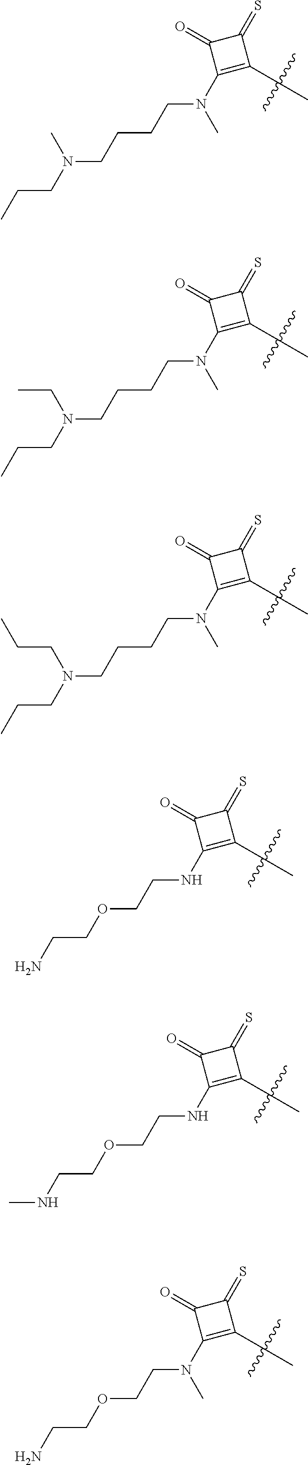 [SMILES: *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S]

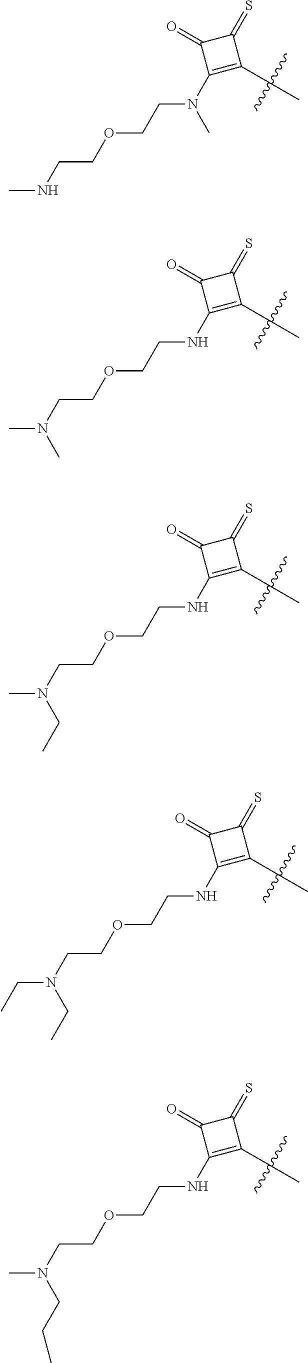 [SMILES: *c1c(N(C)CCOCCNC)c(=O)c1=S ; *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S]

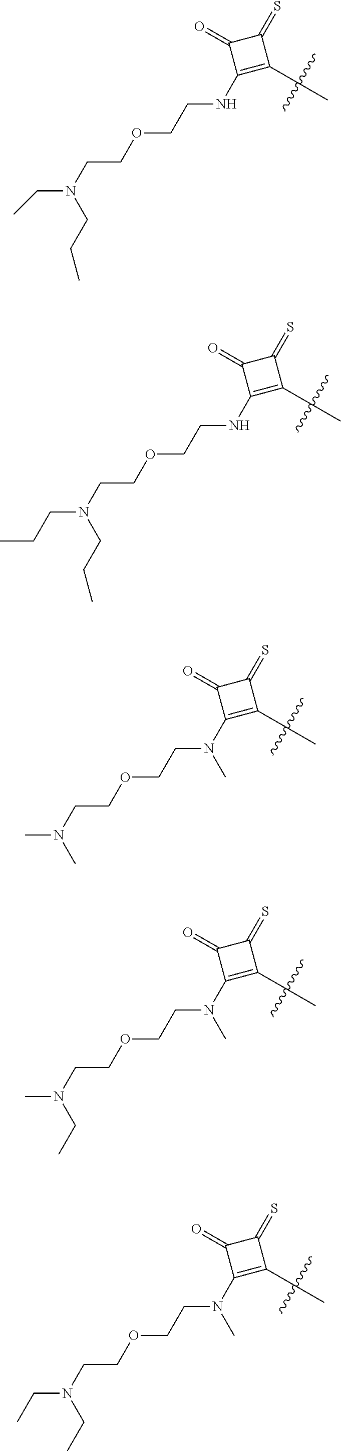 [SMILES: *c1c(NCCOCCN(CC)CCC)c(=O)c1=S ; *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S]

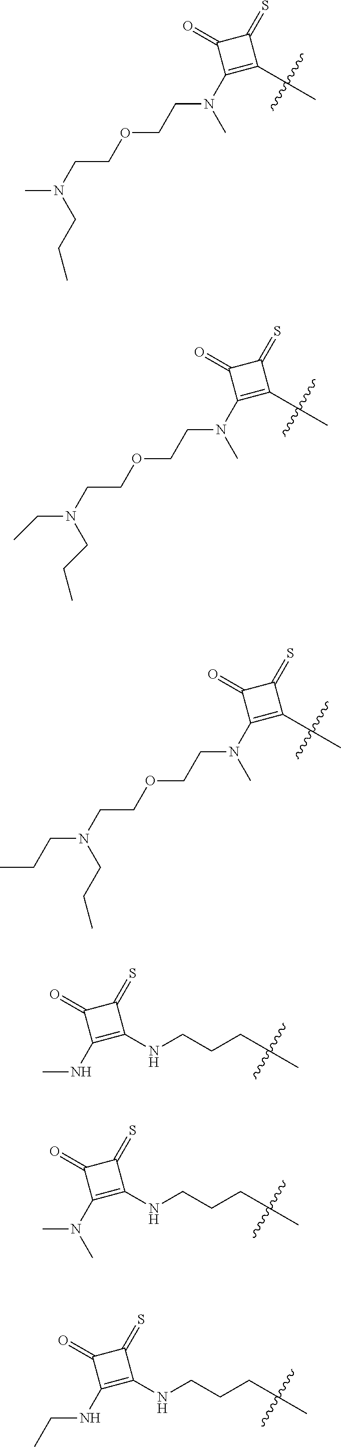 [SMILES: *c1c(N(C)CCOCCN(C)CCC)c(=O)c1=S ; *c1c(N(C)CCOCCN(CC)CCC)c(=O)c1=S ; *c1c(N(C)CCOCCN(CCC)CCC)c(=O)c1=S ; *CCCNc1c(NC)c(=O)c1=S ; *CCCNc1c(N(C)C)c(=O)c1=S ; *CCCNc1c(NCC)c(=O)c1=S]

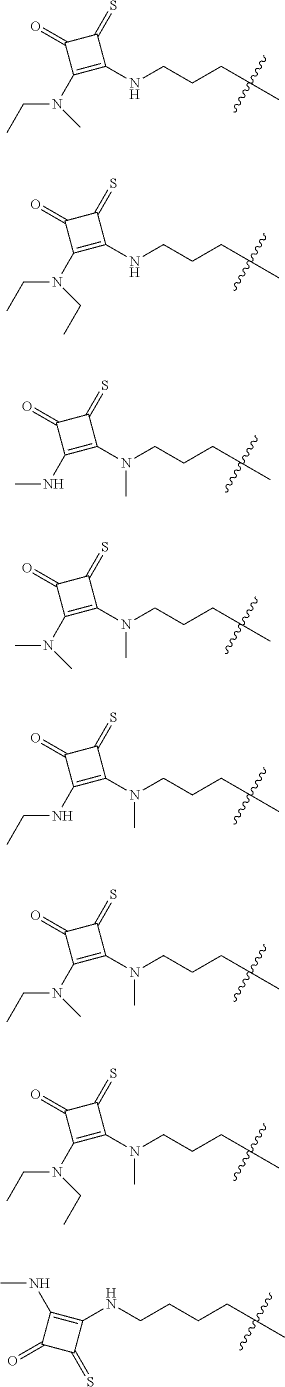

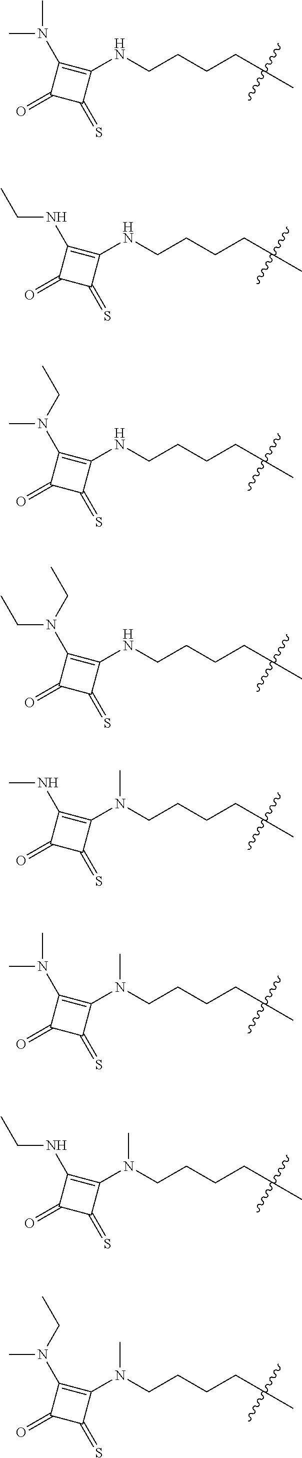

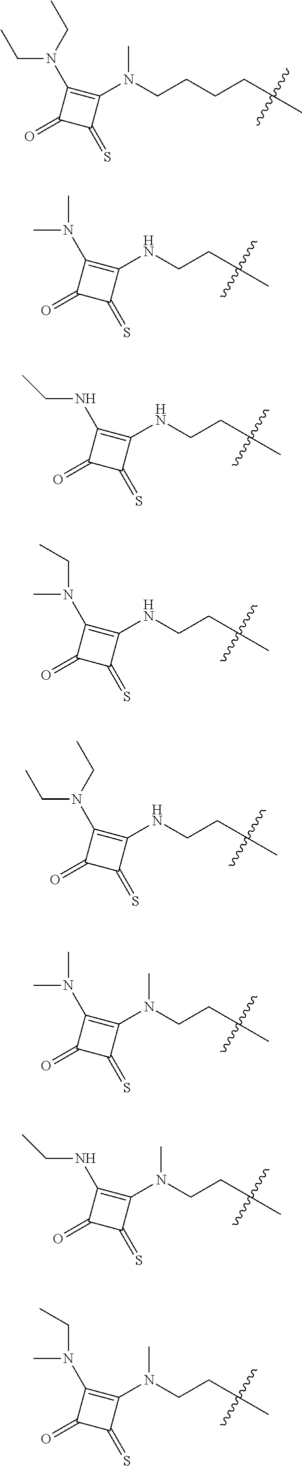

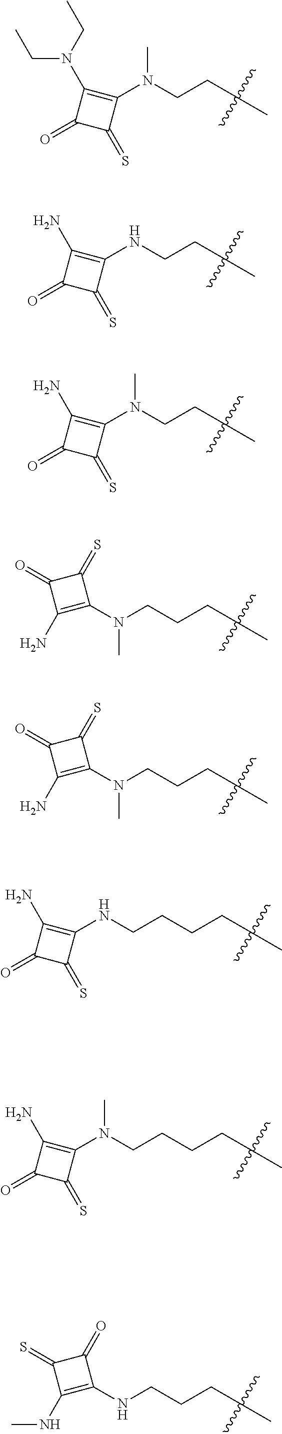

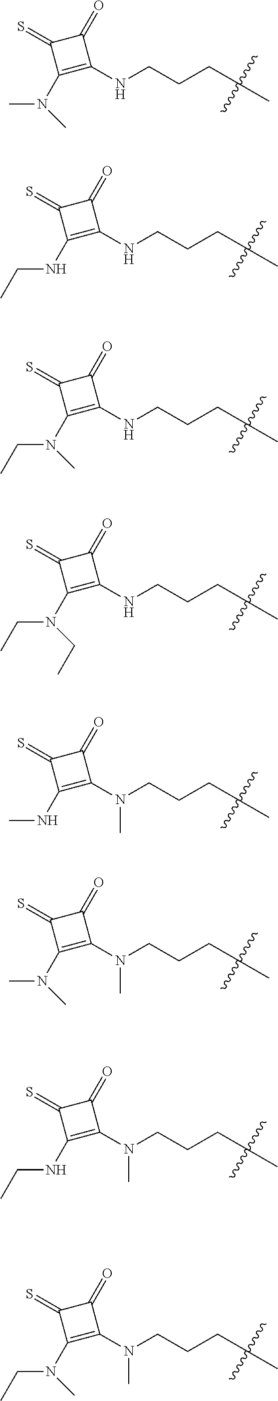

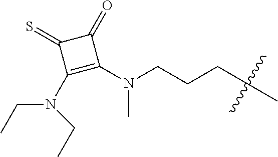 [SMILES: *CCCN(C)c1c(N(CC)CC)c(=S)c1=O]

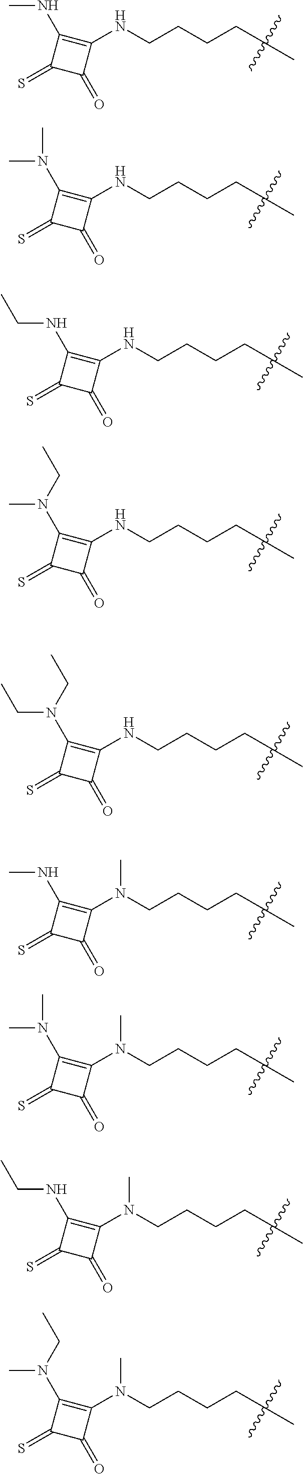

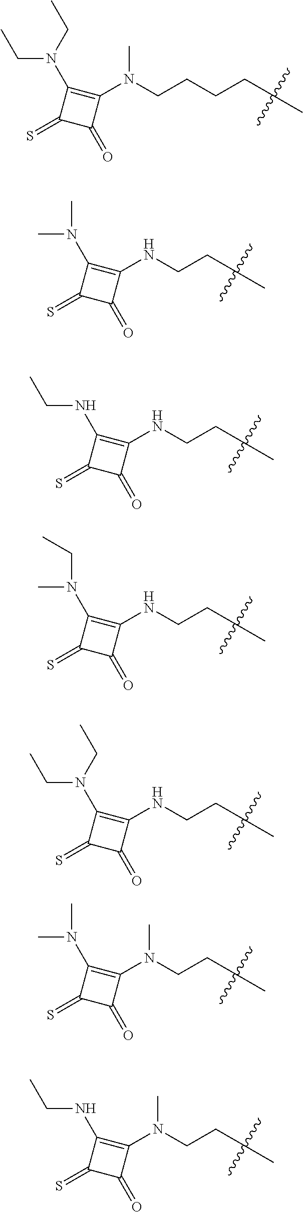

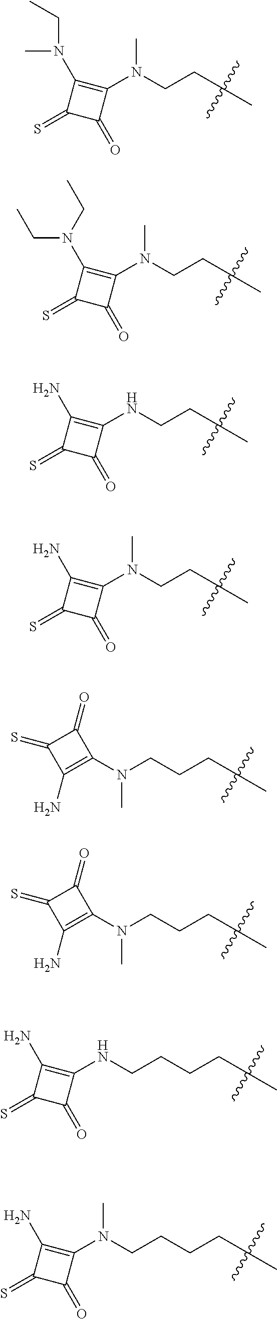

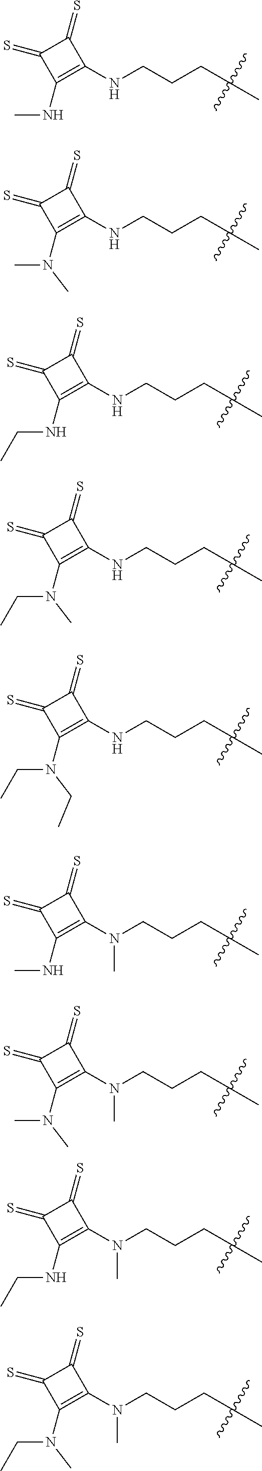

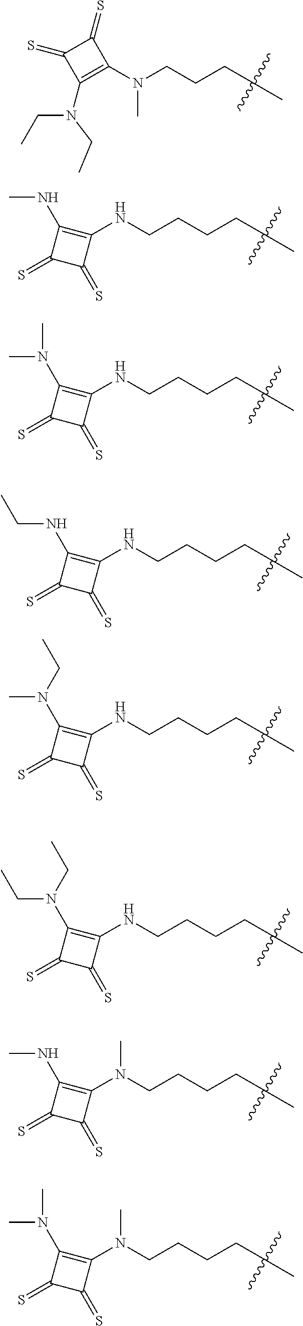

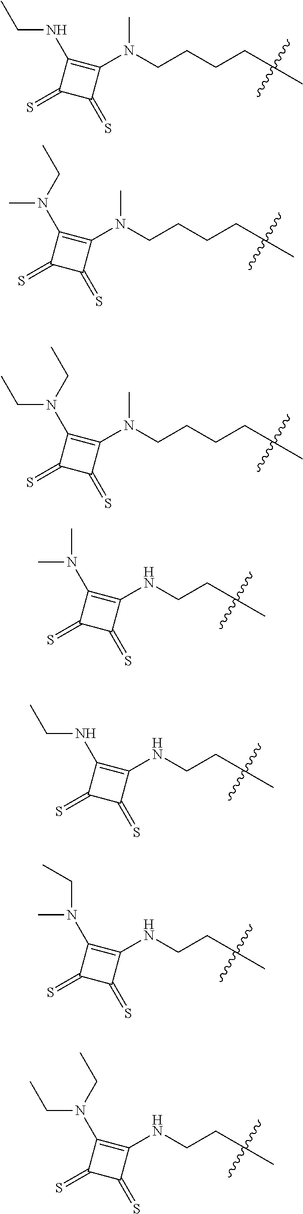

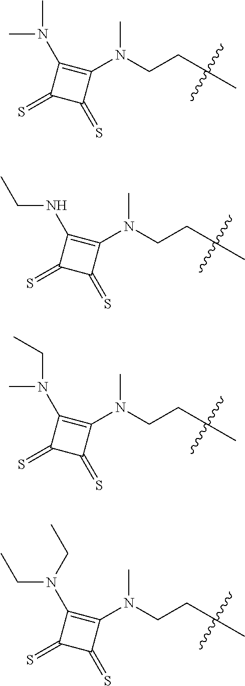 [SMILES: *CCN(C)c1c(N(C)C)c(=S)c1=S ; *CCN(C)c1c(NCC)c(=S)c1=S ; *CCN(C)c1c(N(C)CC)c(=S)c1=S ; *CCN(C)c1c(N(CC)CC)c(=S)c1=S]

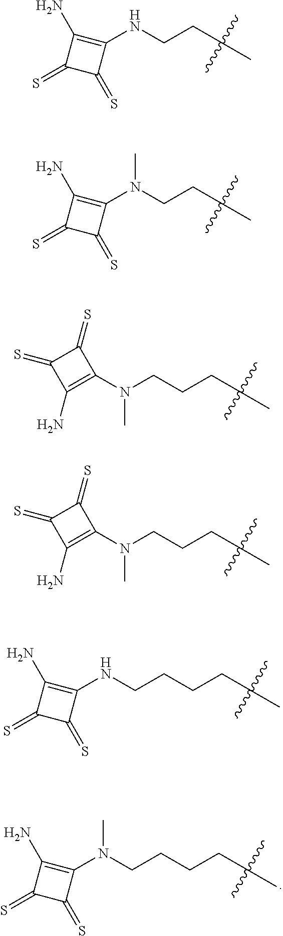 [SMILES: *CCNc1c(N)c(=S)c1=S ; *CCN(C)c1c(N)c(=S)c1=S ; *CCCN(C)c1c(N)c(=S)c1=S ; *CCCN(C)c1c(N)c(=S)c1=S ; *CCCCNc1c(N)c(=S)c1=S ; *CCCCN(C)c1c(N)c(=S)c1=S]

In some embodiments,

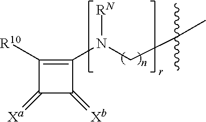

is selected from any of the following groups:

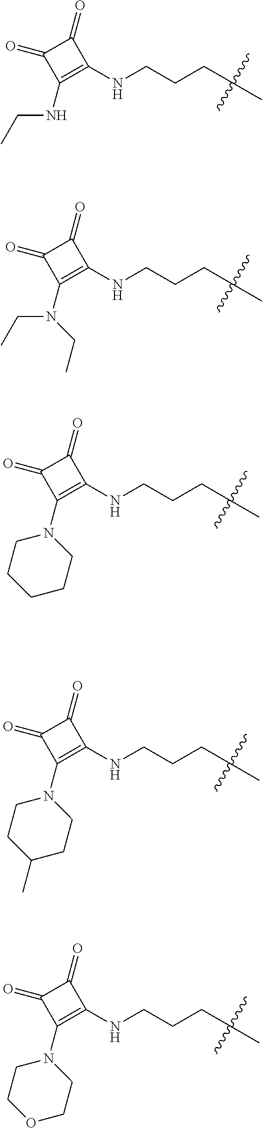 [SMILES: *CCCNc1c(NCC)c(=O)c1=O ; *CCCNc1c(N(CC)CC)c(=O)c1=O ; *CCCNc1c(N2CCCCC2)c(=O)c1=O ; *CCCNc1c(N2CCC(C)CC2)c(=O)c1=O ; *CCCNc1c(N2CCOCC2)c(=O)c1=O]

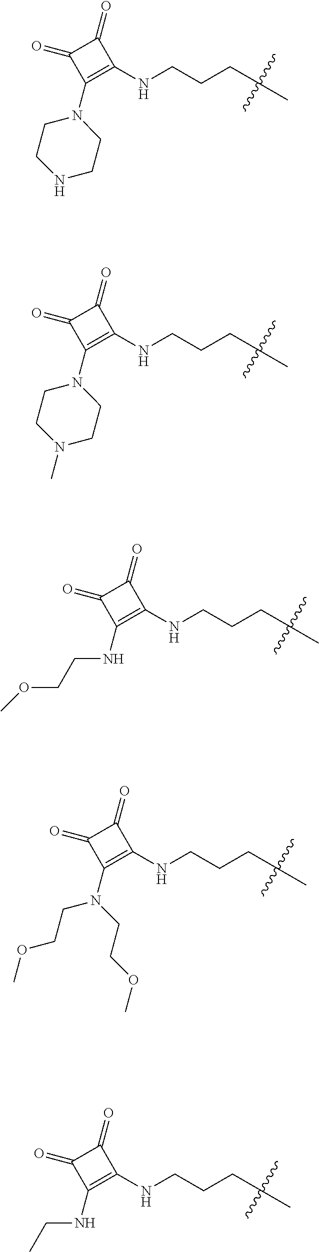 [SMILES: *CCCNc1c(N2CCNCC2)c(=O)c1=O ; *CCCNc1c(N2CCN(C)CC2)c(=O)c1=O ; *CCCNc1c(NCCOC)c(=O)c1=O ; *CCCNc1c(N(CCOC)CCOC)c(=O)c1=O ; *CCCNc1c(NCC)c(=O)c1=O]

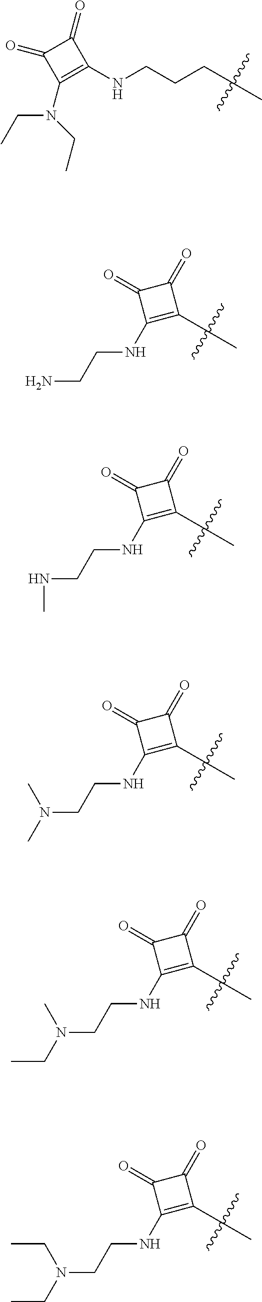 [SMILES: *CCCNc1c(N(CC)CC)c(=O)c1=O ; *c1c(NCCN)c(=O)c1=O ; *c1c(NCCNC)c(=O)c1=O ; *c1c(NCCN(C)C)c(=O)c1=O ; *c1c(NCCN(C)CC)c(=O)c1=O ; *c1c(NCCN(CC)CC)c(=O)c1=O]

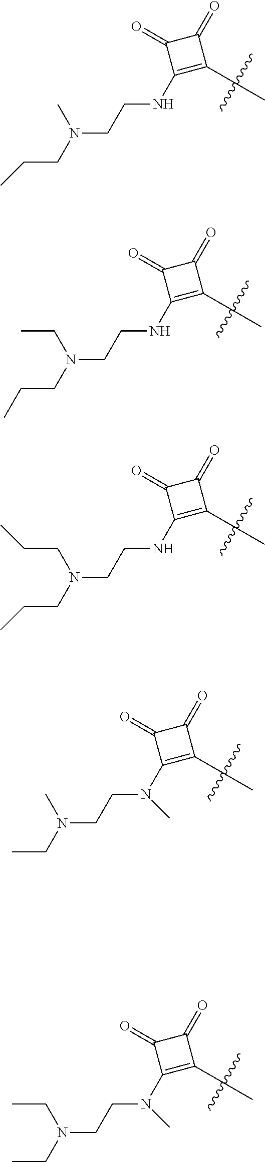 [SMILES: *c1c(NCCN(C)CCC)c(=O)c1=O ; *c1c(NCCN(CC)CCC)c(=O)c1=O ; *c1c(NCCN(CCC)CCC)c(=O)c1=O ; *c1c(N(C)CCN(C)CC)c(=O)c1=O ; *c1c(N(C)CCN(CC)CC)c(=O)c1=O]

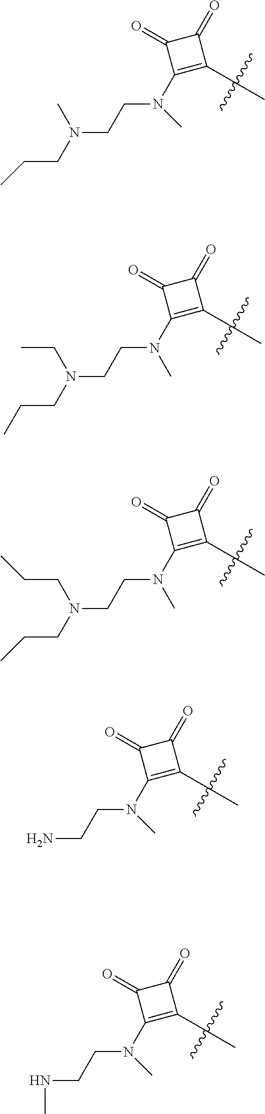 [SMILES: *c1c(N(C)CCN(C)CCC)c(=O)c1=O ; *c1c(N(C)CCN(CC)CCC)c(=O)c1=O ; *c1c(N(C)CCN(CCC)CCC)c(=O)c1=O ; *c1c(N(C)CCN)c(=O)c1=O ; *c1c(N(C)CCNC)c(=O)c1=O]

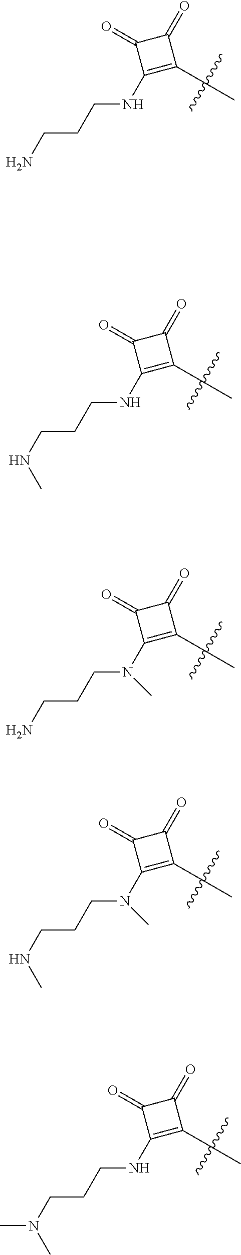 [SMILES: *c1c(NCCCN)c(=O)c1=O ; *c1c(NCCCNC)c(=O)c1=O ; *c1c(N(C)CCCN)c(=O)c1=O ; *c1c(N(C)CCCNC)c(=O)c1=O ; *c1c(NCCCN(C)C)c(=O)c1=O]

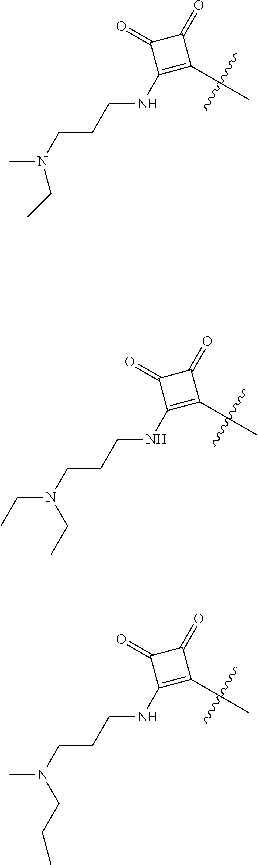 [SMILES: *c1c(NCCCN(C)CC)c(=O)c1=O ; *c1c(NCCCN(CC)CC)c(=O)c1=O ; *c1c(NCCCN(C)CCC)c(=O)c1=O]

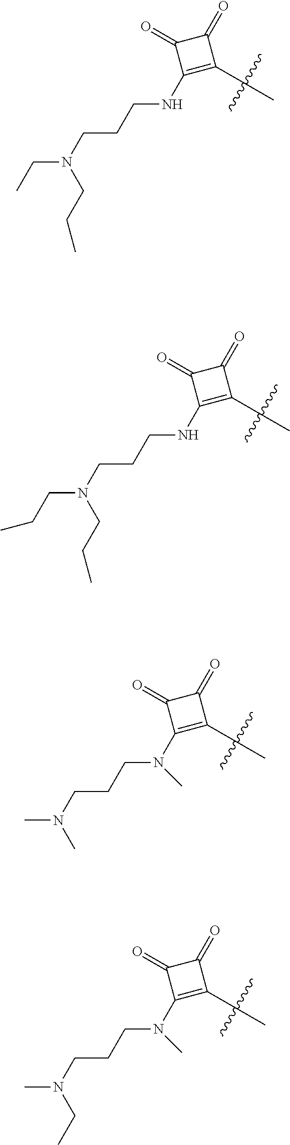 [SMILES: *c1c(NCCCN(CC)CCC)c(=O)c1=O ; *c1c(NCCCN(CCC)CCC)c(=O)c1=O ; *c1c(N(C)CCCN(C)C)c(=O)c1=O ; *c1c(N(C)CCCN(C)CC)c(=O)c1=O]

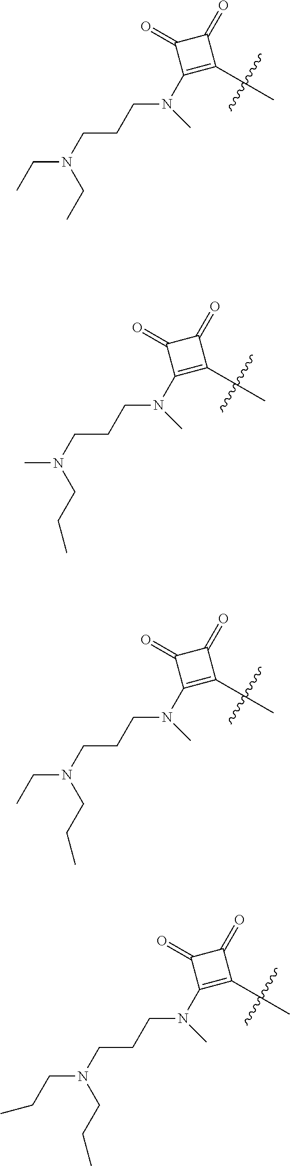 [SMILES: *c1c(N(C)CCCN(CC)CC)c(=O)c1=O ; *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O]

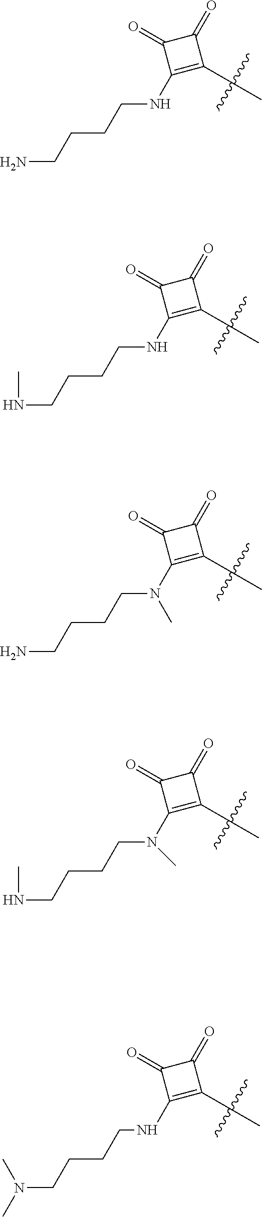 [SMILES: *c1c(NCCCCN)c(=O)c1=O ; *c1c(NCCCCNC)c(=O)c1=O ; *c1c(N(C)CCCCN)c(=O)c1=O ; *c1c(N(C)CCCCNC)c(=O)c1=O ; *c1c(NCCCCN(C)C)c(=O)c1=O]

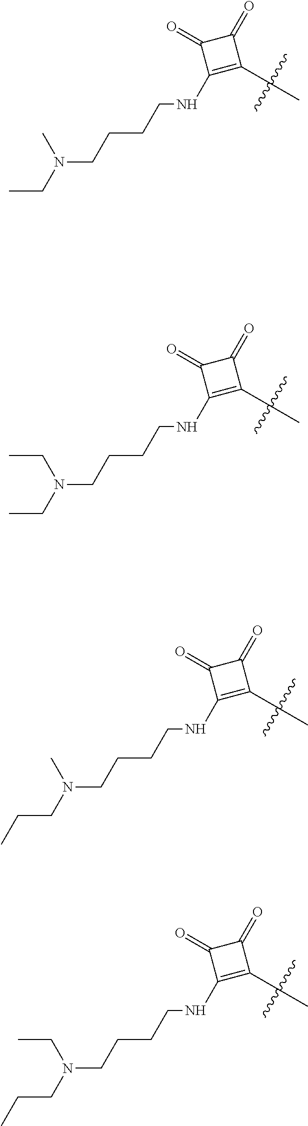 [SMILES: *c1c(NCCCCN(C)CC)c(=O)c1=O ; *c1c(NCCCCN(CC)CC)c(=O)c1=O ; *c1c(NCCCCN(C)CCC)c(=O)c1=O ; *c1c(NCCCCN(CC)CCC)c(=O)c1=O]

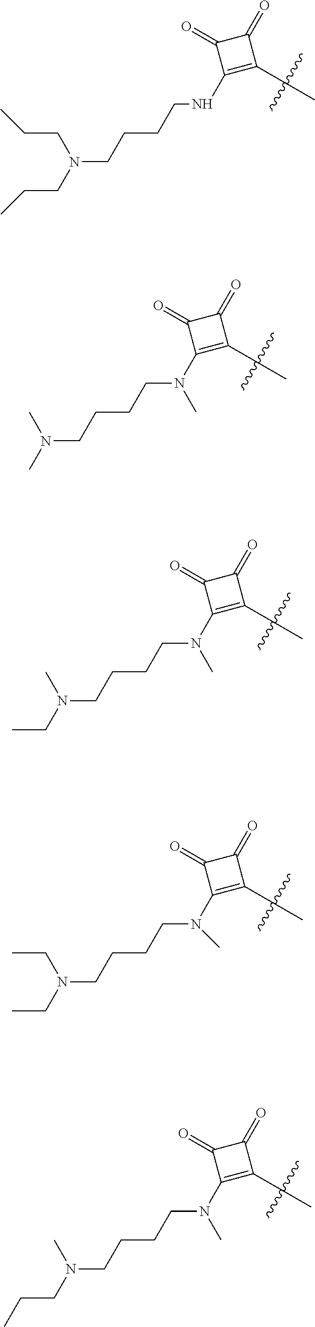 [SMILES: *c1c(NCCCCN(CCC)CCC)c(=O)c1=O ; *c1c(N(C)CCCCN(C)C)c(=O)c1=O ; *c1c(N(C)CCCCN(C)CC)c(=O)c1=O ; *c1c(N(C)CCCCN(CC)CC)c(=O)c1=O ; *c1c(N(C)CCCCN(C)CCC)c(=O)c1=O]

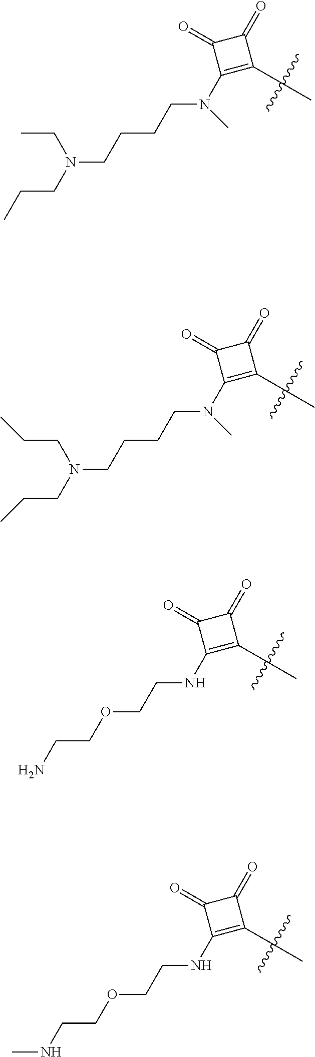 [SMILES: *c1c(N(C)CCCCN(CC)CCC)c(=O)c1=O ; *c1c(N(C)CCCCN(CCC)CCC)c(=O)c1=O ; *c1c(NCCOCCN)c(=O)c1=O ; *c1c(NCCOCCNC)c(=O)c1=O]

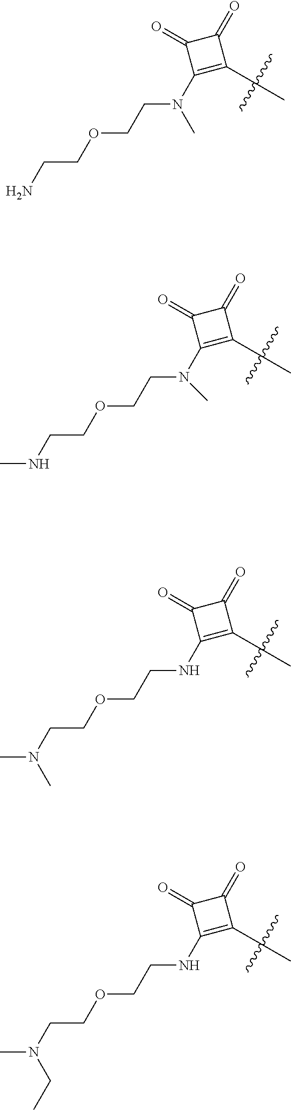 [SMILES: *c1c(N(C)CCOCCN)c(=O)c1=O ; *c1c(N(C)CCOCCNC)c(=O)c1=O ; *c1c(NCCOCCN(C)C)c(=O)c1=O ; *c1c(NCCOCCN(C)CC)c(=O)c1=O]

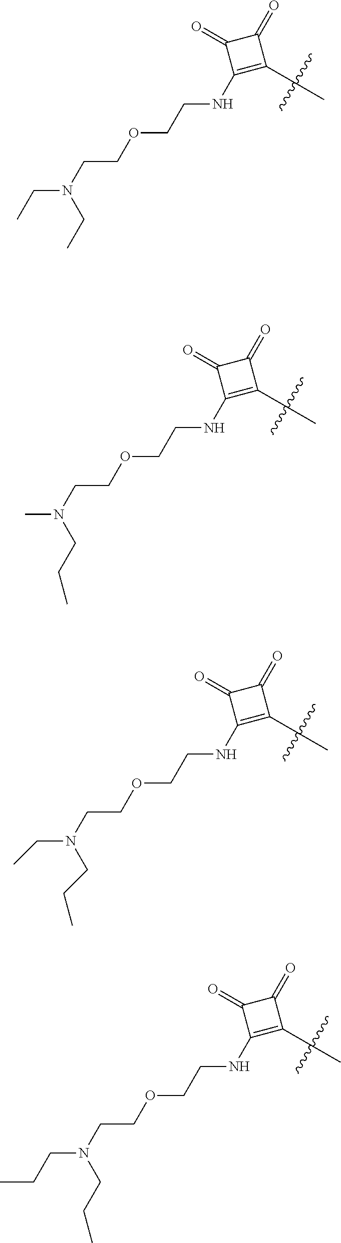 [SMILES: *c1c(NCCOCCN(CC)CC)c(=O)c1=O ; *c1c(NCCOCCN(C)CCC)c(=O)c1=O ; *c1c(NCCOCCN(CC)CCC)c(=O)c1=O ; *c1c(NCCOCCN(CCC)CCC)c(=O)c1=O]

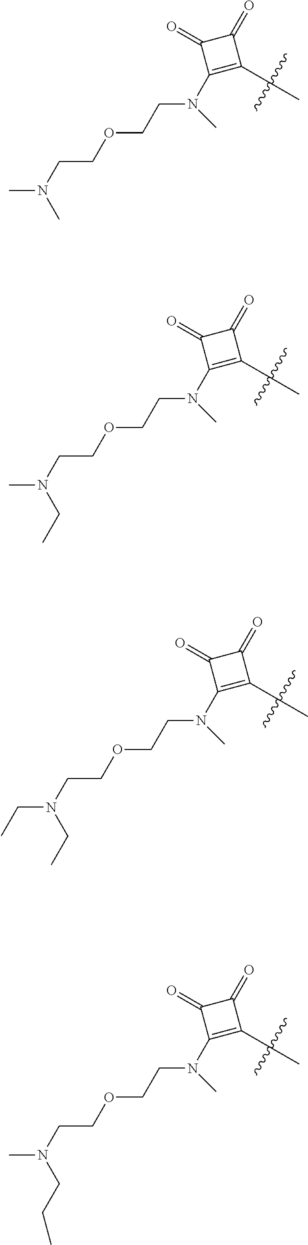 [SMILES: *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O ; *c1c(N(C)CCOCCN(C)CCC)c(=O)c1=O]

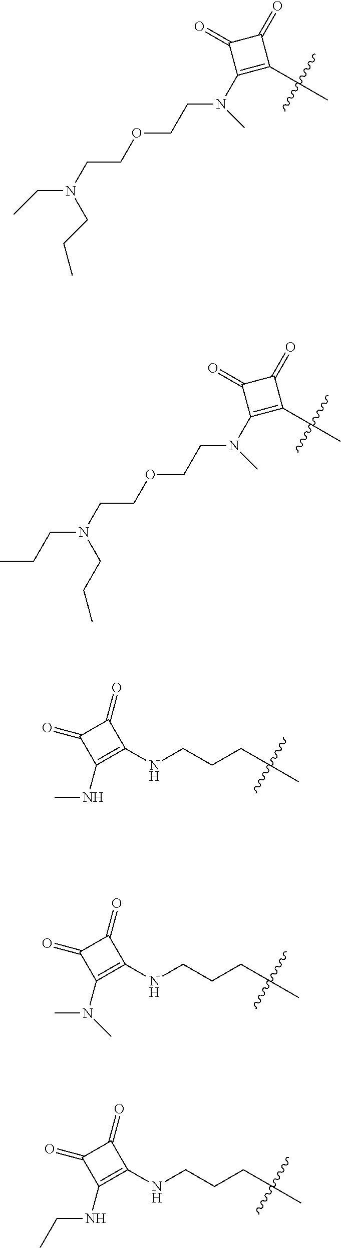 [SMILES: *c1c(N(C)CCOCCN(CC)CCC)c(=O)c1=O ; *c1c(N(C)CCOCCN(CCC)CCC)c(=O)c1=O ; *CCCNc1c(NC)c(=O)c1=O ; *CCCNc1c(N(C)C)c(=O)c1=O ; *CCCNc1c(NCC)c(=O)c1=O]

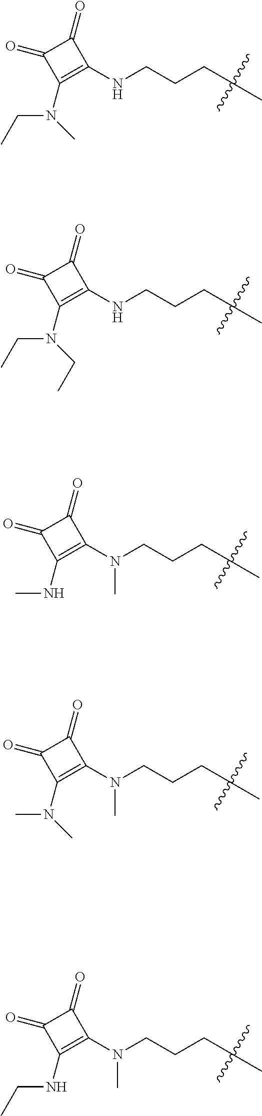 [SMILES: *CCCNc1c(N(C)CC)c(=O)c1=O ; *CCCNc1c(N(CC)CC)c(=O)c1=O ; *CCCN(C)c1c(NC)c(=O)c1=O ; *CCCN(C)c1c(N(C)C)c(=O)c1=O ; *CCCN(C)c1c(NCC)c(=O)c1=O]

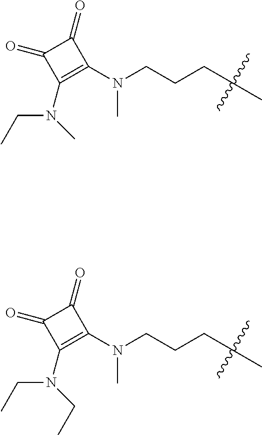 [SMILES: *CCCN(C)c1c(N(C)CC)c(=O)c1=O ; *CCCN(C)c1c(N(CC)CC)c(=O)c1=O]

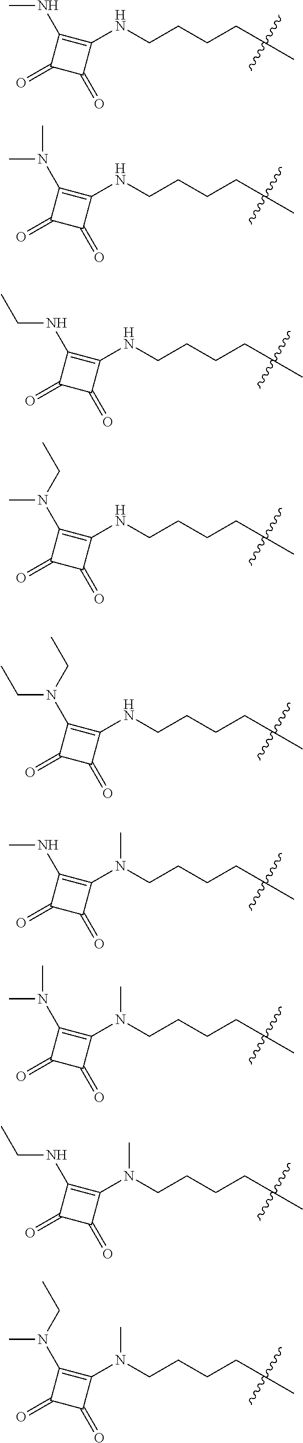

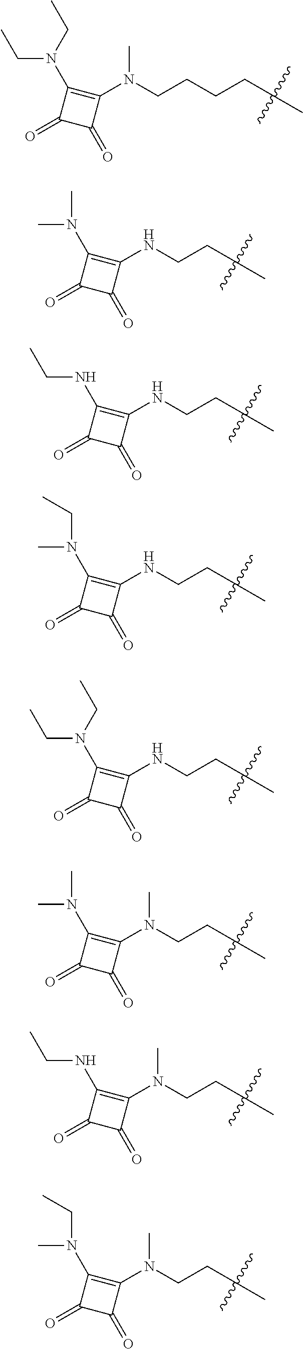

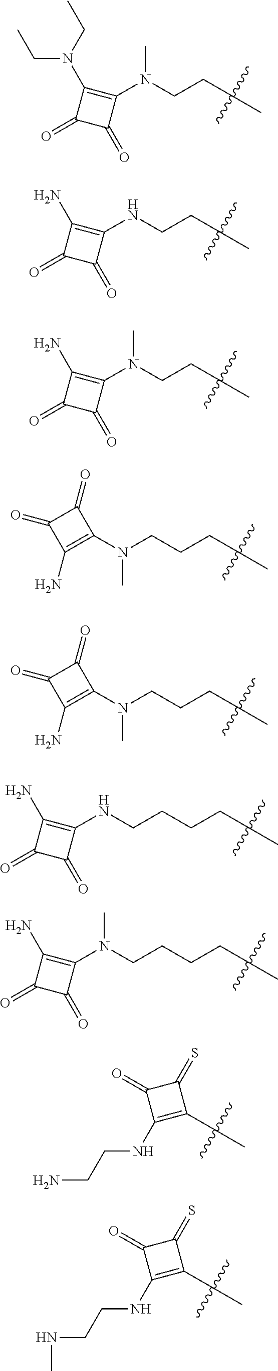

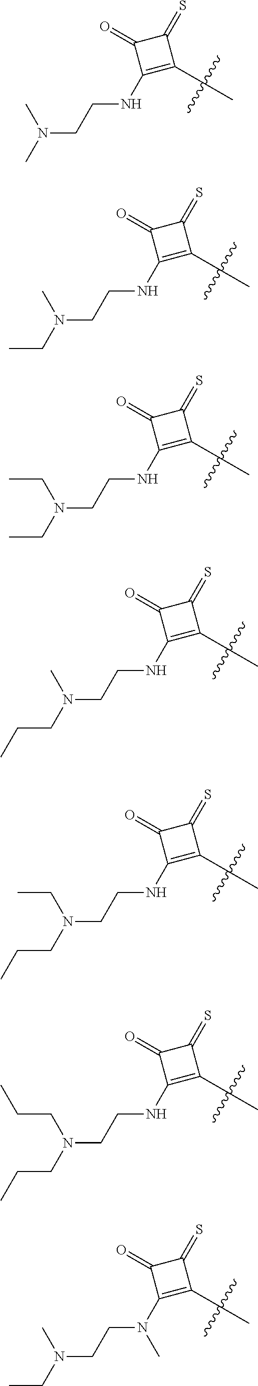

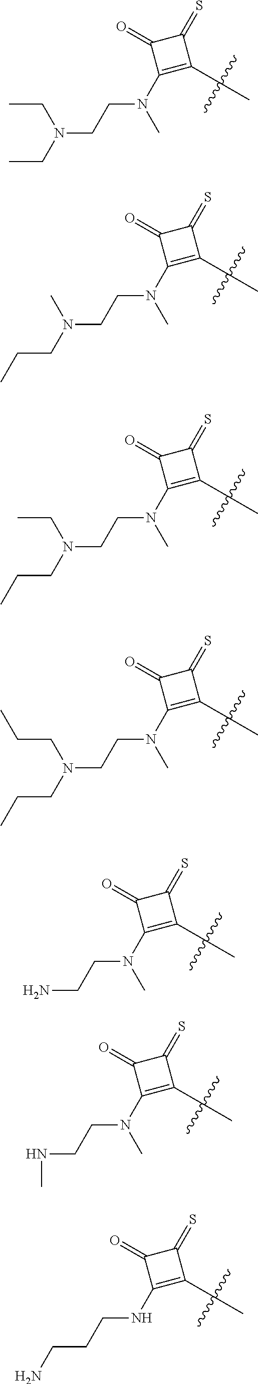

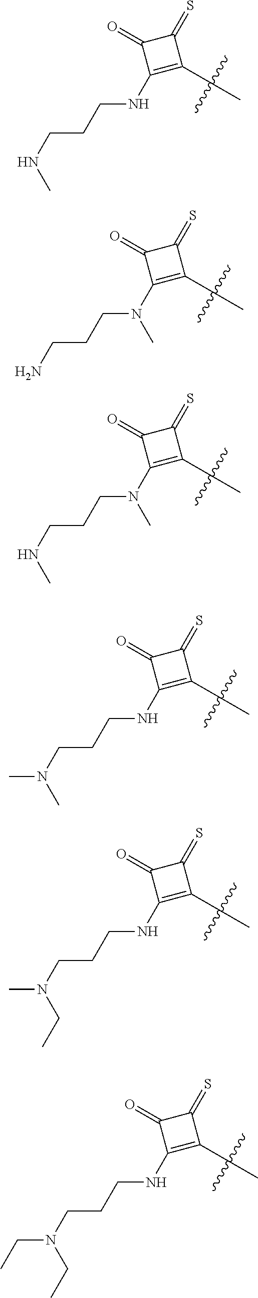 [SMILES: *c1c(NCCCNC)c(=O)c1=S ; *c1c(N(C)CCCN)c(=O)c1=S ; *c1c(N(C)CCCNC)c(=O)c1=S ; *c1c(NCCCN(C)C)c(=O)c1=S ; *c1c(NCCCN(C)CC)c(=O)c1=S ; *c1c(NCCCN(CC)CC)c(=O)c1=S]

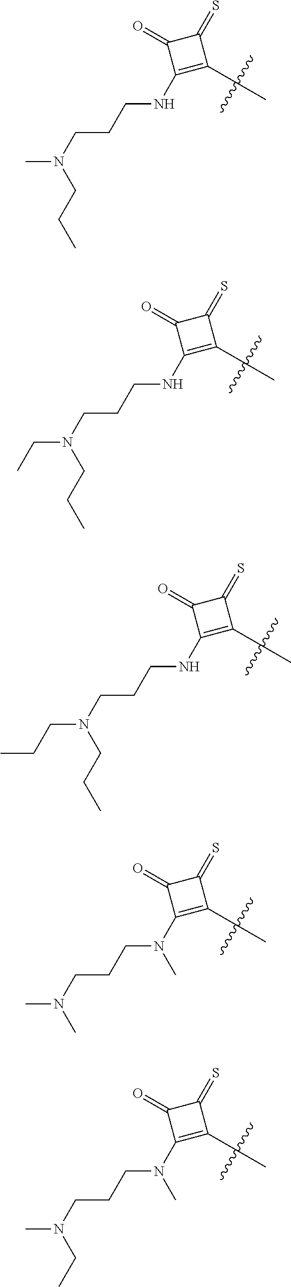 [SMILES: *c1c(NCCCN(C)CCC)c(=O)c1=S ; *c1c(NCCCN(CC)CCC)c(=O)c1=S ; *c1c(NCCCN(CCC)CCC)c(=O)c1=S ; *c1c(N(C)CCCN(C)C)c(=O)c1=S ; *c1c(N(C)CCCN(C)CC)c(=O)c1=S]

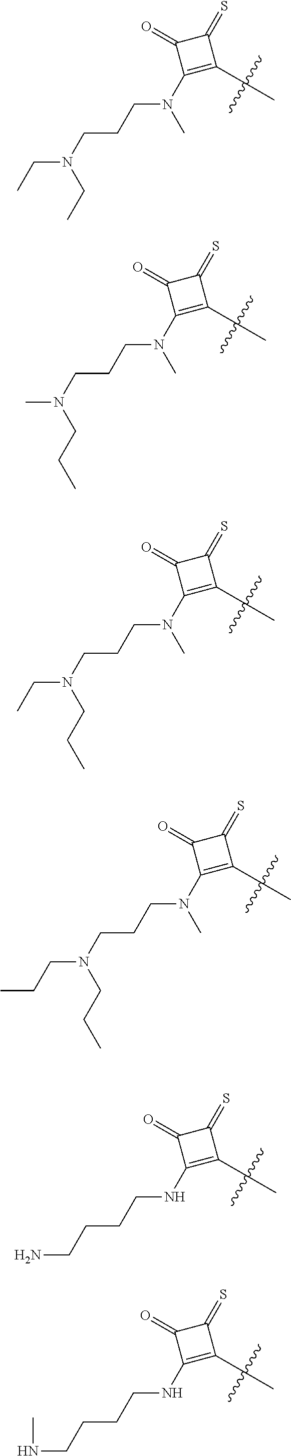 [SMILES: *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S ; *c1c(NCCCCN)c(=O)c1=S ; *c1c(NCCCCNC)c(=O)c1=S]

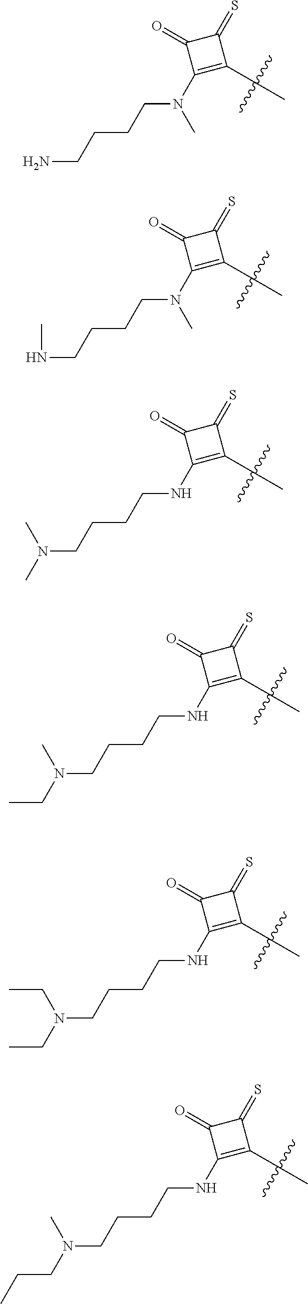 [SMILES: *c1c(N(C)CCCCN)c(=O)c1=S ; *c1c(N(C)CCCCNC)c(=O)c1=S ; *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S]

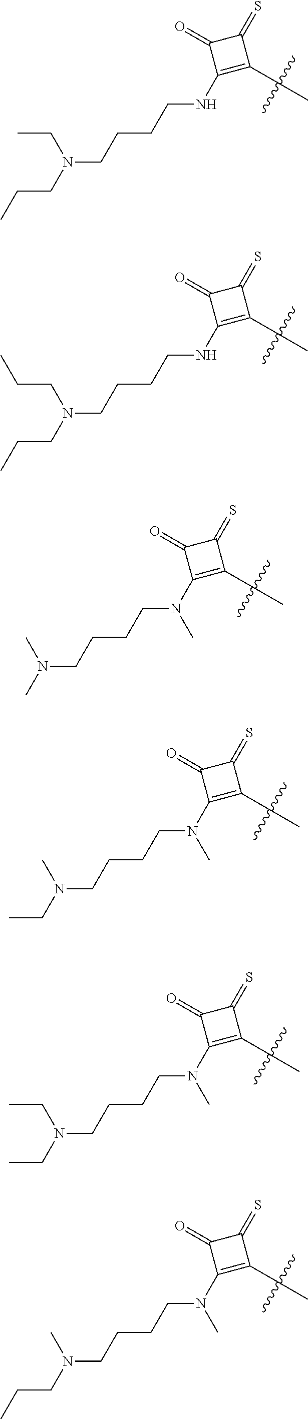 [SMILES: *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S]

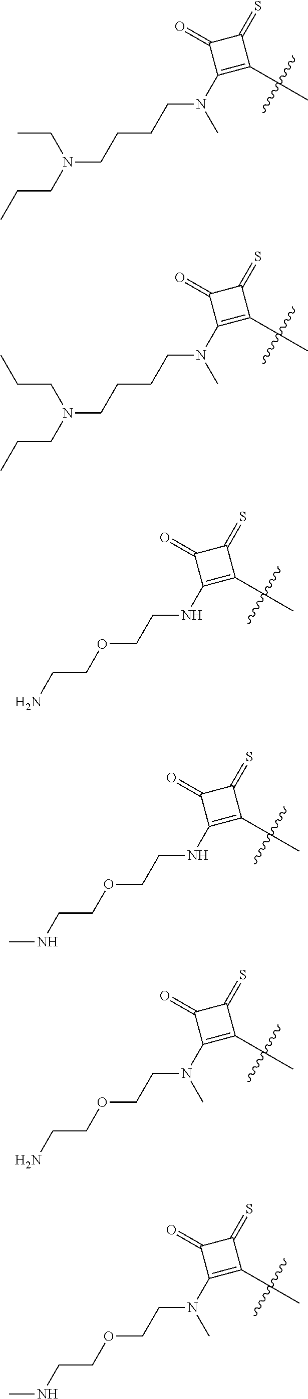 [SMILES: *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

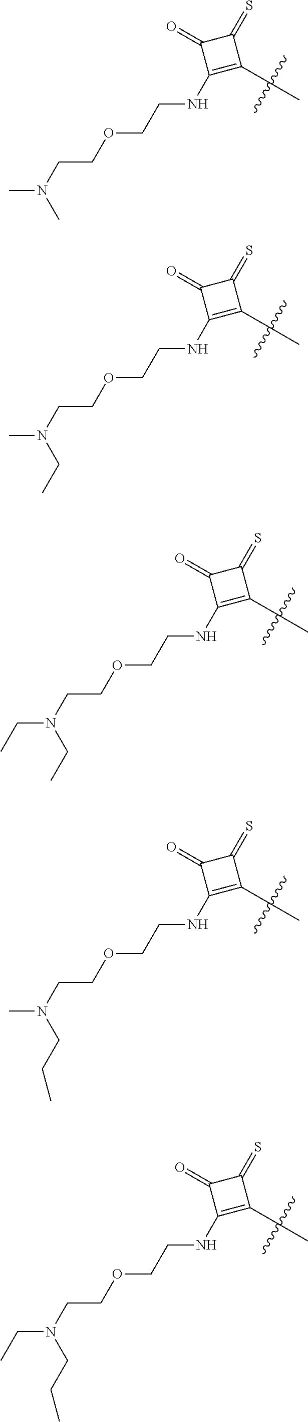 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

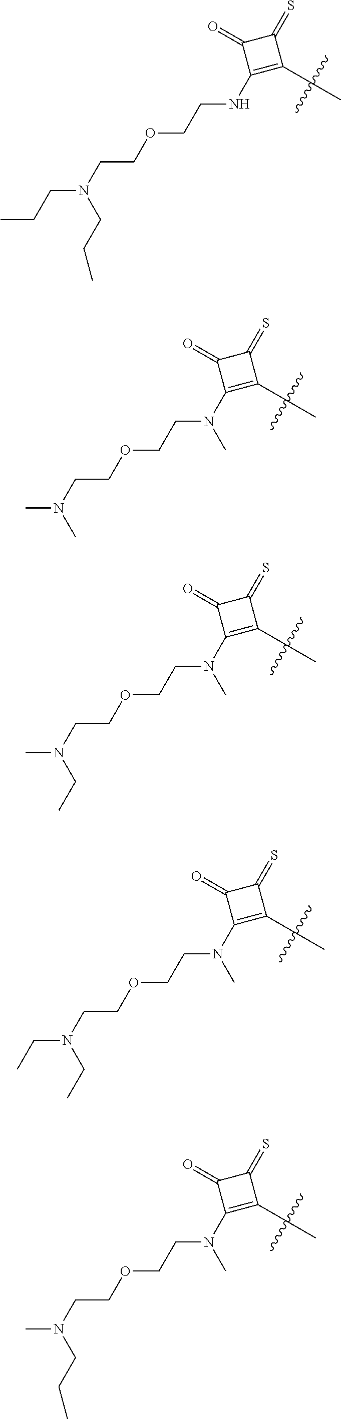 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

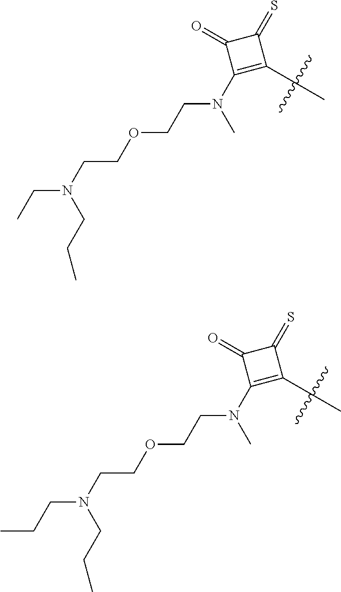 [SMILES: *c1c(N(C)CCOCCN(CC)CCC)c(=O)c1=S ; *c1c(N(C)CCOCCN(CCC)CCC)c(=O)c1=S]

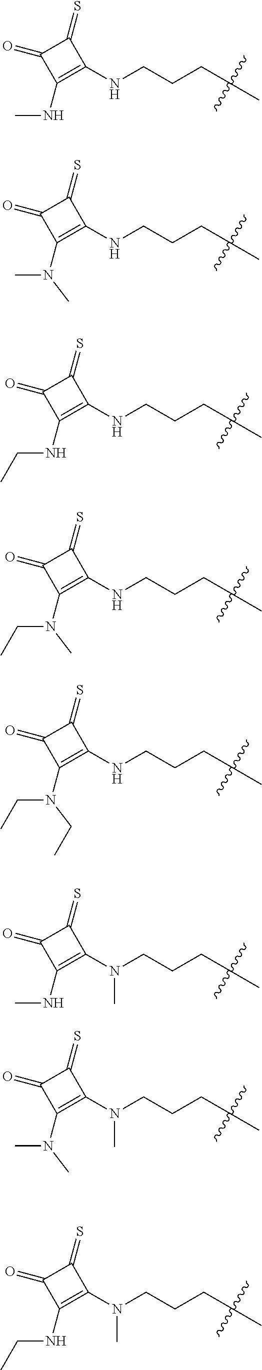

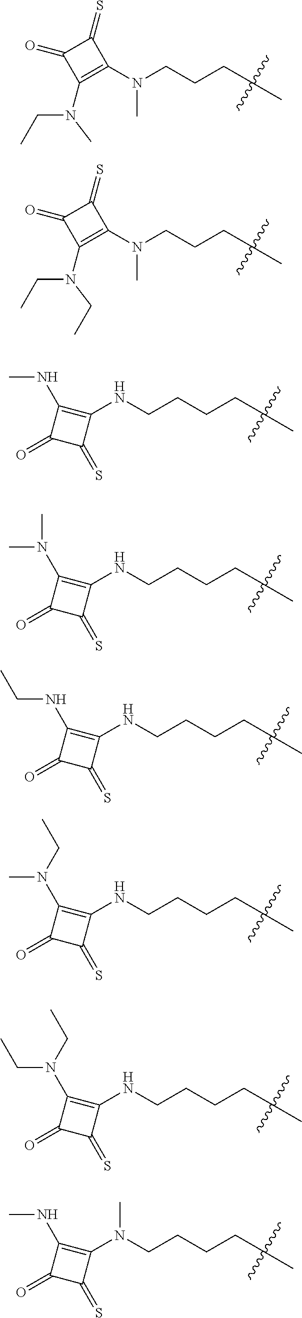

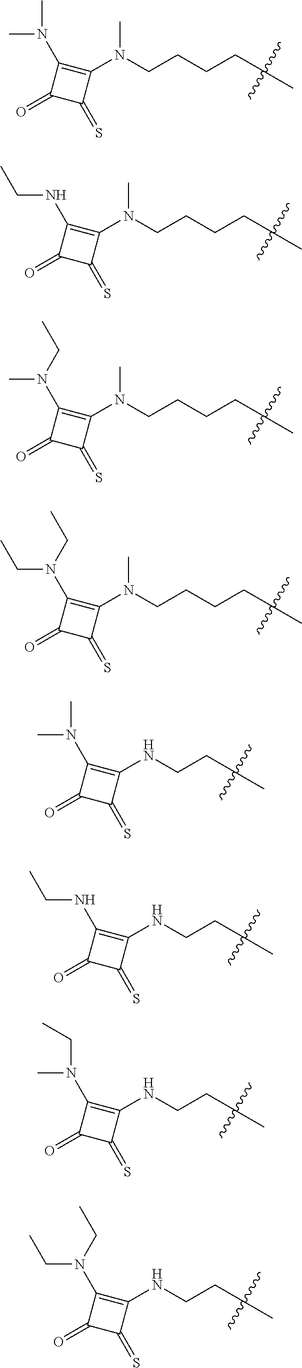

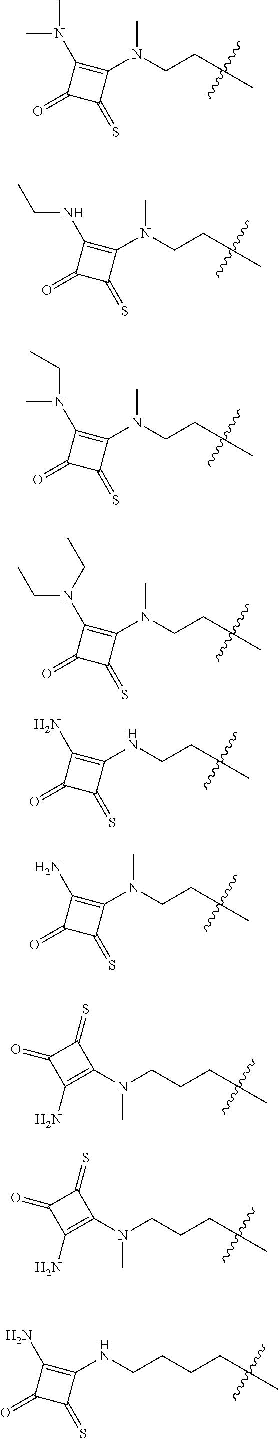

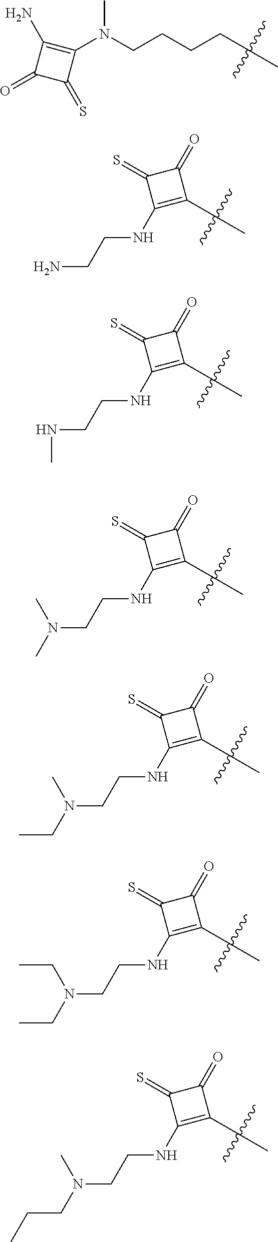

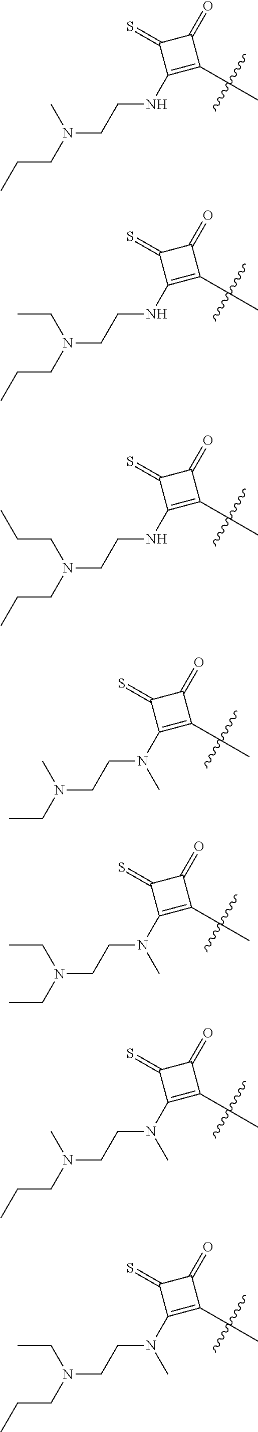

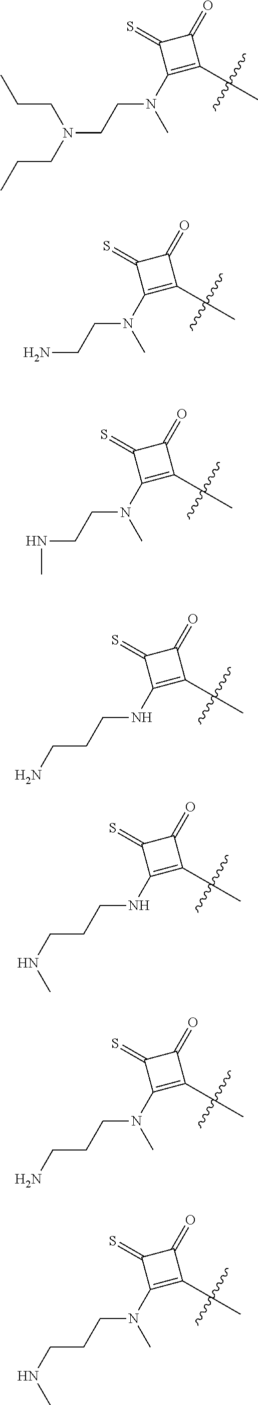

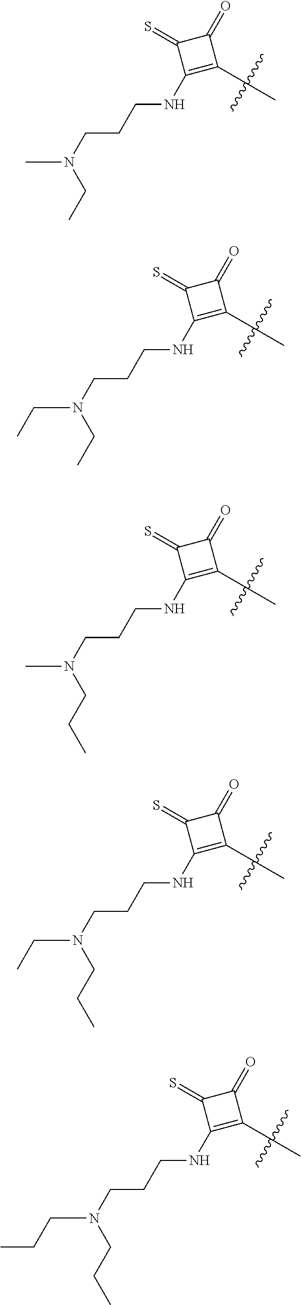 [SMILES: *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O ; *c1c(NCCCN(CCC)CCC)c(=S)c1=O]

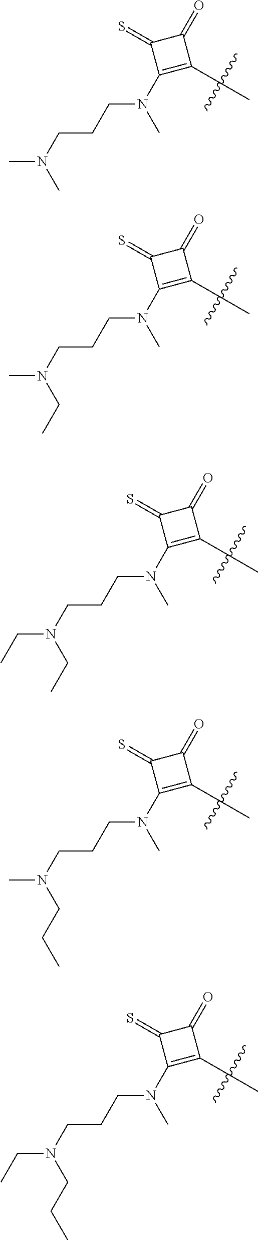 [SMILES: *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O]

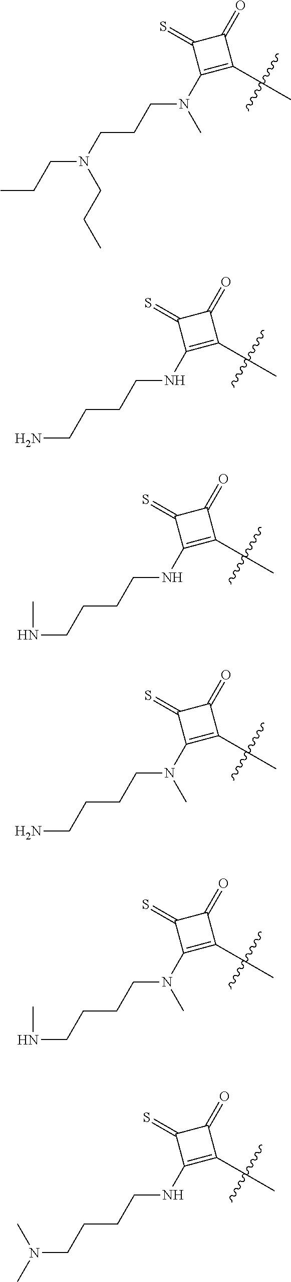 [SMILES: *c1c(N(C)CCCN(CCC)CCC)c(=S)c1=O ; *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O ; *c1c(NCCCCN(C)C)c(=S)c1=O]

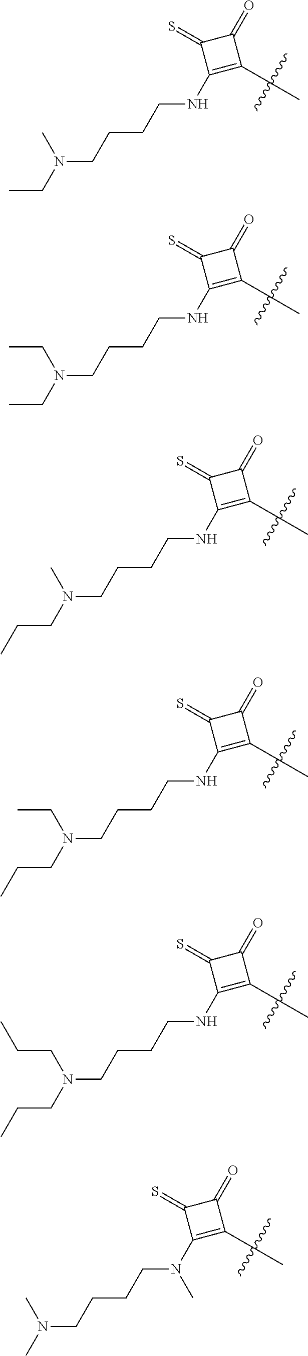 [SMILES: *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O ; *c1c(N(C)CCCCN(C)C)c(=S)c1=O]

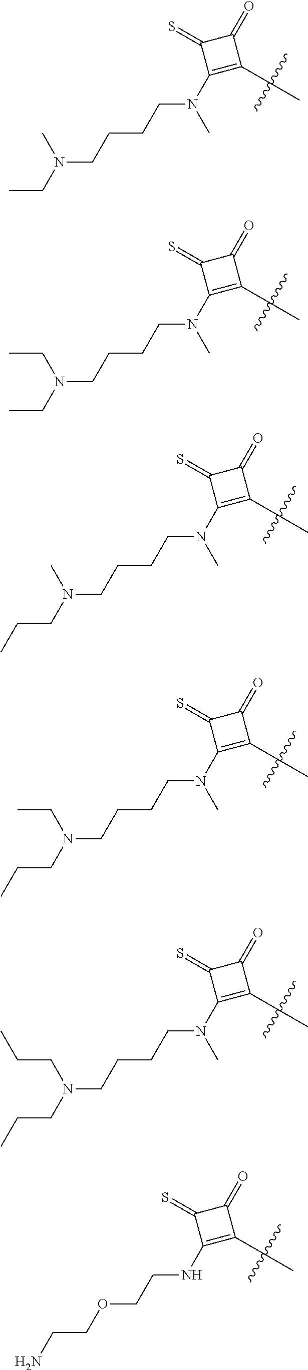 [SMILES: *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O ; *c1c(NCCOCCN)c(=S)c1=O]

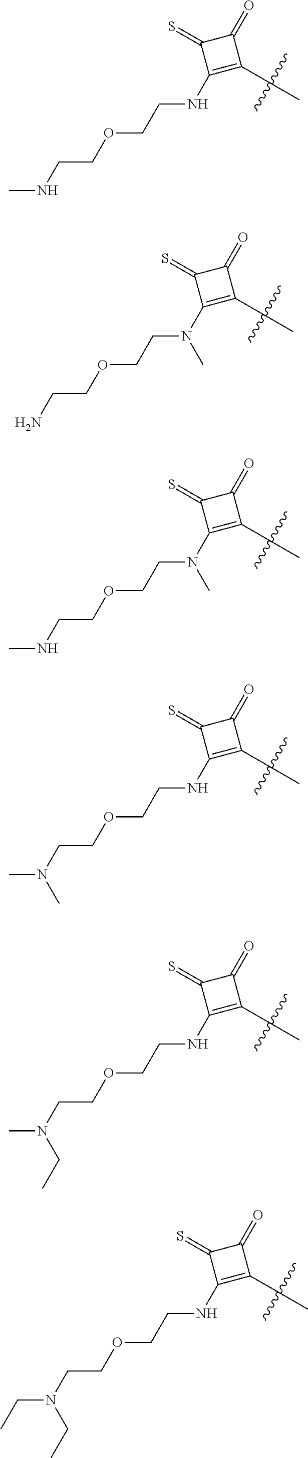 [SMILES: *c1c(NCCOCCNC)c(=S)c1=O ; *c1c(N(C)CCOCCN)c(=S)c1=O ; *c1c(N(C)CCOCCNC)c(=S)c1=O ; *c1c(NCCOCCN(C)C)c(=S)c1=O ; *c1c(NCCOCCN(C)CC)c(=S)c1=O ; *c1c(NCCOCCN(CC)CC)c(=S)c1=O]

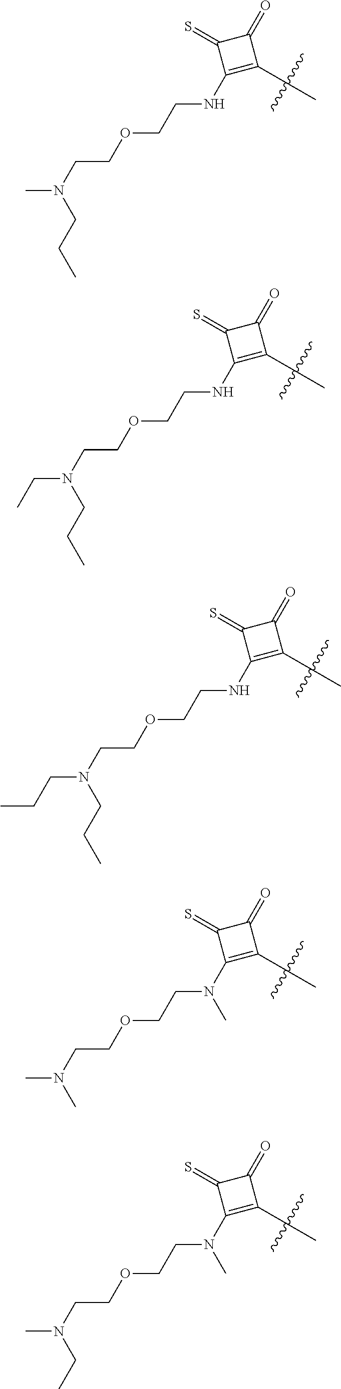 [SMILES: *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O ; *c1c(N(C)CCOCCN(C)C)c(=S)c1=O ; *c1c(N(C)CCOCCN(C)CC)c(=S)c1=O]

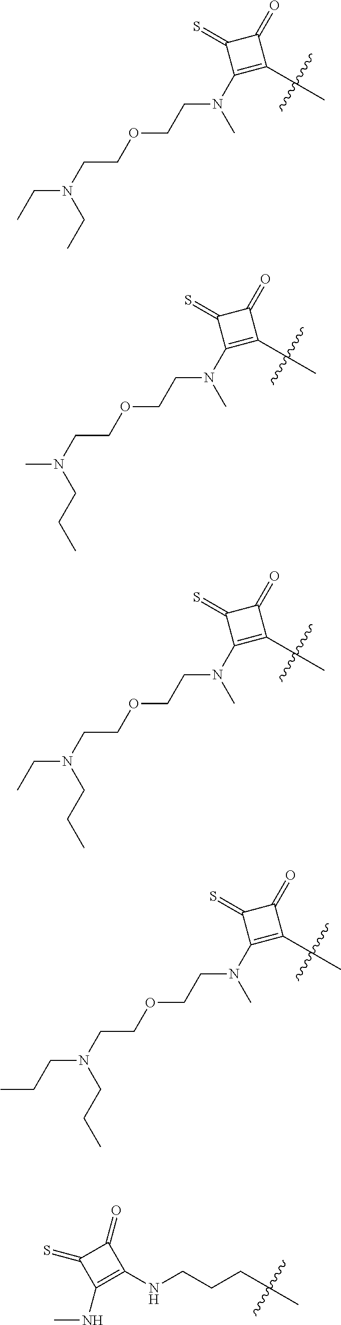 [SMILES: *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O ; *c1c(N(C)CCOCCN(CCC)CCC)c(=S)c1=O ; *CCCNc1c(NC)c(=S)c1=O]

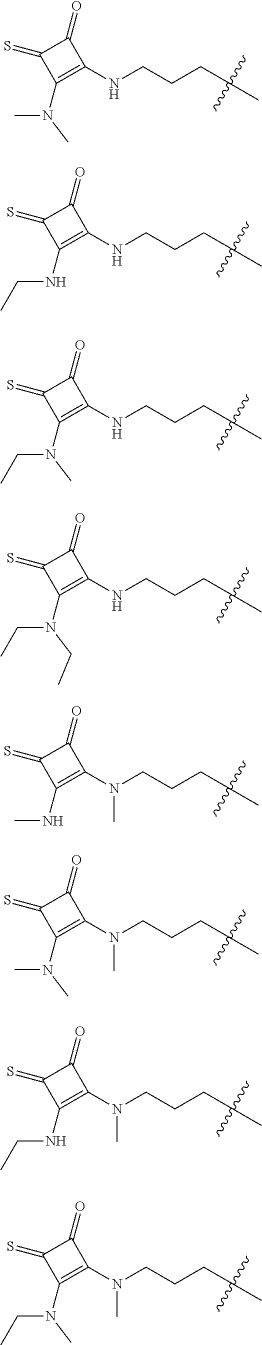

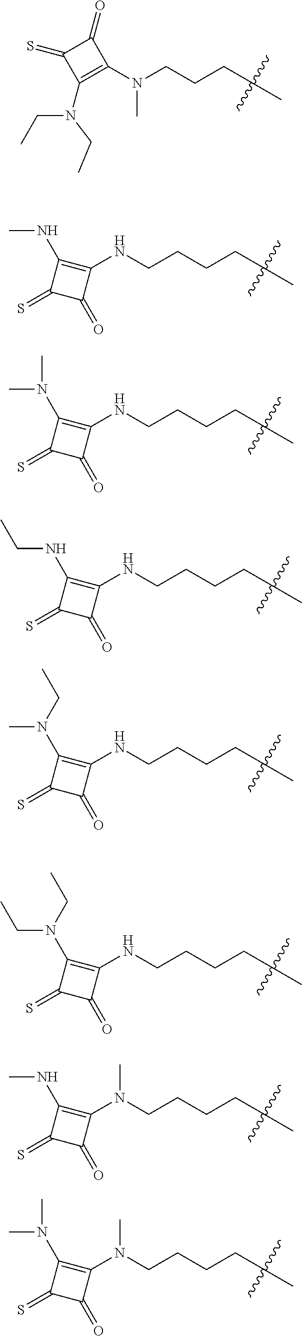

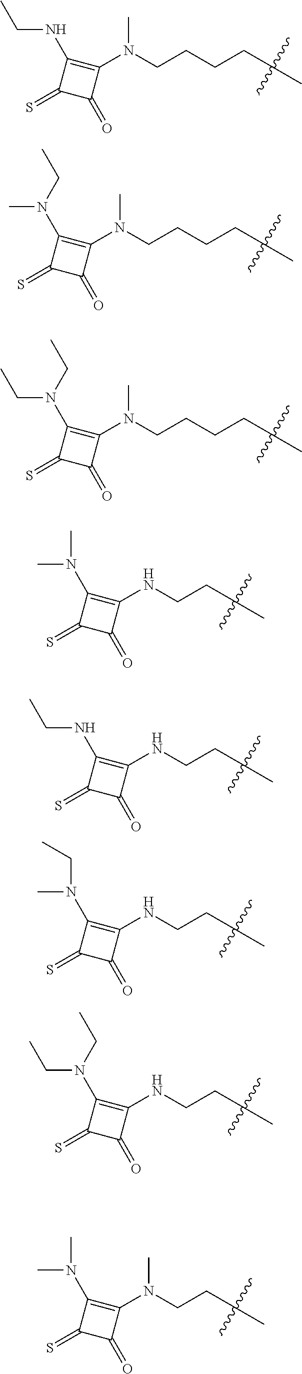

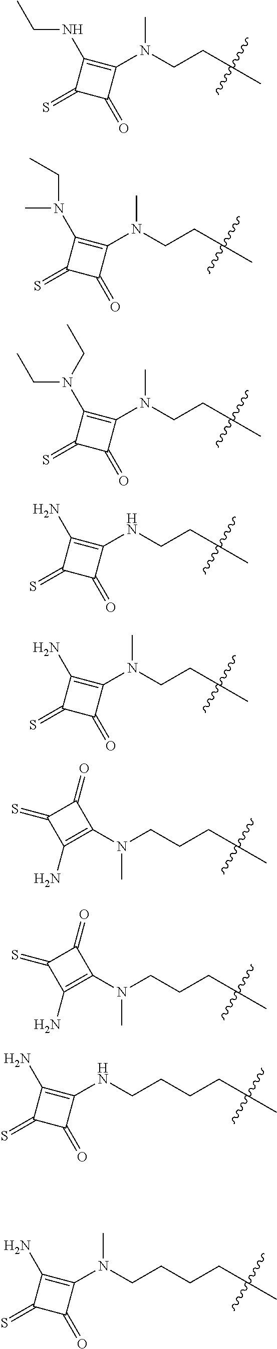

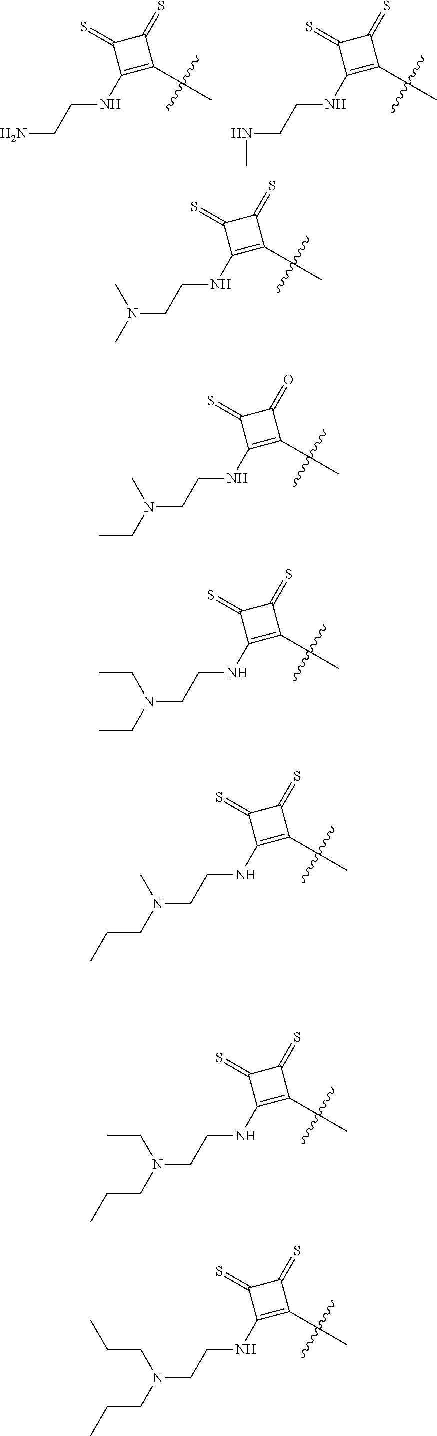

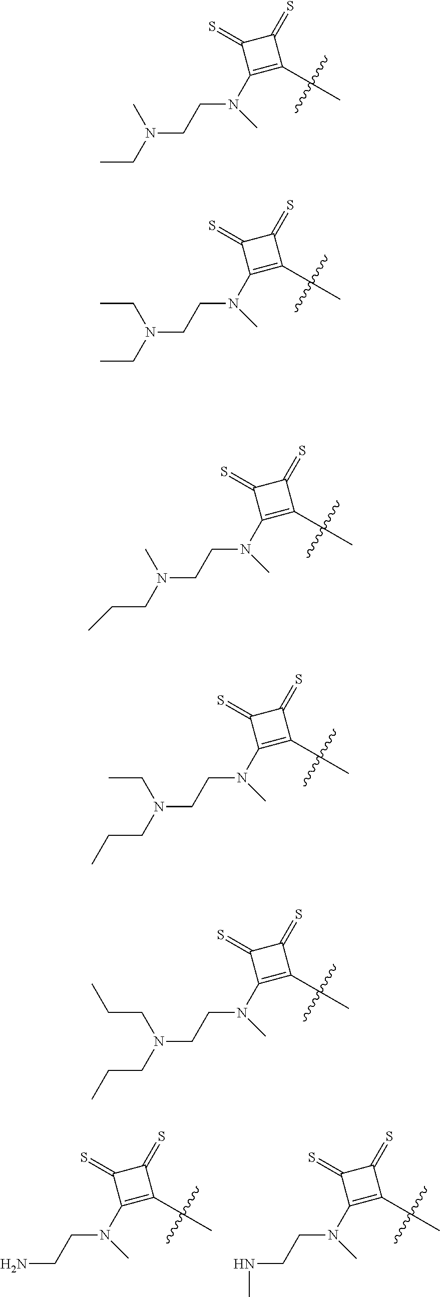

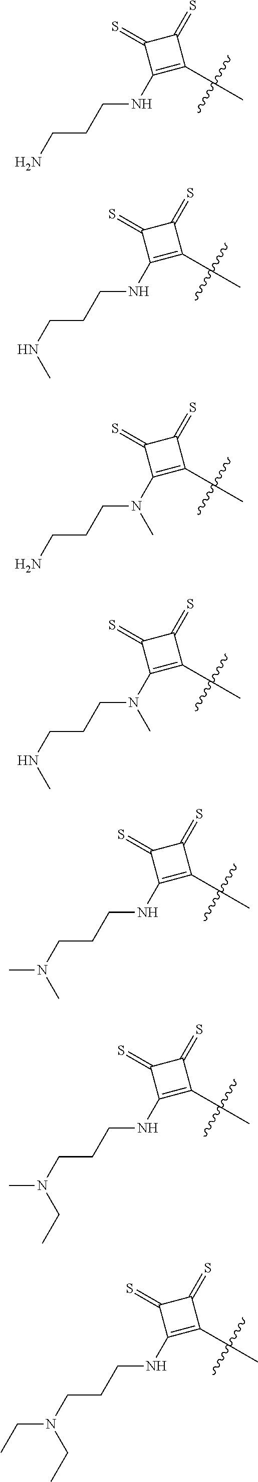

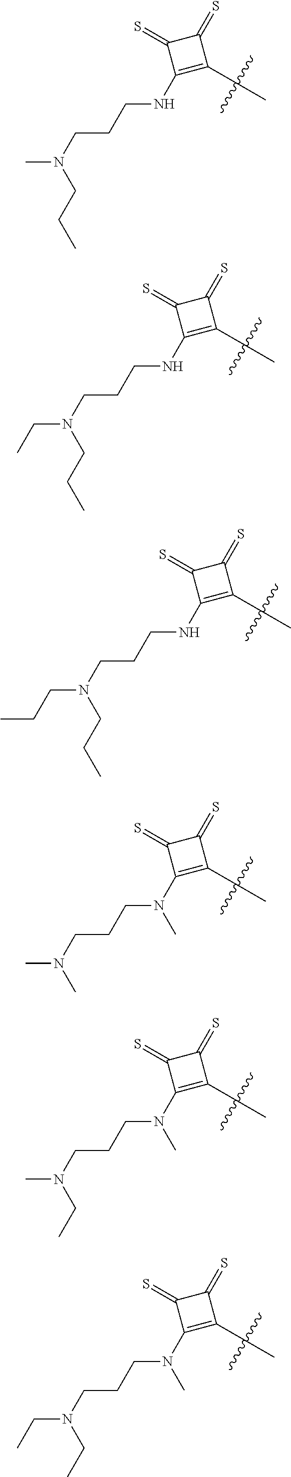 [SMILES: *c1c(NCCCN(C)CCC)c(=S)c1=S ; *c1c(NCCCN(CC)CCC)c(=S)c1=S ; *c1c(NCCCN(CCC)CCC)c(=S)c1=S ; *c1c(N(C)CCCN(C)C)c(=S)c1=S ; *c1c(N(C)CCCN(C)CC)c(=S)c1=S ; *c1c(N(C)CCCN(CC)CC)c(=S)c1=S]

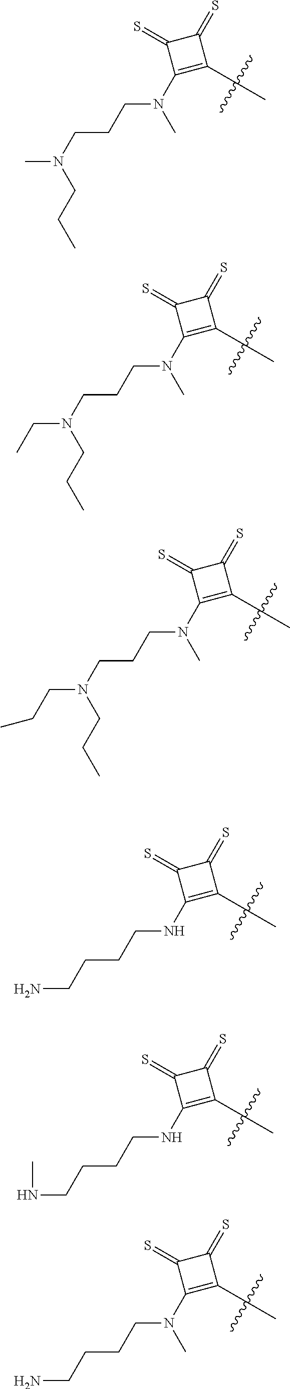 [SMILES: *c1c(N(C)CCCN(C)CCC)c(=S)c1=S ; *c1c(N(C)CCCN(CC)CCC)c(=S)c1=S ; *c1c(N(C)CCCN(CCC)CCC)c(=S)c1=S ; *c1c(NCCCCN)c(=S)c1=S ; *c1c(NCCCCNC)c(=S)c1=S ; *c1c(N(C)CCCCN)c(=S)c1=S]

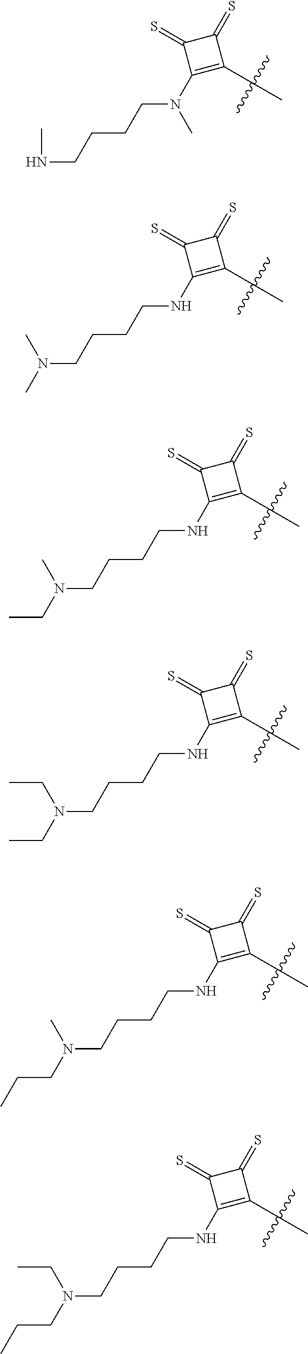 [SMILES: *c1c(N(C)CCCCNC)c(=S)c1=S ; *c1c(NCCCCN(C)C)c(=S)c1=S ; *c1c(NCCCCN(C)CC)c(=S)c1=S ; *c1c(NCCCCN(CC)CC)c(=S)c1=S ; *c1c(NCCCCN(C)CCC)c(=S)c1=S ; *c1c(NCCCCN(CC)CCC)c(=S)c1=S]

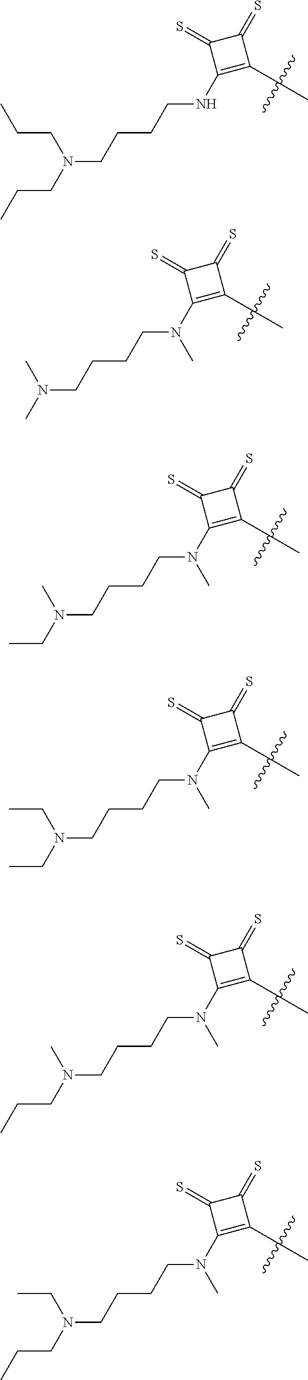 [SMILES: *c1c(NCCCCN(CCC)CCC)c(=S)c1=S ; *c1c(N(C)CCCCN(C)C)c(=S)c1=S ; *c1c(N(C)CCCCN(C)CC)c(=S)c1=S ; *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S]

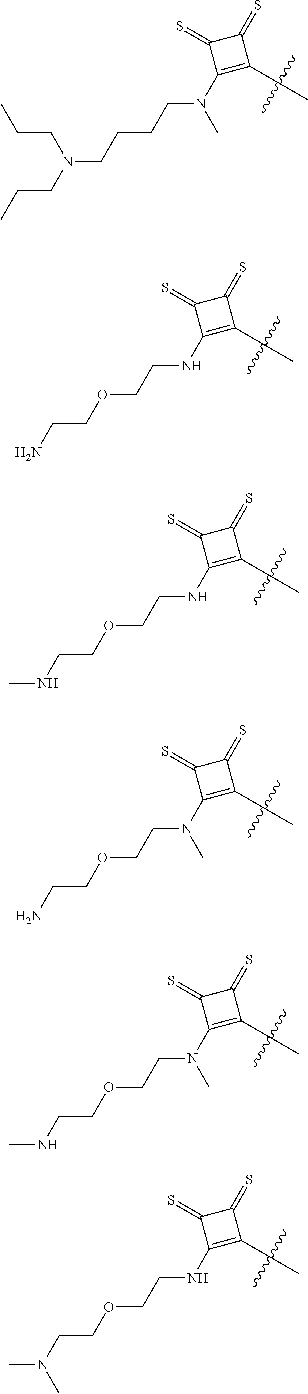 [SMILES: *c1c(N(C)CCCCN(CCC)CCC)c(=S)c1=S ; *c1c(NCCOCCN)c(=S)c1=S ; *c1c(NCCOCCNC)c(=S)c1=S ; *c1c(N(C)CCOCCN)c(=S)c1=S ; *c1c(N(C)CCOCCNC)c(=S)c1=S ; *c1c(NCCOCCN(C)C)c(=S)c1=S]

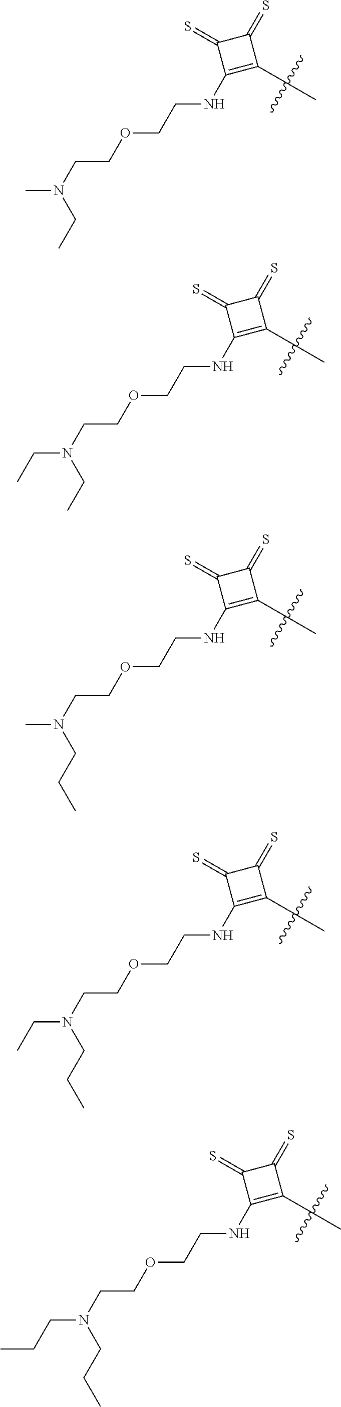 [SMILES: *c1c(NCCOCCN(C)CC)c(=S)c1=S ; *c1c(NCCOCCN(CC)CC)c(=S)c1=S ; *c1c(NCCOCCN(C)CCC)c(=S)c1=S ; *c1c(NCCOCCN(CC)CCC)c(=S)c1=S ; *c1c(NCCOCCN(CCC)CCC)c(=S)c1=S]

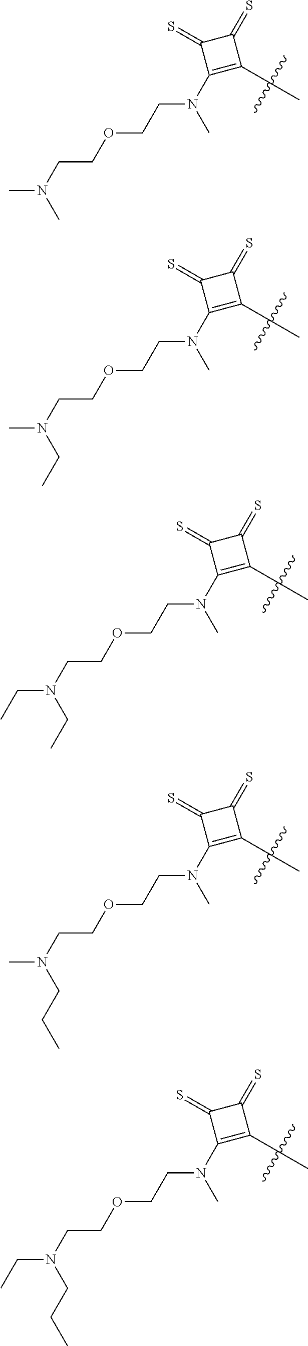 [SMILES: *c1c(N(C)CCOCCN(C)C)c(=S)c1=S ; *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S]

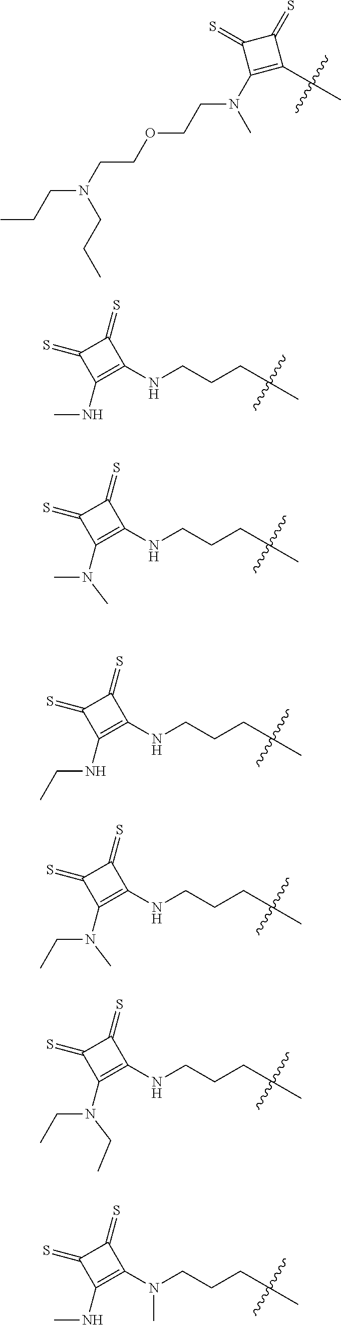

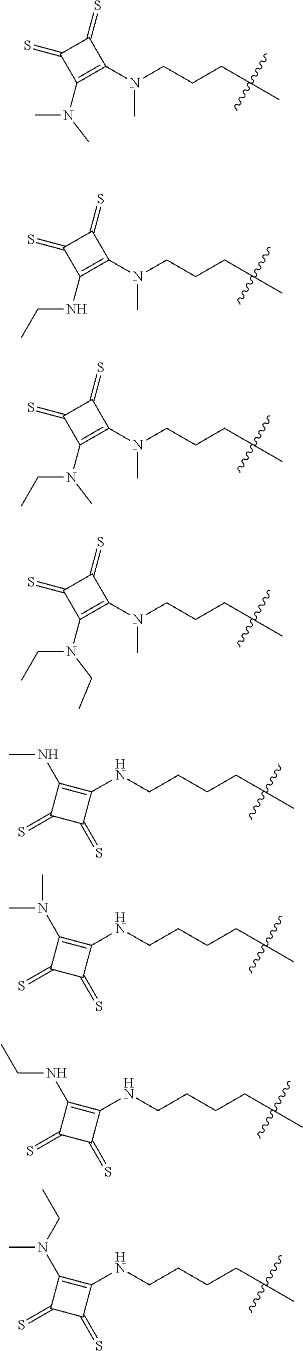

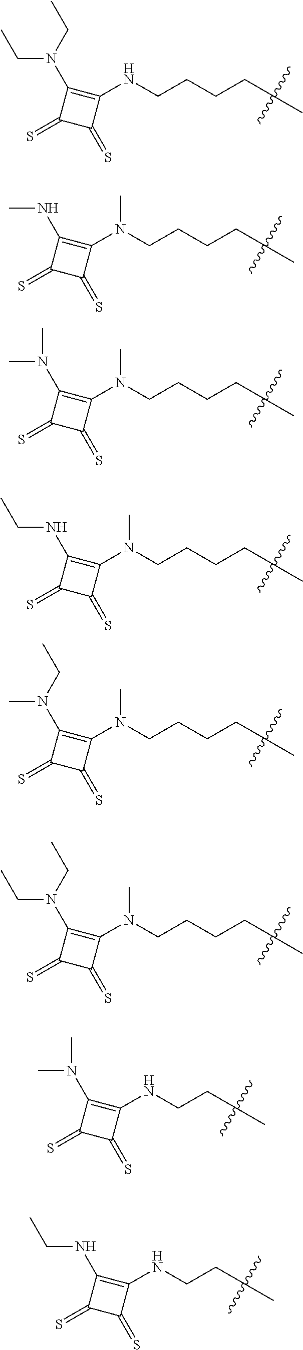

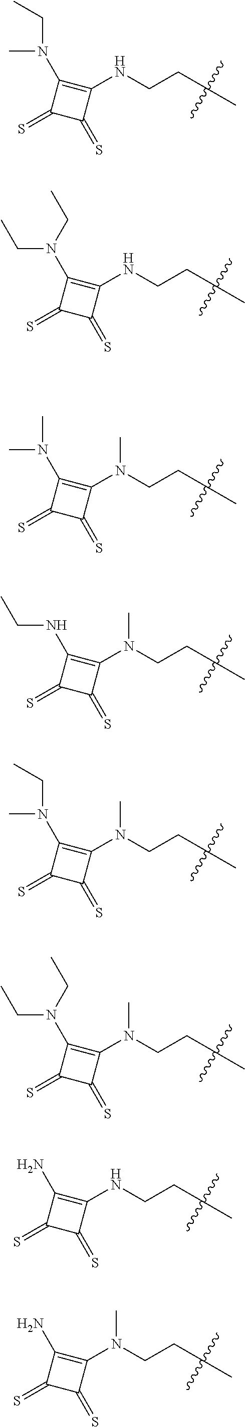

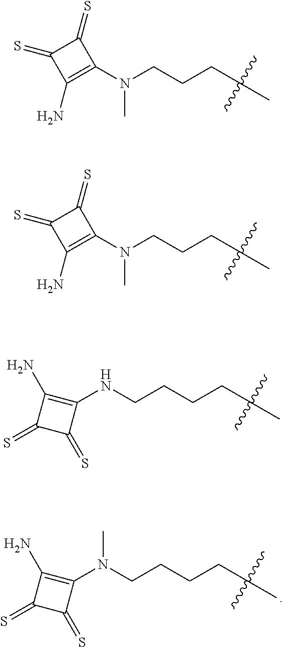 [SMILES: *CCCN(C)c1c(N)c(=S)c1=S ; *CCCN(C)c1c(N)c(=S)c1=S ; *CCCCNc1c(N)c(=S)c1=S ; *CCCCN(C)c1c(N)c(=S)c1=S]

In some embodiments, R⁴ is selected from any of the following groups:

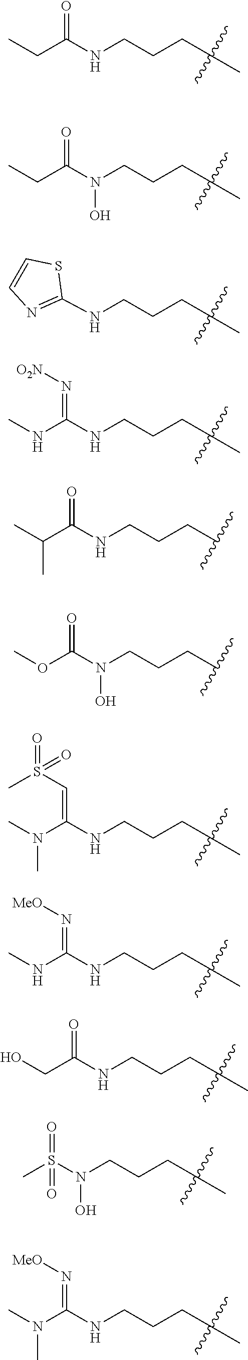

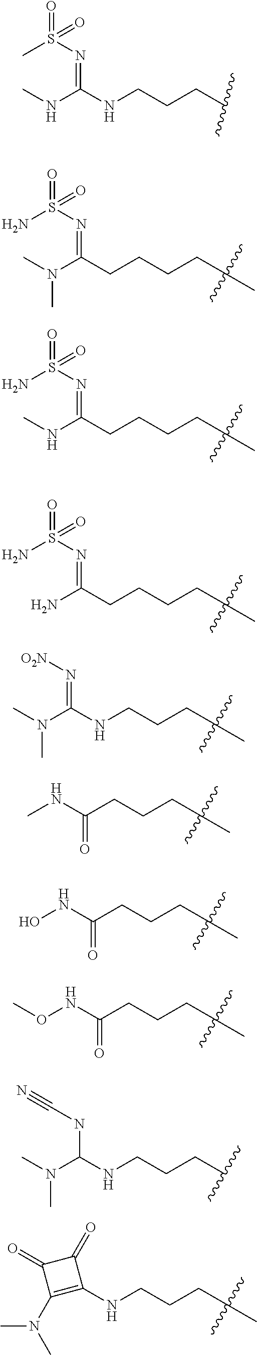

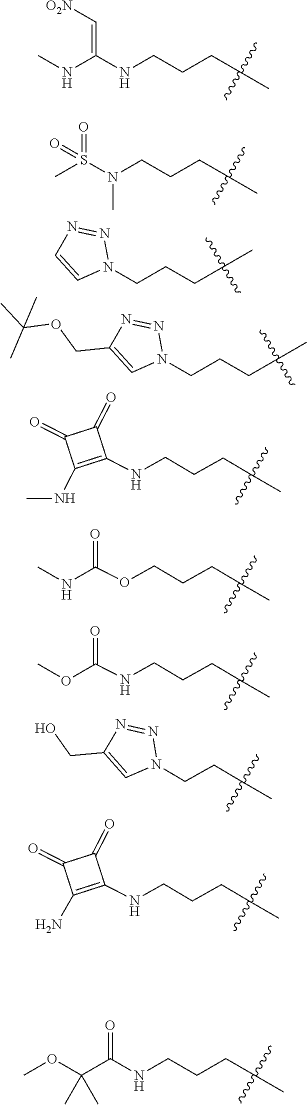

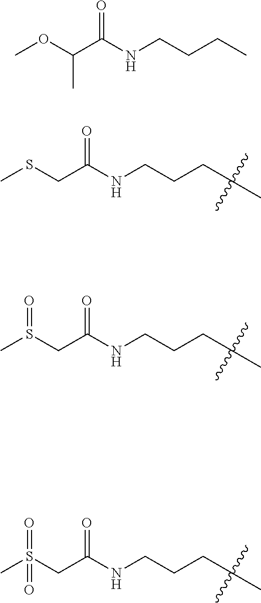

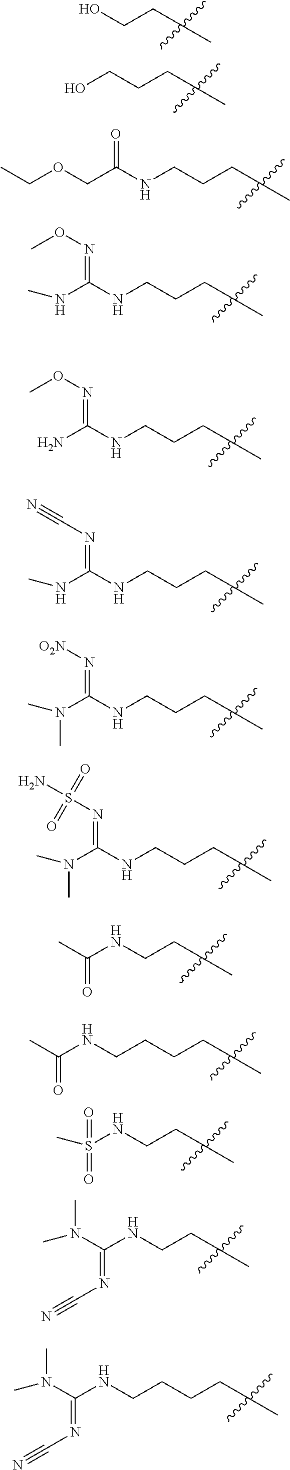

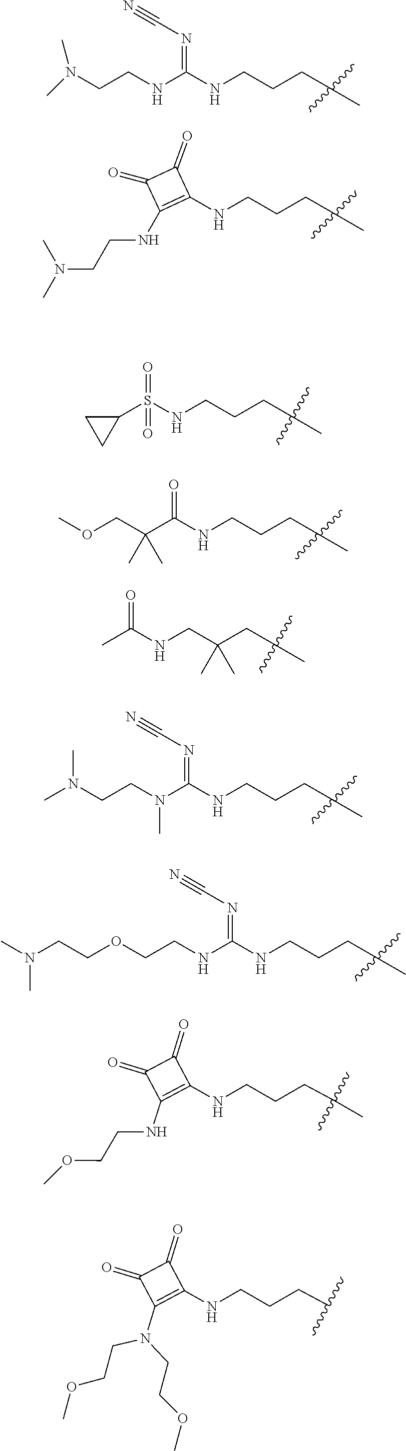

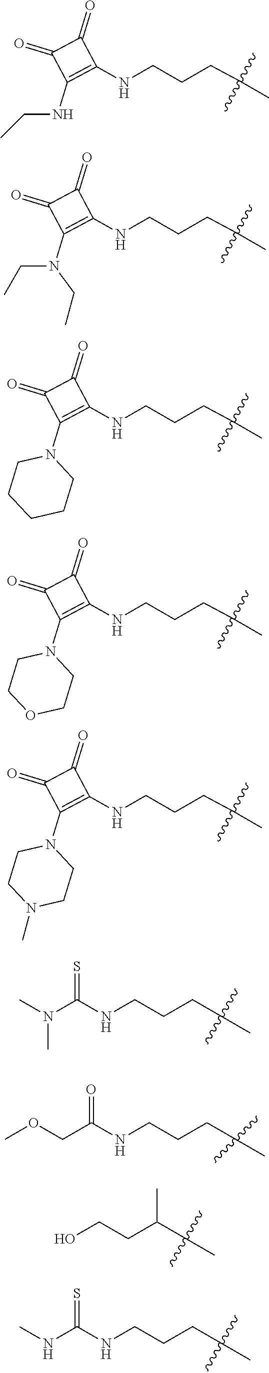

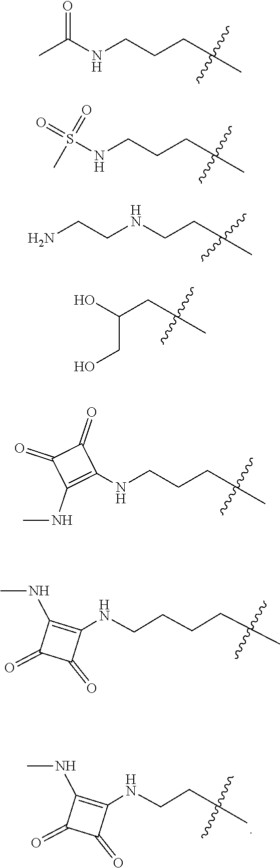

In some embodiments

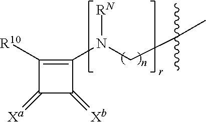

is selected from any of the following groups:

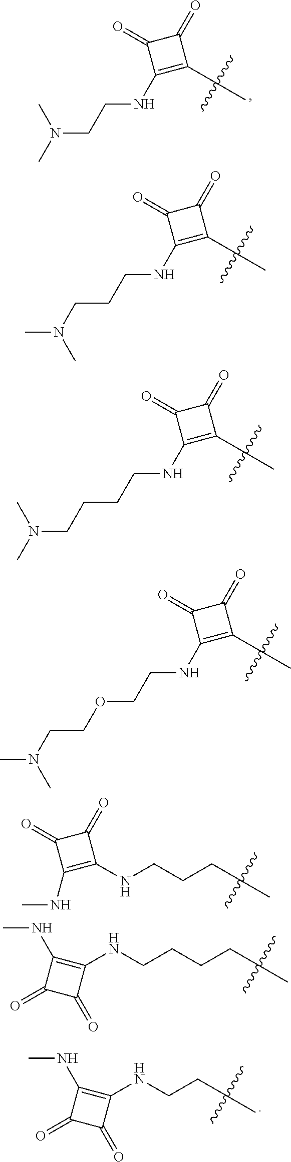

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formulae (VI), (VI-a), (VII), (VIIa), (VIIb), (VIIb-1), (VIIb-2), (VIIb-3), (VIIb-4), (VIIb-5), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments R^{N} is C₁ alkyl. For example, in some embodiments R^{N} is C₂ alkyl. For example, in some embodiments R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is-NH(CH₂)ₒN(R)₂, o is 2, 3, or 4.

In some embodiments wherein —NH(CH₂)ₚ₁O(CH₂)_{q1}N(R)₂, p¹ is 2. In some embodiments wherein —NH(CH₂)ₚ₁O(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is-NH(CH₂)ₒN(R)₂, —NH(CH₂)ₚO(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is-NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methylpiperazinyl.

In some embodiments, each occurrence of R⁵ and R⁶ is H. In some embodiments, the compound of Formula (I) is selected from the group consisting of:

| Cpd | Structure |
|---|---|
| I 1 | 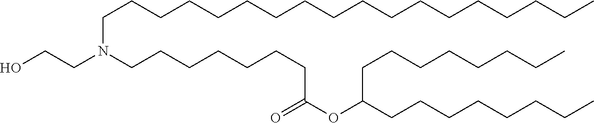 [SMILES: CCCCCCCCCCCCCCCCCCN(CCO)CCCCCCCC(=O)OC(CCCCCCCC)CCCCCCCC] |
| I 2 | 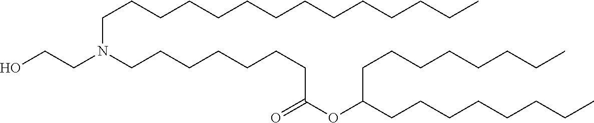 [SMILES: CCCCCCCCCCCCCCN(CCO)CCCCCCCC(=O)OC(CCCCCCCC)CCCCCCCC] |
| I 3 | 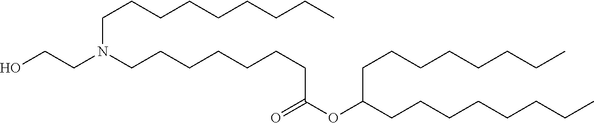 [SMILES: CCCCCCCCCN(CCO)CCCCCCCC(=O)OC(CCCCCCCC)CCCCCCCC] |
| I 4 | 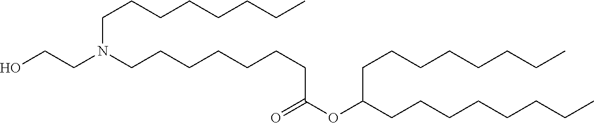 [SMILES: CCCCCCCCC(CCCCCCCC)OC(=O)CCCCCCCN(CCO)CCCCCCCC] |
| I 5 | 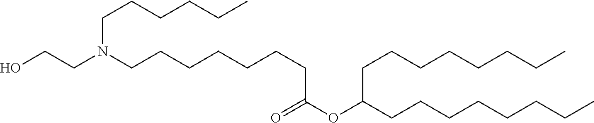 [SMILES: CCCCCCCCC(CCCCCCCC)OC(=O)CCCCCCCN(CCO)CCCCCC] |
| I 6 | 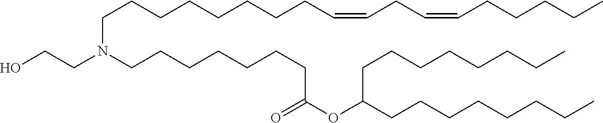 [SMILES: CCCCC/C=C\C/C=C\CCCCCCCCN(CCO)CCCCCCCC(=O)OC(CCCCCCCC)CCCCCCCC] |
| I 7 | 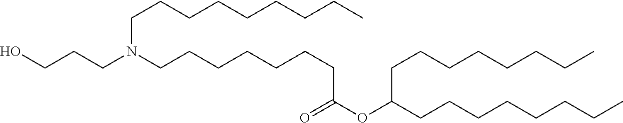 [SMILES: CCCCCCCCCN(CCCO)CCCCCCCC(=O)OC(CCCCCCCC)CCCCCCCC] |
| I 8 | 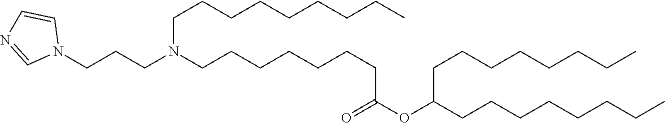 [SMILES: CCCCCCCCCN(CCCCCCCC(=O)OC(CCCCCCCC)CCCCCCCC)CCCn1ccnc1] |
| I 9 | 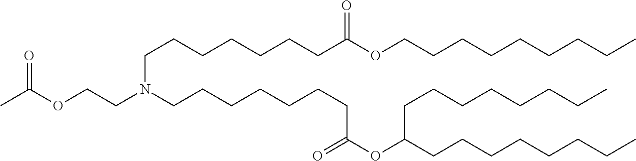 [SMILES: CCCCCCCCCOC(=O)CCCCCCCN(CCCCCCCC(=O)OC(CCCCCCCC)CCCCCCCC)CCOC(C)=O] |
| I 10 | 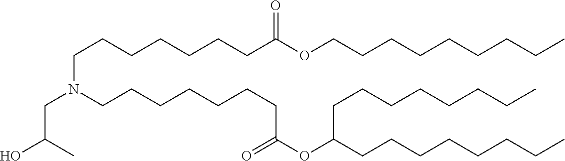 [SMILES: CCCCCCCCCOC(=O)CCCCCCCN(CCCCCCCC(=O)OC(CCCCCCCC)CCCCCCCC)CC(C)O] |
| I 11 | 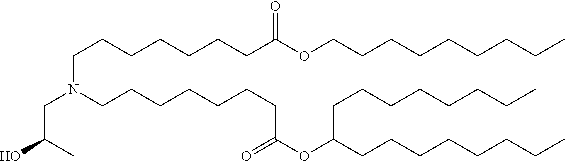 [SMILES: CCCCCCCCCOC(=O)CCCCCCCN(CCCCCCCC(=O)OC(CCCCCCCC)CCCCCCCC)C[C@@H](C)O] |
| I 12 | 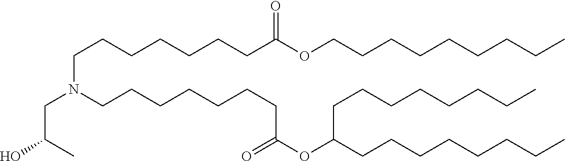 [SMILES: CCCCCCCCCOC(=O)CCCCCCCN(CCCCCCCC(=O)OC(CCCCCCCC)CCCCCCCC)C[C@H](C)O] |
| I 13 | 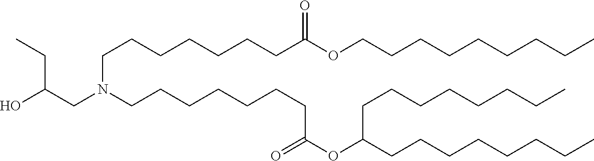 [SMILES: CCCCCCCCCOC(=O)CCCCCCCN(CCCCCCCC(=O)OC(CCCCCCCC)CCCCCCCC)CC(O)CC] |
| I 14 | 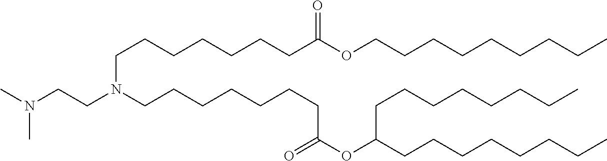 [SMILES: CCCCCCCCCOC(=O)CCCCCCCN(CCCCCCCC(=O)OC(CCCCCCCC)CCCCCCCC)CCN(C)C] |
| I 15 | 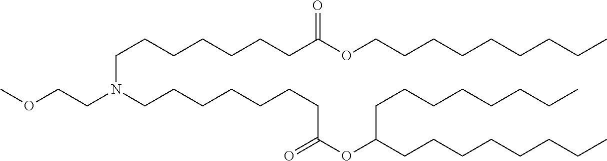 [SMILES: CCCCCCCCCOC(=O)CCCCCCCN(CCCCCCCC(=O)OC(CCCCCCCC)CCCCCCCC)CCOC] |
| I 16 | 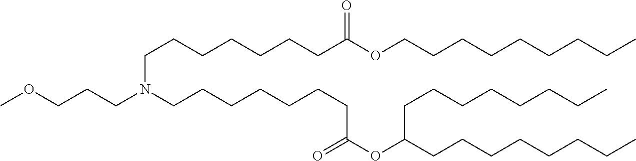 [SMILES: CCCCCCCCCOC(=O)CCCCCCCN(CCCCCCCC(=O)OC(CCCCCCCC)CCCCCCCC)CCCOC] |
| I 17 | 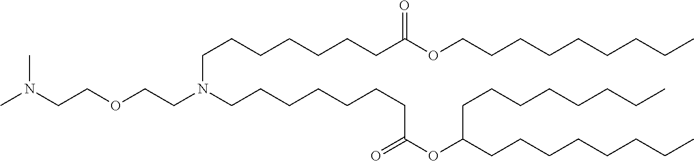 [SMILES: CCCCCCCCCOC(=O)CCCCCCCN(CCCCCCCC(=O)OC(CCCCCCCC)CCCCCCCC)CCOCCN(C)C] |
| I 18 | 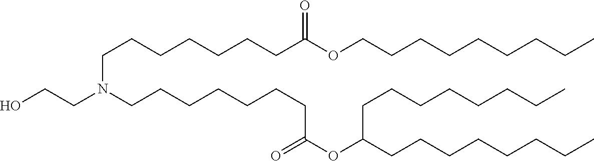 [SMILES: CCCCCCCCCOC(=O)CCCCCCCN(CCO)CCCCCCCC(=O)OC(CCCCCCCC)CCCCCCCC] |
| I 19 | 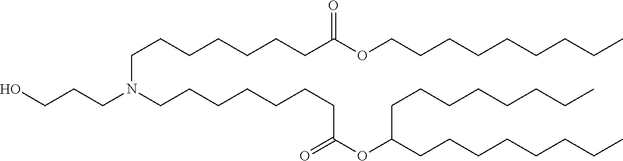 [SMILES: CCCCCCCCCOC(=O)CCCCCCCN(CCCO)CCCCCCCC(=O)OC(CCCCCCCC)CCCCCCCC] |
| I 20 | 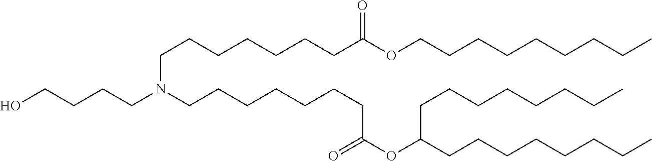 [SMILES: CCCCCCCCCOC(=O)CCCCCCCN(CCCCO)CCCCCCCC(=O)OC(CCCCCCCC)CCCCCCCC] |
| I 21 | 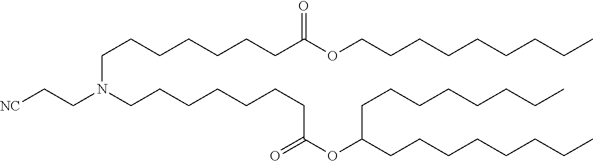 [SMILES: CCCCCCCCCOC(=O)CCCCCCCN(CCC#N)CCCCCCCC(=O)OC(CCCCCCCC)CCCCCCCC] |
| I 22 | 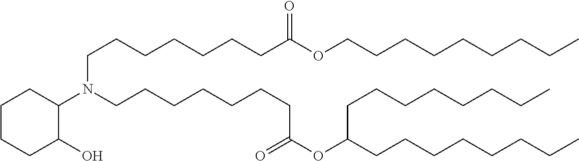 [SMILES: CCCCCCCCCOC(=O)CCCCCCCN(CCCCCCCC(=O)OC(CCCCCCCC)CCCCCCCC)C1CCCCC1O] |
| I 23 | 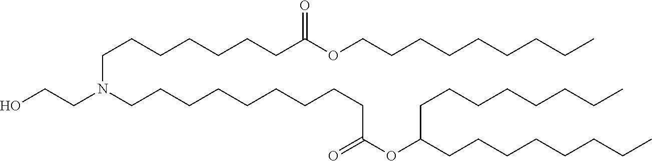 [SMILES: CCCCCCCCCOC(=O)CCCCCCCN(CCO)CCCCCCCCCC(=O)OC(CCCCCCCC)CCCCCCCC] |
| I 24 | 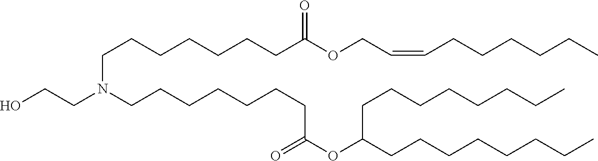 [SMILES: CCCCCC/C=C\COC(=O)CCCCCCCN(CCO)CCCCCCCC(=O)OC(CCCCCCCC)CCCCCCCC] |
| I 25 | 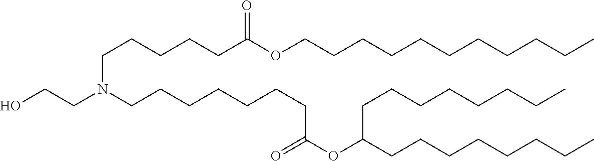 [SMILES: CCCCCCCCCCCOC(=O)CCCCCN(CCO)CCCCCCCC(=O)OC(CCCCCCCC)CCCCCCCC] |
| I 26 | 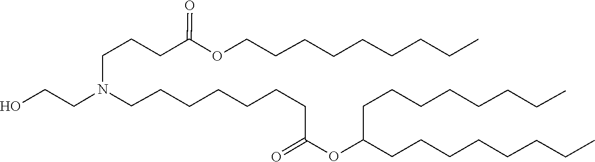 [SMILES: CCCCCCCCCOC(=O)CCCN(CCO)CCCCCCCC(=O)OC(CCCCCCCC)CCCCCCCC] |
| I 27 | 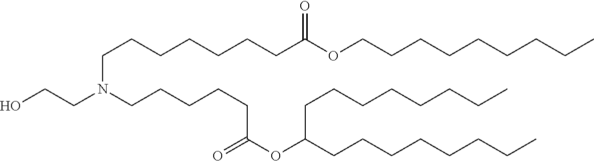 [SMILES: CCCCCCCCCOC(=O)CCCCCCCN(CCO)CCCCCC(=O)OC(CCCCCCCC)CCCCCCCC] |
| I 28 | 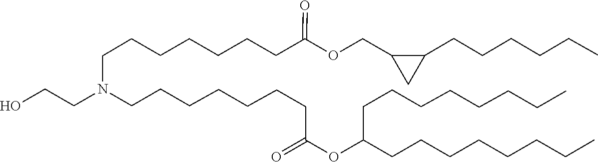 [SMILES: CCCCCCCCC(CCCCCCCC)OC(=O)CCCCCCCN(CCO)CCCCCCCC(=O)OCC1CC1CCCCCC] |
| I 29 | 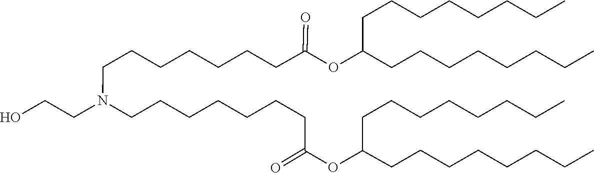 [SMILES: CCCCCCCCC(CCCCCCCC)OC(=O)CCCCCCCN(CCO)CCCCCCCC(=O)OC(CCCCCCCC)CCCCCCCC] |
| I 30 | 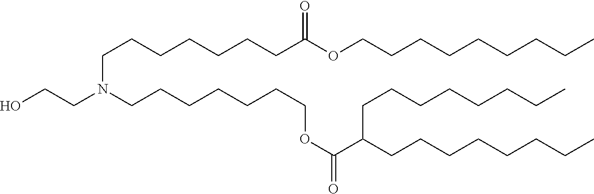 [SMILES: CCCCCCCCCOC(=O)CCCCCCCN(CCO)CCCCCCCOC(=O)C(CCCCCCCC)CCCCCCCC] |
| I 31 | 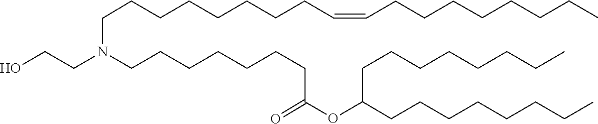 [SMILES: CCCCCCCC/C=C\CCCCCCCCN(CCO)CCCCCCCC(=O)OC(CCCCCCCC)CCCCCCCC] |
| I 32 | 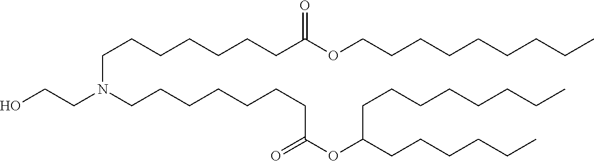 [SMILES: CCCCCCCCCOC(=O)CCCCCCCN(CCO)CCCCCCCC(=O)OC(CCCCCC)CCCCCCCC] |
| I 33 | 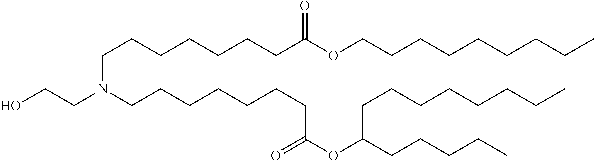 [SMILES: CCCCCCCCCOC(=O)CCCCCCCN(CCO)CCCCCCCC(=O)OC(CCCCC)CCCCCCCC] |
| I 34 | 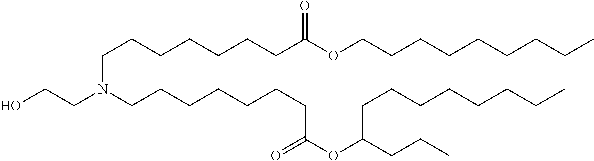 [SMILES: CCCCCCCCCOC(=O)CCCCCCCN(CCO)CCCCCCCC(=O)OC(CCC)CCCCCCCC] |
| I 35 | 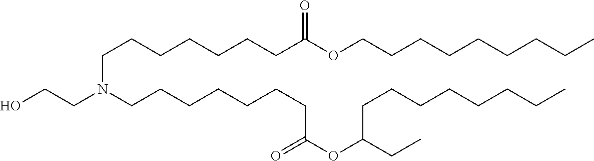 [SMILES: CCCCCCCCCOC(=O)CCCCCCCN(CCO)CCCCCCCC(=O)OC(CC)CCCCCCCC] |
| I 36 | 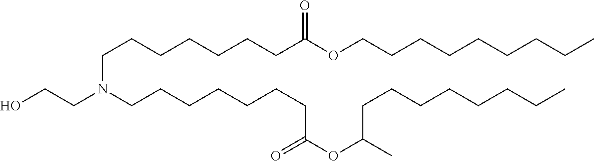 [SMILES: CCCCCCCCCOC(=O)CCCCCCCN(CCO)CCCCCCCC(=O)OC(C)CCCCCCCC] |
| I 37 | 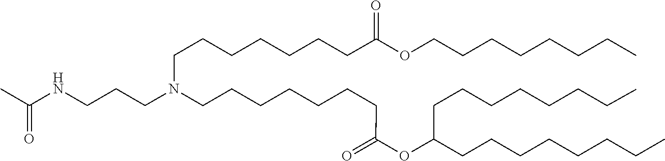 [SMILES: CCCCCCCCOC(=O)CCCCCCCN(CCCCCCCC(=O)OC(CCCCCCCC)CCCCCCCC)CCCNC(C)=O] |
| I 38 | 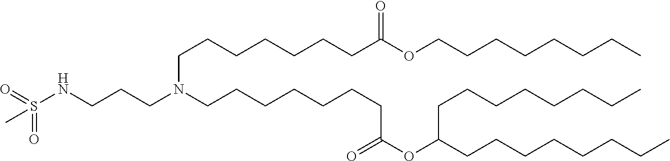 [SMILES: CCCCCCCCOC(=O)CCCCCCCN(CCCCCCCC(=O)OC(CCCCCCCC)CCCCCCCC)CCCNS(C)(=O)=O] |
| I 39 | 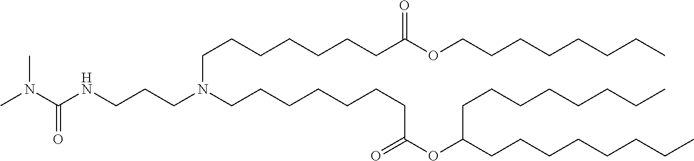 [SMILES: CCCCCCCCOC(=O)CCCCCCCN(CCCCCCCC(=O)OC(CCCCCCCC)CCCCCCCC)CCCNC(=O)N(C)C] |
| I 40 | 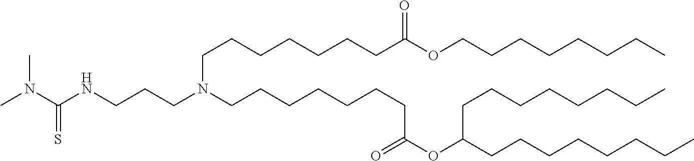 [SMILES: CCCCCCCCOC(=O)CCCCCCCN(CCCCCCCC(=O)OC(CCCCCCCC)CCCCCCCC)CCCNC(=S)N(C)C] |
| I 41 | 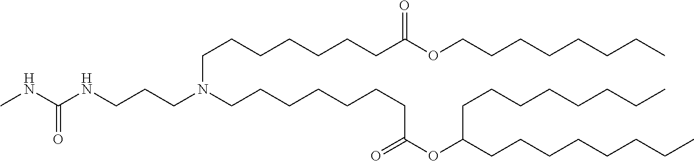 [SMILES: CCCCCCCCOC(=O)CCCCCCCN(CCCCCCCC(=O)OC(CCCCCCCC)CCCCCCCC)CCCNC(=O)NC] |
| I 42 | 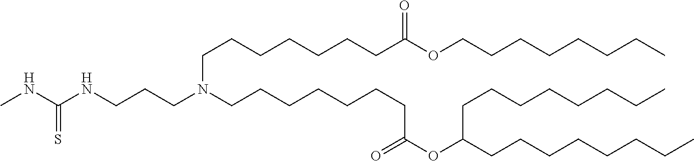 [SMILES: CCCCCCCCOC(=O)CCCCCCCN(CCCCCCCC(=O)OC(CCCCCCCC)CCCCCCCC)CCCNC(=S)NC] |
| I 43 | 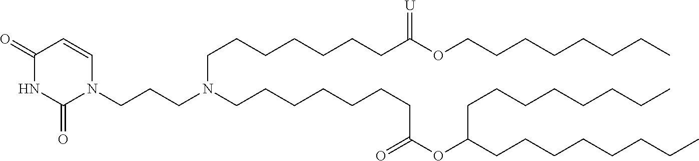 |
| I 44 | 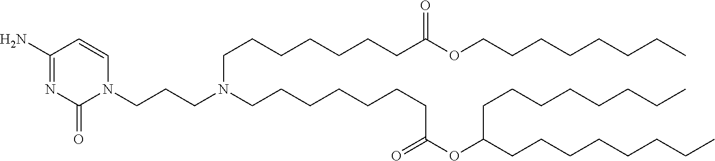 [SMILES: CCCCCCCCOC(=O)CCCCCCCN(CCCCCCCC(=O)OC(CCCCCCCC)CCCCCCCC)CCCn1ccc(N)nc1=O] |
| I 45 | 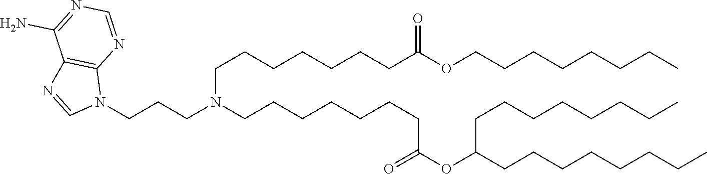 [SMILES: CCCCCCCCOC(=O)CCCCCCCN(CCCCCCCC(=O)OC(CCCCCCCC)CCCCCCCC)CCCn1cnc2c(N)ncnc21] |
| I 46 | 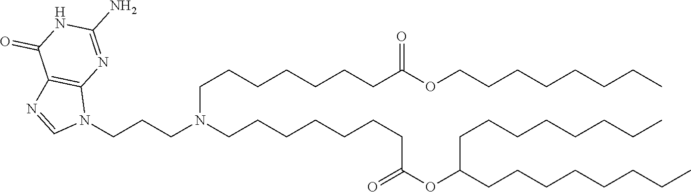 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I 47 | 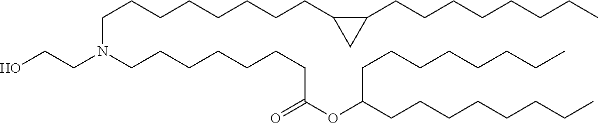 [SMILES: CCCCCCCCC(CCCCCCCC)OC(=O)CCCCCCCN(CCO)CCCCCCCCC1CC1CCCCCCCC] |
| I 48 | 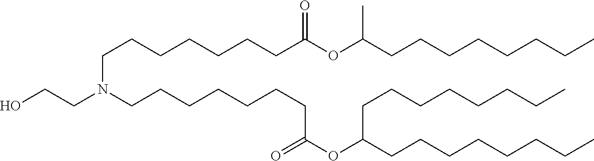 [SMILES: CCCCCCCCC(C)OC(=O)CCCCCCCN(CCO)CCCCCCCC(=O)OC(CCCCCCCC)CCCCCCCC] |
| I 49 | 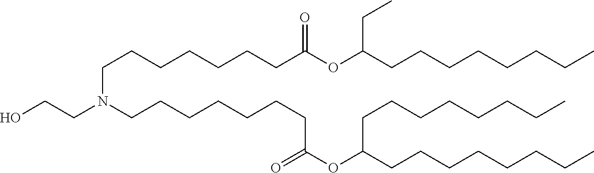 [SMILES: CCCCCCCCC(CC)OC(=O)CCCCCCCN(CCO)CCCCCCCC(=O)OC(CCCCCCCC)CCCCCCCC] |
| I 50 | 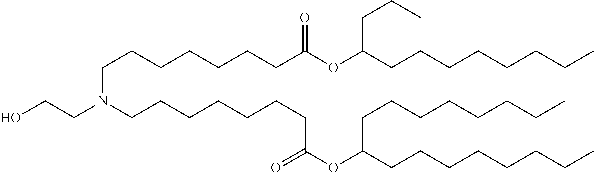 [SMILES: CCCCCCCCC(CCC)OC(=O)CCCCCCCN(CCO)CCCCCCCC(=O)OC(CCCCCCCC)CCCCCCCC] |
| I 51 | 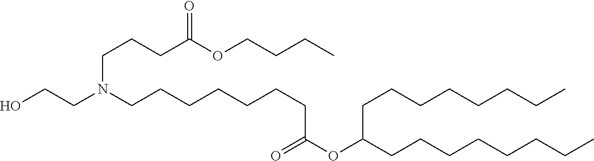 [SMILES: CCCCCCCCC(CCCCCCCC)OC(=O)CCCCCCCN(CCO)CCCC(=O)OCCCC] |
| I 52 | 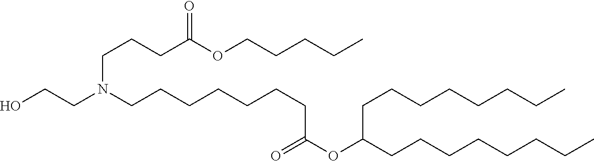 [SMILES: CCCCCCCCC(CCCCCCCC)OC(=O)CCCCCCCN(CCO)CCCC(=O)OCCCCC] |
| I 53 | 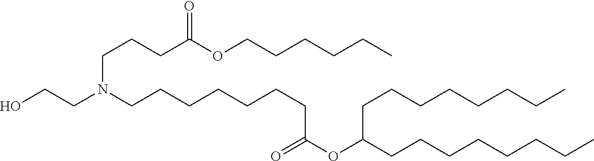 [SMILES: CCCCCCCCC(CCCCCCCC)OC(=O)CCCCCCCN(CCO)CCCC(=O)OCCCCCC] |
| I 54 | 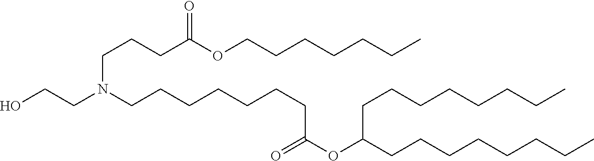 [SMILES: CCCCCCCCC(CCCCCCCC)OC(=O)CCCCCCCN(CCO)CCCC(=O)OCCCCCCC] |
| I 55 | 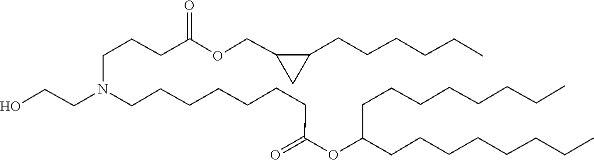 [SMILES: CCCCCCCCC(CCCCCCCC)OC(=O)CCCCCCCN(CCO)CCCC(=O)OCC1CC1CCCCCC] |
| I 56 | 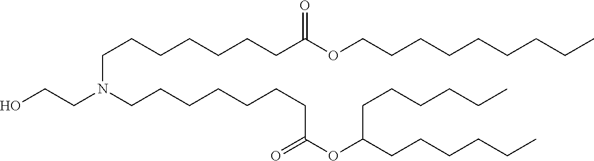 [SMILES: CCCCCCCCCOC(=O)CCCCCCCN(CCO)CCCCCCCC(=O)OC(CCCCCC)CCCCCC] |
| I 57 | 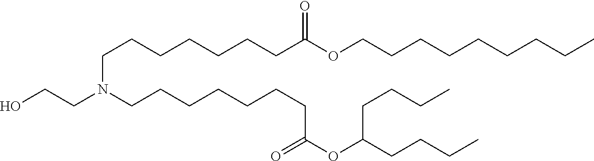 [SMILES: CCCCCCCCCOC(=O)CCCCCCCN(CCO)CCCCCCCC(=O)OC(CCCC)CCCC] |
| I 58 | 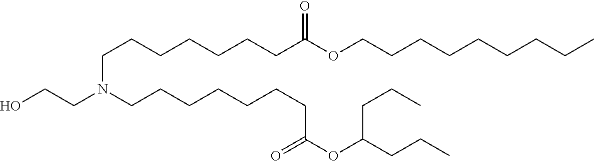 [SMILES: CCCCCCCCCOC(=O)CCCCCCCN(CCO)CCCCCCCC(=O)OC(CCC)CCC] |
| I 59 | 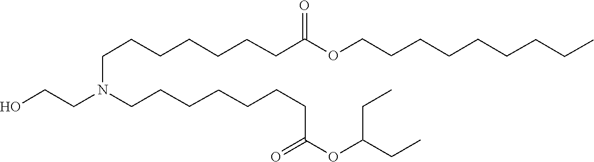 [SMILES: CCCCCCCCCOC(=O)CCCCCCCN(CCO)CCCCCCCC(=O)OC(CC)CC] |
| I 60 | 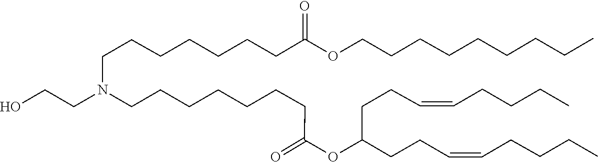 [SMILES: CCCC/C=C\CCC(CC/C=C\CCCC)OC(=O)CCCCCCCN(CCO)CCCCCCCC(=O)OCCCCCCCCC] |
| I 61 | 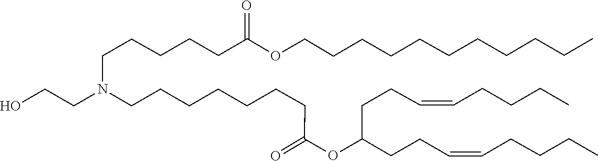 [SMILES: CCCC/C=C\CCC(CC/C=C\CCCC)OC(=O)CCCCCCCN(CCO)CCCCCC(=O)OCCCCCCCCCCC] |

In further embodiments, the compound of Formula (I I) is selected from the group consisting of:

| Cpd | Structure |
|---|---|
| I 62 | 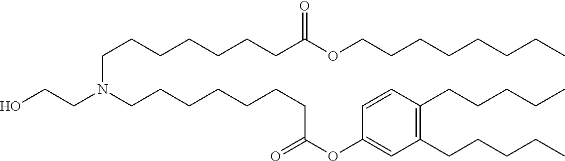 [SMILES: CCCCCCCCOC(=O)CCCCCCCN(CCO)CCCCCCCC(=O)Oc1ccc(CCCCC)c(CCCCC)c1] |
| I 63 | 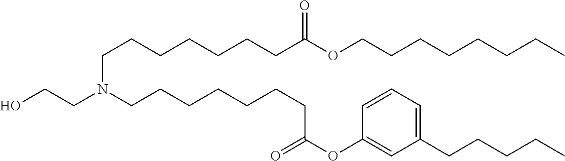 [SMILES: CCCCCCCCOC(=O)CCCCCCCN(CCO)CCCCCCCC(=O)Oc1cccc(CCCCC)c1] |
| I 64 | 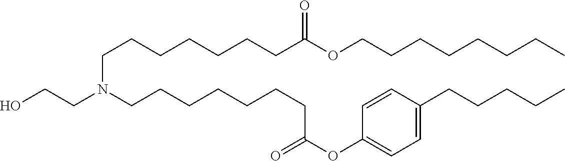 [SMILES: CCCCCCCCOC(=O)CCCCCCCN(CCO)CCCCCCCC(=O)Oc1ccc(CCCCC)cc1] |

In some embodiments, the compound of Formula (I I) or Formula (I IV) is selected from the group consisting of:

| Cpd | Structure |
|---|---|
| I 65 | 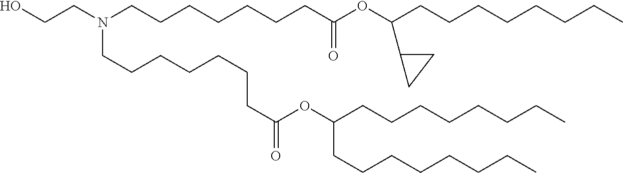 [SMILES: CCCCCCCCC(CCCCCCCC)OC(=O)CCCCCCCN(CCO)CCCCCCCC(=O)OC(CCCCCCCC)C1CC1] |
| I 66 | 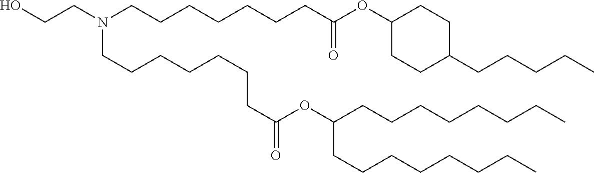 [SMILES: CCCCCCCCC(CCCCCCCC)OC(=O)CCCCCCCN(CCO)CCCCCCCC(=O)OC1CCC(CCCCC)CC1] |
| I 67 | 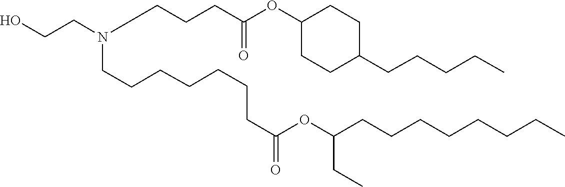 [SMILES: CCCCCCCCC(CC)OC(=O)CCCCCCCN(CCO)CCCC(=O)OC1CCC(CCCCC)CC1] |
| I 68 | 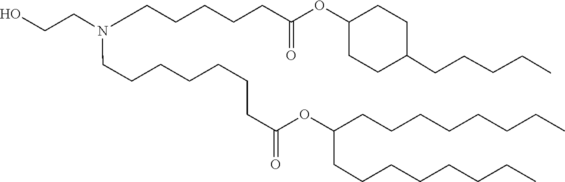 [SMILES: CCCCCCCCC(CCCCCCCC)OC(=O)CCCCCCCN(CCO)CCCCCC(=O)OC1CCC(CCCCC)CC1] |
| I 69 | 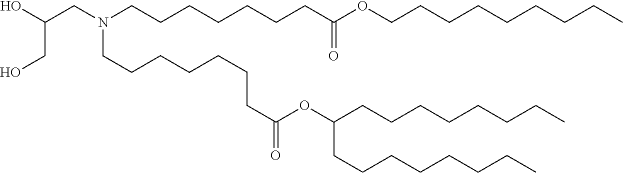 [SMILES: CCCCCCCCCOC(=O)CCCCCCCN(CCCCCCCC(=O)OC(CCCCCCCC)CCCCCCCC)CC(O)CO] |
| I 70 | 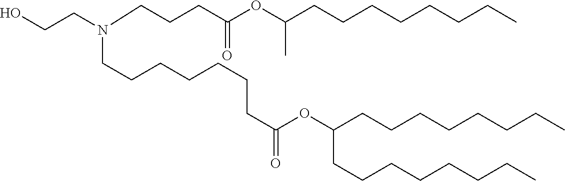 [SMILES: CCCCCCCCC(C)OC(=O)CCCN(CCO)CCCCCCCC(=O)OC(CCCCCCCC)CCCCCCCC] |
| I 71 | 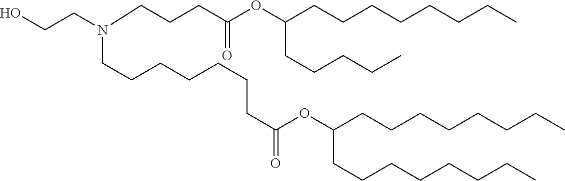 [SMILES: CCCCCCCCC(CCCCCCCC)OC(=O)CCCCCCCN(CCO)CCCC(=O)OC(CCCCC)CCCCCCCC] |
| I 72 | 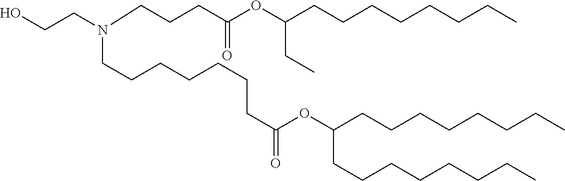 [SMILES: CCCCCCCCC(CC)OC(=O)CCCN(CCO)CCCCCCCC(=O)OC(CCCCCCCC)CCCCCCCC] |
| I 73 | 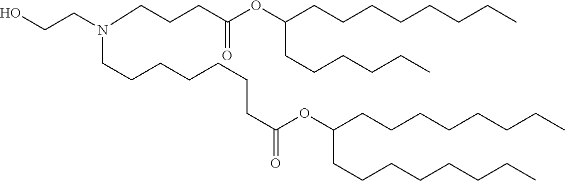 [SMILES: CCCCCCCCC(CCCCCCCC)OC(=O)CCCCCCCN(CCO)CCCC(=O)OC(CCCCCC)CCCCCCCC] |
| I 74 | 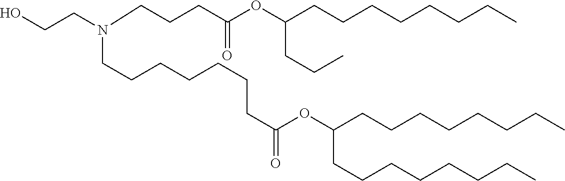 [SMILES: CCCCCCCCC(CCC)OC(=O)CCCN(CCO)CCCCCCCC(=O)OC(CCCCCCCC)CCCCCCCC] |
| I 75 | 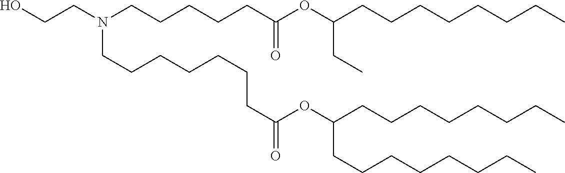 [SMILES: CCCCCCCCC(CC)OC(=O)CCCCCN(CCO)CCCCCCCC(=O)OC(CCCCCCCC)CCCCCCCC] |
| I 76 | 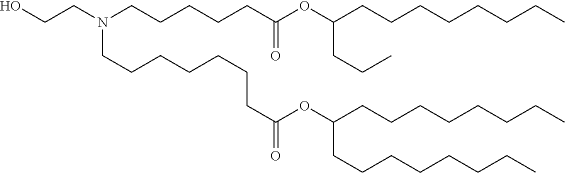 [SMILES: CCCCCCCCC(CCC)OC(=O)CCCCCN(CCO)CCCCCCCC(=O)OC(CCCCCCCC)CCCCCCCC] |
| I 77 | 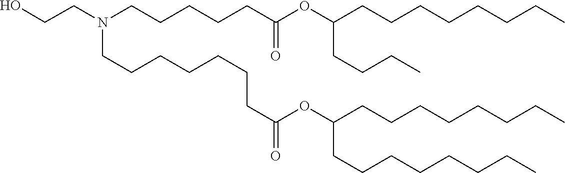 [SMILES: CCCCCCCCC(CCCC)OC(=O)CCCCCN(CCO)CCCCCCCC(=O)OC(CCCCCCCC)CCCCCCCC] |
| I 78 | 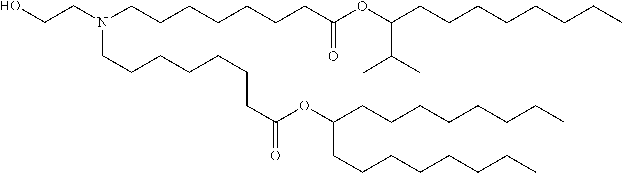 [SMILES: CCCCCCCCC(CCCCCCCC)OC(=O)CCCCCCCN(CCO)CCCCCCCC(=O)OC(CCCCCCCC)C(C)C] |
| I 79 | 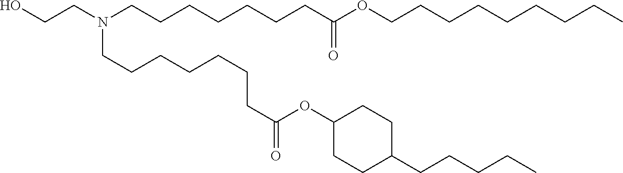 [SMILES: CCCCCCCCCOC(=O)CCCCCCCN(CCO)CCCCCCCC(=O)OC1CCC(CCCCC)CC1] |
| I 80 | 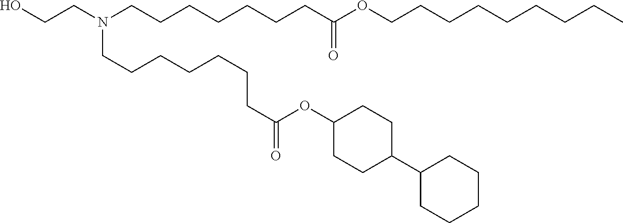 [SMILES: CCCCCCCCCOC(=O)CCCCCCCN(CCO)CCCCCCCC(=O)OC1CCC(C2CCCCC2)CC1] |
| I 81 | 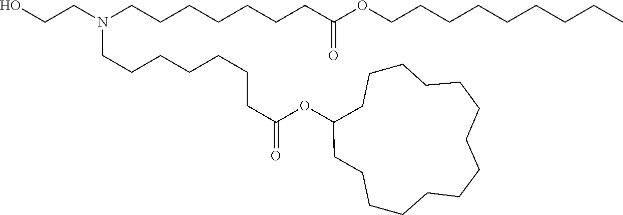 [SMILES: CCCCCCCCCOC(=O)CCCCCCCN(CCO)CCCCCCCC(=O)OC1CCCCCCCCCCCCCC1] |
| I 82 | 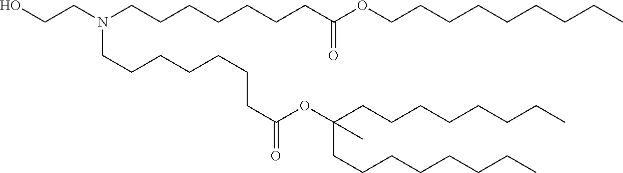 [SMILES: CCCCCCCCCOC(=O)CCCCCCCN(CCO)CCCCCCCC(=O)OC(C)(CCCCCCCC)CCCCCCCC] |
| I 83 | 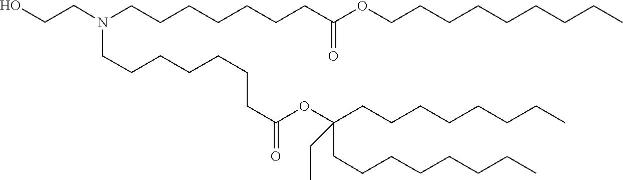 [SMILES: CCCCCCCCCOC(=O)CCCCCCCN(CCO)CCCCCCCC(=O)OC(CC)(CCCCCCCC)CCCCCCCC] |
| I 84 | 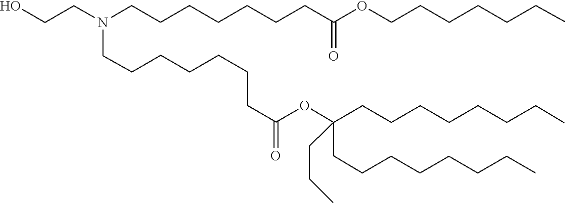 [SMILES: CCCCCCCCC(CCC)(CCCCCCCC)OC(=O)CCCCCCCN(CCO)CCCCCCCC(=O)OCCCCCCC] |
| I 85 | 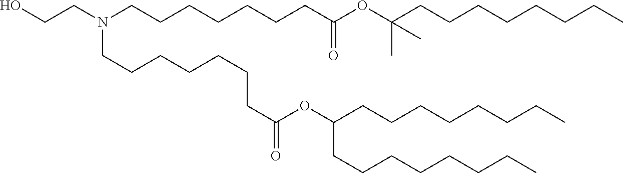 [SMILES: CCCCCCCCC(CCCCCCCC)OC(=O)CCCCCCCN(CCO)CCCCCCCC(=O)OC(C)(C)CCCCCCCC] |
| I 86 | 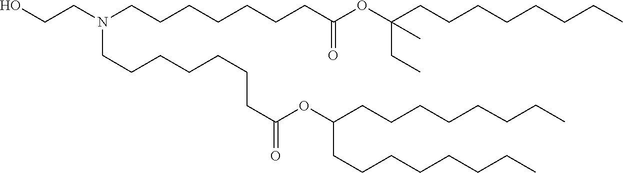 [SMILES: CCCCCCCCC(CCCCCCCC)OC(=O)CCCCCCCN(CCO)CCCCCCCC(=O)OC(C)(CC)CCCCCCCC] |
| I 87 | 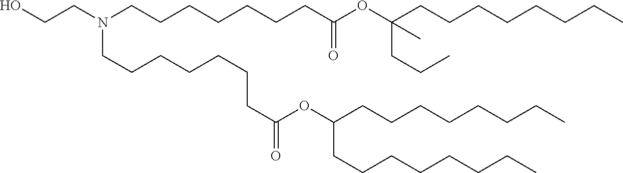 [SMILES: CCCCCCCCC(CCCCCCCC)OC(=O)CCCCCCCN(CCO)CCCCCCCC(=O)OC(C)(CCC)CCCCCCCC] |
| I 88 | 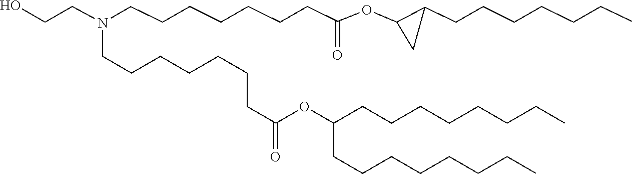 [SMILES: CCCCCCCCC(CCCCCCCC)OC(=O)CCCCCCCN(CCO)CCCCCCCC(=O)OC1CC1CCCCCCC] |
| I 89 | 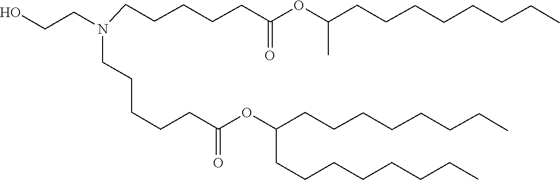 [SMILES: CCCCCCCCC(C)OC(=O)CCCCCN(CCO)CCCCCC(=O)OC(CCCCCCCC)CCCCCCCC] |
| I 90 | 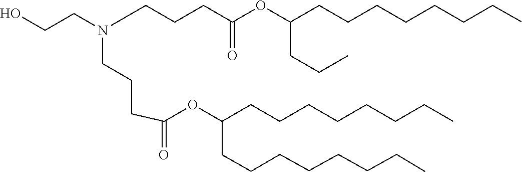 [SMILES: CCCCCCCCC(CCC)OC(=O)CCCN(CCO)CCCC(=O)OC(CCCCCCCC)CCCCCCCC] |
| I 91 | 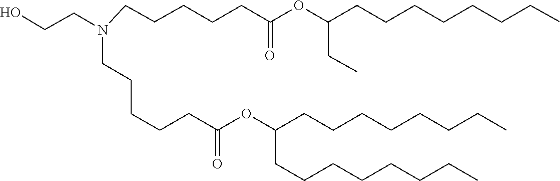 [SMILES: CCCCCCCCC(CC)OC(=O)CCCCCN(CCO)CCCCCC(=O)OC(CCCCCCCC)CCCCCCCC] |
| I 92 | 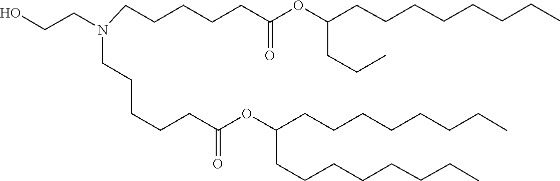 [SMILES: CCCCCCCCC(CCC)OC(=O)CCCCCN(CCO)CCCCCC(=O)OC(CCCCCCCC)CCCCCCCC] |
| I 93 | 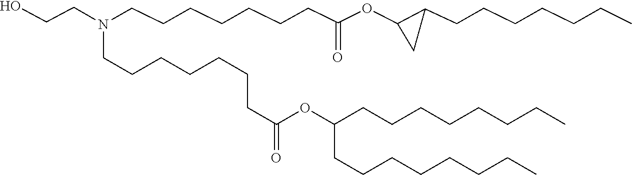 [SMILES: CCCCCCCCC(CCCCCCCC)OC(=O)CCCCCCCN(CCO)CCCCCCCC(=O)OC1CC1CCCCCCC] |
| I 94 | 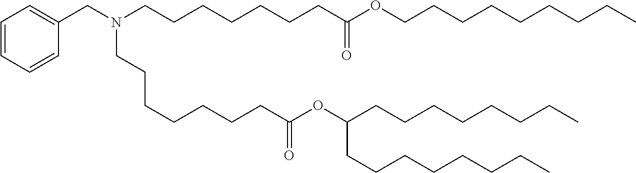 [SMILES: CCCCCCCCCOC(=O)CCCCCCCN(CCCCCCCC(=O)OC(CCCCCCCC)CCCCCCCC)Cc1ccccc1] |
| I 95 | 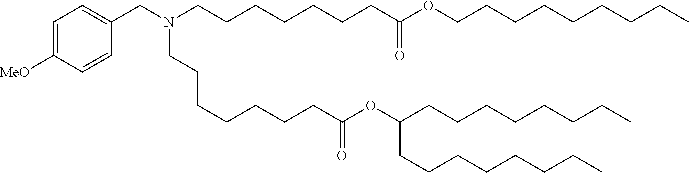 [SMILES: CCCCCCCCCOC(=O)CCCCCCCN(CCCCCCCC(=O)OC(CCCCCCCC)CCCCCCCC)Cc1ccc(OC)cc1] |
| I 96 | 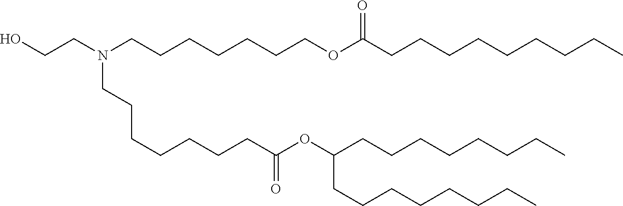 [SMILES: CCCCCCCCCC(=O)OCCCCCCCN(CCO)CCCCCCCC(=O)OC(CCCCCCCC)CCCCCCCC] |
| I 97 | 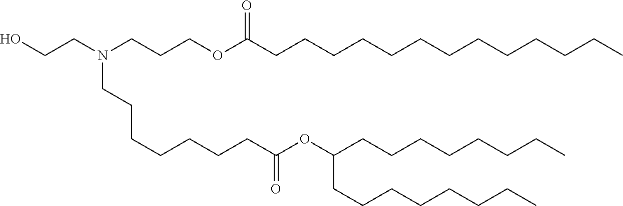 [SMILES: CCCCCCCCCCCCCC(=O)OCCCN(CCO)CCCCCCCC(=O)OC(CCCCCCCC)CCCCCCCC] |
| I 98 | 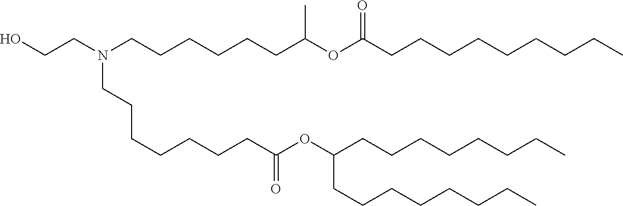 [SMILES: CCCCCCCCCC(=O)OC(C)CCCCCCN(CCO)CCCCCCCC(=O)OC(CCCCCCCC)CCCCCCCC] |
| I 99 | 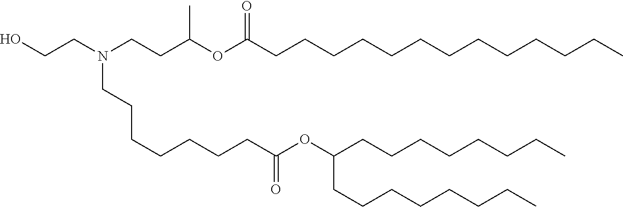 [SMILES: CCCCCCCCCCCCCC(=O)OC(C)CCN(CCO)CCCCCCCC(=O)OC(CCCCCCCC)CCCCCCCC] |
| I 100 | 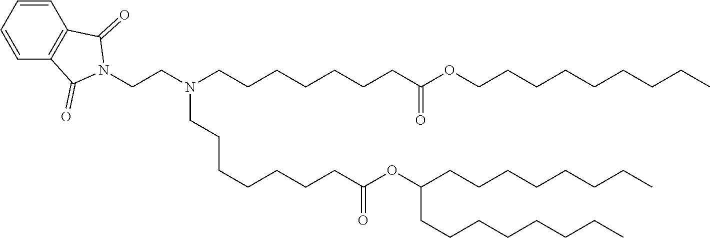 [SMILES: CCCCCCCCCOC(=O)CCCCCCCN(CCCCCCCC(=O)OC(CCCCCCCC)CCCCCCCC)CCN1C(=O)c2ccccc2C1=O] |
| I 101 | 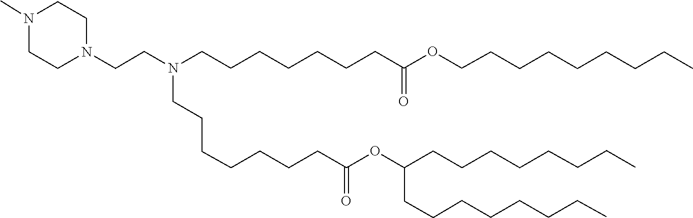 [SMILES: CCCCCCCCCOC(=O)CCCCCCCN(CCCCCCCC(=O)OC(CCCCCCCC)CCCCCCCC)CCN1CCN(C)CC1] |
| I 102 | 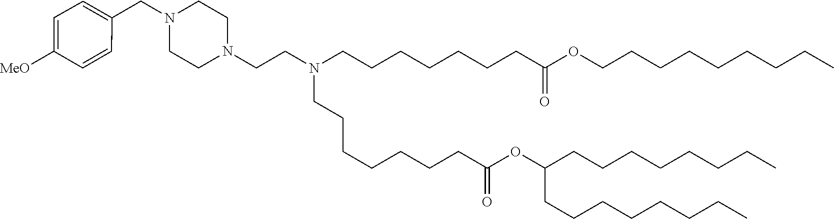 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I 103 | 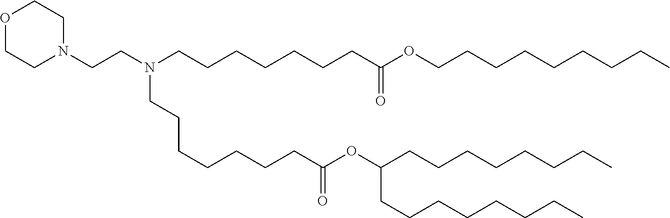 [SMILES: CCCCCCCCCOC(=O)CCCCCCCN(CCCCCCCC(=O)OC(CCCCCCCC)CCCCCCCC)CCN1CCOCC1] |
| I 104 | 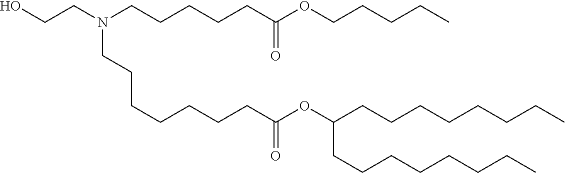 [SMILES: CCCCCCCCC(CCCCCCCC)OC(=O)CCCCCCCN(CCO)CCCCCC(=O)OCCCCC] |
| I 105 | 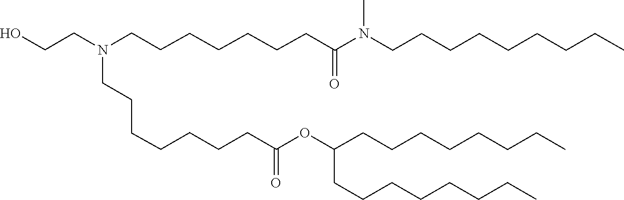 [SMILES: CCCCCCCCCN(C)C(=O)CCCCCCCN(CCO)CCCCCCCC(=O)OC(CCCCCCCC)CCCCCCCC] |
| I 106 | 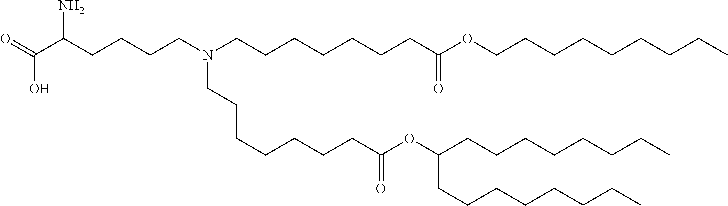 [SMILES: CCCCCCCCCOC(=O)CCCCCCCN(CCCCCCCC(=O)OC(CCCCCCCC)CCCCCCCC)CCCCC(N)C(=O)O] |
| I 107 | 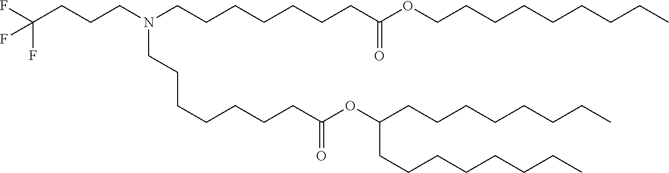 [SMILES: CCCCCCCCCOC(=O)CCCCCCCN(CCCCCCCC(=O)OC(CCCCCCCC)CCCCCCCC)CCCC(F)(F)F] |
| I 108 | 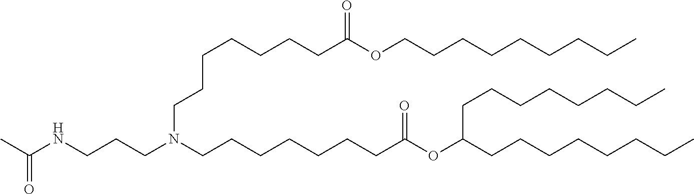 [SMILES: CCCCCCCCCOC(=O)CCCCCCCN(CCCCCCCC(=O)OC(CCCCCCCC)CCCCCCCC)CCCNC(C)=O] |
| I 109 | 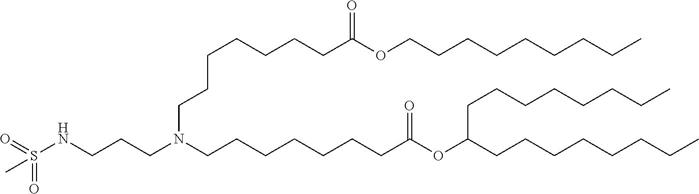 [SMILES: CCCCCCCCCOC(=O)CCCCCCCN(CCCCCCCC(=O)OC(CCCCCCCC)CCCCCCCC)CCCNS(C)(=O)=O] |
| I 110 | 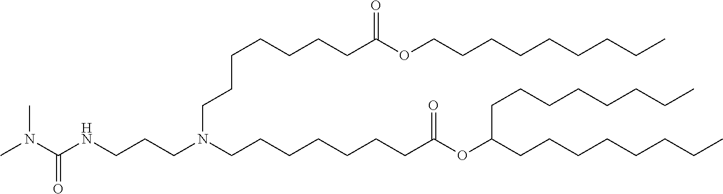 [SMILES: CCCCCCCCCOC(=O)CCCCCCCN(CCCCCCCC(=O)OC(CCCCCCCC)CCCCCCCC)CCCNC(=O)N(C)C] |
| I 111 | 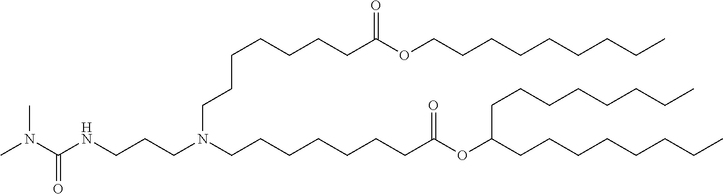 [SMILES: CCCCCCCCCOC(=O)CCCCCCCN(CCCCCCCC(=O)OC(CCCCCCCC)CCCCCCCC)CCCNC(=O)N(C)C] |
| I 112 | 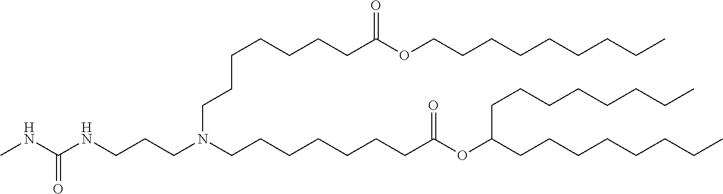 [SMILES: CCCCCCCCCOC(=O)CCCCCCCN(CCCCCCCC(=O)OC(CCCCCCCC)CCCCCCCC)CCCNC(=O)NC] |
| I 113 | 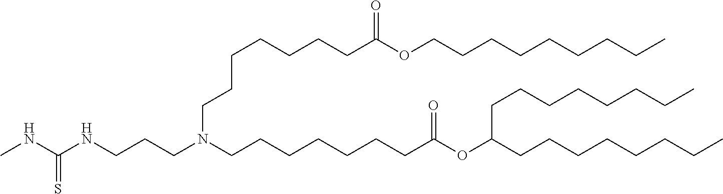 [SMILES: CCCCCCCCCOC(=O)CCCCCCCN(CCCCCCCC(=O)OC(CCCCCCCC)CCCCCCCC)CCCNC(=S)NC] |
| I 114 | 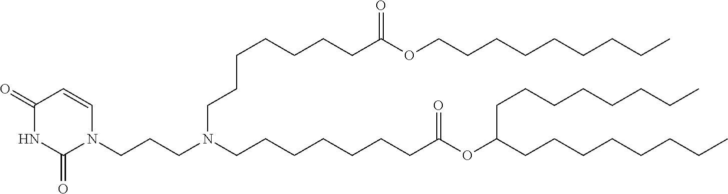 [SMILES: CCCCCCCCCOC(=O)CCCCCCCN(CCCCCCCC(=O)OC(CCCCCCCC)CCCCCCCC)CCCn1ccc(=O)[nH]c1=O] |
| I 115 | 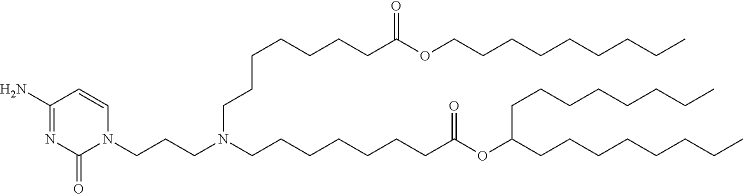 [SMILES: CCCCCCCCCOC(=O)CCCCCCCN(CCCCCCCC(=O)OC(CCCCCCCC)CCCCCCCC)CCCn1ccc(N)nc1=O] |
| I 116 | 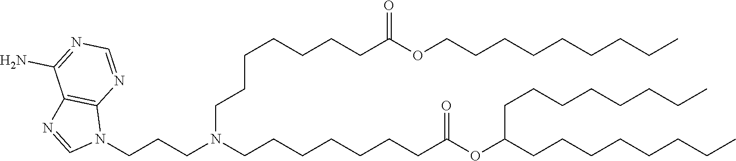 [SMILES: CCCCCCCCCOC(=O)CCCCCCCN(CCCCCCCC(=O)OC(CCCCCCCC)CCCCCCCC)CCCn1cnc2c(N)ncnc21] |
| I 117 | 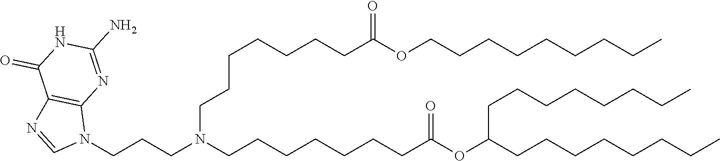 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I 118 | 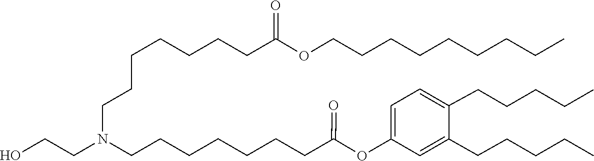 [SMILES: CCCCCCCCCOC(=O)CCCCCCCN(CCO)CCCCCCCC(=O)Oc1ccc(CCCCC)c(CCCCC)c1] |
| I 119 | 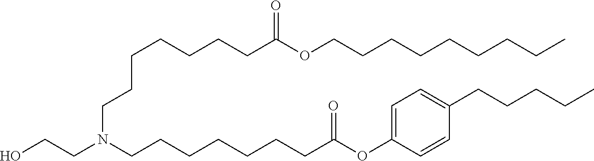 [SMILES: CCCCCCCCCOC(=O)CCCCCCCN(CCO)CCCCCCCC(=O)Oc1ccc(CCCCC)cc1] |
| I 120 | 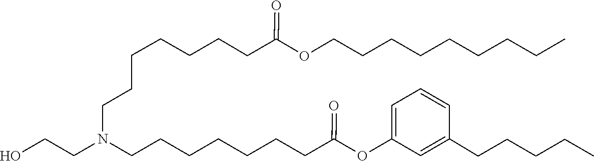 [SMILES: CCCCCCCCCOC(=O)CCCCCCCN(CCO)CCCCCCCC(=O)Oc1cccc(CCCCC)c1] |
| I 121 | 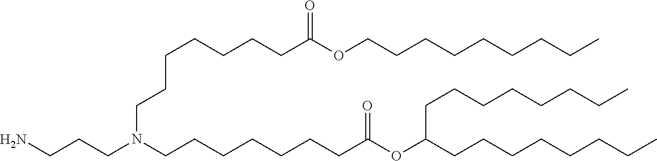 [SMILES: CCCCCCCCCOC(=O)CCCCCCCN(CCCN)CCCCCCCC(=O)OC(CCCCCCCC)CCCCCCCC] |
| I 122 | 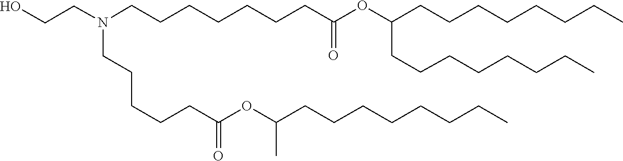 [SMILES: CCCCCCCCC(C)OC(=O)CCCCCN(CCO)CCCCCCCC(=O)OC(CCCCCCCC)CCCCCCCC] |
| I 123 | 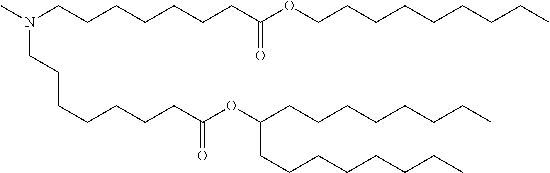 [SMILES: CCCCCCCCCOC(=O)CCCCCCCN(C)CCCCCCCC(=O)OC(CCCCCCCC)CCCCCCCC] |
| I 124 | 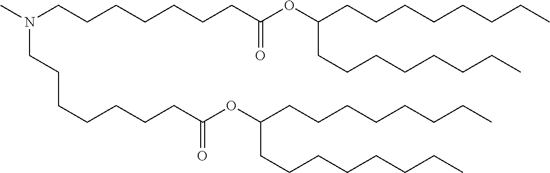 [SMILES: CCCCCCCCC(CCCCCCCC)OC(=O)CCCCCCCN(C)CCCCCCCC(=O)OC(CCCCCCCC)CCCCCCCC] |
| I 125 | 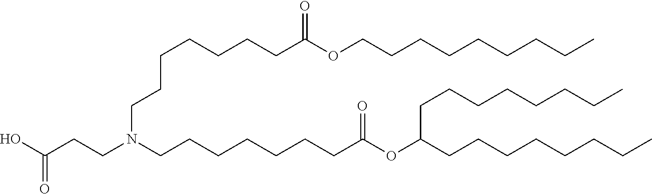 [SMILES: CCCCCCCCCOC(=O)CCCCCCCN(CCCCCCCC(=O)OC(CCCCCCCC)CCCCCCCC)CCC(=O)O] |
| I 126 | 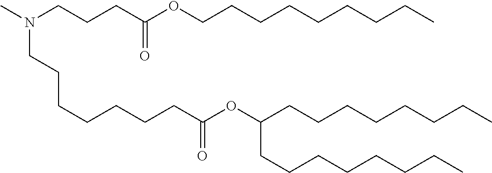 [SMILES: CCCCCCCCCOC(=O)CCCN(C)CCCCCCCC(=O)OC(CCCCCCCC)CCCCCCCC] |
| I 127 | 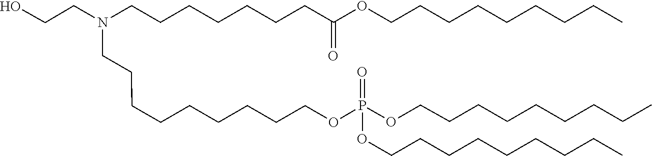 [SMILES: CCCCCCCCCOC(=O)CCCCCCCN(CCO)CCCCCCCCCOP(=O)(OCCCCCCCCC)OCCCCCCCCC] |
| I 128 | 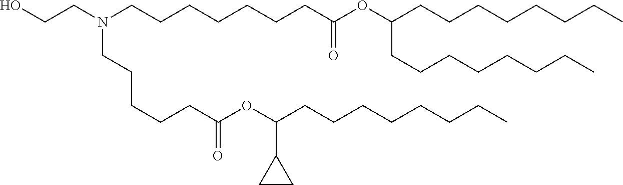 [SMILES: CCCCCCCCC(CCCCCCCC)OC(=O)CCCCCCCN(CCO)CCCCCC(=O)OC(CCCCCCCC)C1CC1] |
| I 129 | 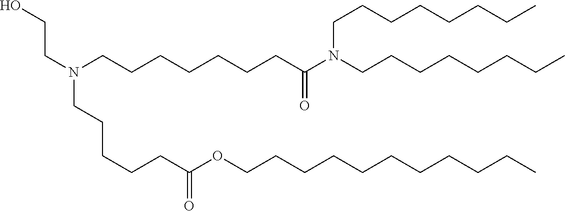 [SMILES: CCCCCCCCCCCOC(=O)CCCCCN(CCO)CCCCCCCC(=O)N(CCCCCCCC)CCCCCCCC] |
| I 130 | 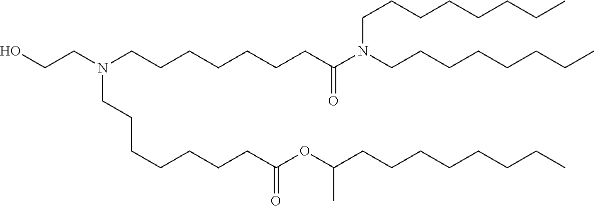 [SMILES: CCCCCCCCC(C)OC(=O)CCCCCCCN(CCO)CCCCCCCC(=O)N(CCCCCCCC)CCCCCCCC] |
| I 131 | 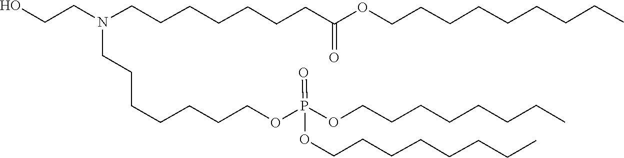 [SMILES: CCCCCCCCCOC(=O)CCCCCCCN(CCO)CCCCCCCOP(=O)(OCCCCCCCC)OCCCCCCCC] |
| I 132 | 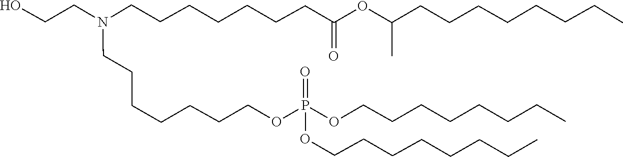 [SMILES: CCCCCCCCOP(=O)(OCCCCCCCC)OCCCCCCCN(CCO)CCCCCCCC(=O)OC(C)CCCCCCCC] |
| I 133 | 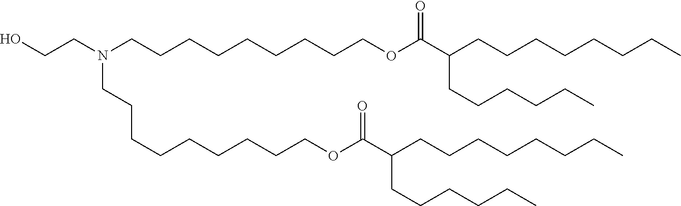 [SMILES: CCCCCCCCC(CCCCCC)C(=O)OCCCCCCCCCN(CCO)CCCCCCCCCOC(=O)C(CCCCCC)CCCCCCCC] |
| I 134 | 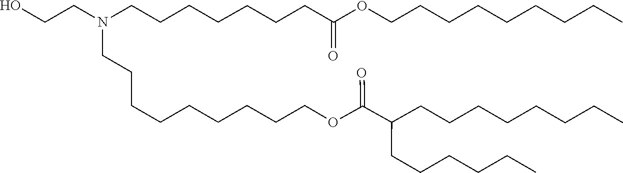 [SMILES: CCCCCCCCCOC(=O)CCCCCCCN(CCO)CCCCCCCCCOC(=O)C(CCCCCC)CCCCCCCC] |
| I 135 | 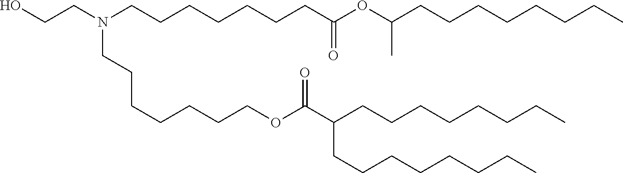 [SMILES: CCCCCCCCC(C)OC(=O)CCCCCCCN(CCO)CCCCCCCOC(=O)C(CCCCCCCC)CCCCCCCC] |
| I 136 | 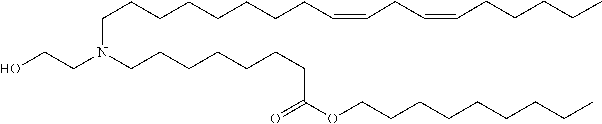 [SMILES: CCCCC/C=C\C/C=C\CCCCCCCCN(CCO)CCCCCCCC(=O)OCCCCCCCCC] |
| I 137 | 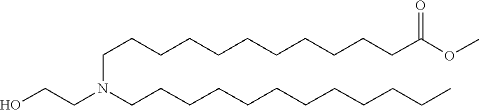 [SMILES: CCCCCCCCCCCCN(CCO)CCCCCCCCCCCC(=O)OC] |
| I 138 | 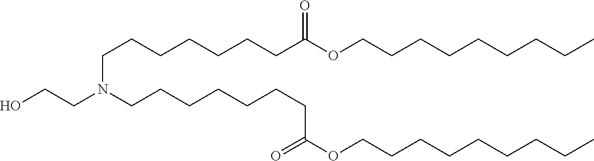 [SMILES: CCCCCCCCCOC(=O)CCCCCCCN(CCO)CCCCCCCC(=O)OCCCCCCCCC] |
| I 139 | 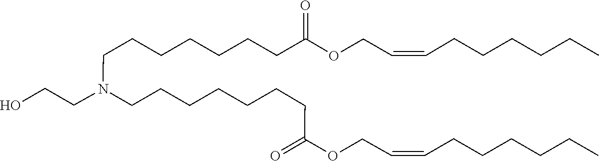 [SMILES: CCCCCC/C=C\COC(=O)CCCCCCCN(CCO)CCCCCCCC(=O)OC/C=C\CCCCCC] |
| I 140 | 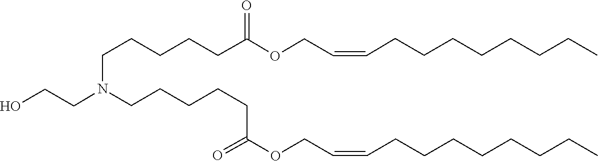 [SMILES: CCCCCCCC/C=C\COC(=O)CCCCCN(CCO)CCCCCC(=O)OC/C=C\CCCCCCCC] |
| I 141 | 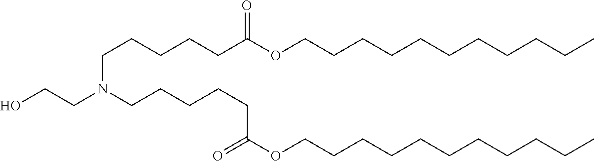 [SMILES: CCCCCCCCCCCOC(=O)CCCCCN(CCO)CCCCCC(=O)OCCCCCCCCCCC] |
| I 142 | 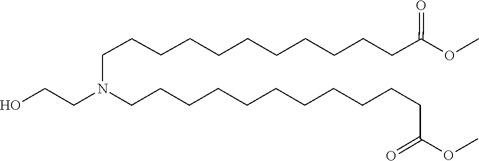 [SMILES: COC(=O)CCCCCCCCCCCN(CCO)CCCCCCCCCCCC(=O)OC] |
| I 143 | 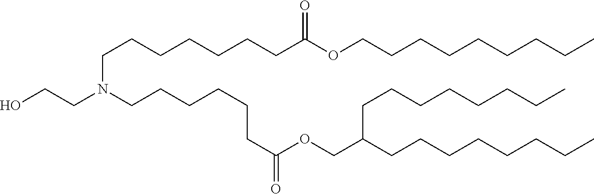 [SMILES: CCCCCCCCCOC(=O)CCCCCCCN(CCO)CCCCCCC(=O)OCC(CCCCCCCC)CCCCCCCC] |
| I 144 | 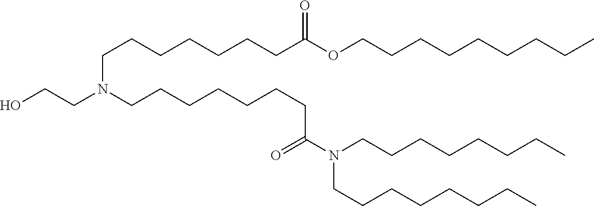 [SMILES: CCCCCCCCCOC(=O)CCCCCCCN(CCO)CCCCCCCC(=O)N(CCCCCCCC)CCCCCCCC] |
| I 145 | 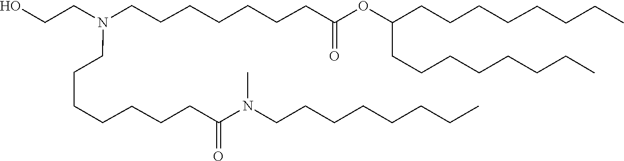 [SMILES: CCCCCCCCC(CCCCCCCC)OC(=O)CCCCCCCN(CCO)CCCCCCCC(=O)N(C)CCCCCCCC] |
| I 146 | 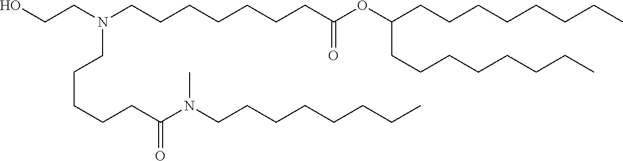 [SMILES: CCCCCCCCC(CCCCCCCC)OC(=O)CCCCCCCN(CCO)CCCCCC(=O)N(C)CCCCCCCC] |
| I 147 | 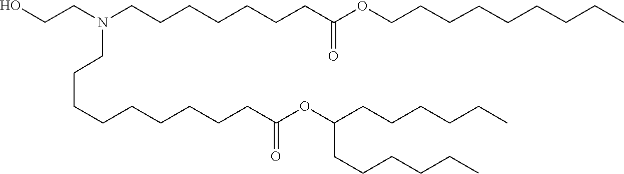 [SMILES: CCCCCCCCCOC(=O)CCCCCCCN(CCO)CCCCCCCCCC(=O)OC(CCCCCC)CCCCCC] |
| I 148 | 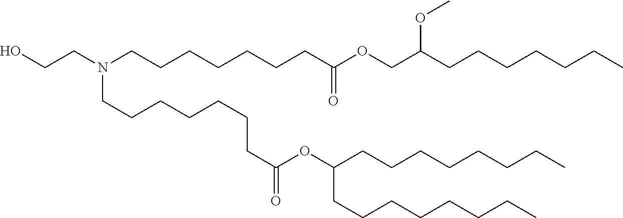 [SMILES: CCCCCCCCC(CCCCCCCC)OC(=O)CCCCCCCN(CCO)CCCCCCCC(=O)OCC(CCCCCCC)OC] |
| I 149 | 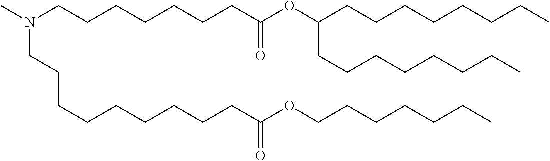 [SMILES: CCCCCCCCC(CCCCCCCC)OC(=O)CCCCCCCN(C)CCCCCCCCCC(=O)OCCCCCCC] |
| I 150 | 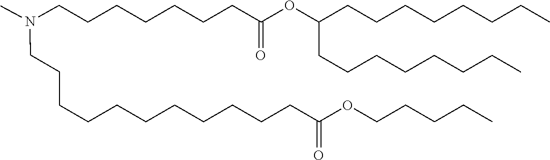 [SMILES: CCCCCCCCC(CCCCCCCC)OC(=O)CCCCCCCN(C)CCCCCCCCCCCC(=O)OCCCCC] |
| I 151 | 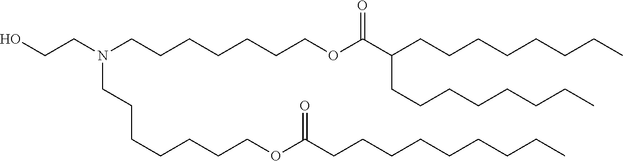 [SMILES: CCCCCCCCCC(=O)OCCCCCCCN(CCO)CCCCCCCOC(=O)C(CCCCCCCC)CCCCCCCC] |
| I 152 | 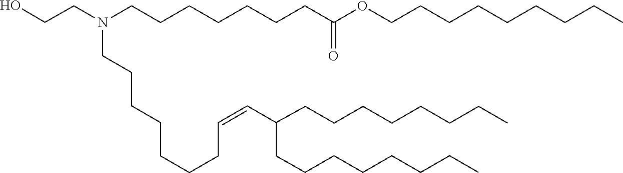 [SMILES: CCCCCCCCCOC(=O)CCCCCCCN(CCO)CCCCCCC/C=C\C(CCCCCCCC)CCCCCCCC] |
| I 153 | 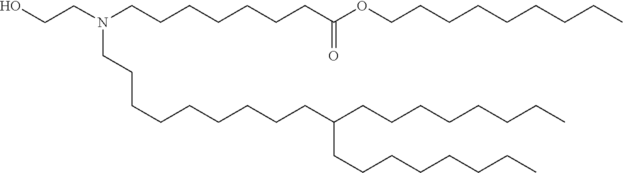 [SMILES: CCCCCCCCCOC(=O)CCCCCCCN(CCO)CCCCCCCCCC(CCCCCCCC)CCCCCCCC] |
| I 154 | 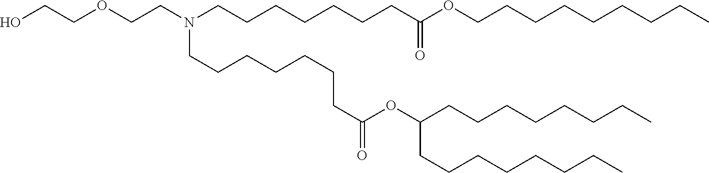 [SMILES: CCCCCCCCCOC(=O)CCCCCCCN(CCCCCCCC(=O)OC(CCCCCCCC)CCCCCCCC)CCOCCO] |
| I 155 | 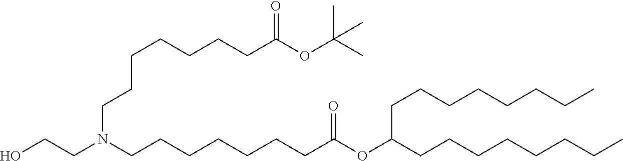 [SMILES: CCCCCCCCC(CCCCCCCC)OC(=O)CCCCCCCN(CCO)CCCCCCCC(=O)OC(C)(C)C] |
| I 156 | 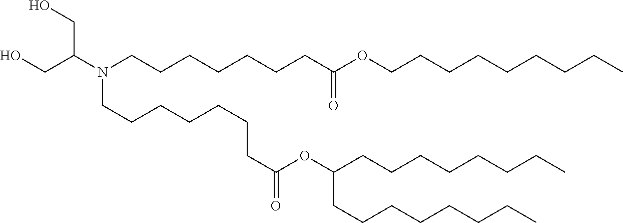 [SMILES: CCCCCCCCCOC(=O)CCCCCCCN(CCCCCCCC(=O)OC(CCCCCCCC)CCCCCCCC)C(CO)CO] |
| I 157 | 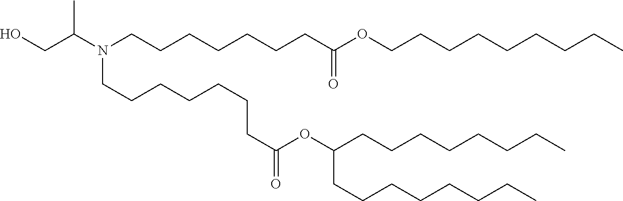 [SMILES: CCCCCCCCCOC(=O)CCCCCCCN(CCCCCCCC(=O)OC(CCCCCCCC)CCCCCCCC)C(C)CO] |
| I 158 | 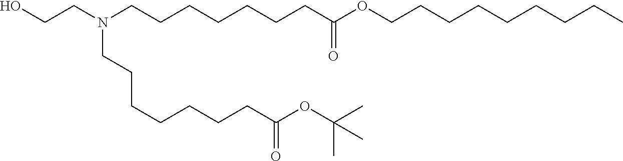 [SMILES: CCCCCCCCCOC(=O)CCCCCCCN(CCO)CCCCCCCC(=O)OC(C)(C)C] |
| I 159 | 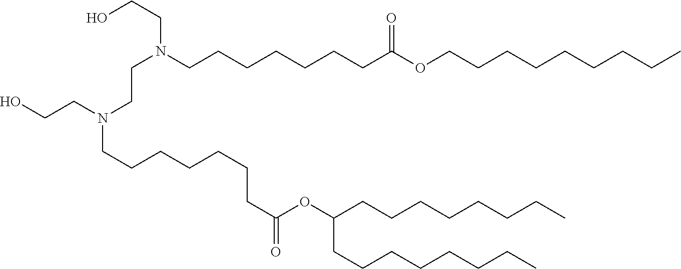 [SMILES: CCCCCCCCCOC(=O)CCCCCCCN(CCO)CCN(CCO)CCCCCCCC(=O)OC(CCCCCCCC)CCCCCCCC] |
| I 160 | 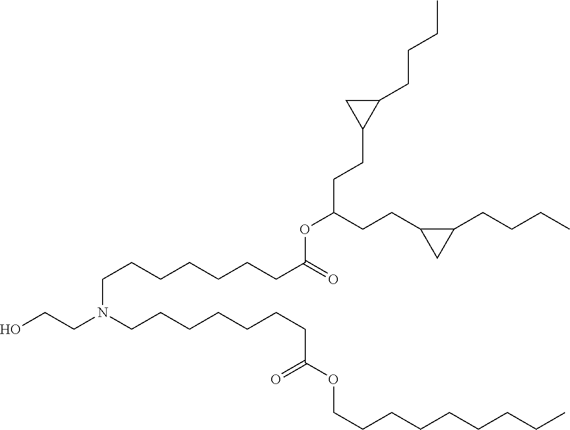 [SMILES: CCCCCCCCCOC(=O)CCCCCCCN(CCO)CCCCCCCC(=O)OC(CCC1CC1CCCC)CCC1CC1CCCC] |
| I 161 | 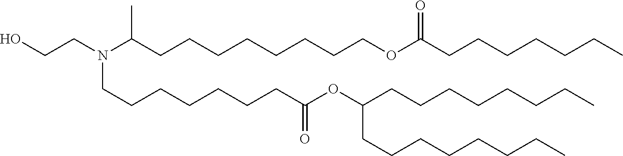 [SMILES: CCCCCCCCC(CCCCCCCC)OC(=O)CCCCCCCN(CCO)C(C)CCCCCCCCOC(=O)CCCCCCC] |
| I 162 | 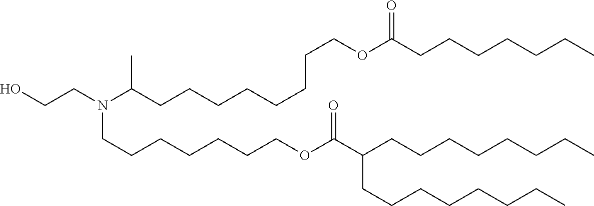 [SMILES: CCCCCCCCC(CCCCCCCC)C(=O)OCCCCCCCN(CCO)C(C)CCCCCCCCOC(=O)CCCCCCC] |
| I 163 | 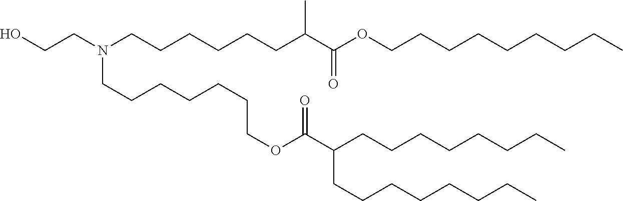 [SMILES: CCCCCCCCCOC(=O)C(C)CCCCCCN(CCO)CCCCCCCOC(=O)C(CCCCCCCC)CCCCCCCC] |
| I 164 | 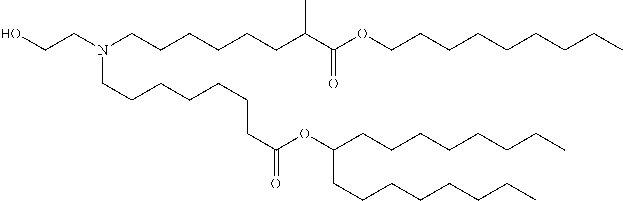 [SMILES: CCCCCCCCCOC(=O)C(C)CCCCCCN(CCO)CCCCCCCC(=O)OC(CCCCCCCC)CCCCCCCC] |
| I 165 | 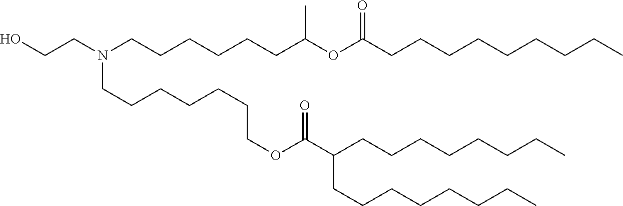 [SMILES: CCCCCCCCCC(=O)OC(C)CCCCCCN(CCO)CCCCCCCOC(=O)C(CCCCCCCC)CCCCCCCC] |
| I 166 | 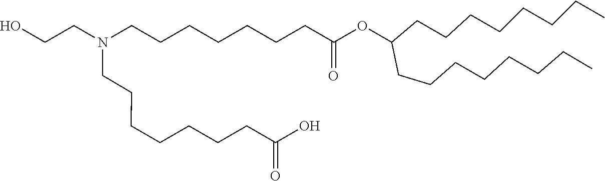 [SMILES: CCCCCCCCC(CCCCCCCC)OC(=O)CCCCCCCN(CCO)CCCCCCCC(=O)O] |
| I 167 | 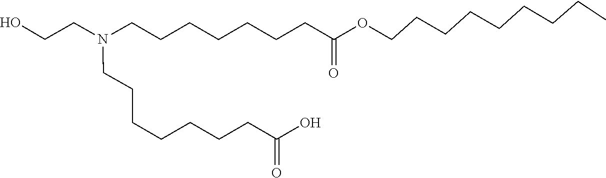 [SMILES: CCCCCCCCCOC(=O)CCCCCCCN(CCO)CCCCCCCC(=O)O] |
| I 168 | 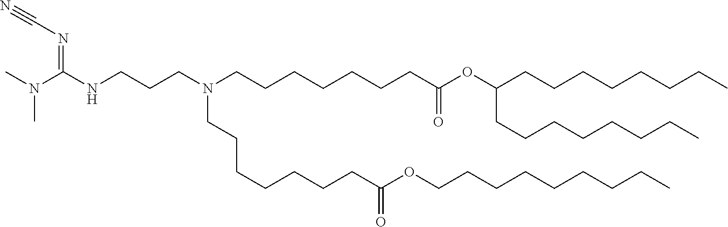 [SMILES: CCCCCCCCCOC(=O)CCCCCCCN(CCCCCCCC(=O)OC(CCCCCCCC)CCCCCCCC)CCCN/C(=N/C#N)N(C)C] |
| I 169 | 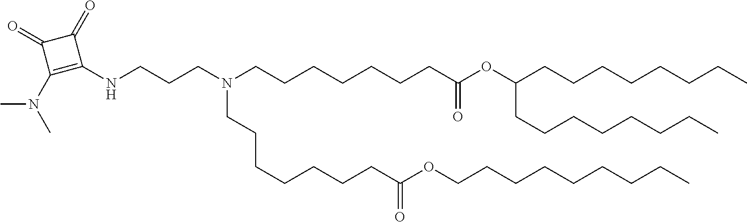 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I 170 | 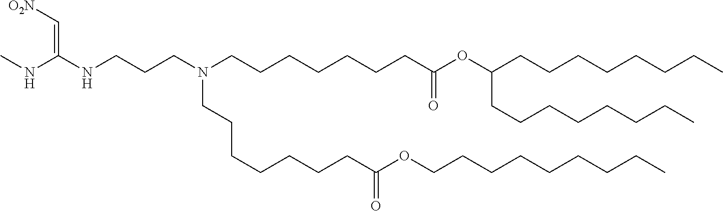 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I 171 | 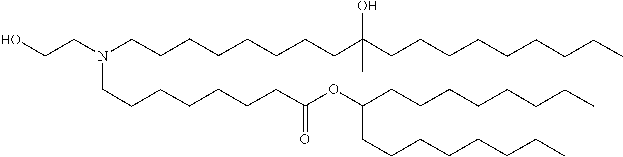 [SMILES: CCCCCCCCCC(C)(O)CCCCCCCCN(CCO)CCCCCCCC(=O)OC(CCCCCCCC)CCCCCCCC] |
| I 172 | 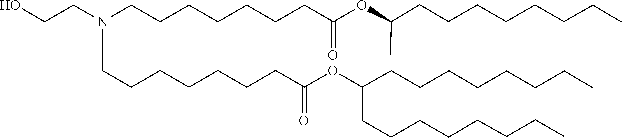 [SMILES: CCCCCCCCC(CCCCCCCC)OC(=O)CCCCCCCN(CCO)CCCCCCCC(=O)O[C@H](C)CCCCCCCC] |
| I 173 | 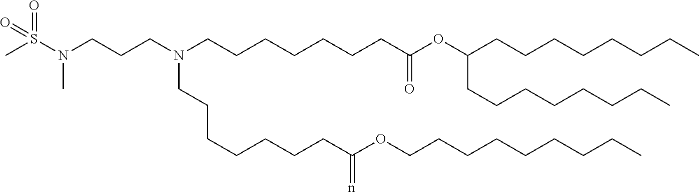 |
| I 174 | 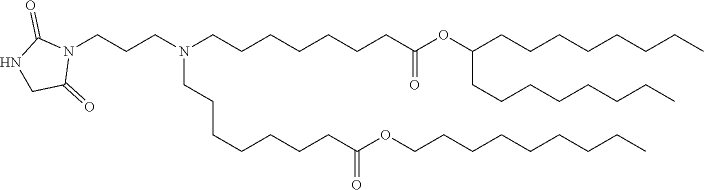 [SMILES: CCCCCCCCCOC(=O)CCCCCCCN(CCCCCCCC(=O)OC(CCCCCCCC)CCCCCCCC)CCCN1C(=O)CNC1=O] |
| I 175 | 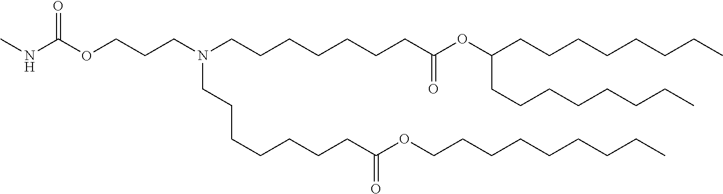 [SMILES: CCCCCCCCCOC(=O)CCCCCCCN(CCCCCCCC(=O)OC(CCCCCCCC)CCCCCCCC)CCCOC(=O)NC] |
| I 176 | 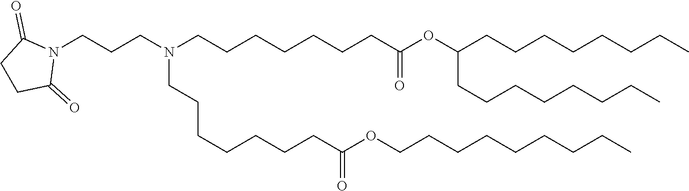 [SMILES: CCCCCCCCCOC(=O)CCCCCCCN(CCCCCCCC(=O)OC(CCCCCCCC)CCCCCCCC)CCCN1C(=O)CCC1=O] |
| I 177 | 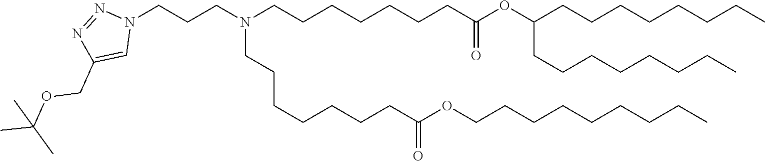 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I 178 | 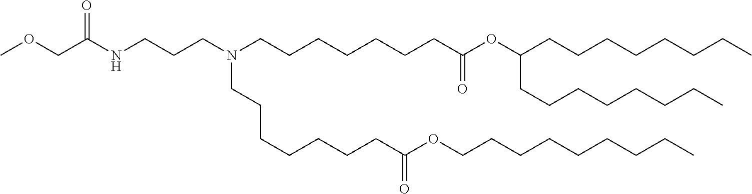 [SMILES: CCCCCCCCCOC(=O)CCCCCCCN(CCCCCCCC(=O)OC(CCCCCCCC)CCCCCCCC)CCCNC(=O)COC] |
| I 179 | 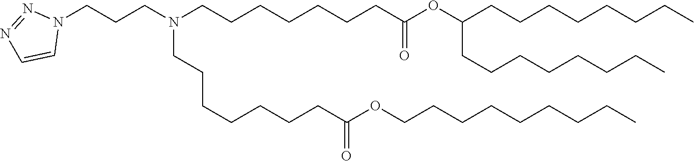 [SMILES: CCCCCCCCCOC(=O)CCCCCCCN(CCCCCCCC(=O)OC(CCCCCCCC)CCCCCCCC)CCCn1ccnn1] |
| I 180 | 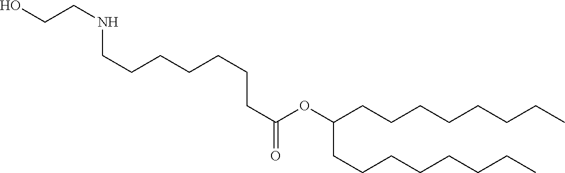 [SMILES: CCCCCCCCC(CCCCCCCC)OC(=O)CCCCCCCNCCO] |
| I 181 | 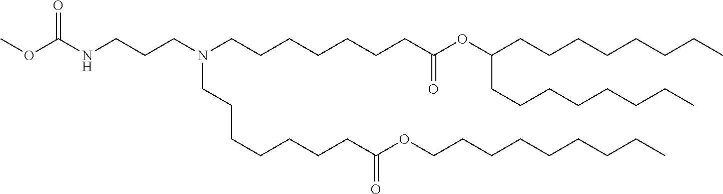 [SMILES: CCCCCCCCCOC(=O)CCCCCCCN(CCCCCCCC(=O)OC(CCCCCCCC)CCCCCCCC)CCCNC(=O)OC] |
| I 182 | 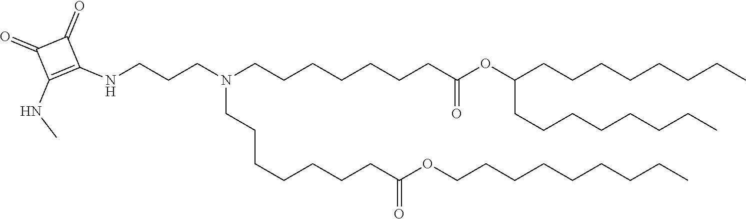 [SMILES: CCCCCCCCCOC(=O)CCCCCCCN(CCCCCCCC(=O)OC(CCCCCCCC)CCCCCCCC)CCCNc1c(NC)c(=O)c1=O] |
| I 183 | 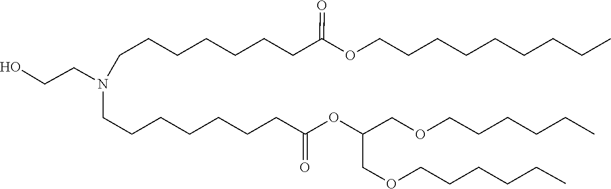 [SMILES: CCCCCCCCCOC(=O)CCCCCCCN(CCO)CCCCCCCC(=O)OC(COCCCCCC)COCCCCCC] |
| I 184 | 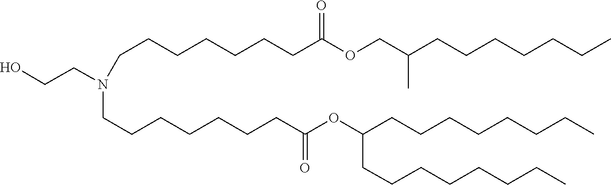 [SMILES: CCCCCCCCC(CCCCCCCC)OC(=O)CCCCCCCN(CCO)CCCCCCCC(=O)OCC(C)CCCCCCC] |
| I 185 | 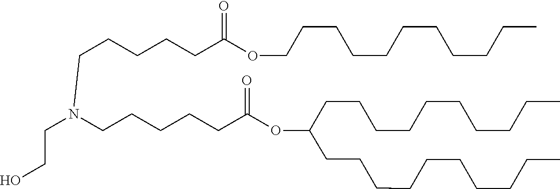 [SMILES: CCCCCCCCCCCOC(=O)CCCCCN(CCO)CCCCCC(=O)OC(CCCCCCCCCC)CCCCCCCCCC] |
| I 186 | 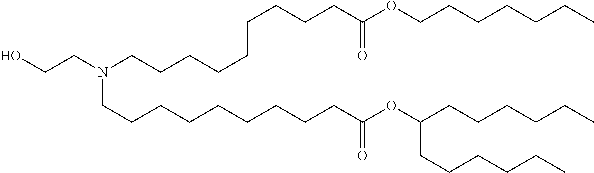 [SMILES: CCCCCCCOC(=O)CCCCCCCCCN(CCO)CCCCCCCCCC(=O)OC(CCCCCC)CCCCCC] |
| I 187 | 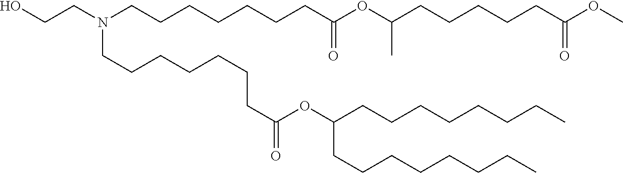 [SMILES: CCCCCCCCC(CCCCCCCC)OC(=O)CCCCCCCN(CCO)CCCCCCCC(=O)OC(C)CCCCCC(=O)OC] |
| I 188 | 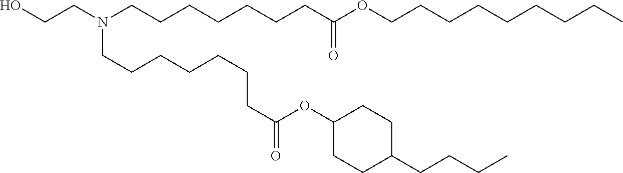 [SMILES: CCCCCCCCCOC(=O)CCCCCCCN(CCO)CCCCCCCC(=O)OC1CCC(CCCC)CC1] |
| I 189 | 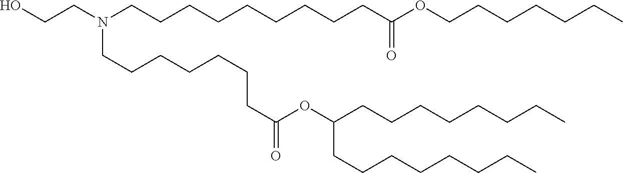 [SMILES: CCCCCCCCC(CCCCCCCC)OC(=O)CCCCCCCN(CCO)CCCCCCCCCC(=O)OCCCCCCC] |
| I 190 | 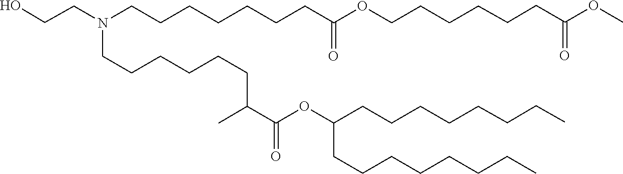 [SMILES: CCCCCCCCC(CCCCCCCC)OC(=O)C(C)CCCCCCN(CCO)CCCCCCCC(=O)OCCCCCCC(=O)OC] |
| I 191 | 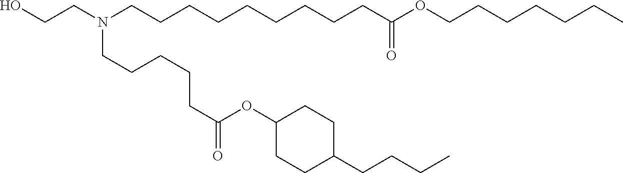 [SMILES: CCCCCCCOC(=O)CCCCCCCCCN(CCO)CCCCCC(=O)OC1CCC(CCCC)CC1] |
| I 192 | 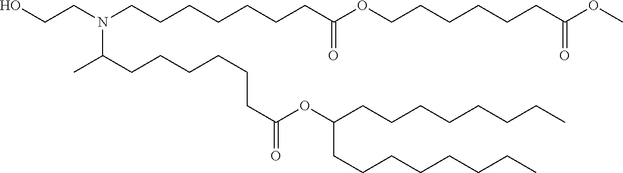 [SMILES: CCCCCCCCC(CCCCCCCC)OC(=O)CCCCCCC(C)N(CCO)CCCCCCCC(=O)OCCCCCCC(=O)OC] |
| I 193 | 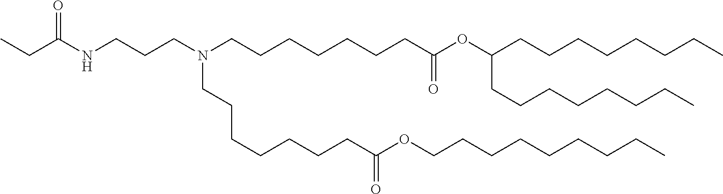 [SMILES: CCCCCCCCCOC(=O)CCCCCCCN(CCCCCCCC(=O)OC(CCCCCCCC)CCCCCCCC)CCCNC(=O)CC] |
| I 194 | 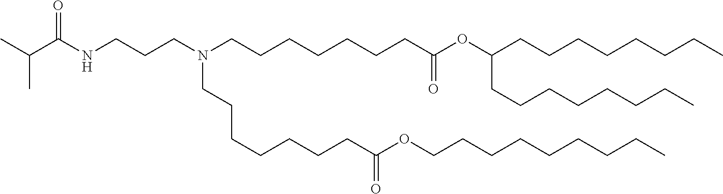 [SMILES: CCCCCCCCCOC(=O)CCCCCCCN(CCCCCCCC(=O)OC(CCCCCCCC)CCCCCCCC)CCCNC(=O)C(C)C] |
| I 195 | 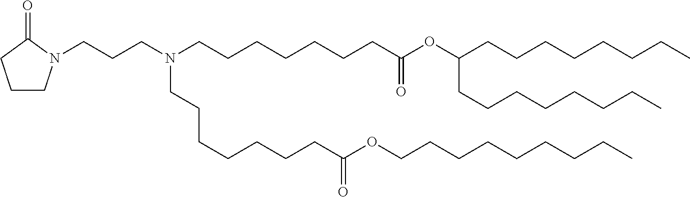 [SMILES: CCCCCCCCCOC(=O)CCCCCCCN(CCCCCCCC(=O)OC(CCCCCCCC)CCCCCCCC)CCCN1CCCC1=O] |
| I 196 | 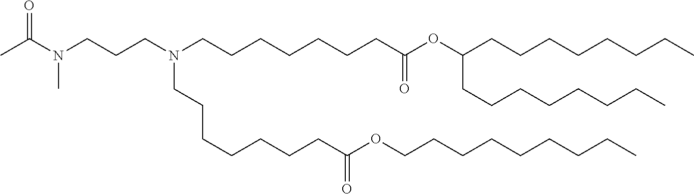 [SMILES: CCCCCCCCCOC(=O)CCCCCCCN(CCCCCCCC(=O)OC(CCCCCCCC)CCCCCCCC)CCCN(C)C(C)=O] |
| I 197 | 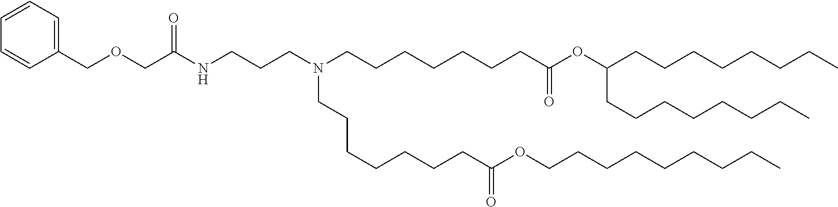 [SMILES: CCCCCCCCCOC(=O)CCCCCCCN(CCCCCCCC(=O)OC(CCCCCCCC)CCCCCCCC)CCCNC(=O)COCc1ccccc1] |
| I 198 | 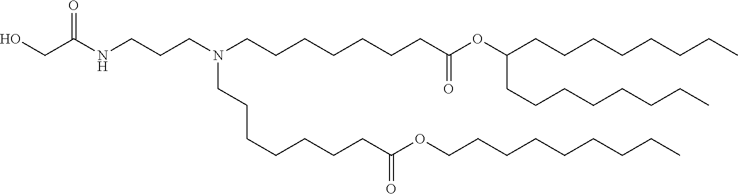 [SMILES: CCCCCCCCCOC(=O)CCCCCCCN(CCCCCCCC(=O)OC(CCCCCCCC)CCCCCCCC)CCCNC(=O)CO] |
| I 199 | 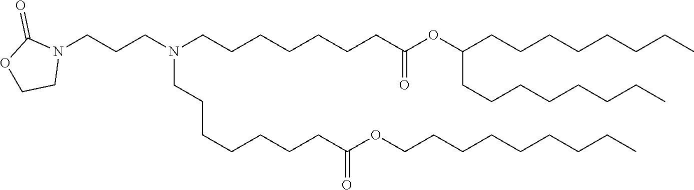 [SMILES: CCCCCCCCCOC(=O)CCCCCCCN(CCCCCCCC(=O)OC(CCCCCCCC)CCCCCCCC)CCCN1CCOC1=O] |
| I 200 | 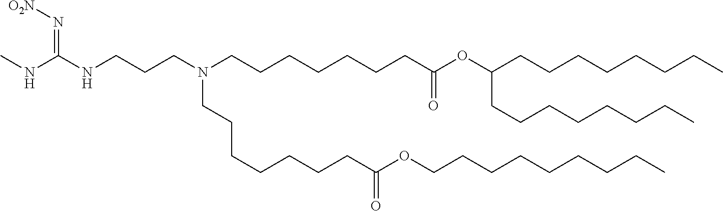 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I 201 | 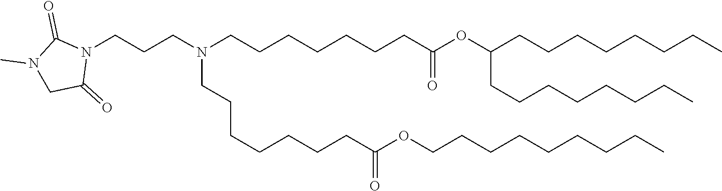 [SMILES: CCCCCCCCCOC(=O)CCCCCCCN(CCCCCCCC(=O)OC(CCCCCCCC)CCCCCCCC)CCCN1C(=O)CN(C)C1=O] |
| I 202 | 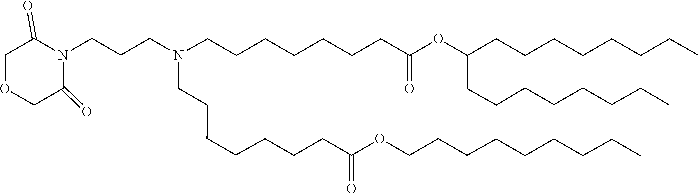 [SMILES: CCCCCCCCCOC(=O)CCCCCCCN(CCCCCCCC(=O)OC(CCCCCCCC)CCCCCCCC)CCCN1C(=O)COCC1=O] |
| I 203 | 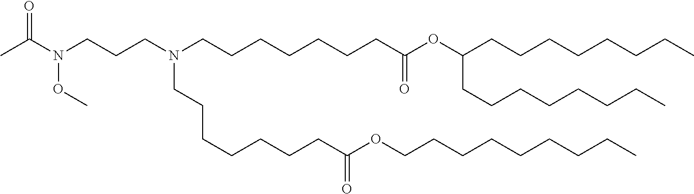 [SMILES: CCCCCCCCCOC(=O)CCCCCCCN(CCCCCCCC(=O)OC(CCCCCCCC)CCCCCCCC)CCCN(OC)C(C)=O] |
| I 204 | 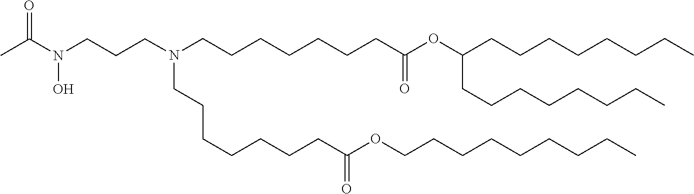 [SMILES: CCCCCCCCCOC(=O)CCCCCCCN(CCCCCCCC(=O)OC(CCCCCCCC)CCCCCCCC)CCCN(O)C(C)=O] |
| I 205 | 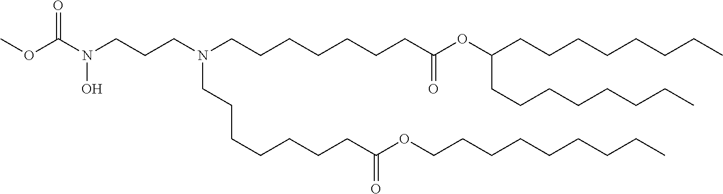 [SMILES: CCCCCCCCCOC(=O)CCCCCCCN(CCCCCCCC(=O)OC(CCCCCCCC)CCCCCCCC)CCCN(O)C(=O)OC] |
| I 206 | 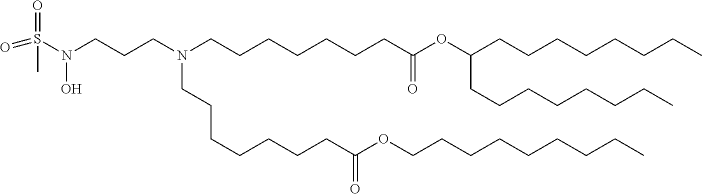 [SMILES: CCCCCCCCCOC(=O)CCCCCCCN(CCCCCCCC(=O)OC(CCCCCCCC)CCCCCCCC)CCCN(O)S(C)(=O)=O] |
| I 207 | 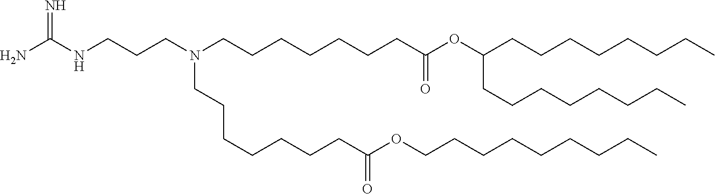 [SMILES: CCCCCCCCCOC(=O)CCCCCCCN(CCCCCCCC(=O)OC(CCCCCCCC)CCCCCCCC)CCCNC(=N)N] |
| I 208 | 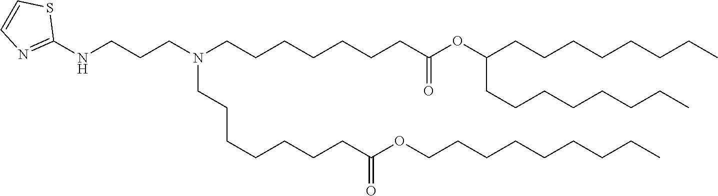 [SMILES: CCCCCCCCCOC(=O)CCCCCCCN(CCCCCCCC(=O)OC(CCCCCCCC)CCCCCCCC)CCCNc1nccs1] |
| I 209 | 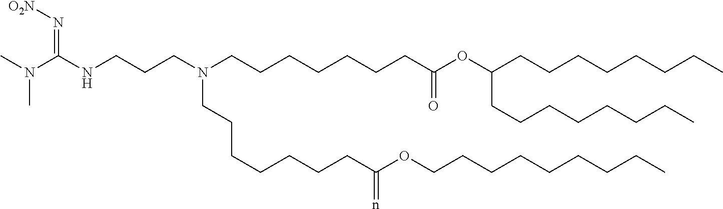 |
| I 210 | 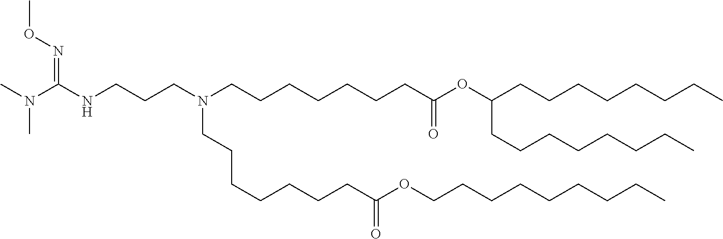 [SMILES: CCCCCCCCCOC(=O)CCCCCCCN(CCCCCCCC(=O)OC(CCCCCCCC)CCCCCCCC)CCCN/C(=N/OC)N(C)C] |
| I 211 | 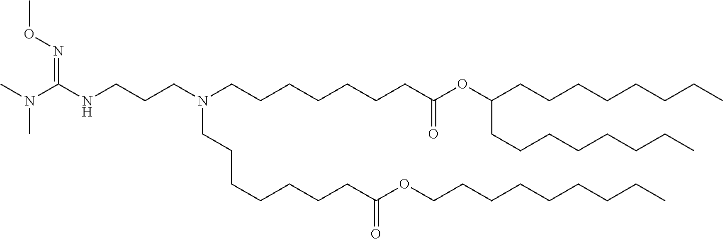 [SMILES: CCCCCCCCCOC(=O)CCCCCCCN(CCCCCCCC(=O)OC(CCCCCCCC)CCCCCCCC)CCCN/C(=N/OC)N(C)C] |
| I 212 | 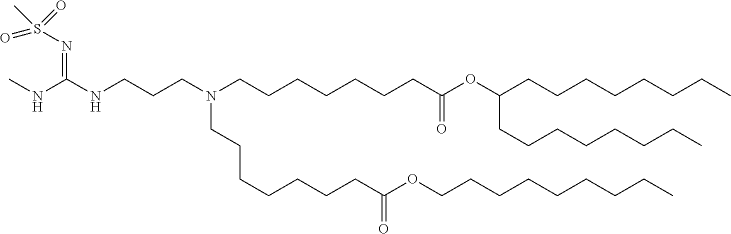 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I 213 | 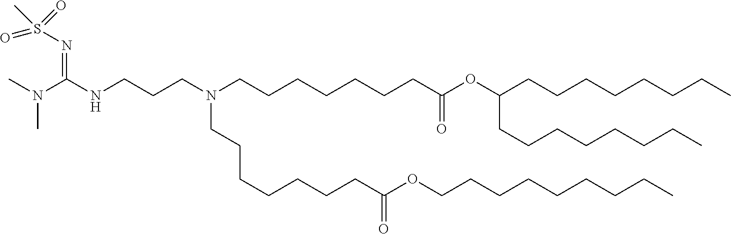 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 214 | 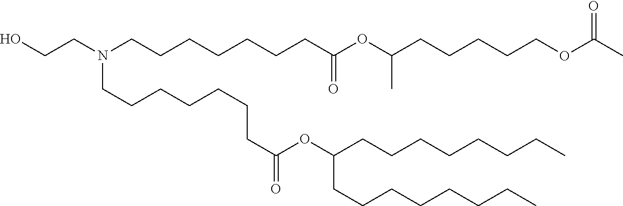 [SMILES: CCCCCCCCC(CCCCCCCC)OC(=O)CCCCCCCN(CCO)CCCCCCCC(=O)OC(C)CCCCCOC(C)=O] |
| I 215 | 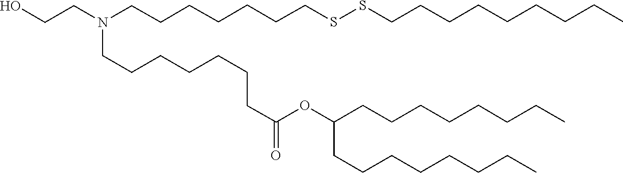 [SMILES: CCCCCCCCCSSCCCCCCCN(CCO)CCCCCCCC(=O)OC(CCCCCCCC)CCCCCCCC] |
| I 216 | 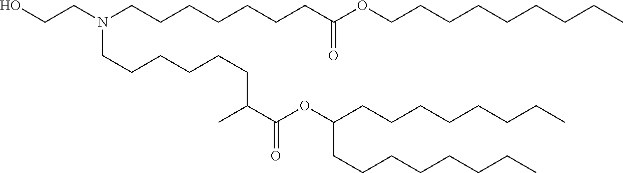 [SMILES: CCCCCCCCCOC(=O)CCCCCCCN(CCO)CCCCCCC(C)C(=O)OC(CCCCCCCC)CCCCCCCC] |
| I 217 | 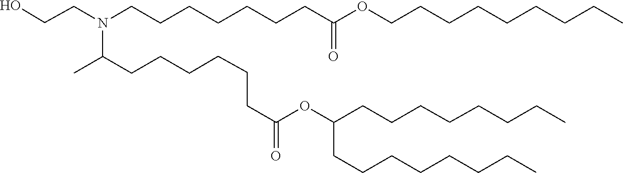 [SMILES: CCCCCCCCCOC(=O)CCCCCCCN(CCO)C(C)CCCCCCC(=O)OC(CCCCCCCC)CCCCCCCC] |
| I 218 | 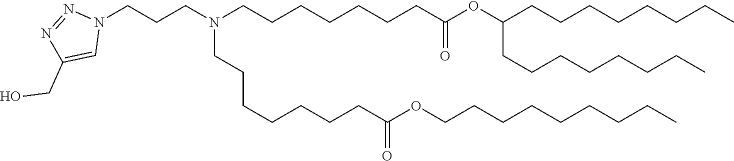 [SMILES: CCCCCCCCCOC(=O)CCCCCCCN(CCCCCCCC(=O)OC(CCCCCCCC)CCCCCCCC)CCCn1cc(CO)nn1] |
| I 219 | 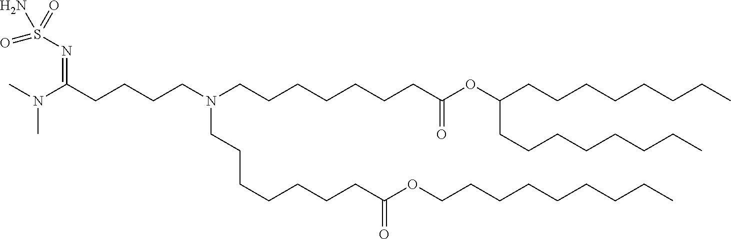 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I 220 | 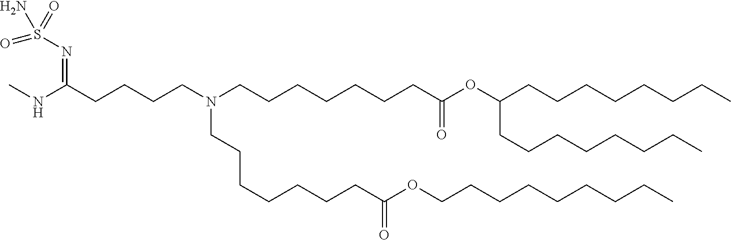 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I 221 | 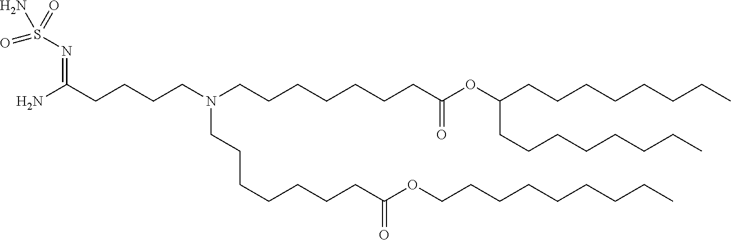 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I 222 | 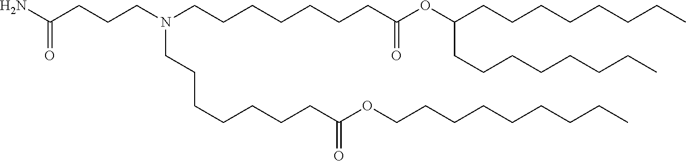 [SMILES: CCCCCCCCCOC(=O)CCCCCCCN(CCCCCCCC(=O)OC(CCCCCCCC)CCCCCCCC)CCCC(N)=O] |
| I 223 | 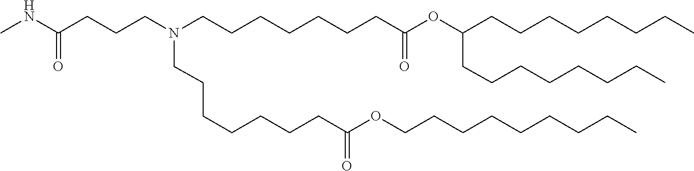 [SMILES: CCCCCCCCCOC(=O)CCCCCCCN(CCCCCCCC(=O)OC(CCCCCCCC)CCCCCCCC)CCCC(=O)NC] |
| I 224 | 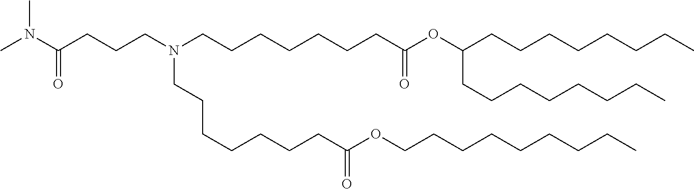 [SMILES: CCCCCCCCCOC(=O)CCCCCCCN(CCCCCCCC(=O)OC(CCCCCCCC)CCCCCCCC)CCCC(=O)N(C)C] |
| I 225 | 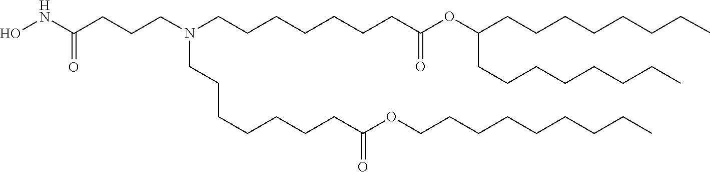 [SMILES: CCCCCCCCCOC(=O)CCCCCCCN(CCCCCCCC(=O)OC(CCCCCCCC)CCCCCCCC)CCCC(=O)NO] |
| I 226 | 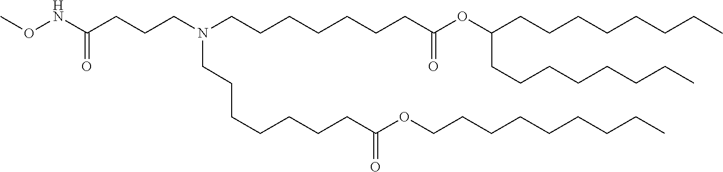 [SMILES: CCCCCCCCCOC(=O)CCCCCCCN(CCCCCCCC(=O)OC(CCCCCCCC)CCCCCCCC)CCCC(=O)NOC] |
| I 227 | 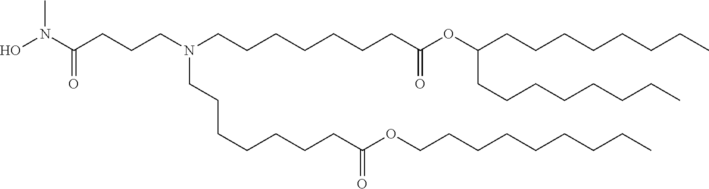 [SMILES: CCCCCCCCCOC(=O)CCCCCCCN(CCCCCCCC(=O)OC(CCCCCCCC)CCCCCCCC)CCCC(=O)N(C)O] |
| I 228 | 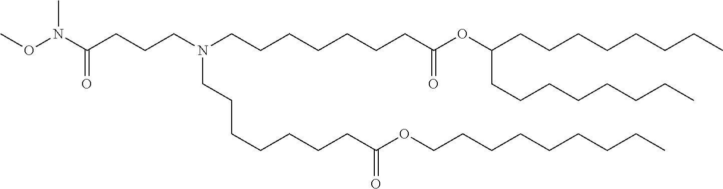 [SMILES: CCCCCCCCCOC(=O)CCCCCCCN(CCCCCCCC(=O)OC(CCCCCCCC)CCCCCCCC)CCCC(=O)N(C)OC] |
| I 229 | 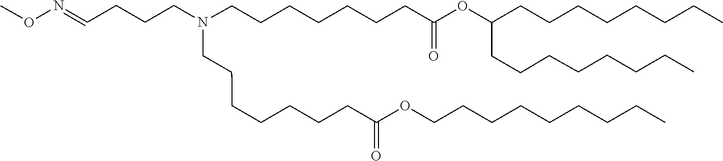 [SMILES: CCCCCCCCCOC(=O)CCCCCCCN(CCC/C=N/OC)CCCCCCCC(=O)OC(CCCCCCCC)CCCCCCCC] |
| I 230 | 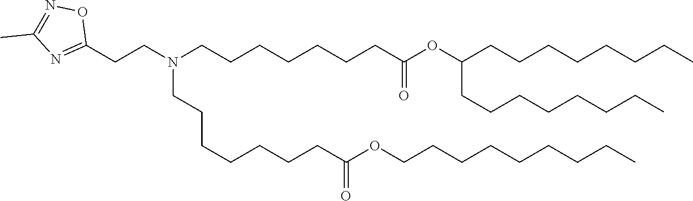 [SMILES: CCCCCCCCCOC(=O)CCCCCCCN(CCCCCCCC(=O)OC(CCCCCCCC)CCCCCCCC)CCc1nc(C)no1] |
| I 231 | 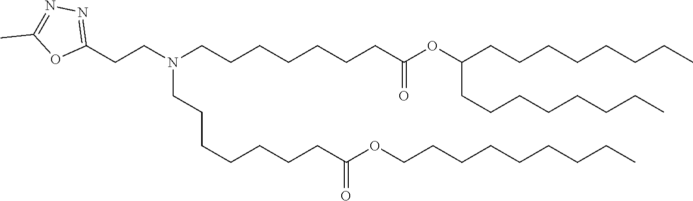 [SMILES: CCCCCCCCCOC(=O)CCCCCCCN(CCCCCCCC(=O)OC(CCCCCCCC)CCCCCCCC)CCc1nnc(C)o1] |
| I 232 | 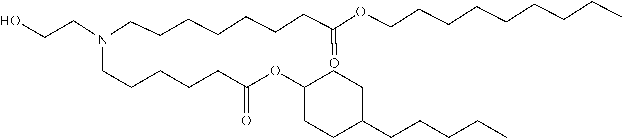 [SMILES: CCCCCCCCCOC(=O)CCCCCCCN(CCO)CCCCCC(=O)OC1CCC(CCCCC)CC1] |
| I 233 | 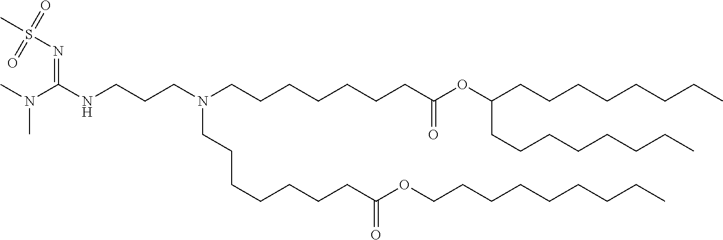 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 234 | 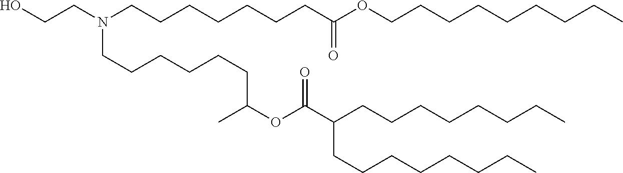 [SMILES: CCCCCCCCCOC(=O)CCCCCCCN(CCO)CCCCCCC(C)OC(=O)C(CCCCCCCC)CCCCCCCC] |
| I 235 | 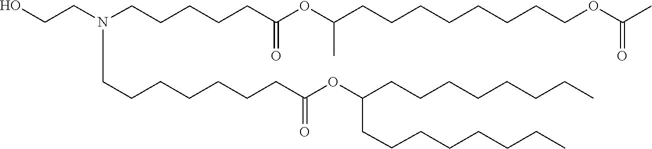 [SMILES: CCCCCCCCC(CCCCCCCC)OC(=O)CCCCCCCN(CCO)CCCCCC(=O)OC(C)CCCCCCCCOC(C)=O] |
| I 236 | 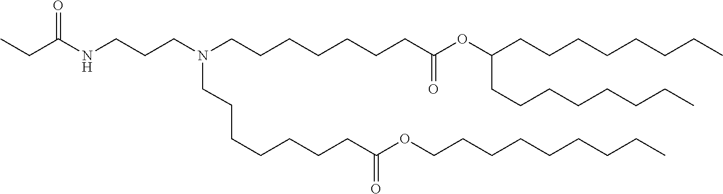 [SMILES: CCCCCCCCCOC(=O)CCCCCCCN(CCCCCCCC(=O)OC(CCCCCCCC)CCCCCCCC)CCCNC(=O)CC] |
| I 237 | 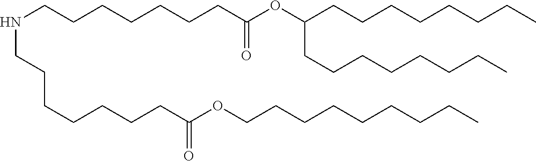 [SMILES: CCCCCCCCCOC(=O)CCCCCCCNCCCCCCCC(=O)OC(CCCCCCCC)CCCCCCCC] |
| I 238 | 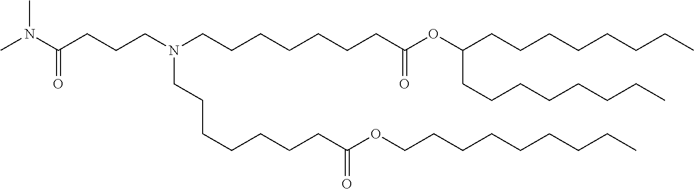 [SMILES: CCCCCCCCCOC(=O)CCCCCCCN(CCCCCCCC(=O)OC(CCCCCCCC)CCCCCCCC)CCCC(=O)N(C)C] |
| I 239 | 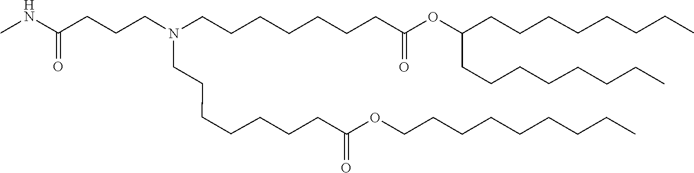 [SMILES: CCCCCCCCCOC(=O)CCCCCCCN(CCCCCCCC(=O)OC(CCCCCCCC)CCCCCCCC)CCCC(=O)NC] |
| I 240 | 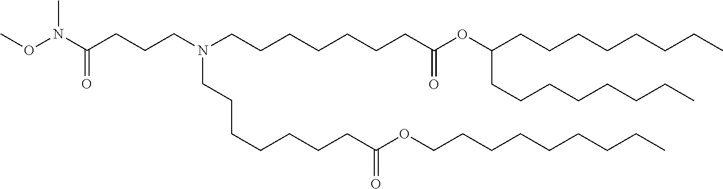 [SMILES: CCCCCCCCCOC(=O)CCCCCCCN(CCCCCCCC(=O)OC(CCCCCCCC)CCCCCCCC)CCCC(=O)N(C)OC] |
| I 241 | 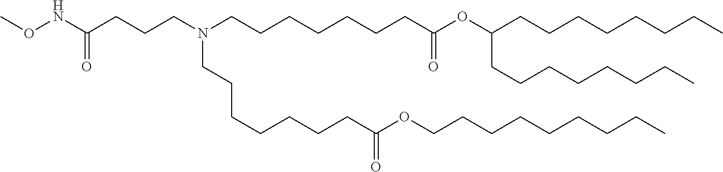 [SMILES: CCCCCCCCCOC(=O)CCCCCCCN(CCCCCCCC(=O)OC(CCCCCCCC)CCCCCCCC)CCCC(=O)NOC] |
| I 242 | 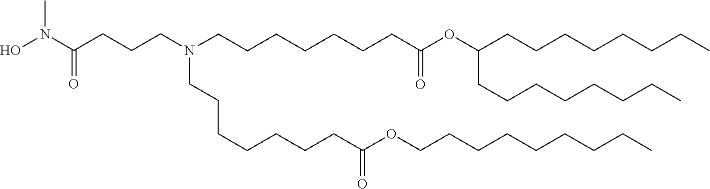 [SMILES: CCCCCCCCCOC(=O)CCCCCCCN(CCCCCCCC(=O)OC(CCCCCCCC)CCCCCCCC)CCCC(=O)N(C)O] |
| I 243 | 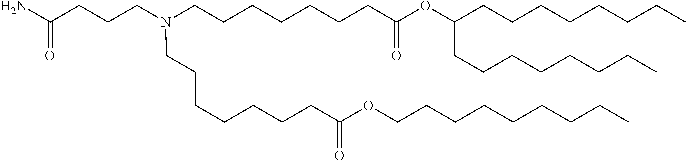 [SMILES: CCCCCCCCCOC(=O)CCCCCCCN(CCCCCCCC(=O)OC(CCCCCCCC)CCCCCCCC)CCCC(N)=O] |
| I 244 | 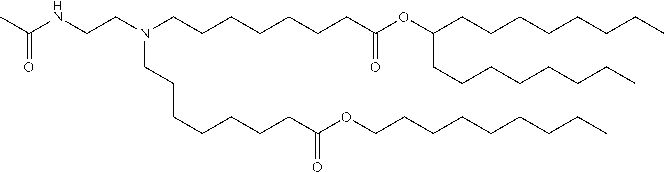 [SMILES: CCCCCCCCCOC(=O)CCCCCCCN(CCCCCCCC(=O)OC(CCCCCCCC)CCCCCCCC)CCNC(C)=O] |
| I 245 | 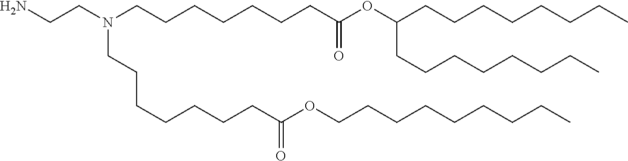 [SMILES: CCCCCCCCCOC(=O)CCCCCCCN(CCN)CCCCCCCC(=O)OC(CCCCCCCC)CCCCCCCC] |
| I 246 | 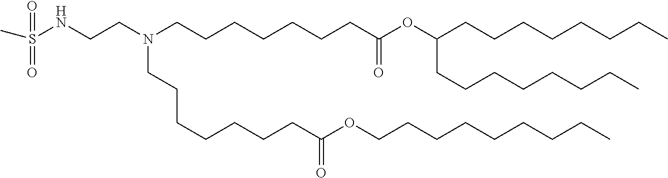 [SMILES: CCCCCCCCCOC(=O)CCCCCCCN(CCCCCCCC(=O)OC(CCCCCCCC)CCCCCCCC)CCNS(C)(=O)=O] |
| I 247 | 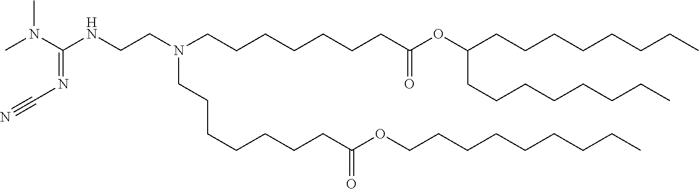 [SMILES: CCCCCCCCCOC(=O)CCCCCCCN(CCCCCCCC(=O)OC(CCCCCCCC)CCCCCCCC)CCN/C(=N/C#N)N(C)C] |
| I 248 | 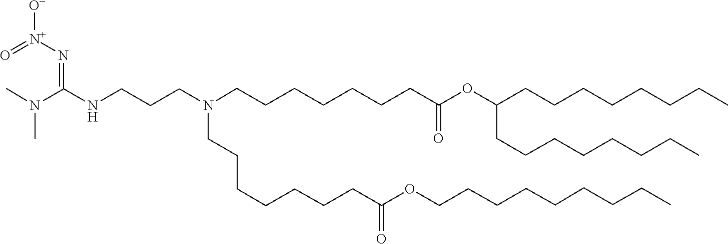 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 249 | 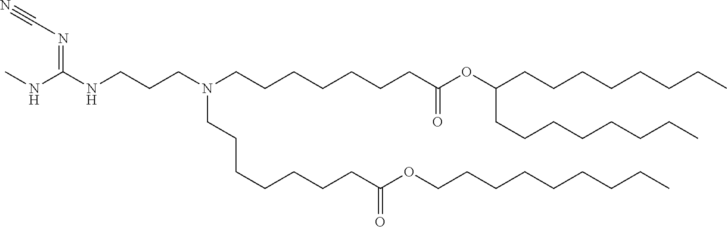 [SMILES: CCCCCCCCCOC(=O)CCCCCCCN(CCCCCCCC(=O)OC(CCCCCCCC)CCCCCCCC)CCCN/C(=N/C#N)NC] |
| I 250 | 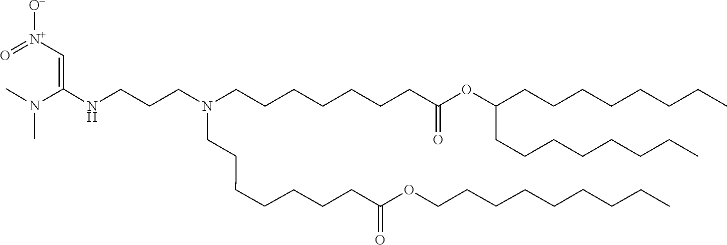 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I 251 | 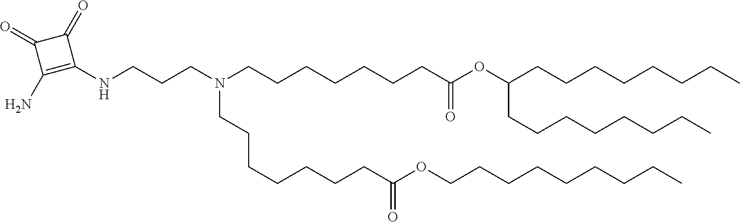 [SMILES: CCCCCCCCCOC(=O)CCCCCCCN(CCCCCCCC(=O)OC(CCCCCCCC)CCCCCCCC)CCCNc1c(N)c(=O)c1=O] |
| I 252 | 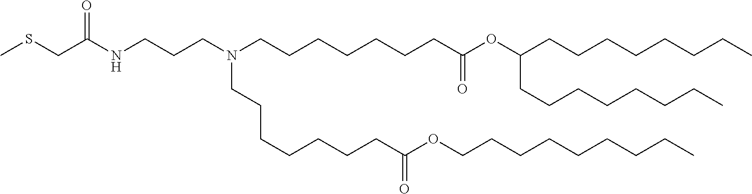 [SMILES: CCCCCCCCCOC(=O)CCCCCCCN(CCCCCCCC(=O)OC(CCCCCCCC)CCCCCCCC)CCCNC(=O)CSC] |
| I 253 | 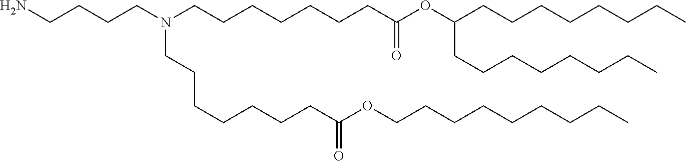 [SMILES: CCCCCCCCCOC(=O)CCCCCCCN(CCCCN)CCCCCCCC(=O)OC(CCCCCCCC)CCCCCCCC] |
| I 254 | 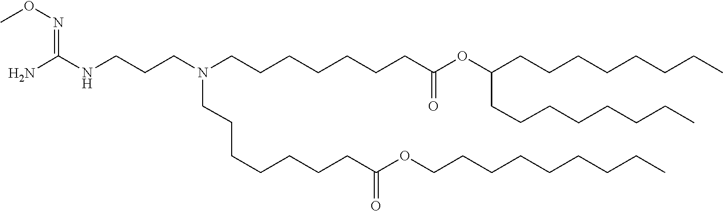 [SMILES: CCCCCCCCCOC(=O)CCCCCCCN(CCCCCCCC(=O)OC(CCCCCCCC)CCCCCCCC)CCCN/C(N)=N/OC] |
| I 255 | 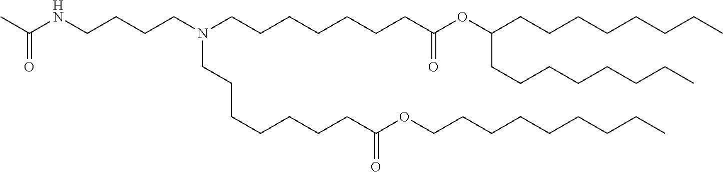 [SMILES: CCCCCCCCCOC(=O)CCCCCCCN(CCCCCCCC(=O)OC(CCCCCCCC)CCCCCCCC)CCCCNC(C)=O] |
| I 256 | 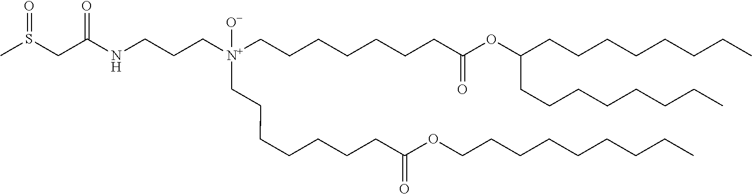 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I 257 | 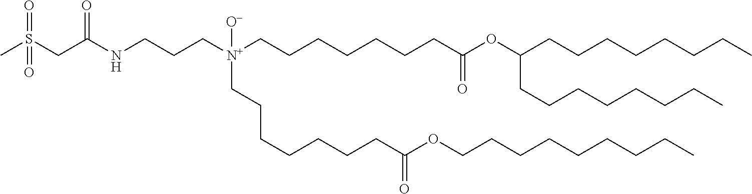 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I 258 | 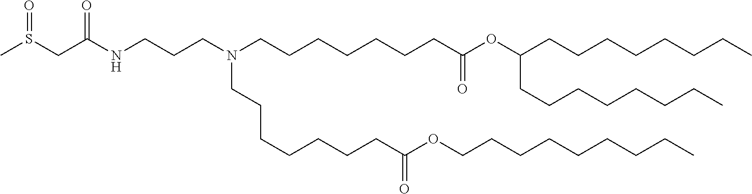 [SMILES: CCCCCCCCCOC(=O)CCCCCCCN(CCCCCCCC(=O)OC(CCCCCCCC)CCCCCCCC)CCCNC(=O)CS(C)=O] |
| I 259 | 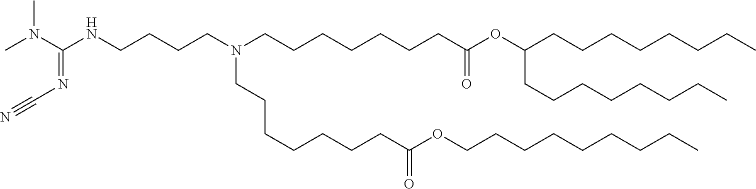 [SMILES: CCCCCCCCCOC(=O)CCCCCCCN(CCCCCCCC(=O)OC(CCCCCCCC)CCCCCCCC)CCCCN/C(=N/C#N)N(C)C] |
| I 260 | 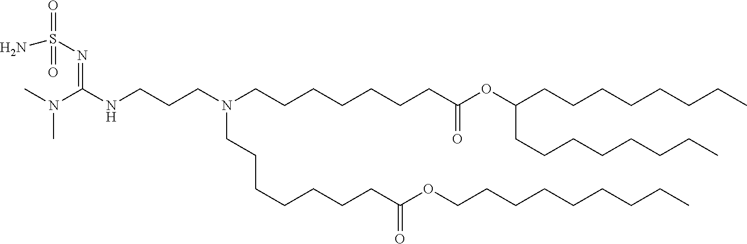 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I 261 | 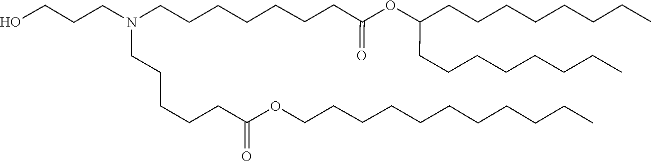 [SMILES: CCCCCCCCCCCOC(=O)CCCCCN(CCCO)CCCCCCCC(=O)OC(CCCCCCCC)CCCCCCCC] |
| I 262 | 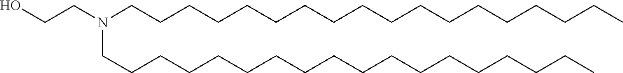 [SMILES: CCCCCCCCCCCCCCCCCCN(CCO)CCCCCCCCCCCCCCCCCC] |
| I 263 | 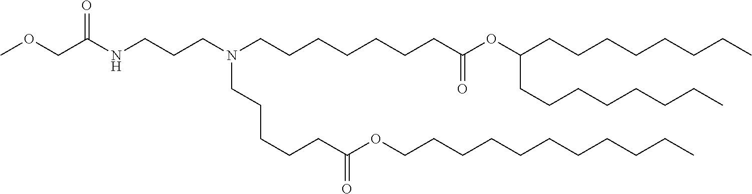 [SMILES: CCCCCCCCCCCOC(=O)CCCCCN(CCCCCCCC(=O)OC(CCCCCCCC)CCCCCCCC)CCCNC(=O)COC] |
| I 264 | 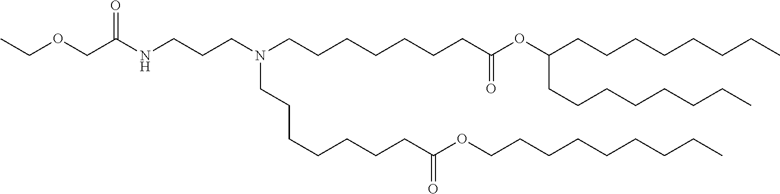 [SMILES: CCCCCCCCCOC(=O)CCCCCCCN(CCCCCCCC(=O)OC(CCCCCCCC)CCCCCCCC)CCCNC(=O)COCC] |
| I 265 | 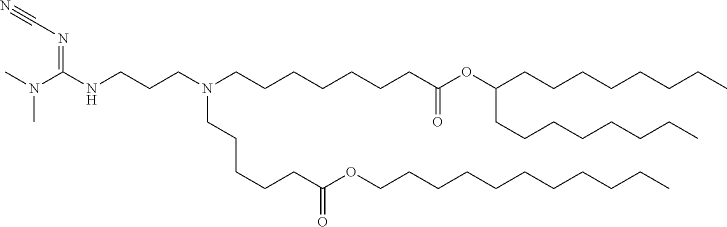 [SMILES: CCCCCCCCCCCOC(=O)CCCCCN(CCCCCCCC(=O)OC(CCCCCCCC)CCCCCCCC)CCCN/C(=N/C#N)N(C)C] |
| I 266 | 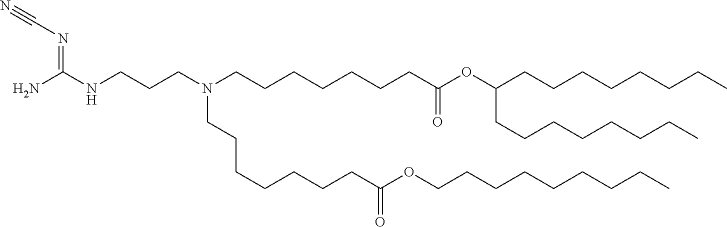 [SMILES: CCCCCCCCCOC(=O)CCCCCCCN(CCCCCCCC(=O)OC(CCCCCCCC)CCCCCCCC)CCCN/C(N)=N/C#N] |
| I 267 | 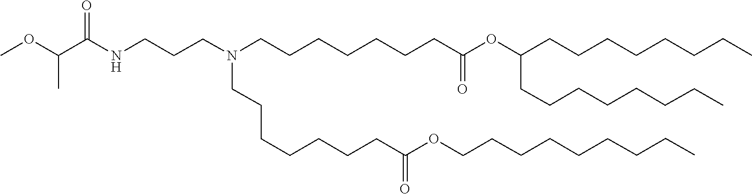 [SMILES: CCCCCCCCCOC(=O)CCCCCCCN(CCCCCCCC(=O)OC(CCCCCCCC)CCCCCCCC)CCCNC(=O)C(C)OC] |
| I 268 | 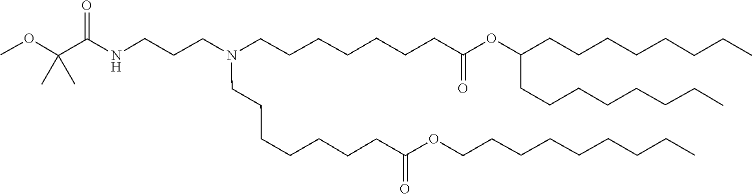 [SMILES: CCCCCCCCCOC(=O)CCCCCCCN(CCCCCCCC(=O)OC(CCCCCCCC)CCCCCCCC)CCCNC(=O)C(C)(C)OC] |
| I 269 | 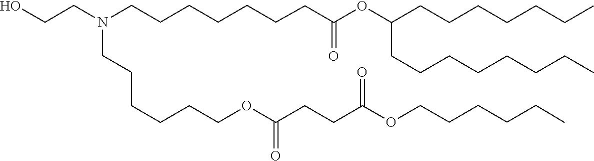 [SMILES: CCCCCCCCC(CCCCCCC)OC(=O)CCCCCCCN(CCO)CCCCCCOC(=O)CCC(=O)OCCCCCC] |
| I 270 | 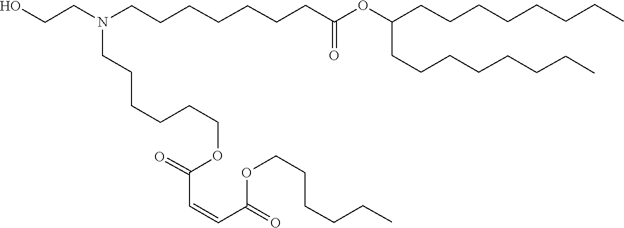 [SMILES: CCCCCCCCC(CCCCCCCC)OC(=O)CCCCCCCN(CCO)CCCCCCOC(=O)/C=C\C(=O)OCCCCCC] |
| I 271 | 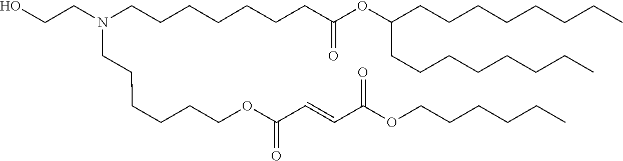 [SMILES: CCCCCCCCC(CCCCCCCC)OC(=O)CCCCCCCN(CCO)CCCCCCOC(=O)/C=C/C(=O)OCCCCCC] |
| I 272 | 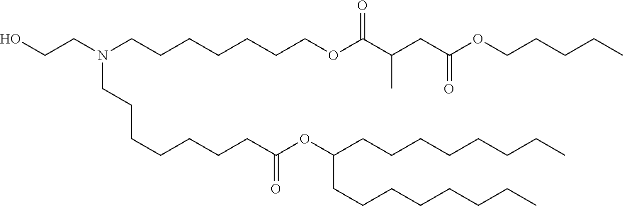 [SMILES: CCCCCCCCC(CCCCCCCC)OC(=O)CCCCCCCN(CCO)CCCCCCCOC(=O)C(C)CC(=O)OCCCCC] |
| I 273 | 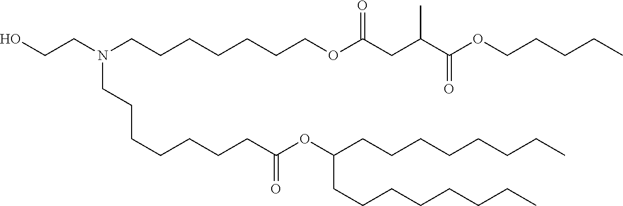 [SMILES: CCCCCCCCC(CCCCCCCC)OC(=O)CCCCCCCN(CCO)CCCCCCCOC(=O)CC(C)C(=O)OCCCCC] |
| I 274 | 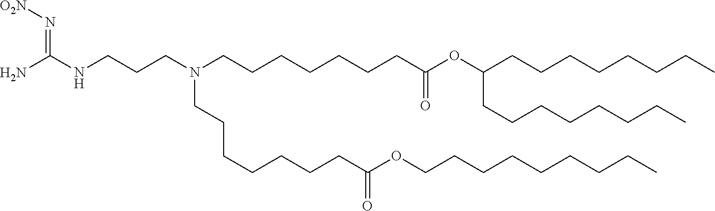 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I 275 | 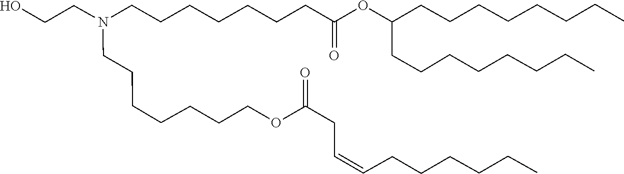 [SMILES: CCCCCC/C=C\CC(=O)OCCCCCCCN(CCO)CCCCCCCC(=O)OC(CCCCCCCC)CCCCCCCC] |
| I 276 | 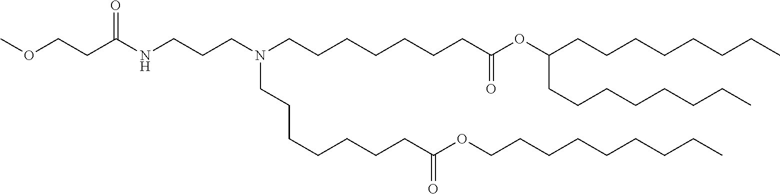 [SMILES: CCCCCCCCCOC(=O)CCCCCCCN(CCCCCCCC(=O)OC(CCCCCCCC)CCCCCCCC)CCCNC(=O)CCOC] |
| I 277 | 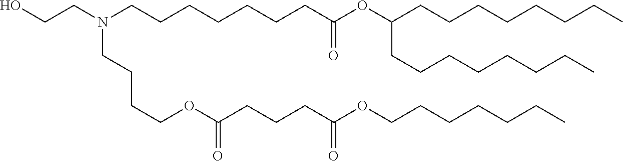 [SMILES: CCCCCCCCC(CCCCCCCC)OC(=O)CCCCCCCN(CCO)CCCCOC(=O)CCCC(=O)OCCCCCCC] |
| I 278 | 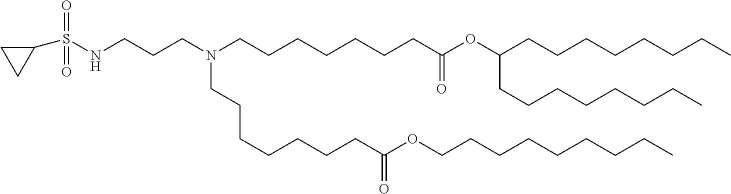 [SMILES: CCCCCCCCCOC(=O)CCCCCCCN(CCCCCCCC(=O)OC(CCCCCCCC)CCCCCCCC)CCCNS(=O)(=O)C1CC1] |
| I 279 | 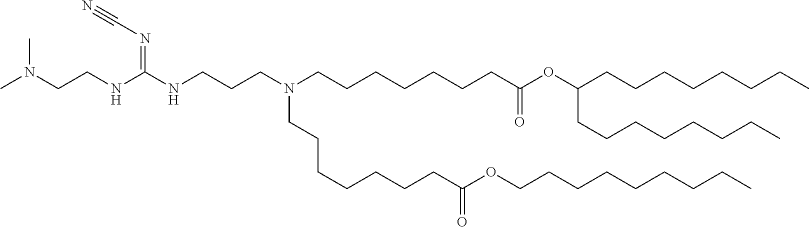 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I 280 | 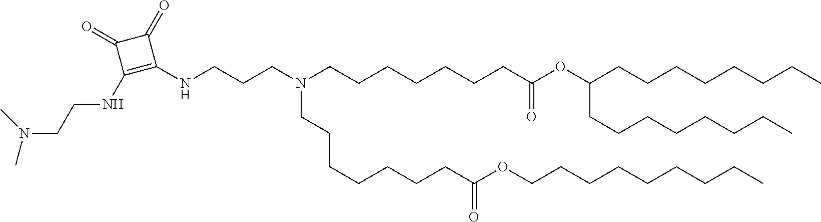 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I 281 | 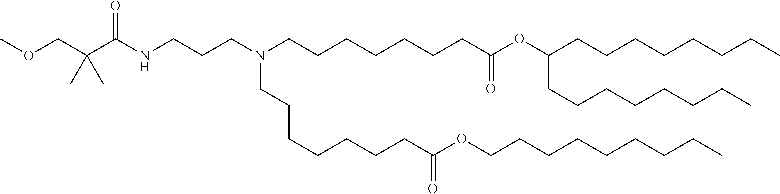 [SMILES: CCCCCCCCCOC(=O)CCCCCCCN(CCCCCCCC(=O)OC(CCCCCCCC)CCCCCCCC)CCCNC(=O)C(C)(C)COC] |
| I 282 | 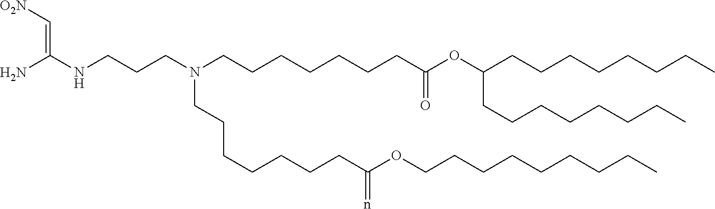 |
| I 283 | 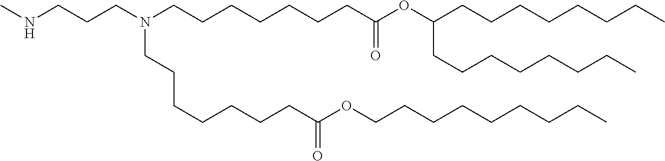 [SMILES: CCCCCCCCCOC(=O)CCCCCCCN(CCCCCCCC(=O)OC(CCCCCCCC)CCCCCCCC)CCCNC] |
| I 284 | 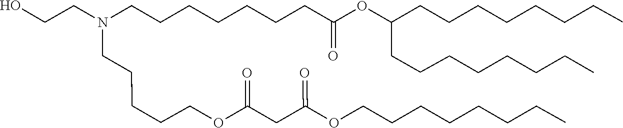 [SMILES: CCCCCCCCOC(=O)CC(=O)OCCCCCN(CCO)CCCCCCCC(=O)OC(CCCCCCCC)CCCCCCCC] |
| I 285 | 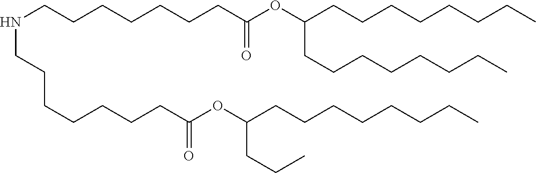 [SMILES: CCCCCCCCC(CCC)OC(=O)CCCCCCCNCCCCCCCC(=O)OC(CCCCCCCC)CCCCCCCC] |
| I 286 | 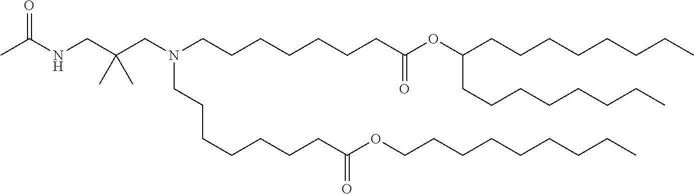 [SMILES: CCCCCCCCCOC(=O)CCCCCCCN(CCCCCCCC(=O)OC(CCCCCCCC)CCCCCCCC)CC(C)(C)CNC(C)=O] |
| I 287 | 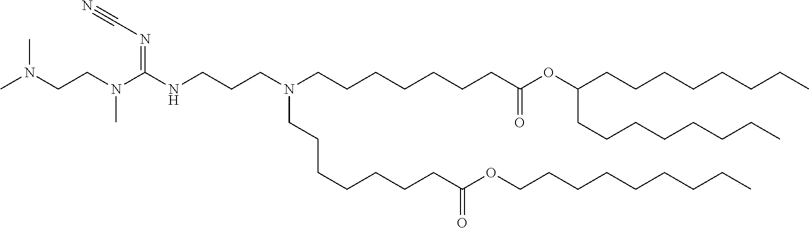 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I 288 | 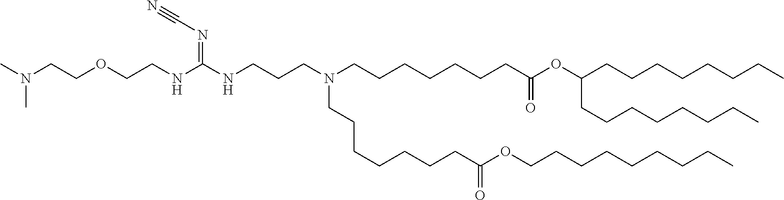 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I 289 | 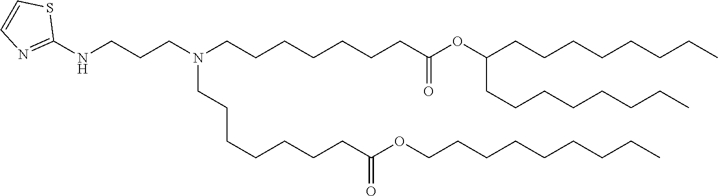 [SMILES: CCCCCCCCCOC(=O)CCCCCCCN(CCCCCCCC(=O)OC(CCCCCCCC)CCCCCCCC)CCCNc1nccs1] |
| I 290 | 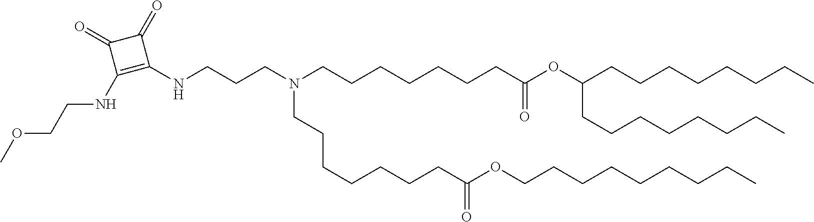 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I 291 | 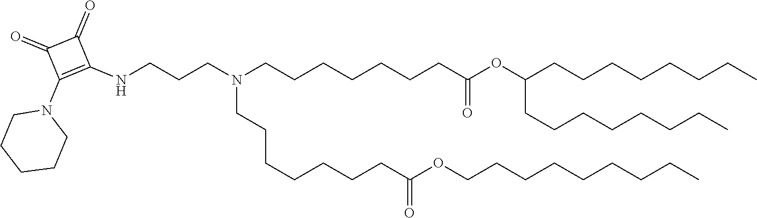 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I 292 | 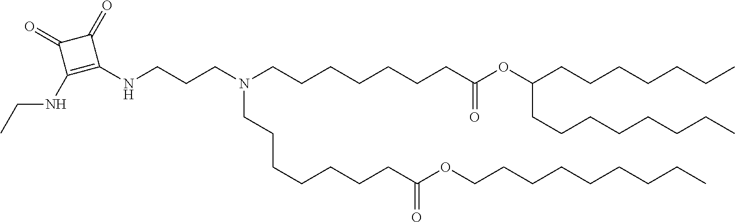 [SMILES: CCCCCCCCCOC(=O)CCCCCCCN(CCCCCCCC(=O)OC(CCCCCCC)CCCCCCCC)CCCNc1c(NCC)c(=O)c1=O] |
| I 293 | 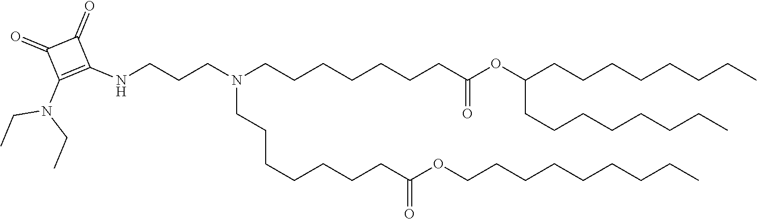 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I 294 | 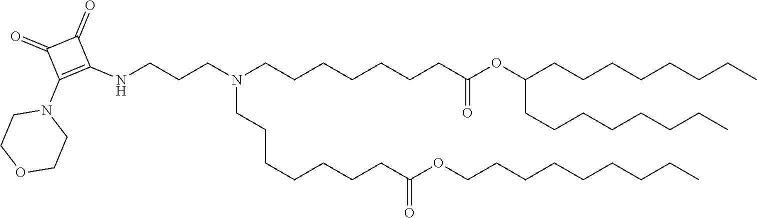 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I 295 | 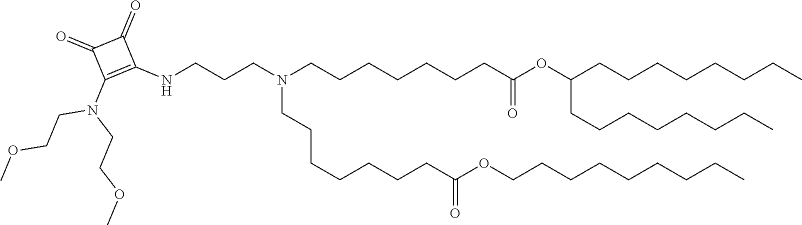 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I 296 | 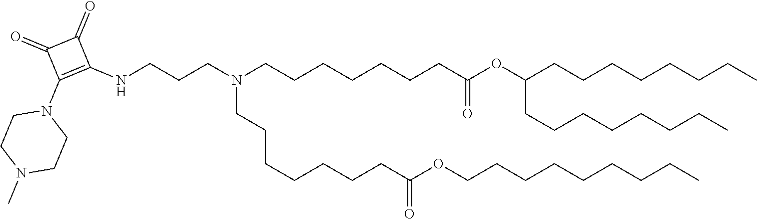 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I 297 | 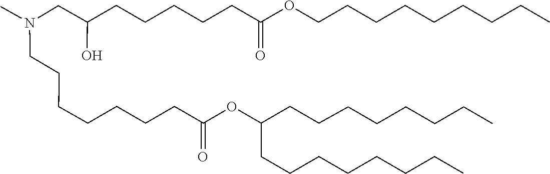 [SMILES: CCCCCCCCCOC(=O)CCCCCC(O)CN(C)CCCCCCCC(=O)OC(CCCCCCCC)CCCCCCCC] |
| I 298 | 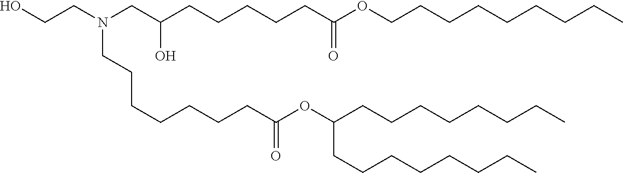 [SMILES: CCCCCCCCCOC(=O)CCCCCC(O)CN(CCO)CCCCCCCC(=O)OC(CCCCCCCC)CCCCCCCC] |
| I 299 | 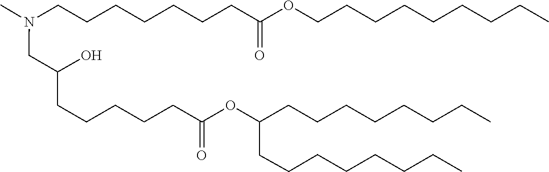 [SMILES: CCCCCCCCCOC(=O)CCCCCCCN(C)CC(O)CCCCCC(=O)OC(CCCCCCCC)CCCCCCCC] |
| I 300 | 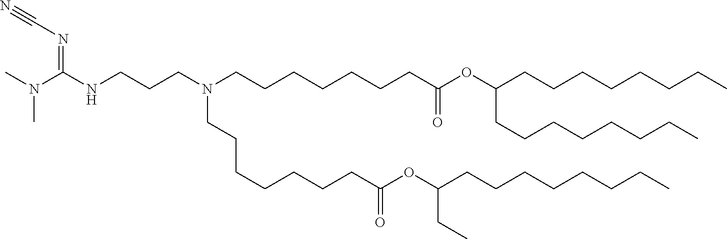 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 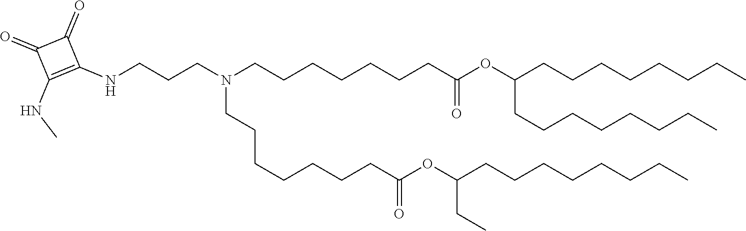 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I 302 | 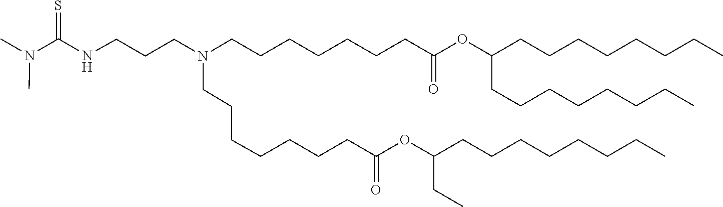 [SMILES: CCCCCCCCC(CC)OC(=O)CCCCCCCN(CCCCCCCC(=O)OC(CCCCCCCC)CCCCCCCC)CCCNC(=S)N(C)C] |
| I 303 | 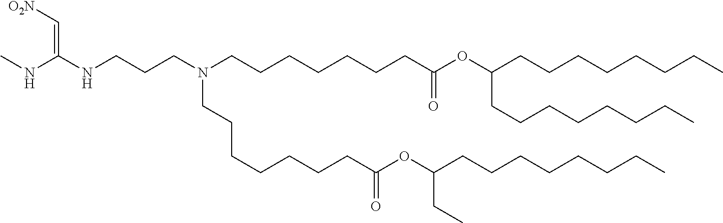 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I 304 | 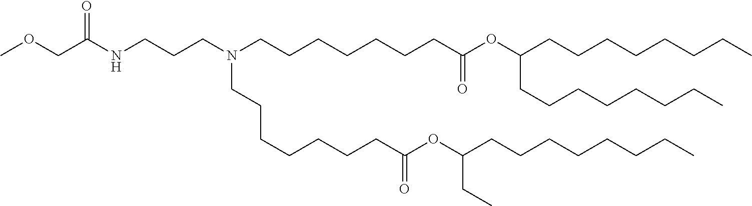 [SMILES: CCCCCCCCC(CC)OC(=O)CCCCCCCN(CCCCCCCC(=O)OC(CCCCCCCC)CCCCCCCC)CCCNC(=O)COC] |
| I 305 | 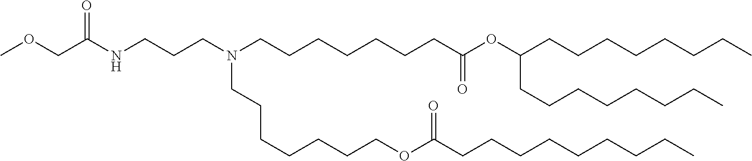 [SMILES: CCCCCCCCCC(=O)OCCCCCCCN(CCCCCCCC(=O)OC(CCCCCCCC)CCCCCCCC)CCCNC(=O)COC] |
| I 306 | 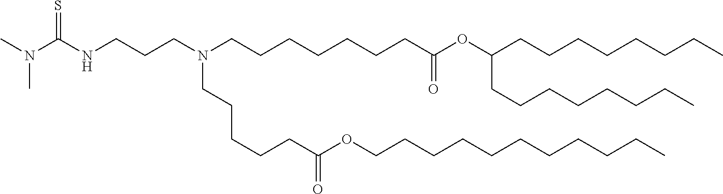 [SMILES: CCCCCCCCCCCOC(=O)CCCCCN(CCCCCCCC(=O)OC(CCCCCCCC)CCCCCCCC)CCCNC(=S)N(C)C] |
| I 307 | 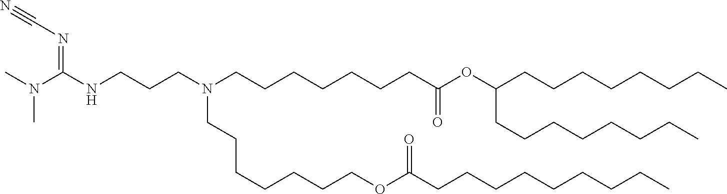 [SMILES: CCCCCCCCCC(=O)OCCCCCCCN(CCCCCCCC(=O)OC(CCCCCCCC)CCCCCCCC)CCCN/C(=N/C#N)N(C)C] |
| I 308 | 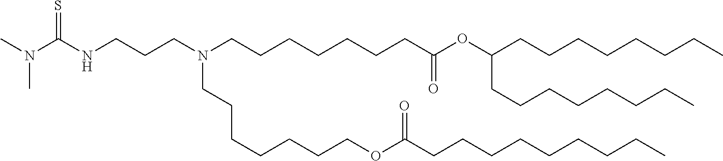 [SMILES: CCCCCCCCCC(=O)OCCCCCCCN(CCCCCCCC(=O)OC(CCCCCCCC)CCCCCCCC)CCCNC(=S)N(C)C] |
| I 309 | 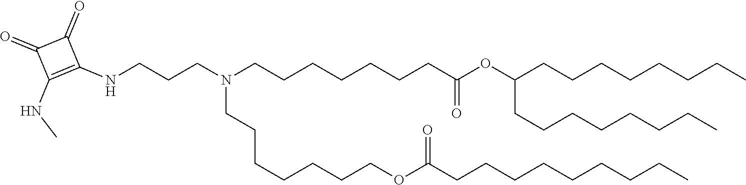 [SMILES: CCCCCCCCCC(=O)OCCCCCCCN(CCCCCCCC(=O)OC(CCCCCCCC)CCCCCCCC)CCCNc1c(NC)c(=O)c1=O] |
| I 310 | 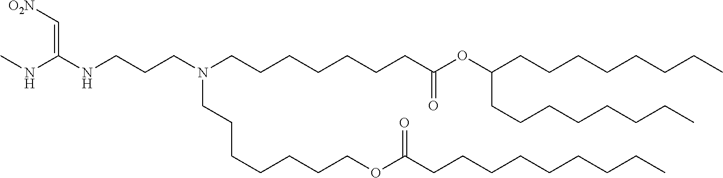 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I 311 | 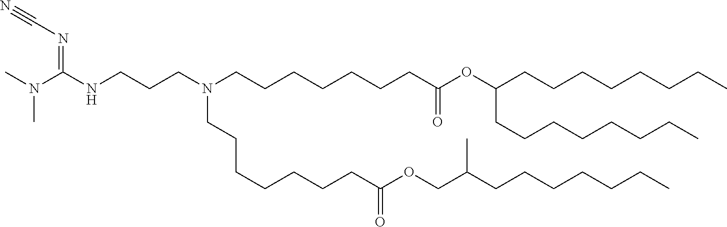 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I 312 | 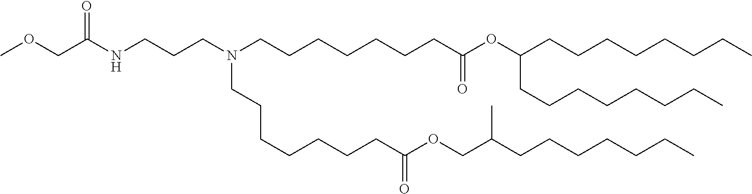 [SMILES: CCCCCCCCC(CCCCCCCC)OC(=O)CCCCCCCN(CCCCCCCC(=O)OCC(C)CCCCCCC)CCCNC(=O)COC] |
| I 313 | 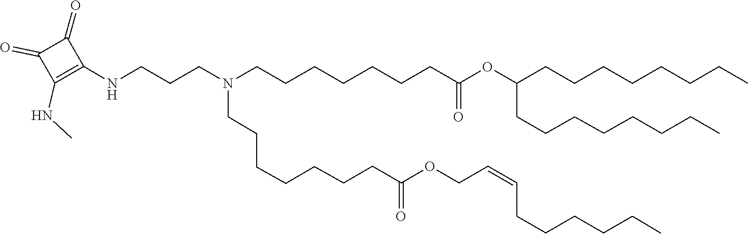 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I 314 | 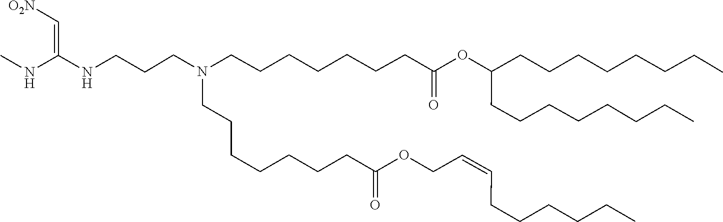 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I 315 | 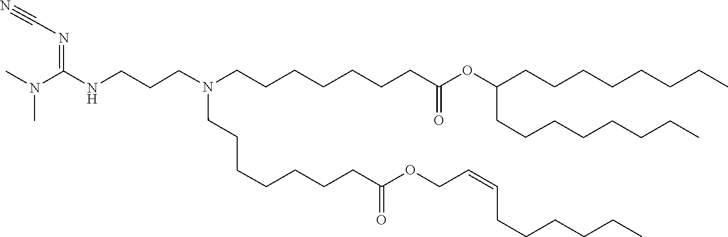 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I 316 | 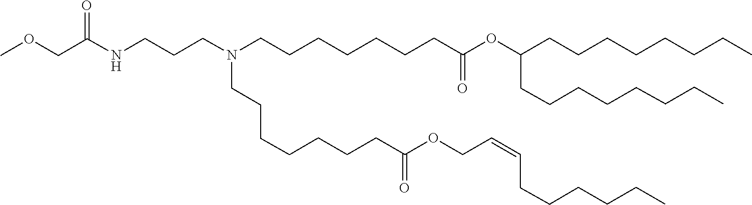 [SMILES: CCCCCC/C=C\COC(=O)CCCCCCCN(CCCCCCCC(=O)OC(CCCCCCCC)CCCCCCCC)CCCNC(=O)COC] |
| I 317 | 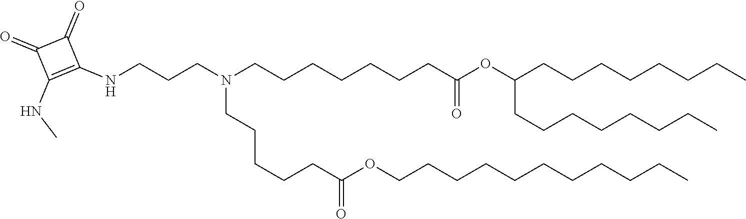 [SMILES: CCCCCCCCCCCOC(=O)CCCCCN(CCCCCCCC(=O)OC(CCCCCCCC)CCCCCCCC)CCCNc1c(NC)c(=O)c1=O] |
| I 318 | 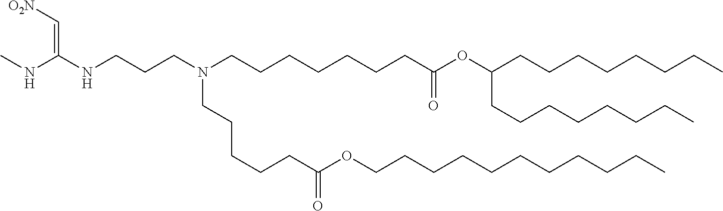 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 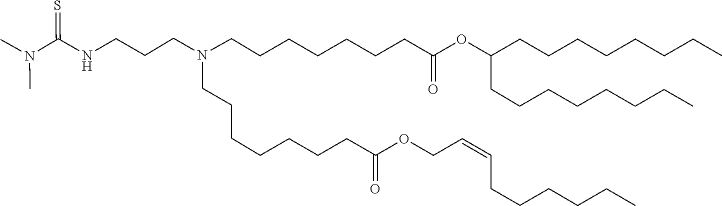 [SMILES: CCCCCC/C=C\COC(=O)CCCCCCCN(CCCCCCCC(=O)OC(CCCCCCCC)CCCCCCCC)CCCNC(=S)N(C)C] |
| I 320 | 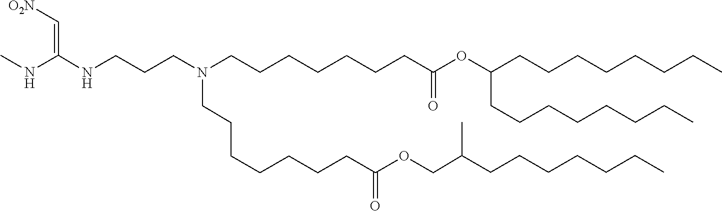 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 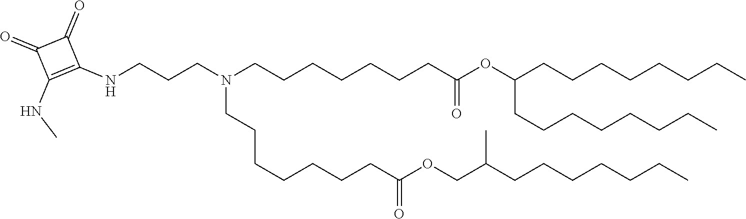 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I 322 | 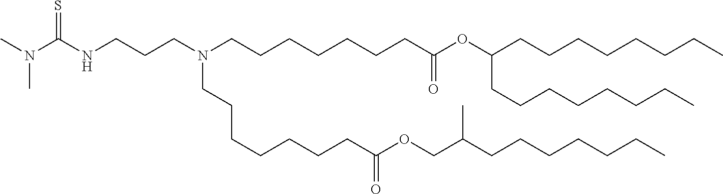 [SMILES: CCCCCCCCC(CCCCCCCC)OC(=O)CCCCCCCN(CCCCCCCC(=O)OCC(C)CCCCCCC)CCCNC(=S)N(C)C] |
| I 323 | 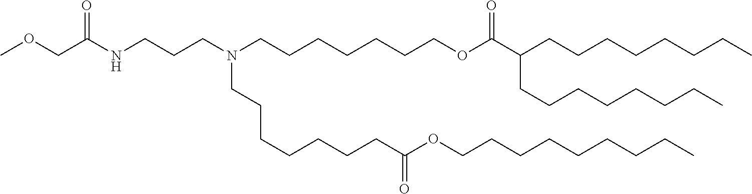 [SMILES: CCCCCCCCCOC(=O)CCCCCCCN(CCCCCCCOC(=O)C(CCCCCCCC)CCCCCCCC)CCCNC(=O)COC] |
| I 324 | 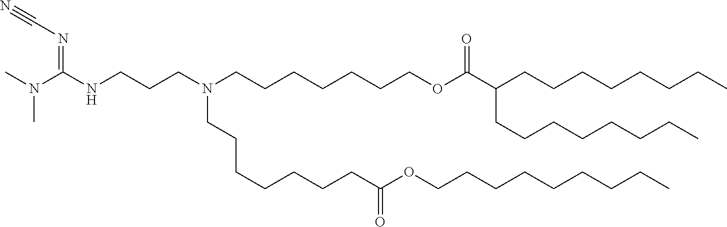 [SMILES: CCCCCCCCCOC(=O)CCCCCCCN(CCCCCCCOC(=O)C(CCCCCCCC)CCCCCCCC)CCCN/C(=N/C#N)N(C)C] |
| I 325 | 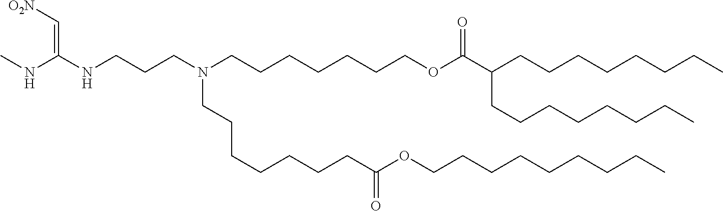 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 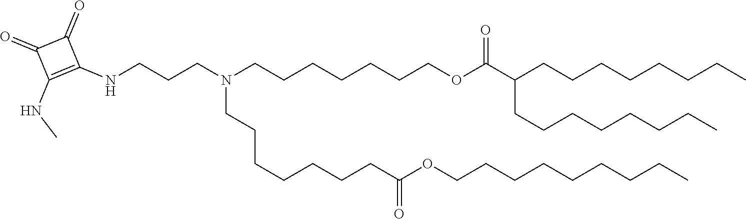 [SMILES: CCCCCCCCCOC(=O)CCCCCCCN(CCCCCCCOC(=O)C(CCCCCCCC)CCCCCCCC)CCCNc1c(NC)c(=O)c1=O] |
| I 327 | 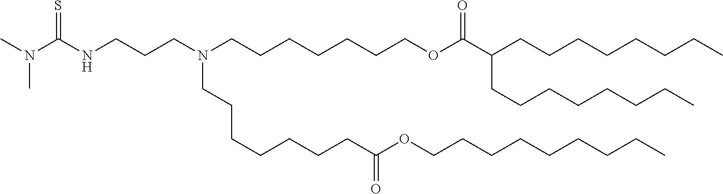 [SMILES: CCCCCCCCCOC(=O)CCCCCCCN(CCCCCCCOC(=O)C(CCCCCCCC)CCCCCCCC)CCCNC(=S)N(C)C] |
| I 328 | 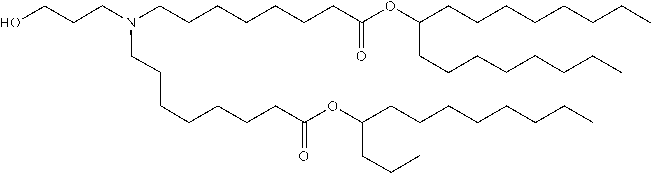 [SMILES: CCCCCCCCC(CCC)OC(=O)CCCCCCCN(CCCO)CCCCCCCC(=O)OC(CCCCCCCC)CCCCCCCC] |
| I 329 | 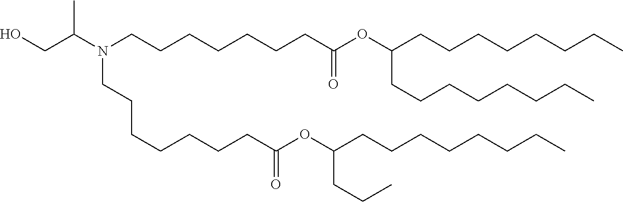 [SMILES: CCCCCCCCC(CCC)OC(=O)CCCCCCCN(CCCCCCCC(=O)OC(CCCCCCCC)CCCCCCCC)C(C)CO] |
| I 330 | 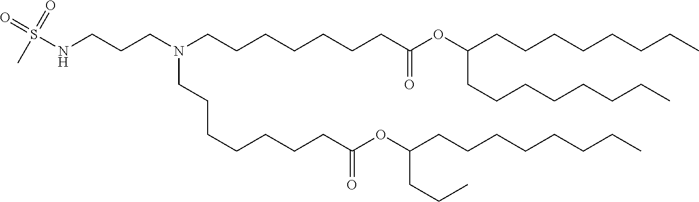 [SMILES: CCCCCCCCC(CCC)OC(=O)CCCCCCCN(CCCCCCCC(=O)OC(CCCCCCCC)CCCCCCCC)CCCNS(C)(=O)=O] |
| I 331 | 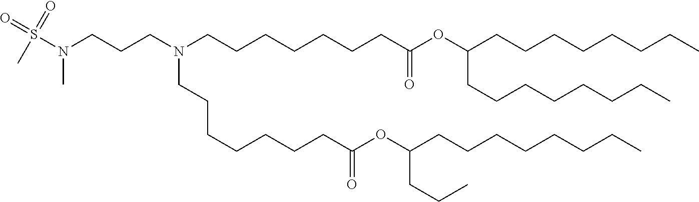 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I 332 | 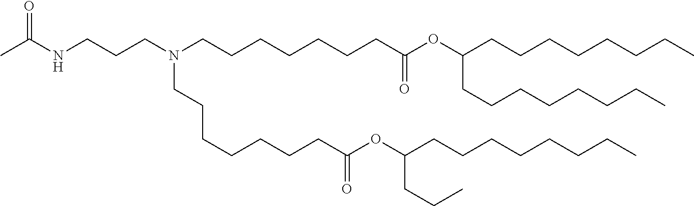 [SMILES: CCCCCCCCC(CCC)OC(=O)CCCCCCCN(CCCCCCCC(=O)OC(CCCCCCCC)CCCCCCCC)CCCNC(C)=O] |
| I 333 | 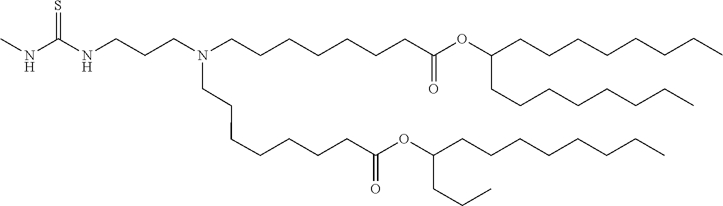 [SMILES: CCCCCCCCC(CCC)OC(=O)CCCCCCCN(CCCCCCCC(=O)OC(CCCCCCCC)CCCCCCCC)CCCNC(=S)NC] |
| I 334 | 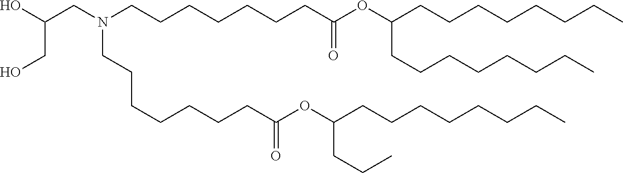 [SMILES: CCCCCCCCC(CCC)OC(=O)CCCCCCCN(CCCCCCCC(=O)OC(CCCCCCCC)CCCCCCCC)CC(O)CO] |
| I 335 | 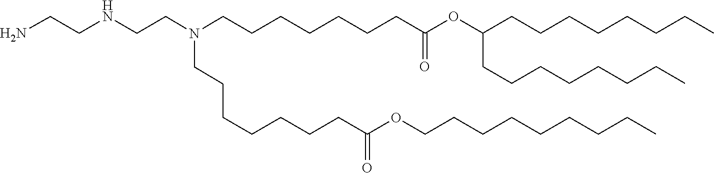 [SMILES: CCCCCCCCCOC(=O)CCCCCCCN(CCCCCCCC(=O)OC(CCCCCCCC)CCCCCCCC)CCNCCN] |
| I 336 | 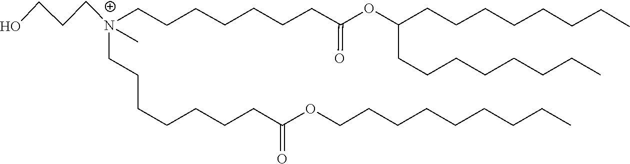 [SMILES: CCCCCCCCCOC(=O)CCCCCCC[N+](C)(CCCO)CCCCCCCC(=O)OC(CCCCCCCC)CCCCCCCC] |
| I 337 | 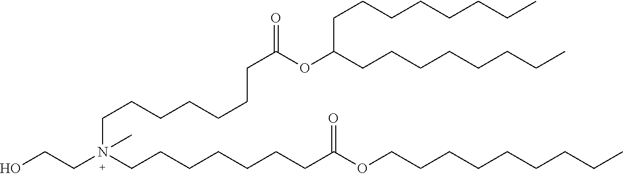 |
| I 338 | 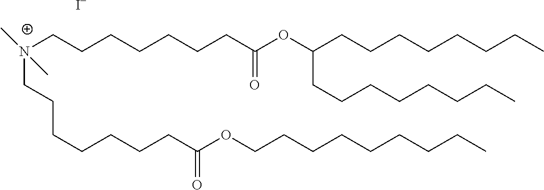 [SMILES: [I-] ; CCCCCCCCCOC(=O)CCCCCCC[N+](C)(C)CCCCCCCC(=O)OC(CCCCCCCC)CCCCCCCC] |
| I 339 | 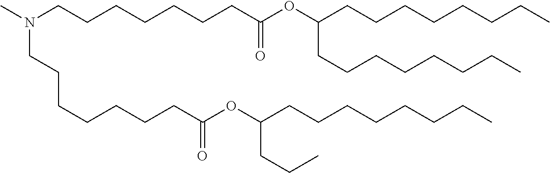 [SMILES: CCCCCCCCC(CCC)OC(=O)CCCCCCCN(C)CCCCCCCC(=O)OC(CCCCCCCC)CCCCCCCC] |
| I 340 | 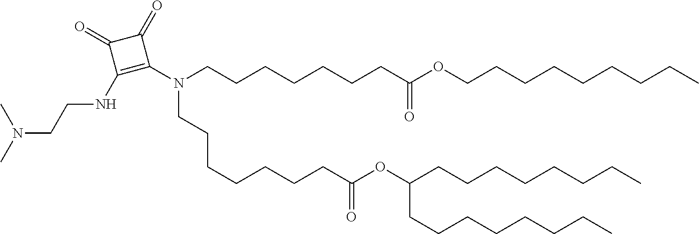 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I 341 | 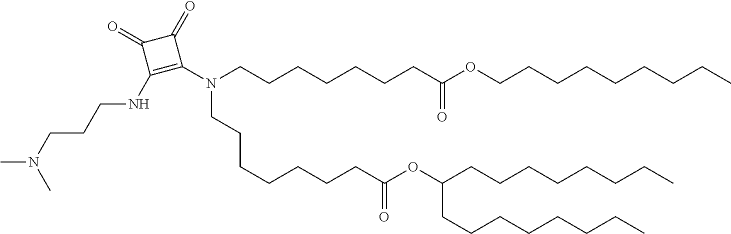 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I 342 | 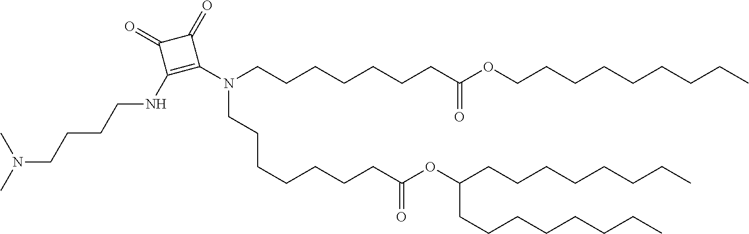 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I 343 | 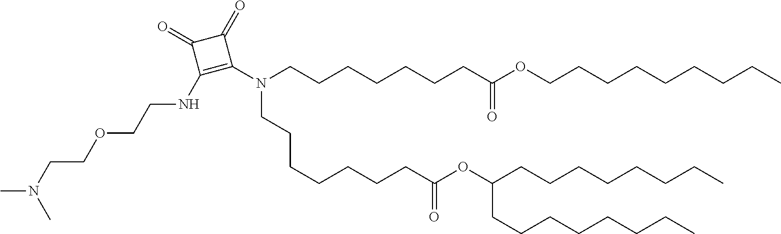 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I 344 | 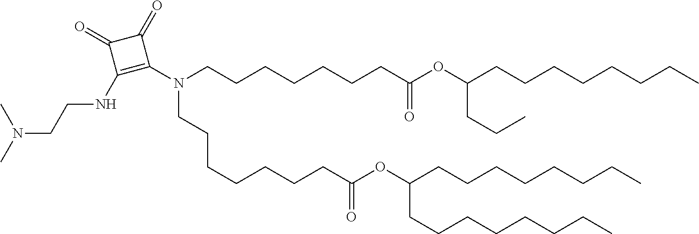 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I 345 | 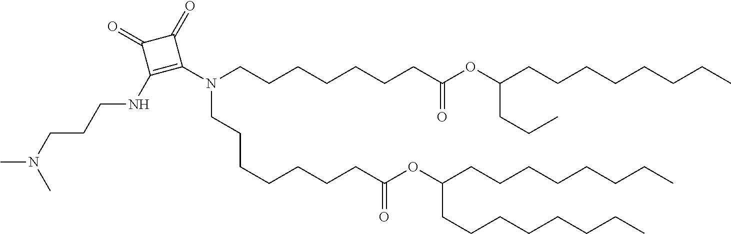 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I 346 | 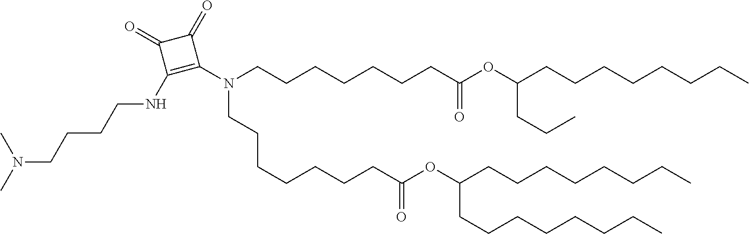 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I 347 | 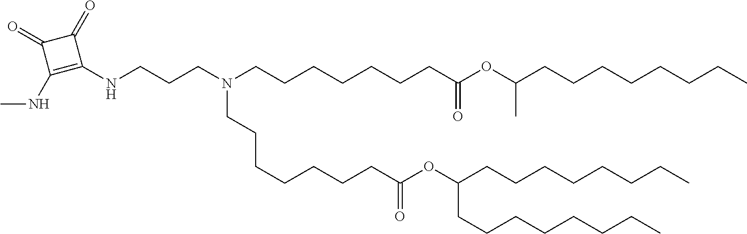 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I 348 | 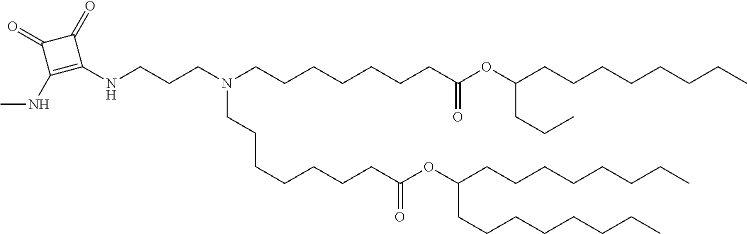 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I 349 | 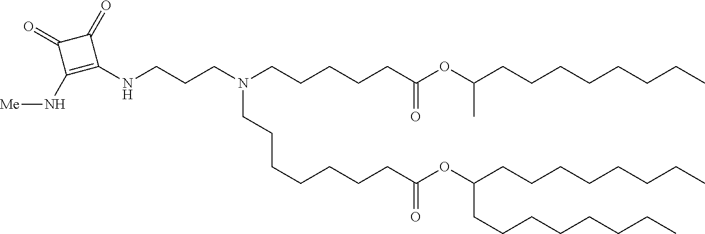 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I 350 | 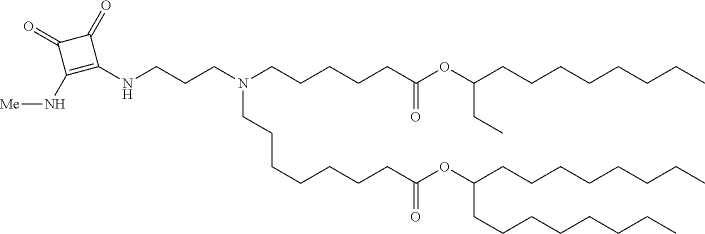 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I 351 | 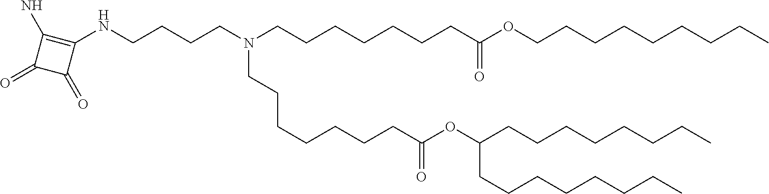 [SMILES: CCCCCCCCCOC(=O)CCCCCCCN(CCCCCCCC(=O)OC(CCCCCCCC)CCCCCCCC)CCCCNc1c(N)c(=O)c1=O] |
| I 352 | 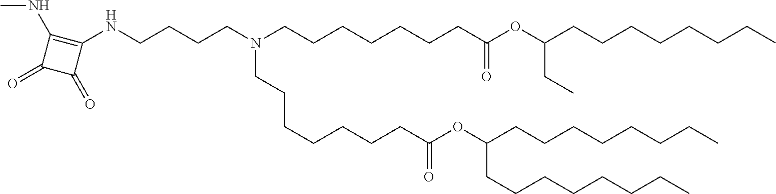 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I 353 | 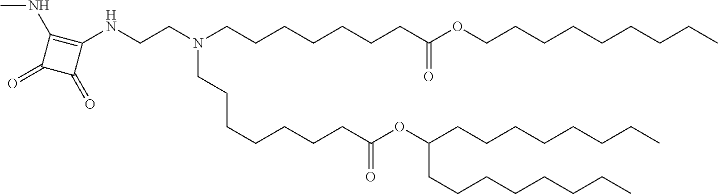 [SMILES: CCCCCCCCCOC(=O)CCCCCCCN(CCCCCCCC(=O)OC(CCCCCCCC)CCCCCCCC)CCNc1c(NC)c(=O)c1=O] |
| I 354 | 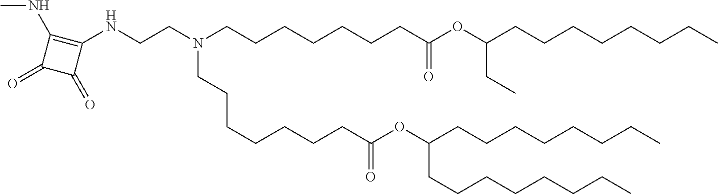 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I 355 | 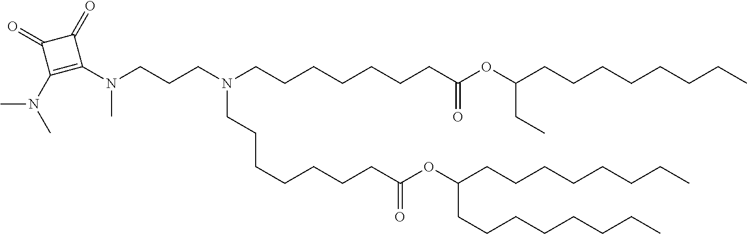 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |

In some embodiments, a lipid of the disclosure comprises Compound I-340A:

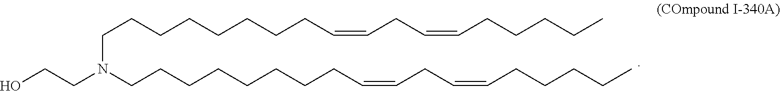 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I IIa), (I IIb), (I IIc), (I IId), (I IIe), (I IIf), (I IIg), (I IIh), (I IIj), (I IIk), (I III), (I VI), (I VI-a), (I VII), (I VIIa), (I VIIb-1), (I VIIb-2), (I VIIb-3), (I VIIb-4), (I VIIb-5), (I VIIc), (I VIId), (I VIII), (I VIIIa), (I VIIIb), (I VIIIc), (I VIIId), (I XI), (I XI-a), or (I XI-b) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

The ionizable lipid may comprise a single enantiomer, or a mixture of enantiomers at a certain ratio. In some embodiments, the ionizable lipid comprises a substantially pure enantiomer. In some embodiments, a substantially pure enantiomer is substantially free from other enantiomers or stereoisomers of the compound (i.e., in enantiomeric excess). In some embodiments, an “S” form of the ionizable lipid is substantially free from the “R” form of the ionizable lipid and is, thus, in enantiomeric excess of the “R” form. In some embodiments, an “R” form of the ionizable lipid is substantially free from the “S” form of the ionizable lipid and is, thus, in enantiomeric excess of the “S” form. In some embodiments, ‘substantially free’, refers to: (i) an aliquot of an “R” form compound that contains less than 2% “S” form; or (ii) an aliquot of an “S” form compound that contains less than 2% “R” form. In some embodiments, a substantially pure enantiomer comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the single enantiomer. In certain embodiments, the weights are based upon total weight of all enantiomers or stereoisomers of the compound. In one embodiment, the ionizable lipid comprises a racemic mixture of the “S” and “R” forms.

In some embodiments, the ionizable lipid comprises a racemic mixture of an amino lipid. In some embodiments, the ionizable lipid comprises a substantially pure enantiomer of an amino lipid. In some embodiments, the ionizable lipid comprises a substantially pure (R)-enantiomer of an amino lipid. In some embodiments, the ionizable lipid comprises a substantially pure (S)-enantiomer of an amino lipid. In some embodiments, the ionizable lipid comprises a substantially pure enantiomer of a compound of any of Formulae (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), (I VIIId), (I XI), (I XI-a), or (I XI-b), and/or a compound selected from the group consisting of Compound I-49, and Compound I-301.

In some embodiments, the ionizable lipid comprises a substantially pure enantiomer of Compound I-49. In some embodiments, the ionizable lipid comprises substantially pure Compound (S)-I-49:

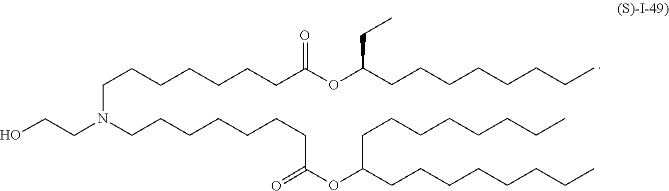 [SMILES: CCCCCCCCC(CCCCCCCC)OC(=O)CCCCCCCN(CCO)CCCCCCCC(=O)O[C@@H](CC)CCCCCCCC]

In some embodiments, the ionizable lipid comprises substantially pure Compound (R)-I-49:

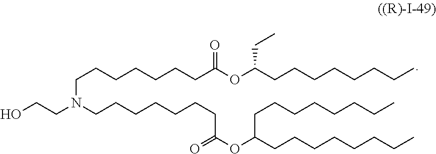 [SMILES: CCCCCCCCC(CCCCCCCC)OC(=O)CCCCCCCN(CCO)CCCCCCCC(=O)O[C@H](CC)CCCCCCCC]

In some embodiments, the ionizable lipid comprises a substantially pure enantiomer of Compound I-301. In some embodiments, the ionizable lipid comprises substantially pure Compound (S)-I-301:

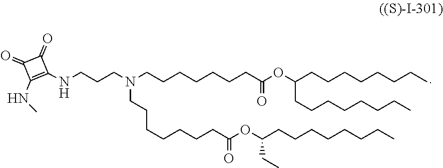 [SMILES: CCCCCCCCC(CCCCCCCC)OC(=O)CCCCCCCN(CCCCCCCC(=O)O[C@@H](CC)CCCCCCCC)CCCNc1c(NC)c(=O)c1=O]

In some embodiments, the ionizable lipid comprises substantially pure Compound (R)-I-301:

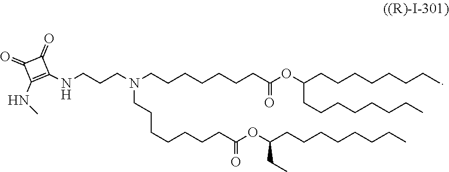 [SMILES: CCCCCCCCC(CCCCCCCC)OC(=O)CCCCCCCN(CCCCCCCC(=O)O[C@H](CC)CCCCCCCC)CCCNc1c(NC)c(=O)c1=O]

In some aspects, the ionizable lipids of the present disclosure may be one or more of compounds of formula (I XII),

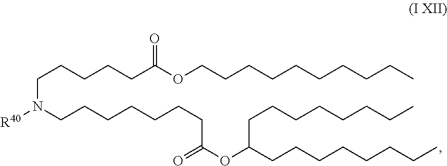

or its N-oxide, or a salt or isomer thereof, wherein:

R⁴⁰ is not a squaramide-substituted group, and is selected from the group consisting of hydrogen, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —O(CH₂)ₙOR, —N(R)C(═NR⁹)N(R)₂, —N(R)C(═CHR⁹)N(R)₂, —OC(O) N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R is independently selected from the group consisting of C₁₋₃ alkyl, C₂₋₃ alkenyl, (CH₂)_{q}OR*, and H, wherein q is independently selected from 1, 2, and 3, and R* is independently selected from the group consisting of C₁₋₁₂ alkyl and C₂₋₁₂ alkenyl;

each R⁹ is independently selected from the group consisting of H, CN, NO₂, C₁₋₆ alkyl, —OR, —S(O)₂R, —S(O)₂N(R)₂, or C₂₋₆ alkenyl;

R¹⁰ is selected from the group consisting of H, OH, C₁₋₃ alkyl, and C₂₋₃ alkenyl; and

X is independently selected from the group consisting of F, Cl, Br, and I.

In some embodiments, R⁴⁰ is not a squaramide-substituted group. In some embodiments, R⁴⁰ is selected from the group consisting of hydrogen, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5.

In some aspects, the ionizable lipids of the present disclosure may be one or more of compounds of formula I (I IX),

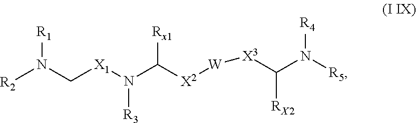

or salts or isomers thereof, wherein

W is

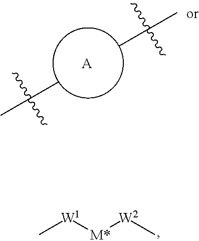

ring A is

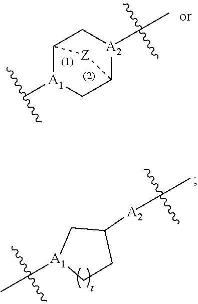

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

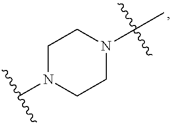 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

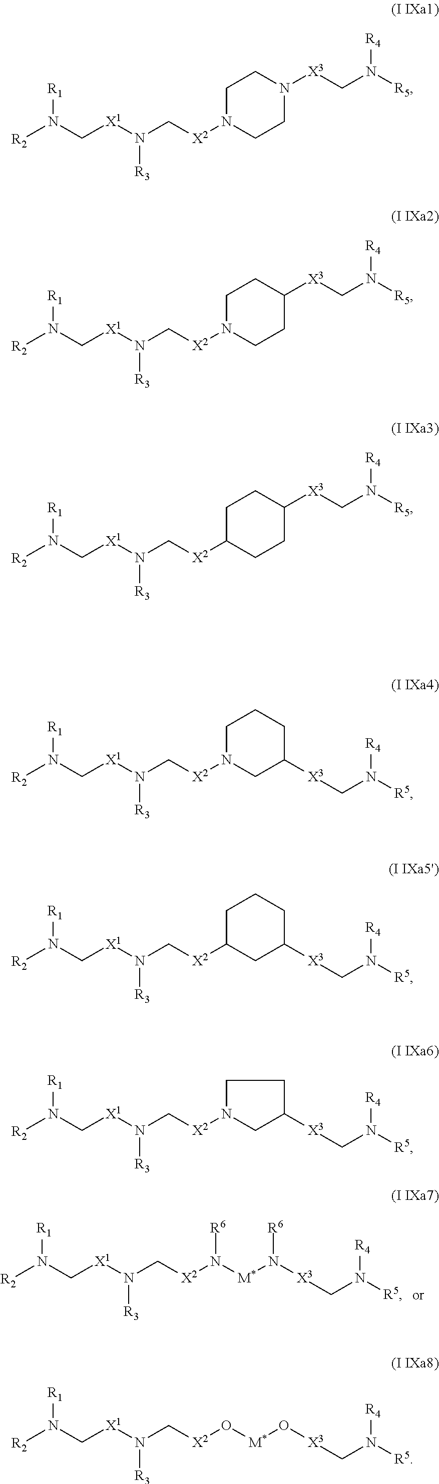

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

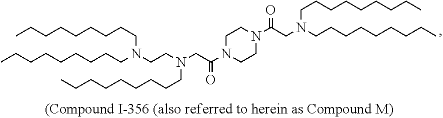 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.
In some embodiments, the ionizable lipid is

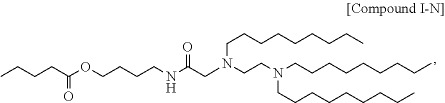 [SMILES: CCCCCCCCCN(CCCCCCCCC)CCN(CCCCCCCCC)CC(=O)NCCCCOC(=O)CCCC]

or a salt thereof.
In some embodiments, the ionizable lipid is

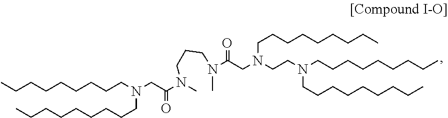 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.
In some embodiments, the ionizable lipid is

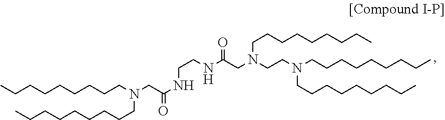 [SMILES: CCCCCCCCCN(CCCCCCCCC)CCN(CCCCCCCCC)CC(=O)NCCNC(=O)CN(CCCCCCCCC)CCCCCCCCC]

or a salt thereof.
In some embodiments, the ionizable lipid is

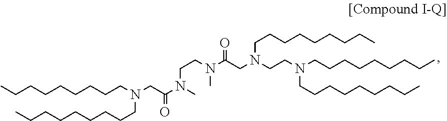 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (I IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b) (each of these preceded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (I VIIb-4), (I VIIb-5), (VIIc), (VIId), (VIIIc), (VIIId), (XI), (XI-a), or (XI-b) (each of these preceded by the letter I for clarity) is about 45 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b) (each of these preceded by the letter I for clarity) is about 40 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), (IIf), (IIg), (IIh), (IIj), (Ilk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety. For example, the ionizable amino lipids include, but not limited to:

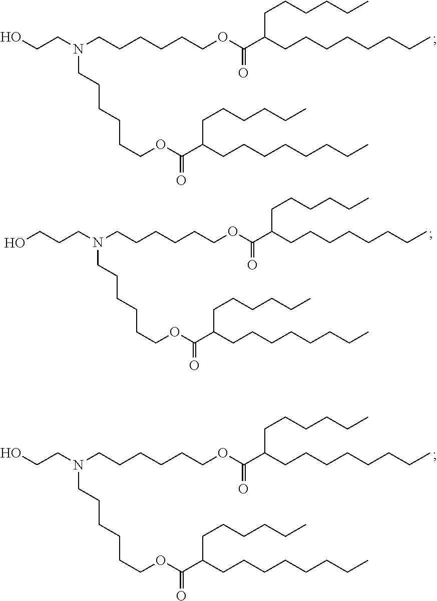 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCO)CCCCCCOC(=O)C(CCCCCC)CCCCCCCC]

and any combination thereof.

Ionizable lipids of the invention can also be the compounds disclosed in International Publication No. WO 2015/199952 A1, hereby incorporated by reference in its entirety. For example, the ionizable amino lipids include, but not limited to:

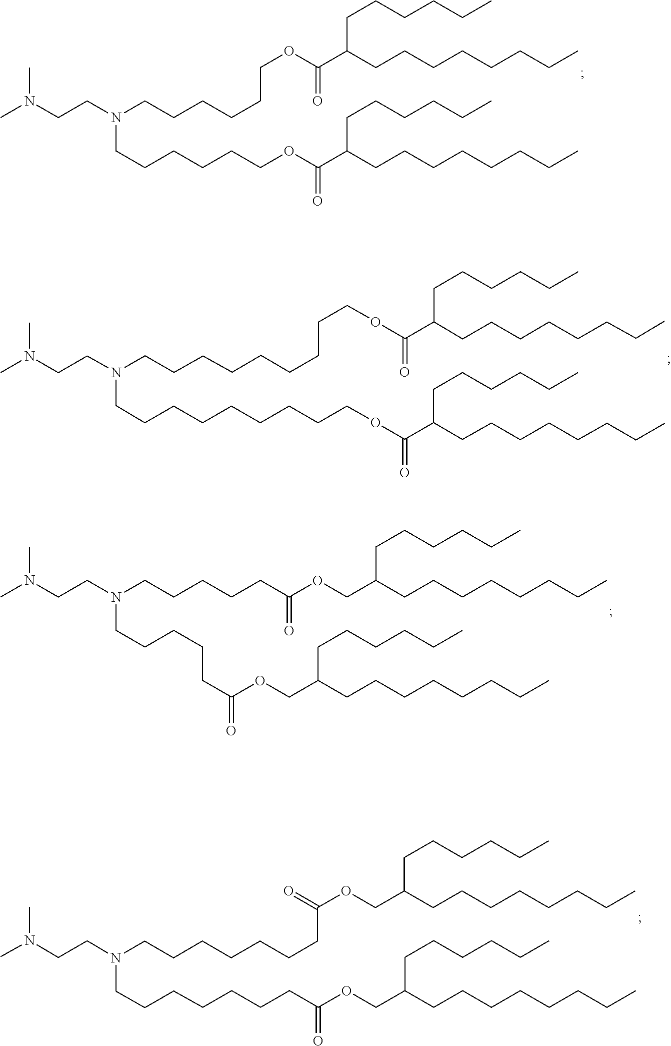

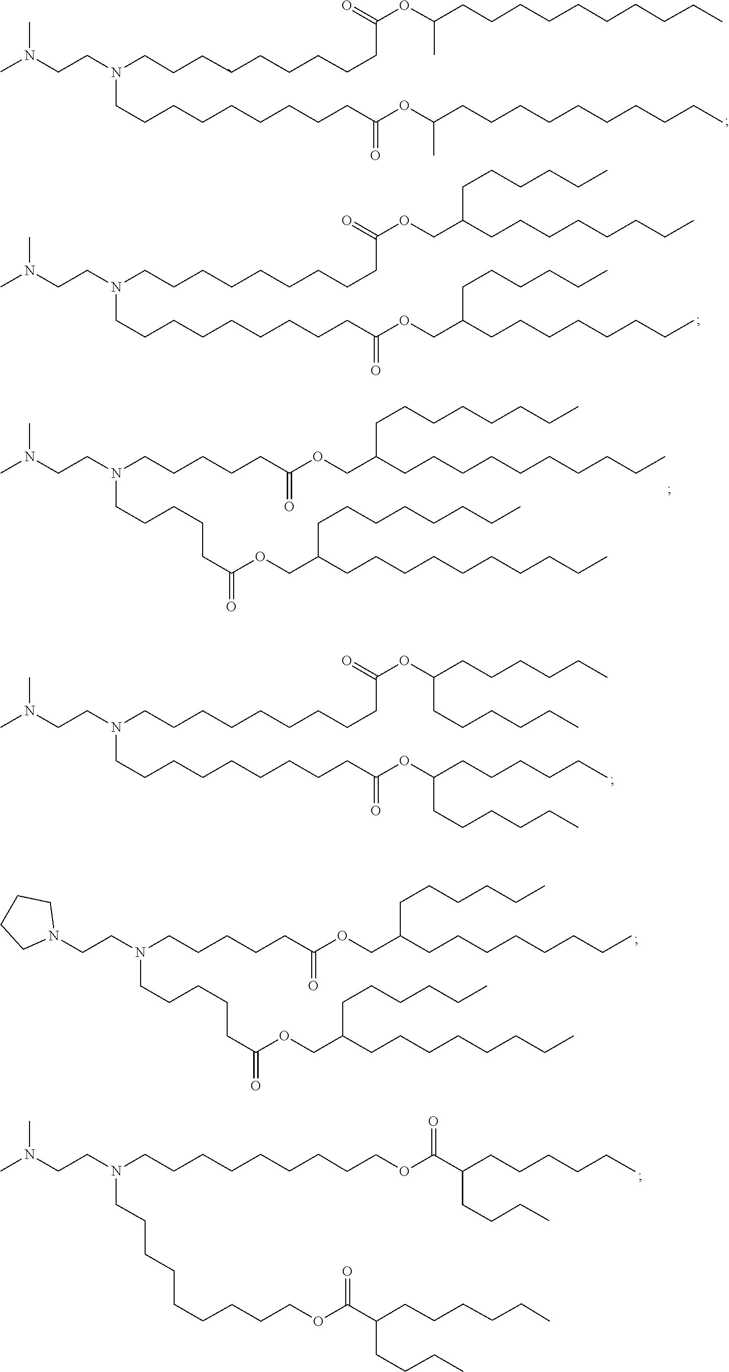

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), (IIf), (IIg), (IIh), (IIj), (IIk), (III), (VI), (VI-a), (VII), (VIII), (VIIa), (VIIIa), (VIIIb), (VIIb-1), (VIIb-2), (VIIb-3), (VIIb-4), (VIIb-5), (VIIc), (VIId), (VIIIc), (VIIId), (XI), (XI-a), or (XI-b), (each of these preceded by the letter I for clarity).

In any of the foregoing or related aspects, the ionizable lipid of the LNP of the disclosure comprises a compound comprising any of Compound Nos. I 1-356.

In any of the foregoing or related aspects, the ionizable lipid of the LNP of the disclosure comprises at least one compound selected from the group consisting of: Compound Nos. I 18 (also referred to as Compound X), I 48, I 49, I 50, I 182, I 184, I 292, I 301, I 309, I 317, I 321, I 326, I 347, I 348, I 349, I 350, and I 352. In another embodiment, the ionizable lipid of the LNP of the disclosure comprises a compound selected from the group consisting of: Compound Nos. I 18 (also referred to as Compound X), I 49, I 182, I 184, I 301, and I 321. In another embodiment, the ionizable lipid of the LNP of the disclosure comprises a compound selected from the group consisting of: Compound Nos. I 49 and I 301.

In any of the foregoing or related aspects, the synthesis of compounds of the invention, e.g. compounds comprising any of Compound Nos. 1-356, follows the synthetic descriptions in U.S. Provisional Patent Application No. 62/733,315, filed Sep. 19, 2018. In some embodiments, the synthesis of a Compound of any of Formulae (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIIa), (I VIIb-1), (I VIIb-2), (I VIIb-3), (I VIIb-4), (I VIIb-5), (I VIIc), (I VIId), (I VIII), (I VIIIa), (I VIIIb), (I VIIIc), (I VIIId), (I XI), (I XI-a), or (I XI-b) (e.g., Compound I-49 or Compound I-301) may be prepared following the general procedures described on pages 181, 190, and 191 of PCT/US2018/022717, which is incorporated herein by reference in its entirety.

### Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

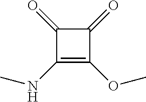

### Chemical Formula: C₆H₇NO₃

### Molecular Weight: 141.13

To a solution of 3,4-dimethoxy-3-cyclobutene-1,2-dione (1 g, 7 mmol) in 100 mL diethyl ether was added a 2M methylamine solution in THE (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

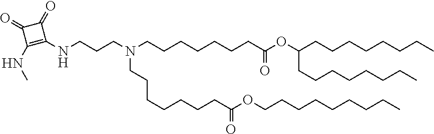 [SMILES: CCCCCCCCCOC(=O)CCCCCCCN(CCCCCCCC(=O)OC(CCCCCCCC)CCCCCCCC)CCCNc1c(NC)c(=O)c1=O]

### Chemical Formula: C₅₀H₉₃N₃O₆

### Molecular Weight: 832.31

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

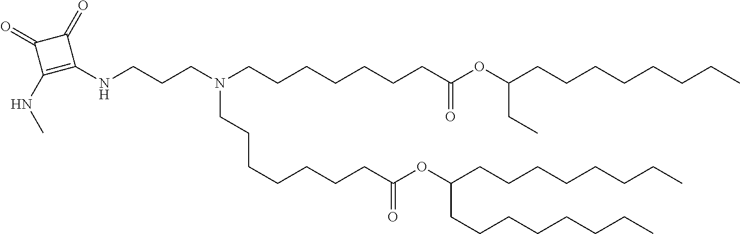 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Chemical Formula: C₅₂H₉₇N₃O₆

### Molecular Weight: 860.36

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆.

¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

### Compound I-49: Heptadecan-9-yl 8-((2-hydroxyethyl)(8-oxo-8-(undecan-3-yloxy)octyl)amino)octanoate

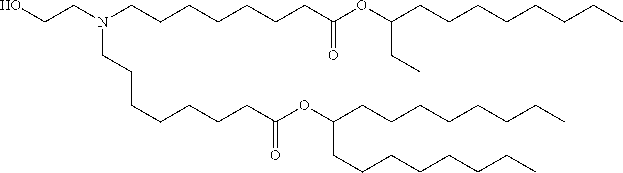 [SMILES: CCCCCCCCC(CC)OC(=O)CCCCCCCN(CCO)CCCCCCCC(=O)OC(CCCCCCCC)CCCCCCCC]

### Chemical Formula: C₄₆H₉₁NO₅

### Molecular Weight: 738.24

Compound I-49 may be prepared following the general procedures described on pages 181, 190, and 191 of PCT/US2018/022717, which is incorporated herein by reference in its entirety. UPLC/ELSD: RT=3.68 min. MS (ES): m/z (MH⁺) 739.21 for C₄₆H₉₁NO₅. ¹H NMR (300 MHz, CDCl₃): δ ppm 4.89 (m, 2H); 3.56 (br. m, 2H); 2.68-2.39 (br. m, 5H); 2.30 (m, 4H); 1.71-1.19 (m, 66H); 0.90 (m, 12H).

(ii) Cholesterol/Structural Lipids

The target cell target cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

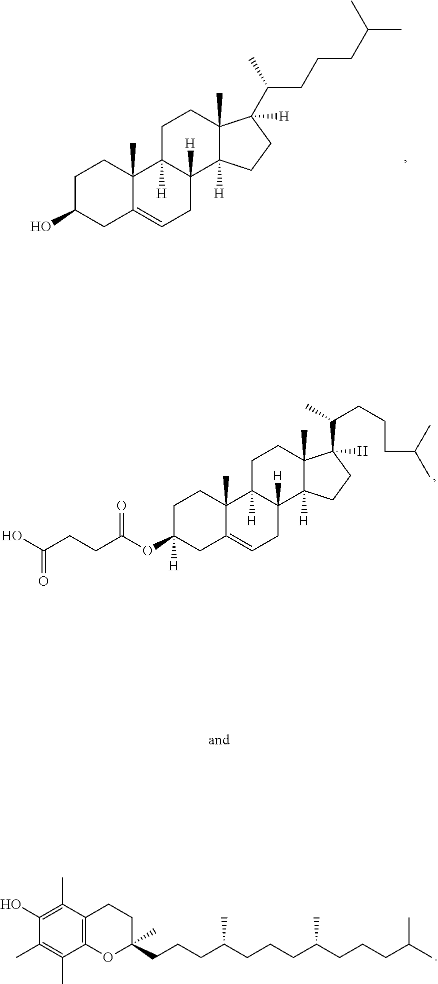

The target cell target cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

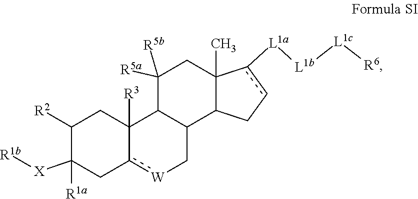

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

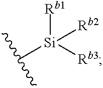

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or;

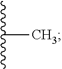

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

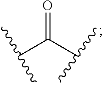 [SMILES: *C(*)=O]

L^{1a} is absent,

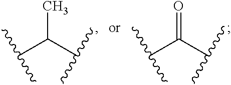

L^{1b} is absent,

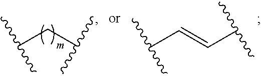

m is 1, 2, or 3;

L^{1c} is absent, or

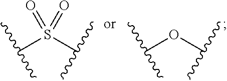

and

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl,

or a pharmaceutically acceptable salt thereof.

In some embodiments, the compound has the structure of Formula SIa:

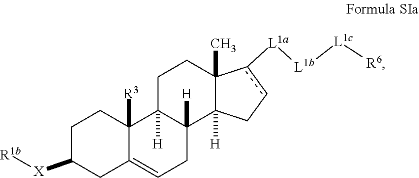

or a pharmaceutically acceptable salt thereof.

In some embodiments, the compound has the structure of Formula SIb:

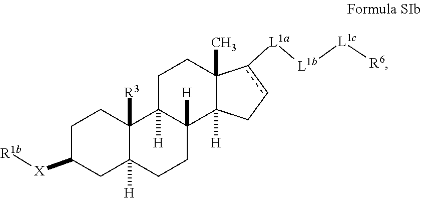

or a pharmaceutically acceptable salt thereof.

In some embodiments, the compound has the structure of Formula SIc:

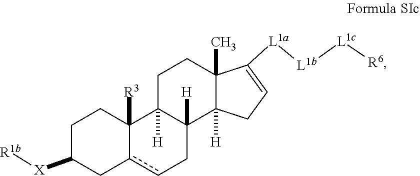

or a pharmaceutically acceptable salt thereof.

In some embodiments, the compound has the structure of Formula SId:

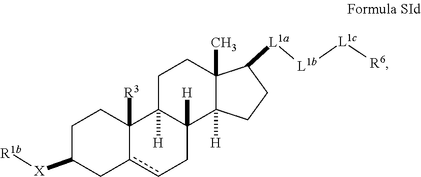

or a pharmaceutically acceptable salt thereof.

In some embodiments, L^{1a} is absent. In some embodiments, L^{1a} is

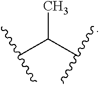 [SMILES: *C(*)C]

In some embodiments, L^{1a} is

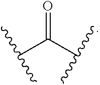 [SMILES: *C(*)=O]

In some embodiments, L^{1b} is absent. In some embodiments, L^{1b} is

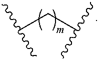

In some embodiments, L^{1b} is

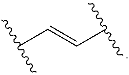 [SMILES: */C=C/*]

In some embodiments, m is 1 or 2. In some embodiments, m is 1. In some embodiments, m is 2.

In some embodiments, L^{1c} is absent. In some embodiments, L^{1c} is

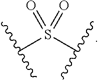 [SMILES: *S(*)(=O)=O]

In some embodiments, L^{1c} is

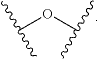 [SMILES: *O*]

In some embodiments, R⁶ is optionally substituted C₆-C₁₀ aryl.

In some embodiments, R⁶ is

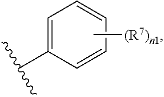

where

n1 is 0, 1, 2, 3, 4, or 5; and

each R⁷ is, independently, halo or optionally substituted C₁-C₆ alkyl.

In some embodiments, each R⁷ is, independently

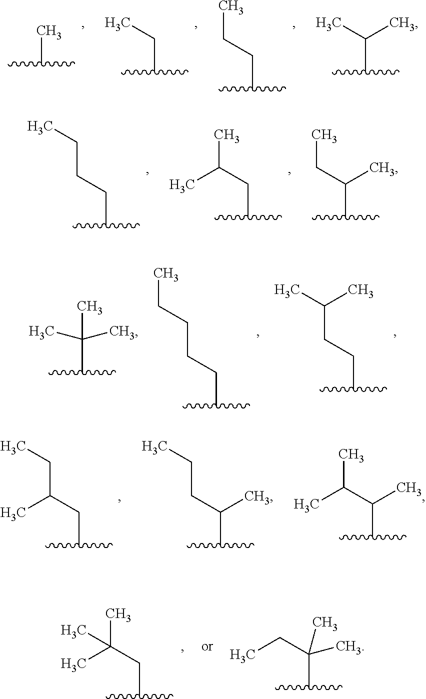

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

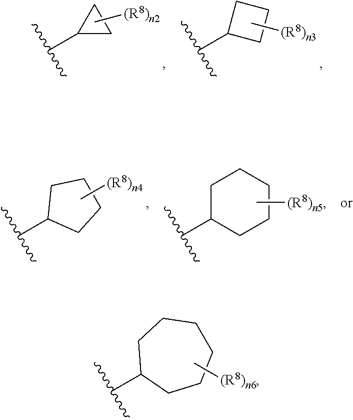

where

n2 is 0, 1, 2, 3, 4, or 5;

n3 is 0, 1, 2, 3, 4, 5, 6, or 7;

n4 is 0, 1, 2, 3, 4, 5, 6, 7, 8, or 9;

n5 is 0, 1, 2, 3, 4, 5, 6, 7, 8, 9, 10, or 11;

n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently,

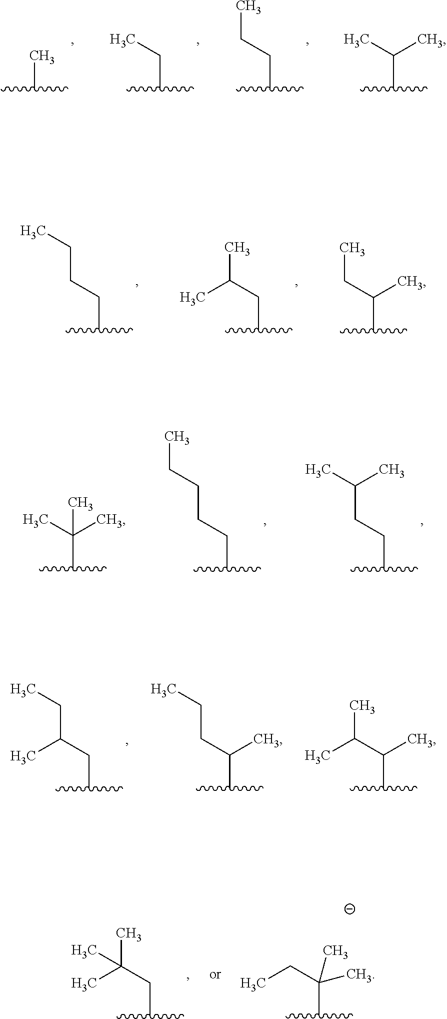

In some embodiments, R⁶ is optionally substituted C₃-C₁₀ polycycloalkyl.

In some embodiments, R⁶ is

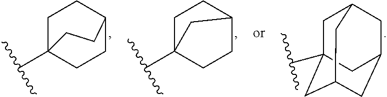

In some embodiments, R⁶ is optionally substituted C₃-C₁₀ cycloalkenyl.

In some embodiments, R⁶ is

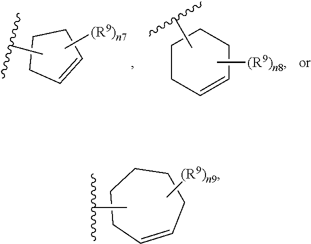

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

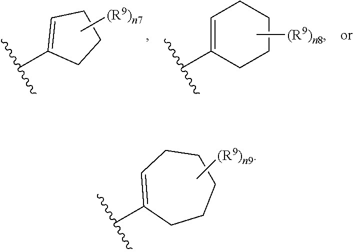

In some embodiments, each R⁹ is, independently,

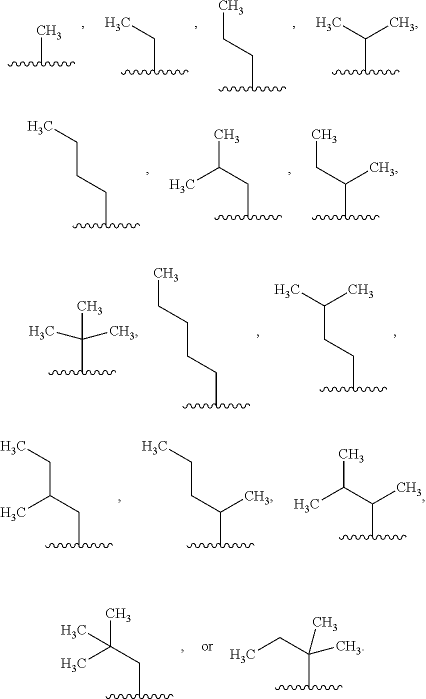

In some embodiments, R⁶ is optionally substituted C₂-C₉ heterocyclyl.

In some embodiments, R⁶ is

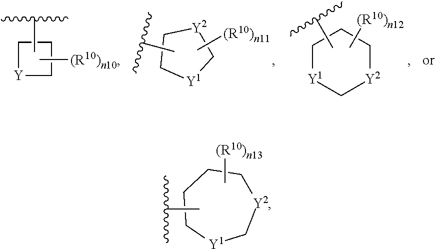

where

n10 is 0, 1, 2, 3, 4, or 5;

n11 is 0, 1, 2, 3, 4, or 5;

n12 is 0, 1, 2, 3, 4, 5, 6, or 7;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;

each of R^{11a} and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O.

In some embodiments, Y² is O. In some embodiments, Y² is CR^{1a}R^{11b}.

In some embodiments, each R¹⁰ is, independently,

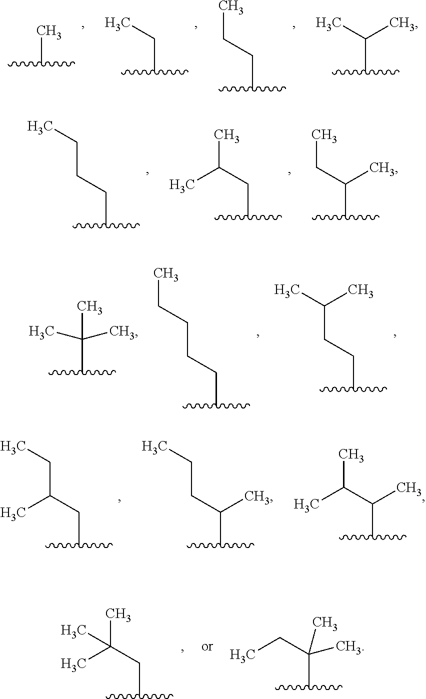

In some embodiments, R⁶ is optionally substituted C₂-C₉ heteroaryl.

In some embodiments, R⁶ is

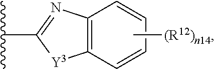

where

Y³ is NR^{C}, O, or S

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, R⁶ is

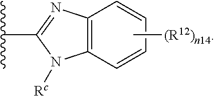

In some embodiments, R⁶ is

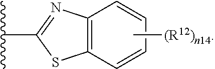

In an aspect, the structural lipid of the invention features a compound having the structure of Formula SII:

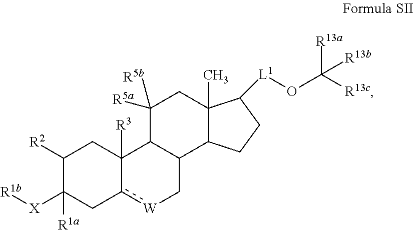

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

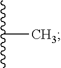

[symbol] represents a single bond or a double bond;

W is CR^{4a} or CR^{4a}CR^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

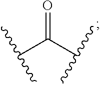 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula SIIa:

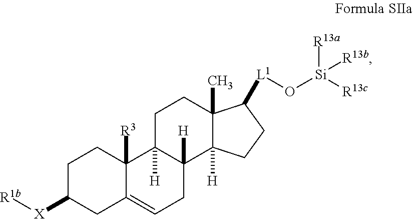

or a pharmaceutically acceptable salt thereof.

In some embodiments, the compound has the structure of Formula SIIb:

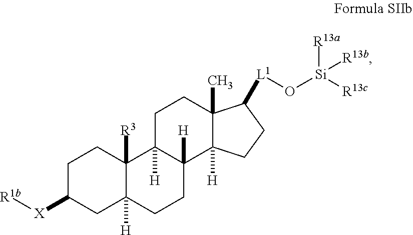

or a pharmaceutically acceptable salt thereof.

In some embodiments, L¹ is

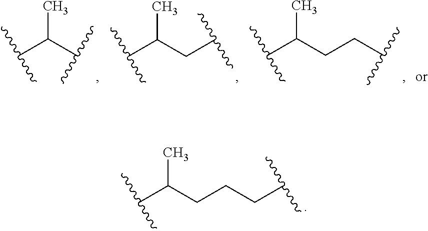

In some embodiments, each of R^{13a}, R^{13b}, and R^{13c} is, independently,

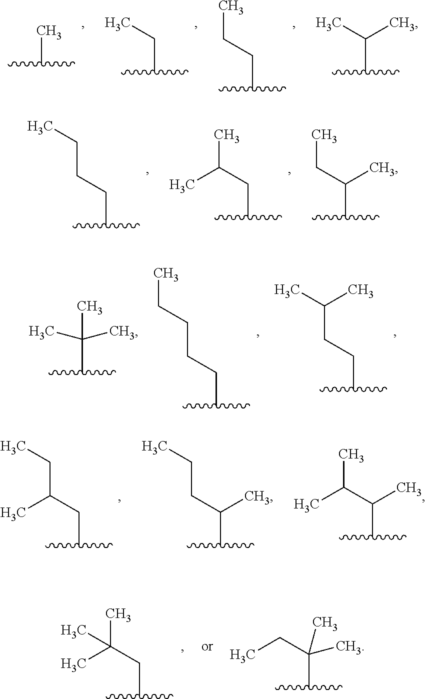

In an aspect, the structural lipid of the invention features a compound having the structure of Formula SIII.

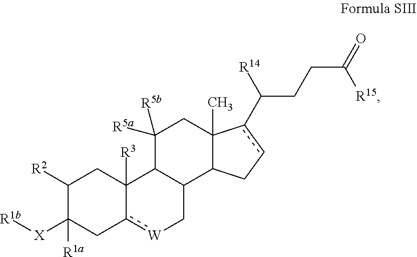

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

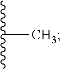

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c}, is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

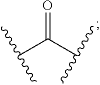 [SMILES: *C(*)=O]

R¹⁴ is H or C₁-C₆ alkyl; and

R¹⁵ is

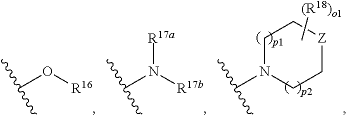

where

R¹⁶ is H or optionally substituted C₁-C₆ alkyl;
R^{17b} is H, OR^{17c}, optionally substituted C₆-C₁₀ aryl, or optionally substituted C₁-C₆ alkyl;

R¹⁷, is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂O, S, or NR^{D}, where R^{D} is H or optionally substituted C₁-C₆ alkyl; and each R¹⁸ is, independently, halo or optionally substituted C₁-C₆ alkyl,

or a pharmaceutically acceptable salt thereof.

In some embodiments, the compound has the structure of Formula SIIIa:

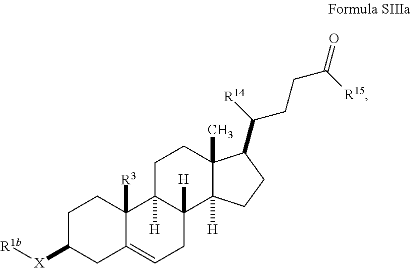

or a pharmaceutically acceptable salt thereof.

In some embodiments, the compound has the structure of Formula SIIIb:

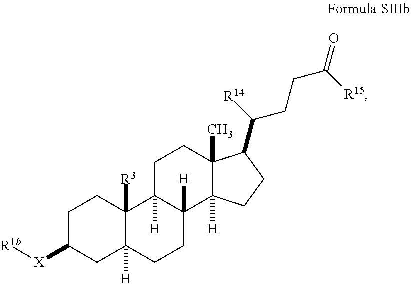

or a pharmaceutically acceptable salt thereof.

In some embodiments, R¹⁴ is H,

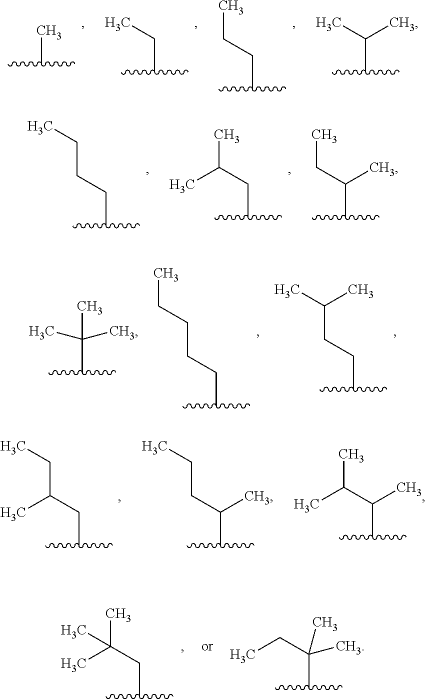

In some embodiments, R¹⁴ is

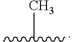

In some embodiments, R¹⁵ is

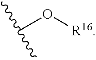

In some embodiments, R¹⁵ is

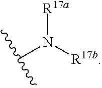

In some embodiments, R¹⁶ is H. In some embodiments, R¹⁶ is

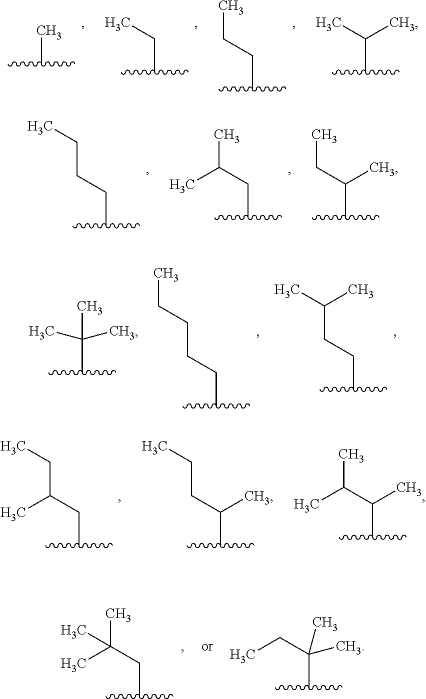

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

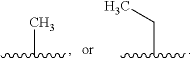

In some embodiments, R^{17c} is H. In some embodiments, R^{17c} is

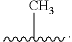

In some embodiments, R¹⁵ is

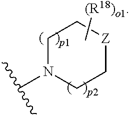

In some embodiments, each R⁸ is, independently,

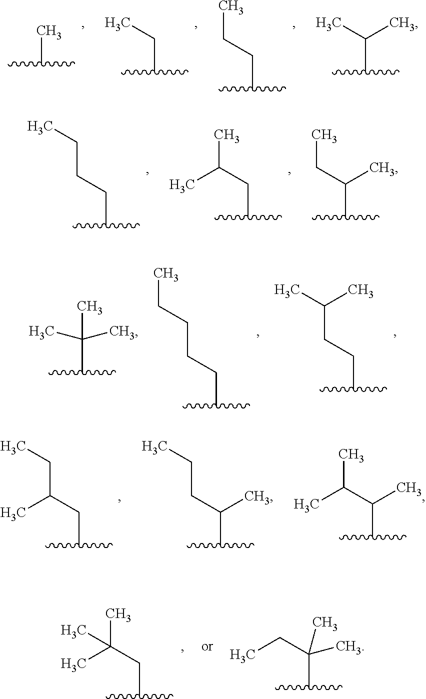

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

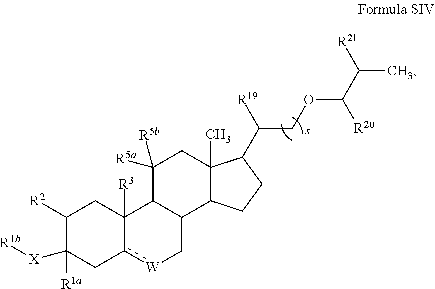

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

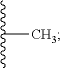

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

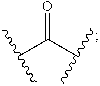 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

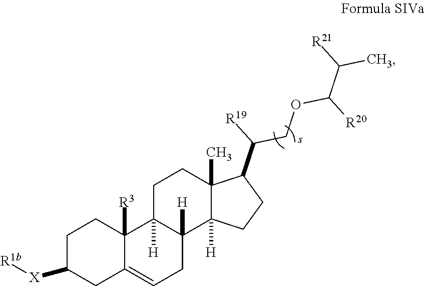

or a pharmaceutically acceptable salt thereof.

In some embodiments, the compound has the structure of Formula SIVb:

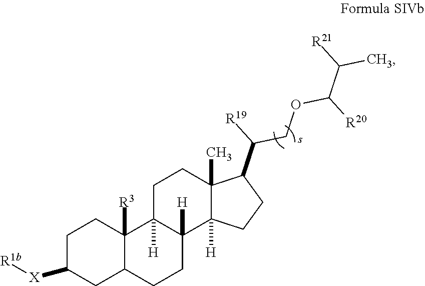

or a pharmaceutically acceptable salt thereof.

In some embodiments, R¹⁹ is

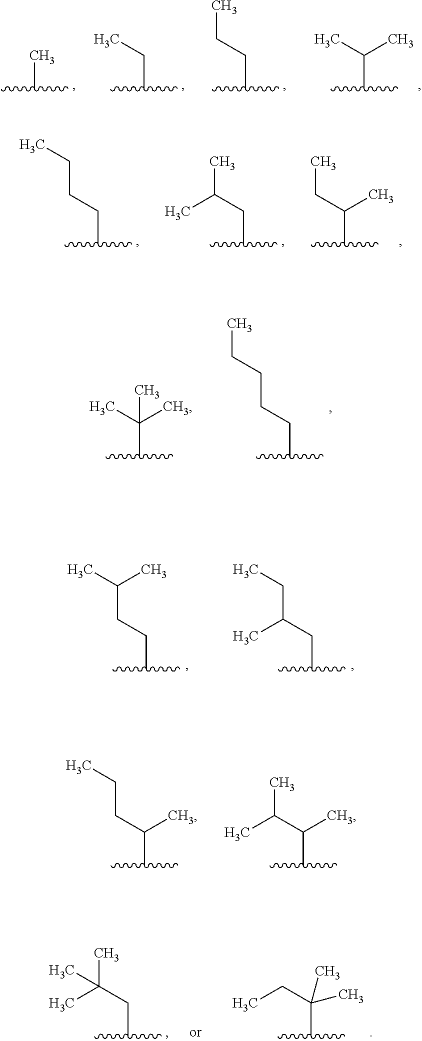

In some embodiments, R¹⁹ is

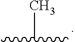

In some embodiments, R²⁰ is,

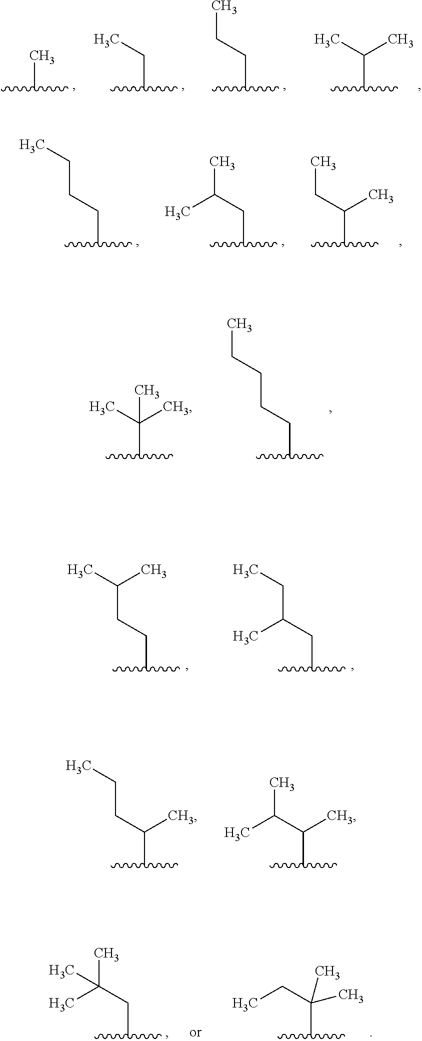

In some embodiments, R²¹ is H,

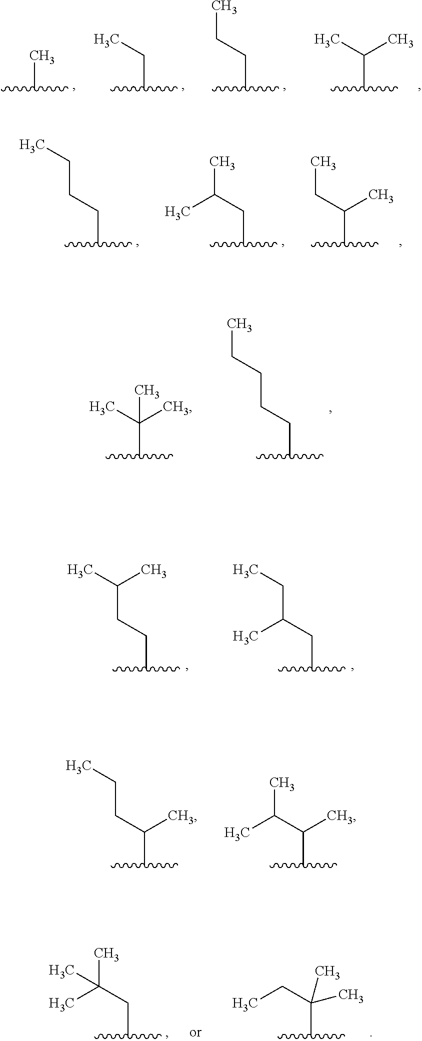

In an aspect, the structural lipid of the invention features, a compound having the structure of Formula SV:

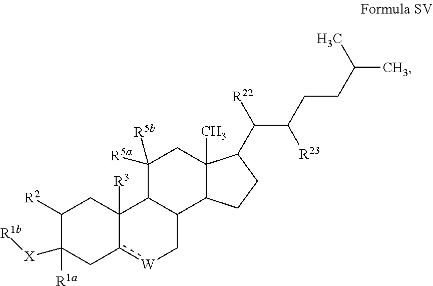

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H

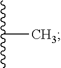

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl; each of R^{5a} and R^{5b} is, independently, H or OR^{A}, or R^{5a} and R^{5b}, together with the atom to which each is attached, combine to form

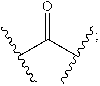 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

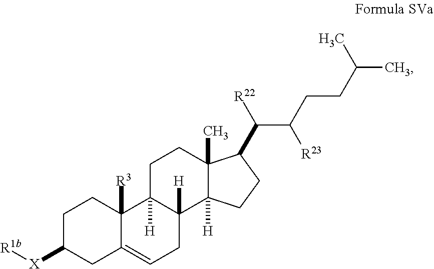

or a pharmaceutically acceptable salt thereof.

In some embodiments, the compound has the structure of Formula SVb:

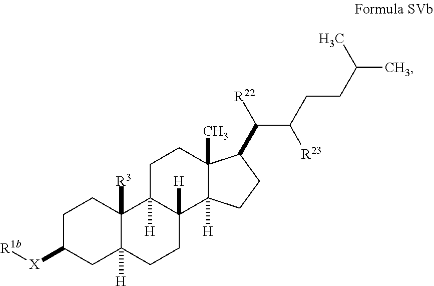

or a pharmaceutically acceptable salt thereof.
In some embodiments, R²² is H,

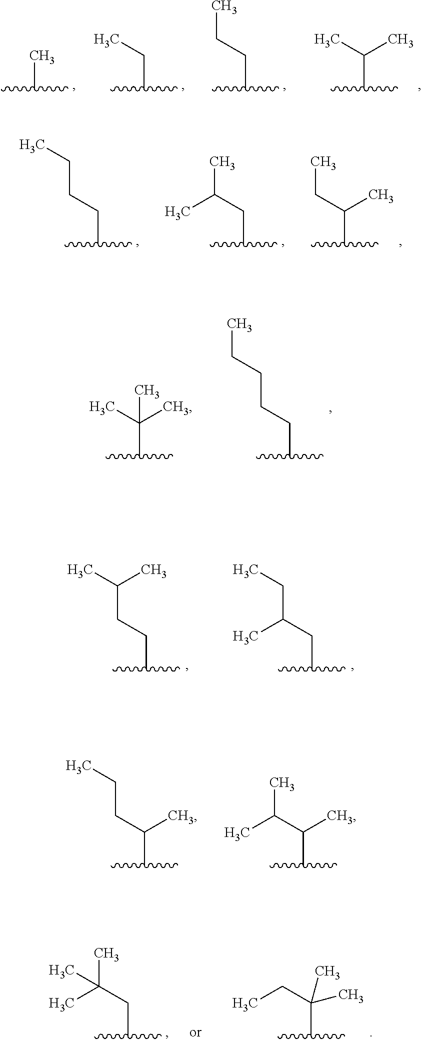

In some embodiments, R²² is

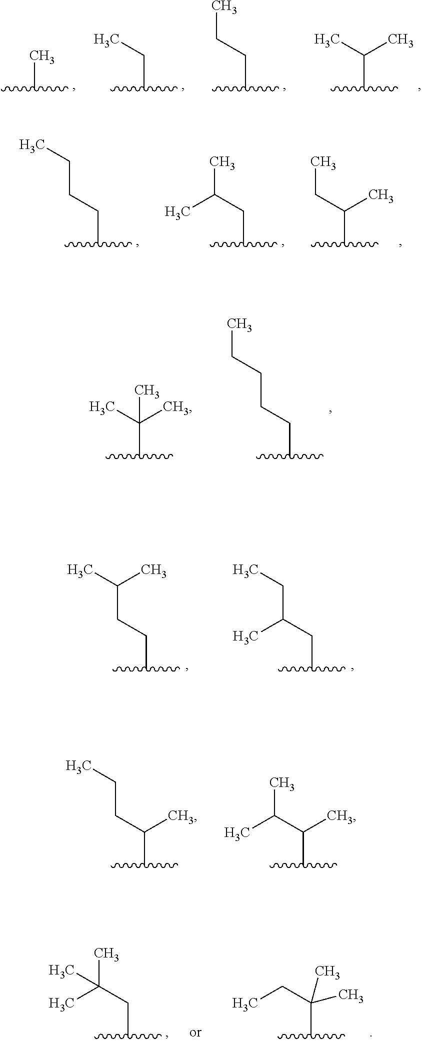

In some embodiments, R²³ is

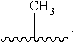

In an aspect, the structural lipid of the invention features a compound having the structure of Formula SVL:

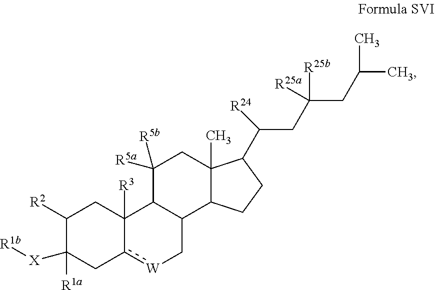

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

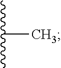

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

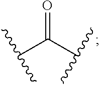 [SMILES: *C(*)=O]

R²⁴ is H or C₁-C₆ alkyl; and

each of R^{25a} and R^{25b} is C₁-C₆ alkyl, or a pharmaceutically acceptable salt thereof.

In some embodiments, the compound has the structure of Formula SVIa:

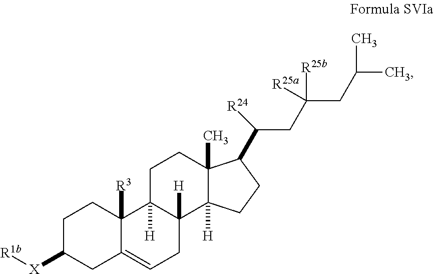

or a pharmaceutically acceptable salt thereof.

In some embodiments, the compound has the structure of Formula SVIb:

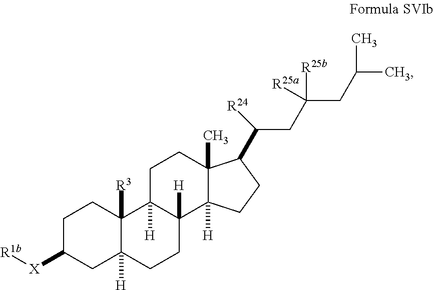

or a pharmaceutically acceptable salt thereof.

In some embodiments, R²⁴ is H,

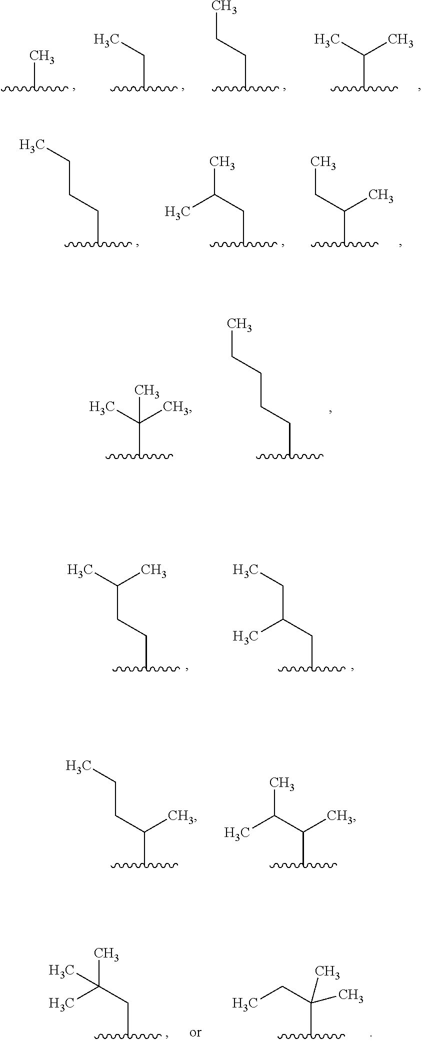

In some embodiments, R²⁴ is

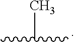

In some embodiments, each of R^{25a} and R^{25b} is, independently,

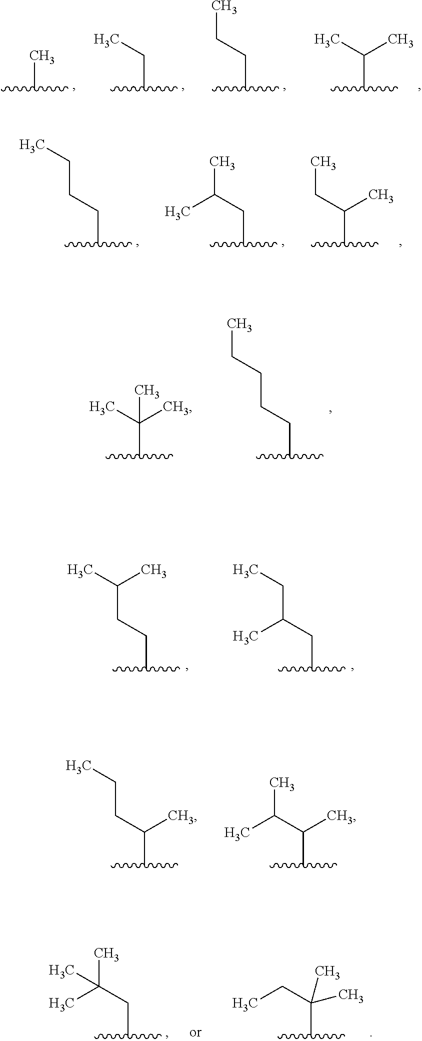

In an aspect, the structural lipid of the invention features a compound having the structure of Formula SVII:

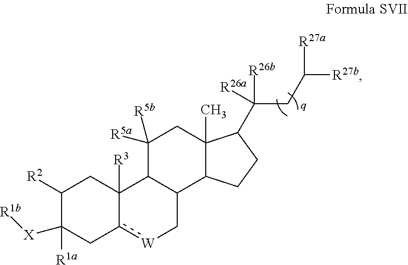

where

R^{1a} is H, optionally substituted C₁-C₆ alkyl, optionally substituted C₂-C₆ alkenyl, optionally substituted C₂-C₆ alkynyl, or

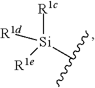

Where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is Ho

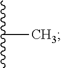

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

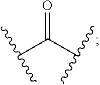 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

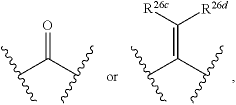

where each of R^{26e} and R²⁶ is, independently, H or optionally substituted C₁-C₆ alkyl; and

each of R^{27a} and R^{27b} is H, hydroxyl, or optionally substituted C₁-C₆ alkyl,

or a pharmaceutically acceptable salt thereof.

In some embodiments, the compound has the structure of Formula SVIIa:

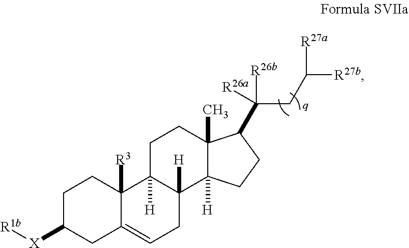

or a pharmaceutically acceptable salt thereof.

In some embodiments, the compound has the structure of Formula SVIIb:

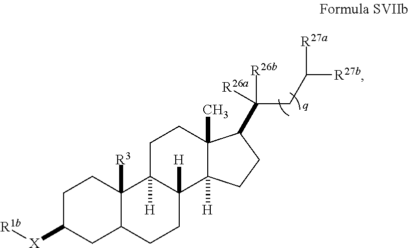

or a pharmaceutically acceptable salt thereof.

In some embodiments, R^{26a} and R^{26b} is, independently, H,

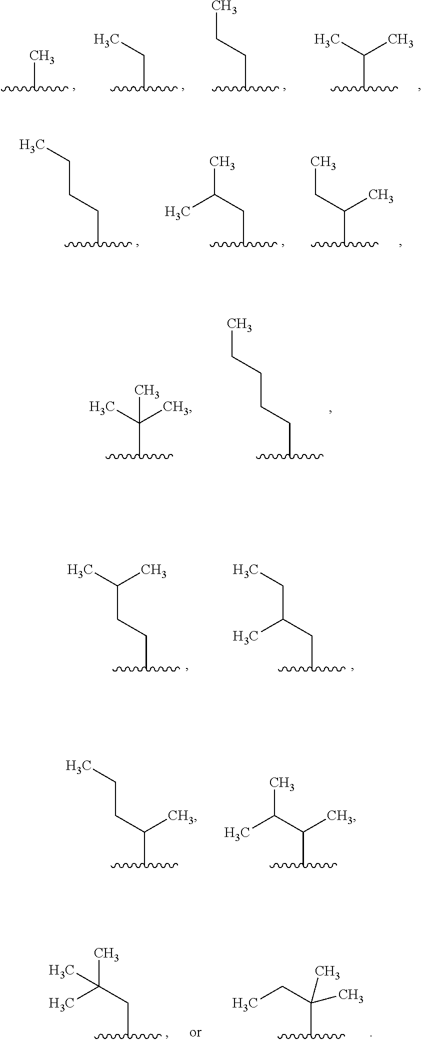

In some embodiments, R^{26a} and R^{26b}, together with the atom to which each is attached, combine to form

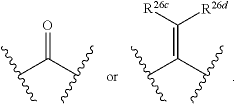

In some embodiments, R^{26a} and R^{26b}, together with the atom to which each is attached, combine to form

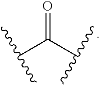 [SMILES: *C(*)=O]

In some embodiments, R^{26a} and R^{26b}, together with the atom to which each is attached, combine to form

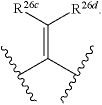

In some embodiments, where each of R^{26c} and R²⁶ is, independently, H,

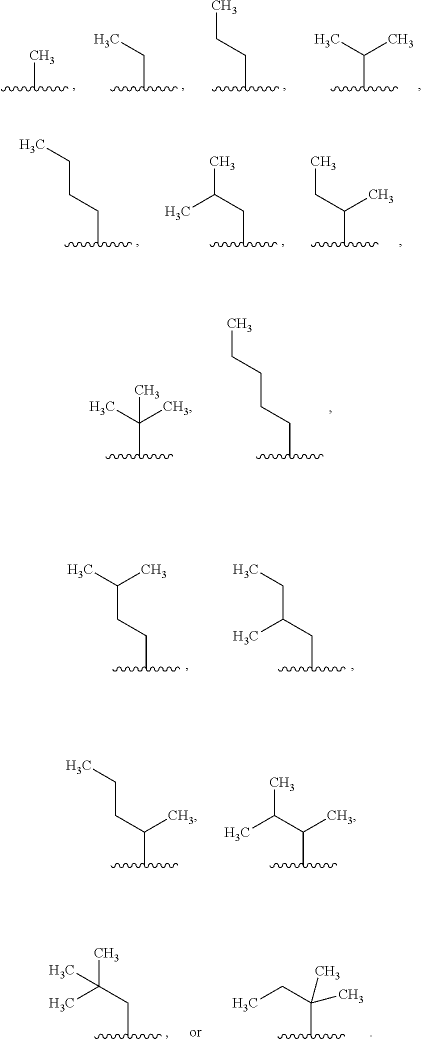

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

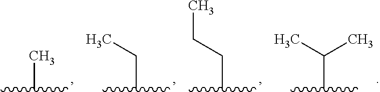

In an aspect, the structural lipid of the invention features a compound having the structure of Formula SVIII.

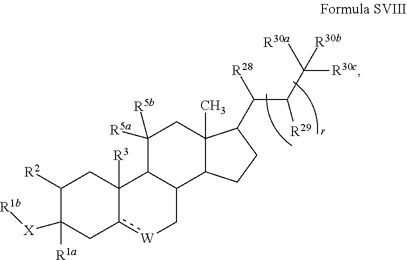

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

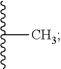

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

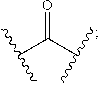 [SMILES: *C(*)=O]

R²⁸ is H or optionally substituted C₁-C₆ alkyl;

r is 1, 2, or 3;
each R²⁹ is, independently, H or optionally substituted C₁-C₆ alkyl; and
each of R^{30a}, R^{30b}, and R³⁰, is C₁-C₆ alkyl,
   or a pharmaceutically acceptable salt thereof.

In some embodiments, the compound has the structure of Formula SVIIIa:

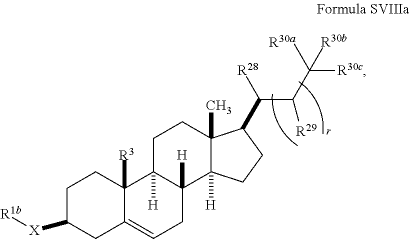

or a pharmaceutically acceptable salt thereof.

In some embodiments, the compound has the structure of Formula SVIIIb:

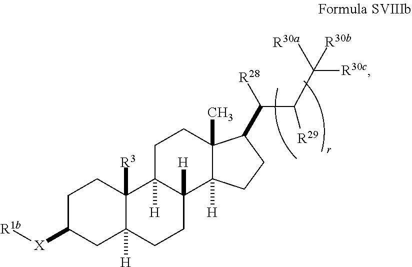

or a pharmaceutically acceptable salt thereof.

In some embodiments, R²⁸ is H,

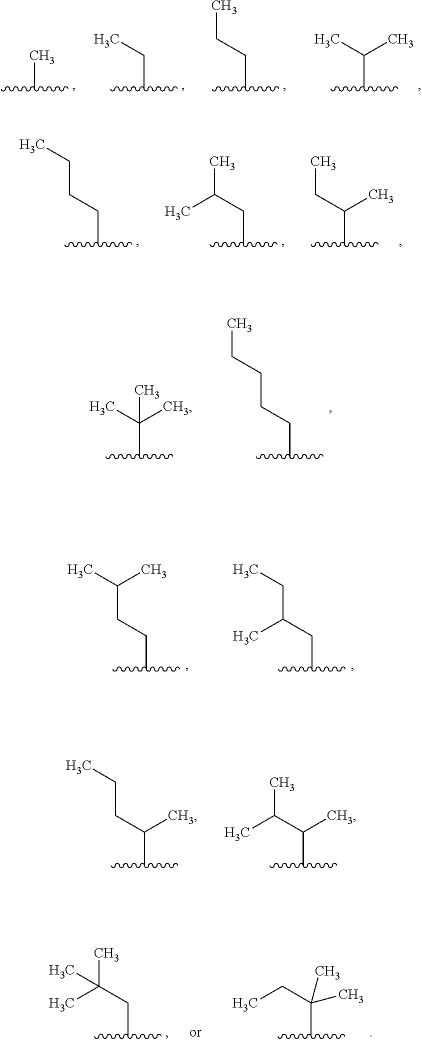

In some embodiments, R²⁸ is

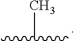

In some embodiments, each of R^{30a}, R^{30b}, and R^{30c} is, independently,

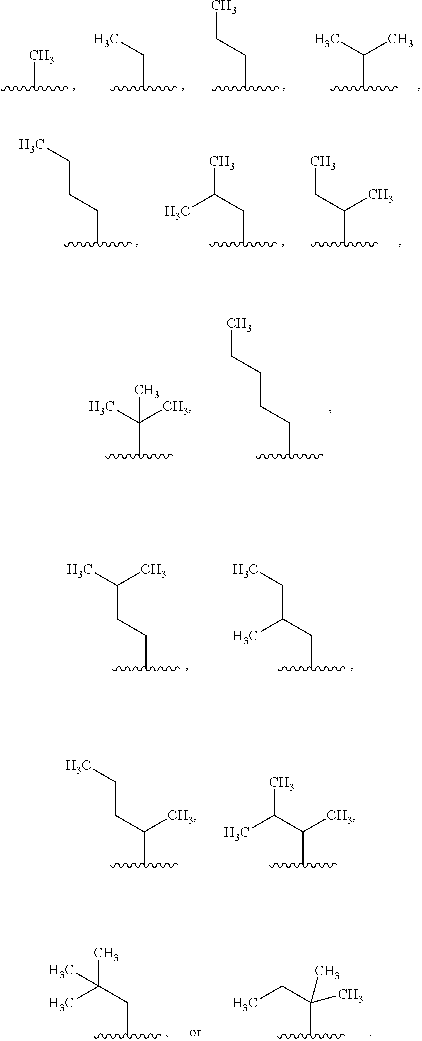

In some embodiments, r is 1. In some embodiments, r is 2. In some embodiments, r is 3.

In some embodiments, each R²⁹ is, independently, H,

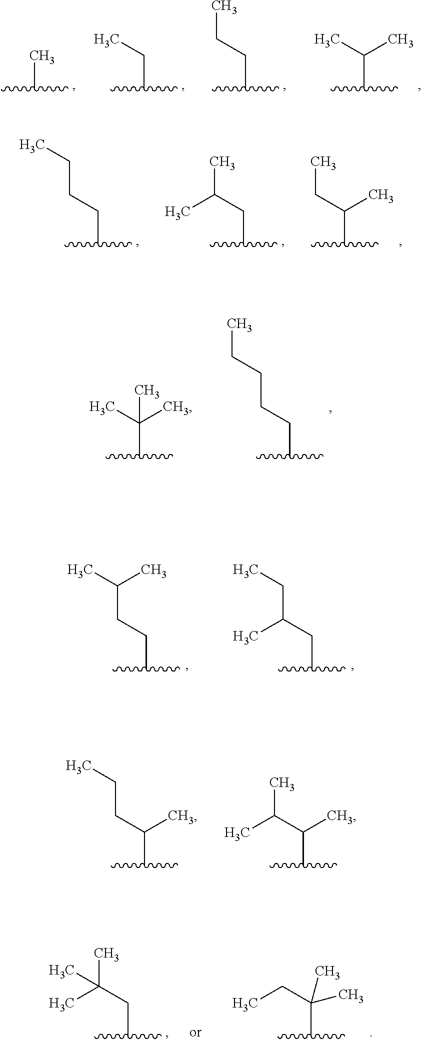

In some embodiments, each R²⁹ is, independently, H or

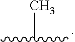

In an aspect, the structural lipid of the invention features a compound having the structure of Formula SIX:

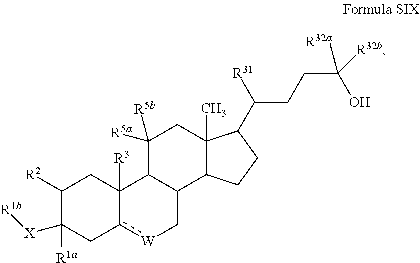

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

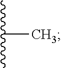

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

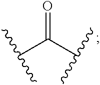 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

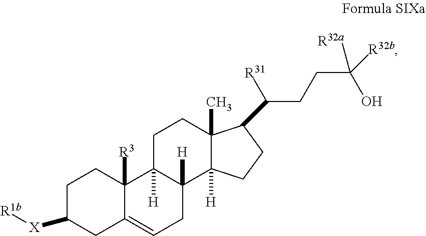

or a pharmaceutically acceptable salt thereof.

In some embodiments, the compound has the structure of Formula SIXb:

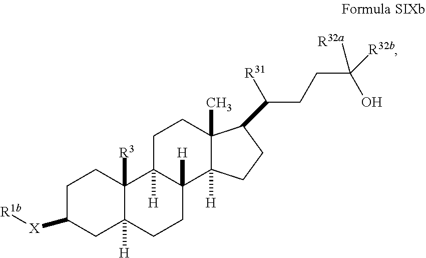

or a pharmaceutically acceptable salt thereof.

In some embodiments, R³¹ is H,

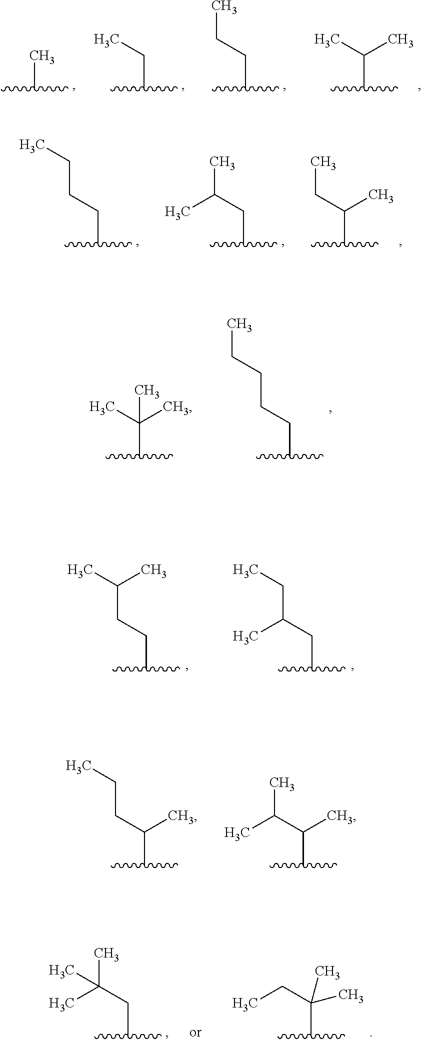

In some embodiments, R³¹ is

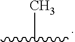

In some embodiments, each of R^{32a} and R^{32b} is, independently,

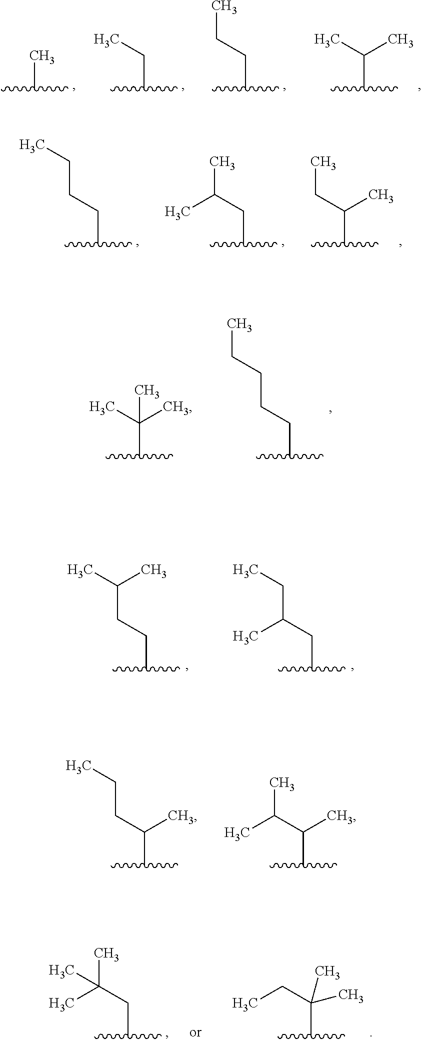

In an aspect, the structural lipid of the invention features a compound having the structure of Formula SX:

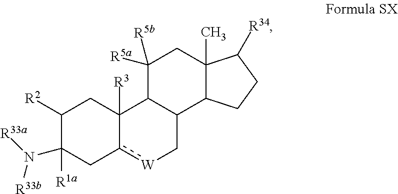

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H

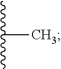

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

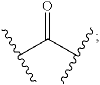 [SMILES: *C(*)=O]

R^{33a} is optionally substituted C₁-C₆ alkyl or

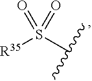

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl,

or a pharmaceutically acceptable salt thereof.

In some embodiments, the compound has the structure of Formula SXa:

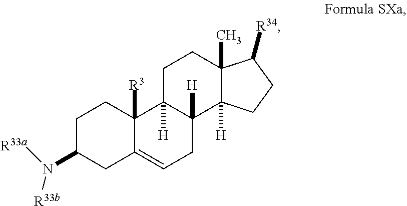

or a pharmaceutically acceptable salt thereof.

In some embodiments, the compound has the structure of Formula SXb:

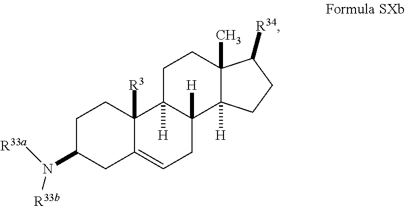

or a pharmaceutically acceptable salt thereof.

In some embodiments, R^{33a} is R³⁵

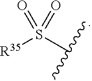

In some embodiments, R³⁵ is

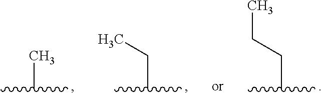

In some embodiments, R³⁵ is

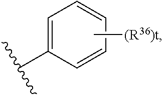

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁴ is

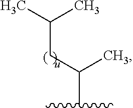

where u is 0, 1, 2, 3, or 4.

In some embodiments, u is 3 or 4.

In an aspect, the structural lipid of the invention features a compound having the structure of Formula SXI:

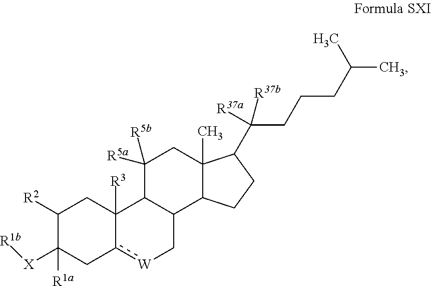

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H

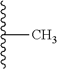

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

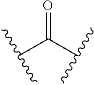 [SMILES: *C(*)=O]

and

each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

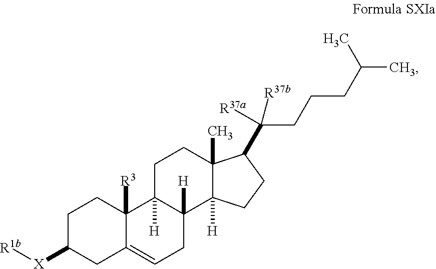

or a pharmaceutically acceptable salt thereof.

In some embodiments, the compound has the structure of Formula SXIb:

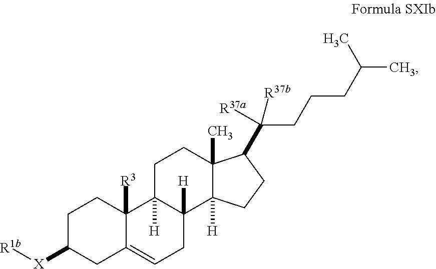

or a pharmaceutically acceptable salt thereof.

In some embodiments, R^{37a} is hydroxyl.

In some embodiments, R^{37b} is

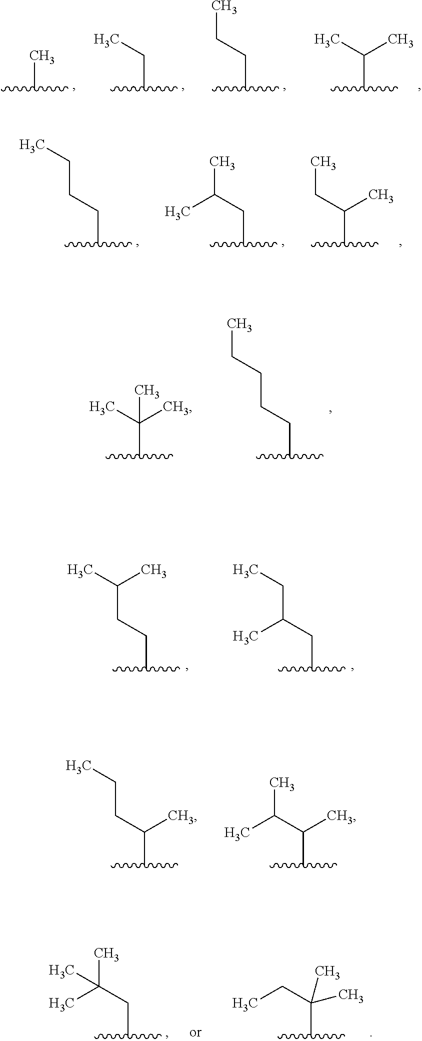

In an aspect, the structural lipid of the invention features a compound having the structure of Formula SXII.

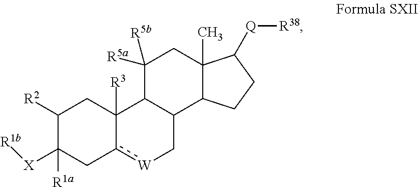

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

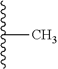

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl; each of R^{5a} and R^{5b} is, independently, H or OR^{A}, or R^{5a} and R^{5b}, together with the atom to which each is attached, combine to form

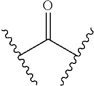 [SMILES: *C(*)=O]

and

Q is 0, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula SXIIa:

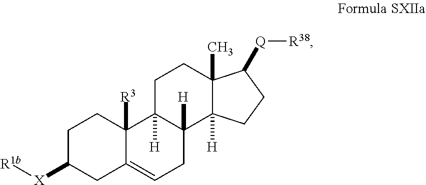

or a pharmaceutically acceptable salt thereof.

In some embodiments, the compound has the structure of Formula SXIIb:

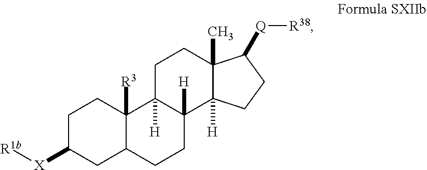

or a pharmaceutically acceptable salt thereof.

In some embodiments, Q is NR^{E}.

In some embodiments, R^{E} is H or

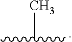

In some embodiments, R^{E} is H. In some embodiments, R^{E} is

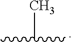

In some embodiments, R³⁸ is

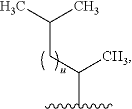

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

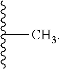

In some embodiments, R⁵, is H.

In some embodiments, R^{5b} is H.

In an aspect, the invention features a compound having the structure of any one of compounds S-1-42, S-150, S-154, S-162-165, S-169-172 and S-184 in Table 1, or any pharmaceutically acceptable salt thereof. As used herein, “CMPD” refers to “compound.”

TABLE 1
Compounds of Formula SI

| CMPD | |
|---|---|
| No. S- | Structure |
| 1 | 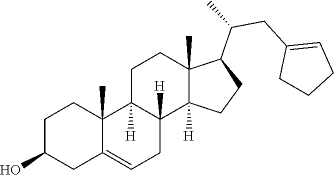 |
| 2 | 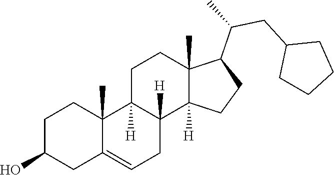 |
| 3 | 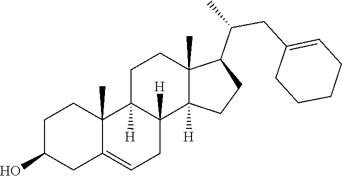 |
| 4 | 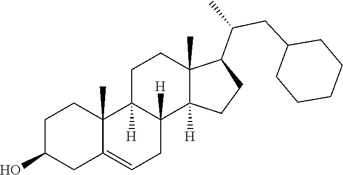 |
| 5 | 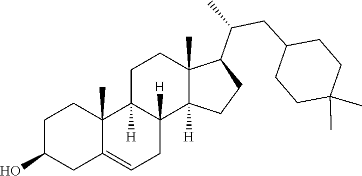 |
| 6 | 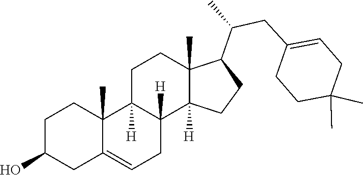 |
| 7 | 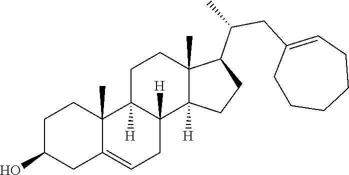 |
| 8 | 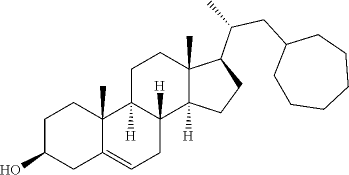 |
| 9 | 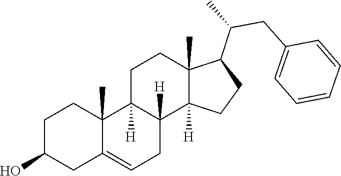 |
| 10 | 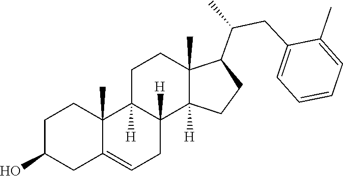 |
| 11 | 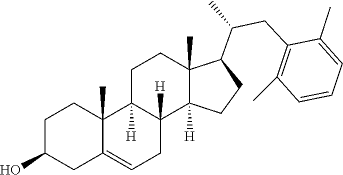 |
| 12 | 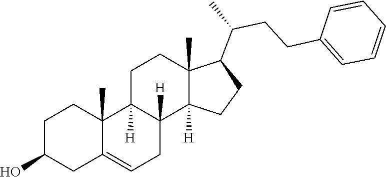 |
| 13 | 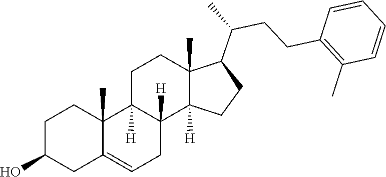 |
| 14 | 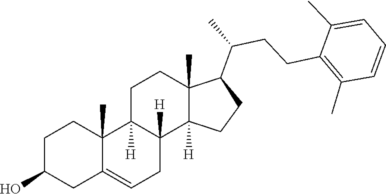 |
| 15 | 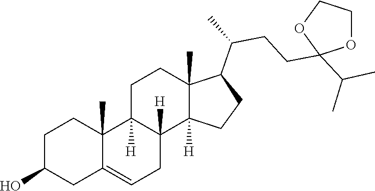 |
| 16 | 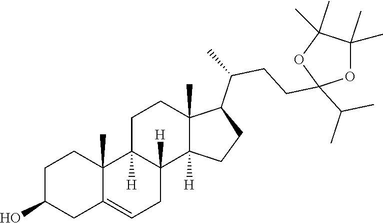 |
| 17 | 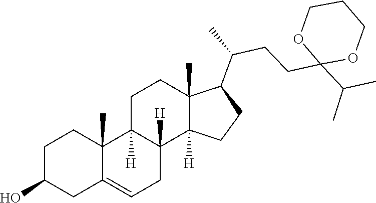 |
| 18 | 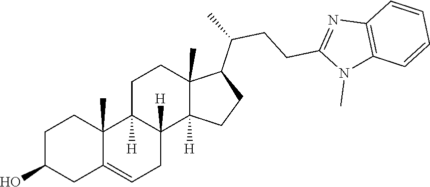 |
| 19 | 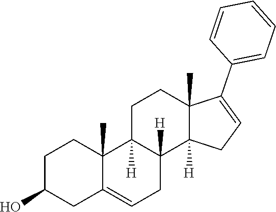 |
| 20 | 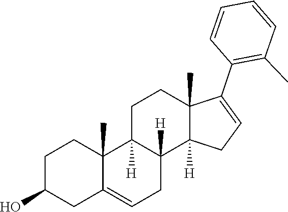 |
| 21 | 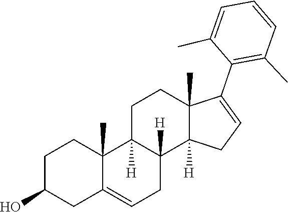 |
| 22 | 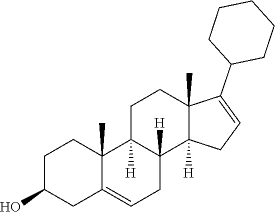 |
| 23 | 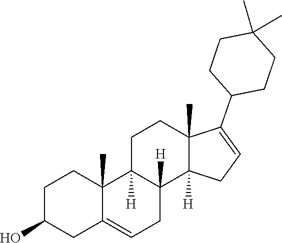 |
| 24 | 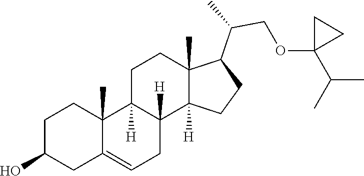 |
| 25 | 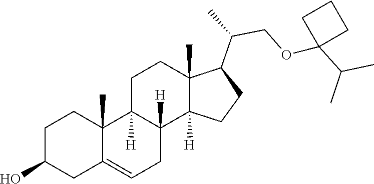 |
| 26 | 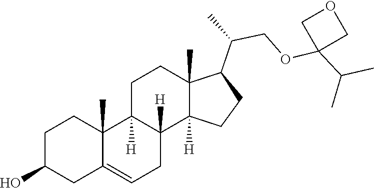 |
| 27 | 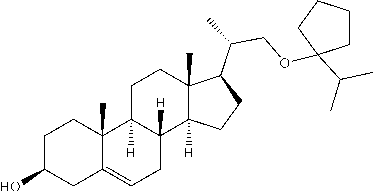 |
| 28 | 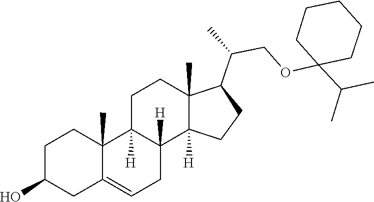 |
| 29 | 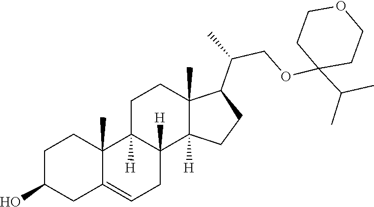 |
| 30 | 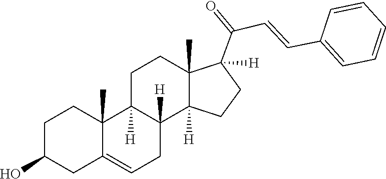 |
| 31 | 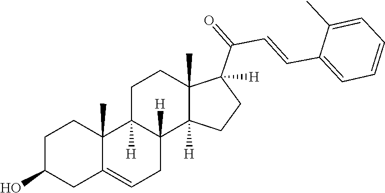 |
| 32 | 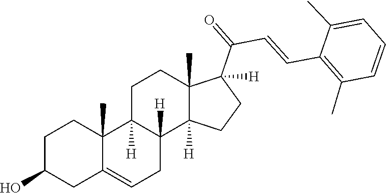 |
| 33 | 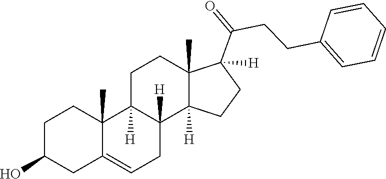 |
| 34 | 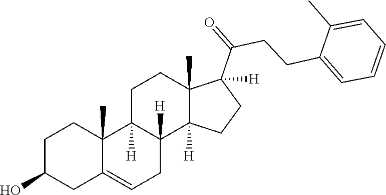 |
| 35 | 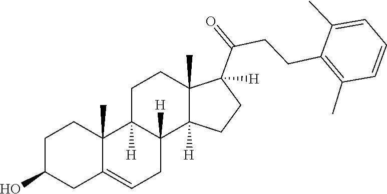 |
| 36 | 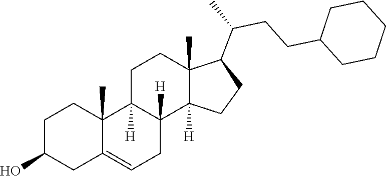 |
| 37 | 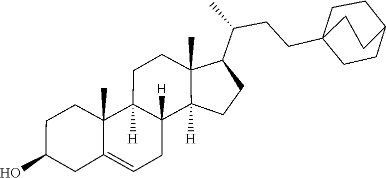 |
| 38 | 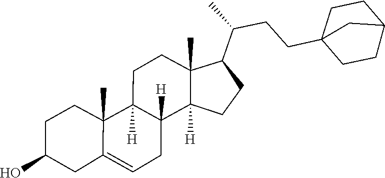 |
| 39 | 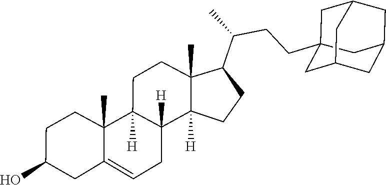 |
| 40 | 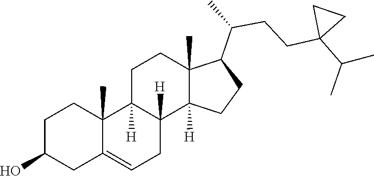 |
| 41 | 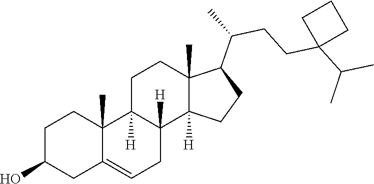 |
| 42 | 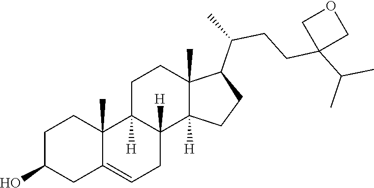 |
| 150 | 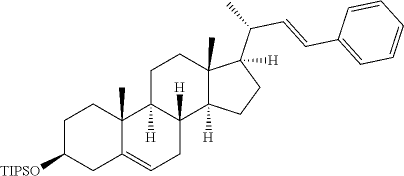 |
| 154 | 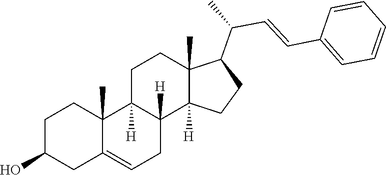 |
| 162 | 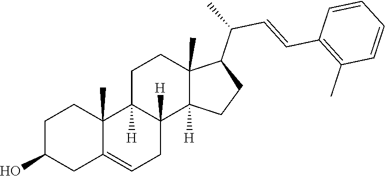 |
| 163 | 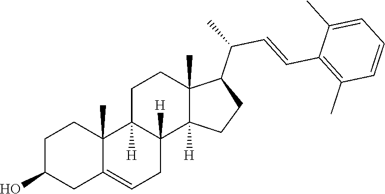 |
| 164 | 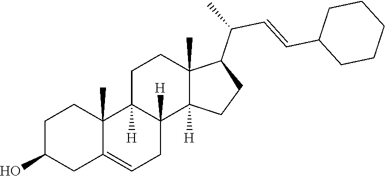 |
| 165 | 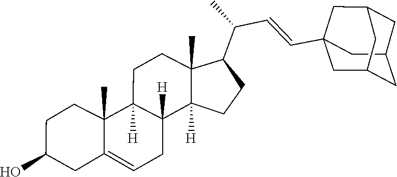 |
| 169 | 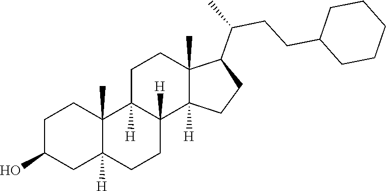 |
| 170 | 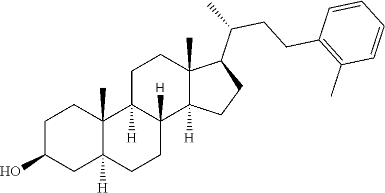 |
| 171 | 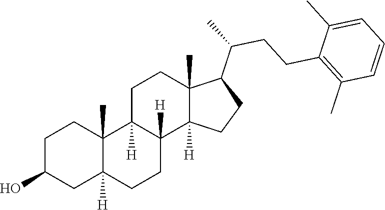 |
| 172 | 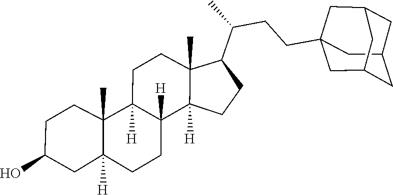 |
| 184 | 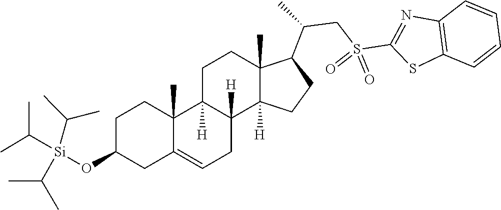 |

In an aspect, the invention features a compound having the structure ofany one of compounds S-43-50 and S-175-178 in Table 2, or any pharmaceutically acceptable salt thereof.

TABLE 2
Compounds of Formula SII

| CMPD | |
|---|---|
| No.S- | Structure |
| 43 | 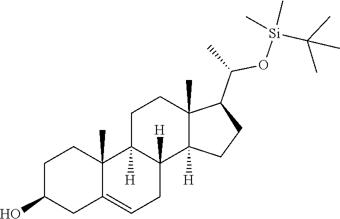 |
| 44 | 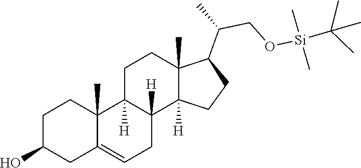 |
| 45 | 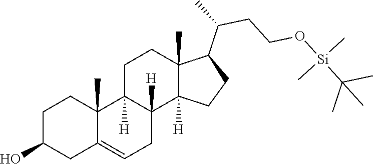 |
| 46 | 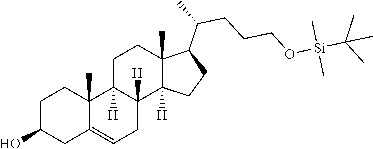 |
| 47 | 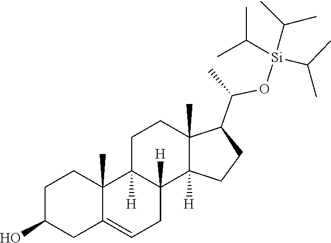 |
| 48 | 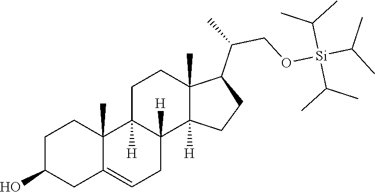 |
| 49 | 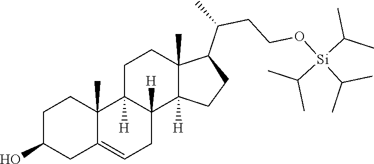 |
| 50 | 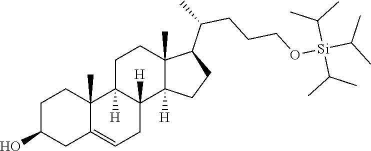 |
| 175 | 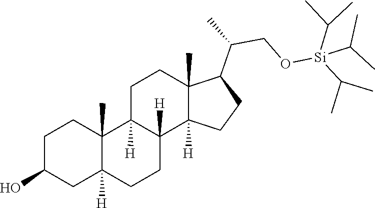 |
| 176 | 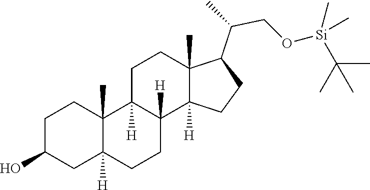 |
| 177 | 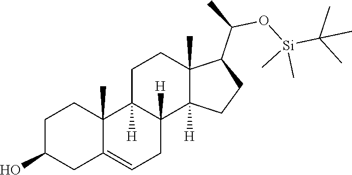 |
| 178 | 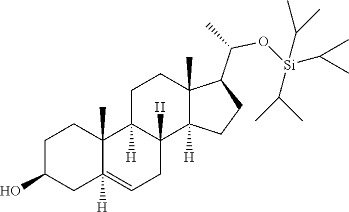 |

In an aspect, the invention features a compound having the structure ofany one of compounds S-51-67, S-149 and S-153 in Table 3, or any pharmaceutically acceptable salt thereof.

TABLE 3
Compounds of Formula SIII

| CMPD | |
|---|---|
| No.S- | Structure |
| 51 | 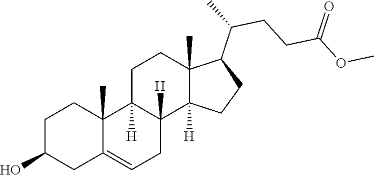 |
| 52 | 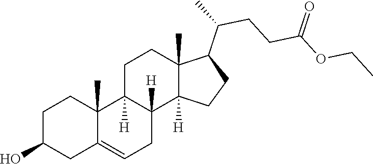 |
| 53 | 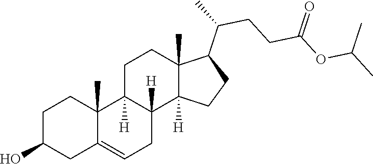 |
| 54 | 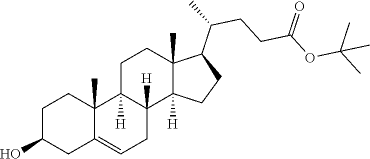 |
| 55 | 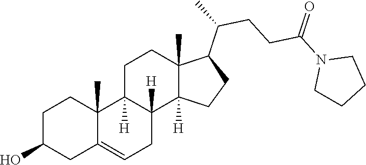 |
| 56 | 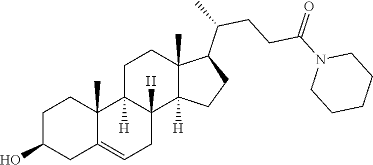 |
| 57 | 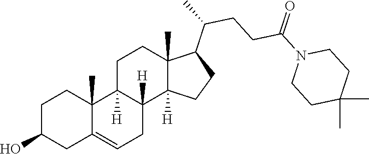 |
| 58 | 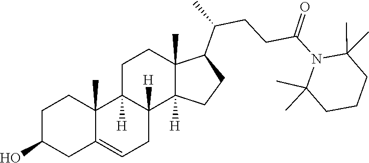 |
| 59 | 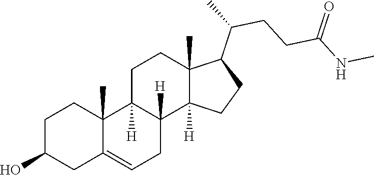 |
| 60 | 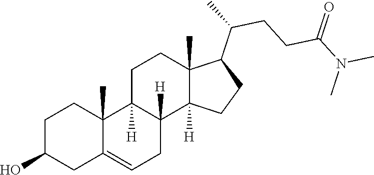 |
| 61 | 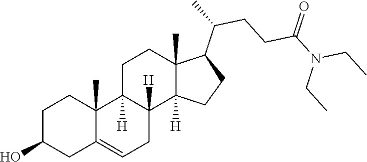 |
| 62 | 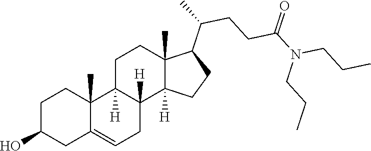 |
| 63 | 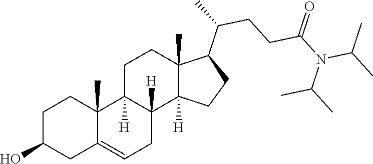 |
| 64 | 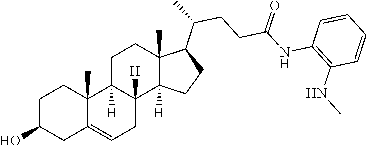 |
| 65 | 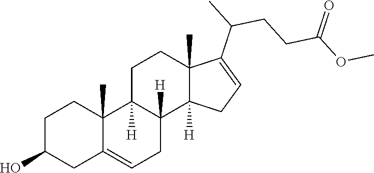 |
| 66 | 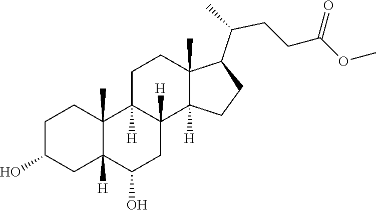 |
| 67 | 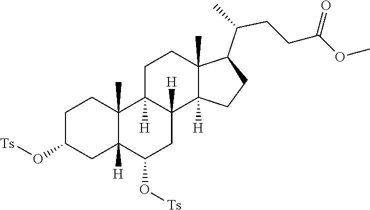 |
| 149 | 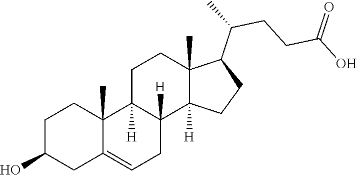 |
| 153 | 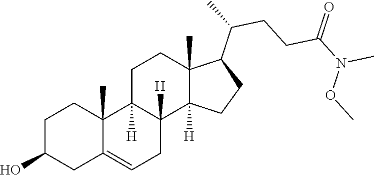 |

In an aspect, the invention features a compound having the structure of any one of compounds S-68-73 in Table 4, or any pharmaceutically acceptable salt thereof.

TABLE 4
Compounds of Formula SIV

| CMPD | |
|---|---|
| No.S- | Structure |
| 68 | 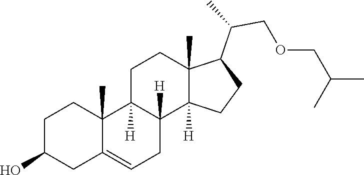 |
| 69 | 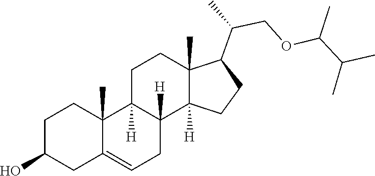 |
| 70 | 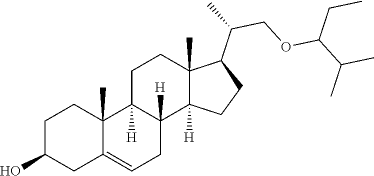 |
| 71 | 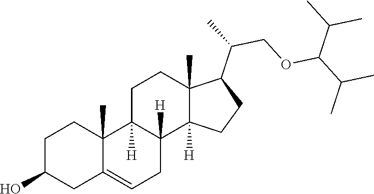 |
| 72 | 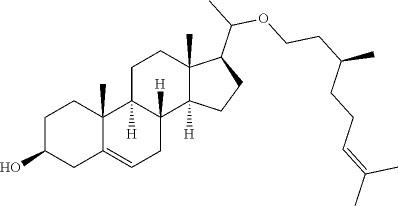 |
| 73 | 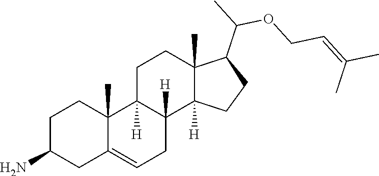 |

In an aspect, the invention features a compound having the structure of any one of compounds S-74-78 in Table 5, or any pharmaceutically acceptable salt thereof.

TABLE 5
Compounds of Formula SV

| CMPD | |
|---|---|
| No. S- | Structure |
| 74 | 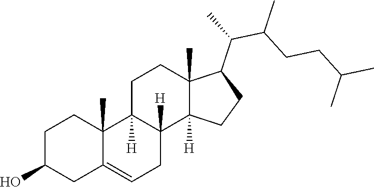 |
| 75 | 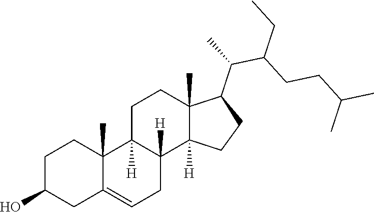 |
| 76 | 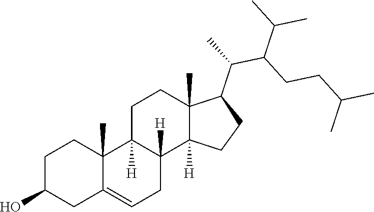 |
| 77 | 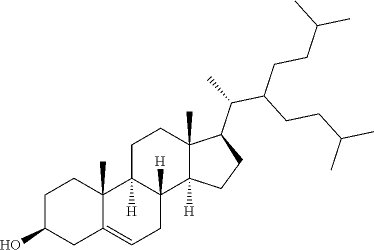 |
| 78 | 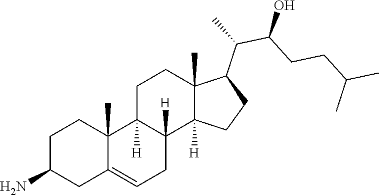 |

In an aspect, the invention features a compound having the structure of any one of compounds S-79 or S-80 in Table 6, or any pharmaceutically acceptable salt thereof.

TABLE 6
Compounds of Formula SVI

| CMPD | |
|---|---|
| No. S- | Structure |
| 79 | 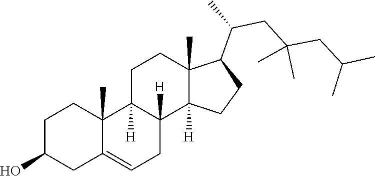 |
| 80 | 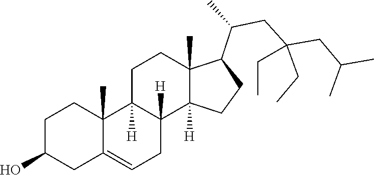 |

In an aspect, the invention features a compound having the structure of any one of compounds S-81-87, S-152 and S-157 in Table 7, or any pharmaceutically acceptable salt thereof.

TABLE 7
Compounds of Formula S-VII

| CMPD | |
|---|---|
| No. S- | Structure |
| 81 | 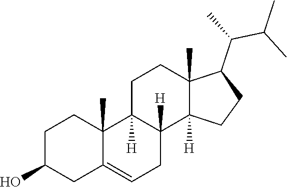 |
| 82 | 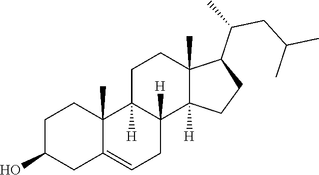 |
| 83 | 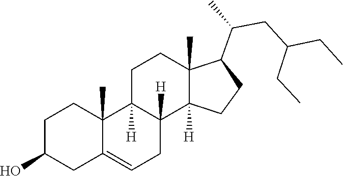 |
| 84 | 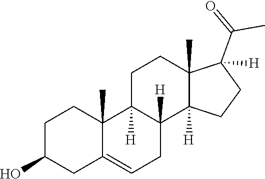 |
| 85 | 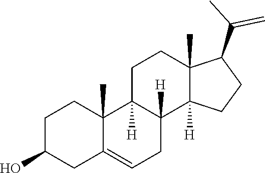 |
| 86 | 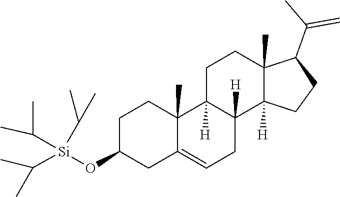 |
| 87 | 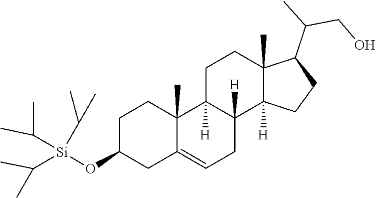 |
| 152 | 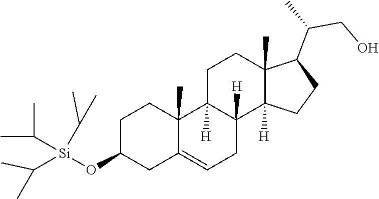 |
| 157 | 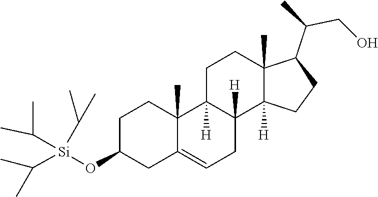 |

In an aspect, the invention features a compound having the structure of any one of compounds S-88-97 in Table 8, or any pharmaceutically acceptable salt thereof.

TABLE 8
Compounds of Formula SVIII

| CMPD | |
|---|---|
| No. S- | Structure |
| 88 | 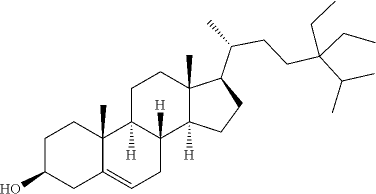 |
| 89 | 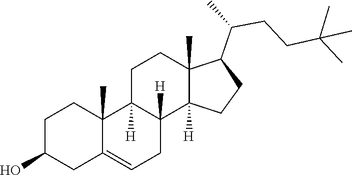 |
| 90 | 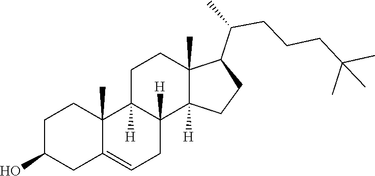 |
| 91 | 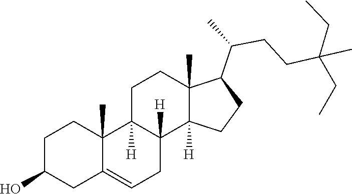 |
| 92 | 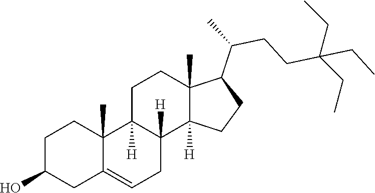 |
| 93 | 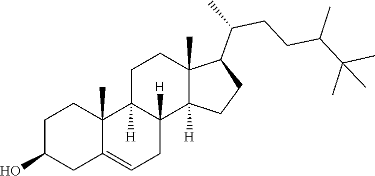 |
| 94 | 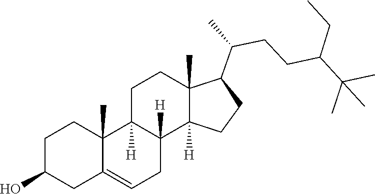 |
| 95 | 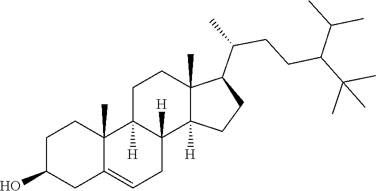 |
| 96 | 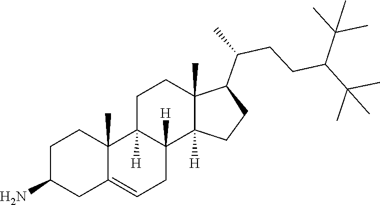 |
| 97 | 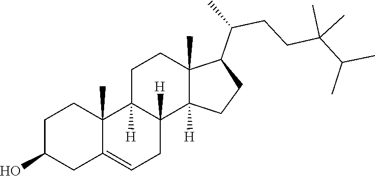 |

In an aspect, the invention features a compound having the structure of any one of compounds S-98-105 and S-180-182 in Table 9, or any pharmaceutically acceptable salt thereof.

TABLE 9
Compounds of Formula SIX

| CMPD | |
|---|---|
| No. S- | Structure |
| 98 | 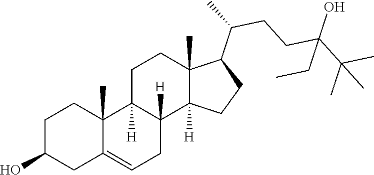 |
| 99 | 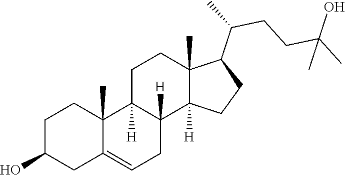 |
| 100 | 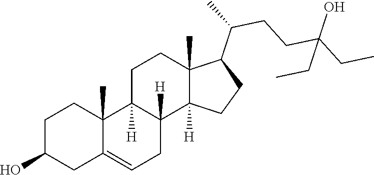 |
| 101 | 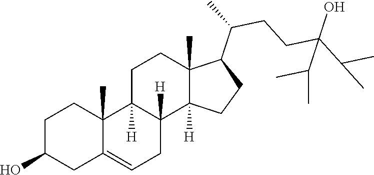 |
| 102 | 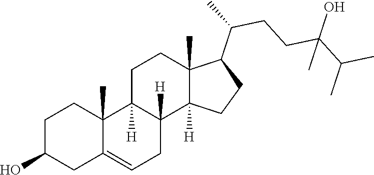 |
| 103 | 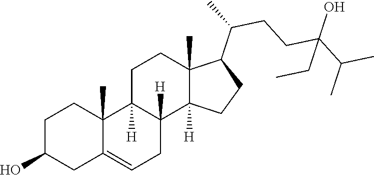 |
| 104 | 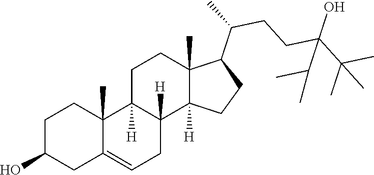 |
| 105 | 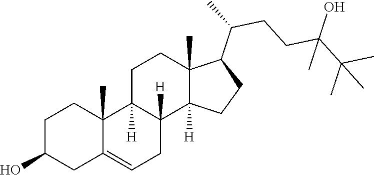 |
| 180 | 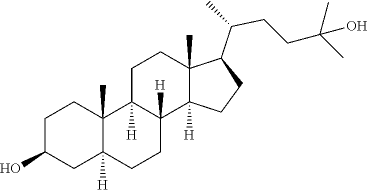 |
| 181 | 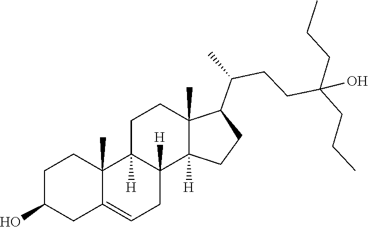 |
| 182 | 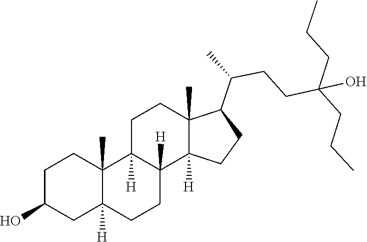 |

In an aspect, the invention features a compound having the structure of compound S-106 in Table 10, or any pharmaceutically acceptable salt thereof.

TABLE 10
Compounds of Formula SX

| CMPD | |
|---|---|
| No. S- | Structure |
| 106 | 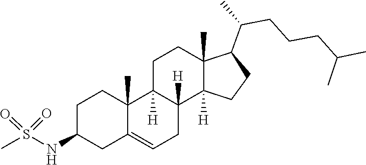 |

In an aspect, the invention features a compound having the structure of compound S-107 or S-108 in Table 11, or any pharmaceutically acceptable salt thereof.

TABLE 11
Compounds of Formula SXI

| CMPD | |
|---|---|
| No. S- | Structure |
| 107 | 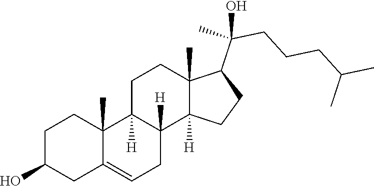 |
| 108 | 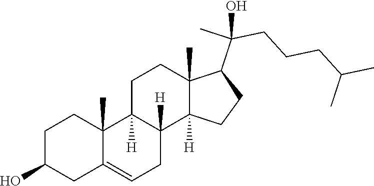 |

In an aspect, the invention features a compound having the structure of compound S-109 in Table 12, or any pharmaceutically acceptable salt thereof.

TABLE 12
Compounds of Formula SXII

| CMPD | |
|---|---|
| No. S- | Structure |
| 109 | 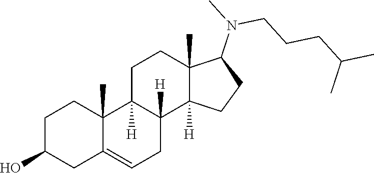 |

In an aspect, the invention features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Invention

| CMPD | |
|---|---|
| No. S- | Structure |
| 110 | 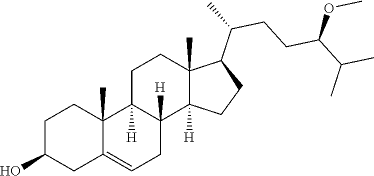 |
| 111 | 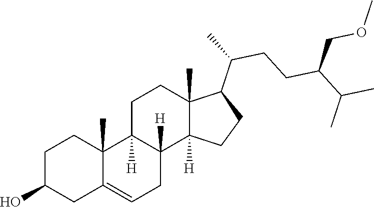 |
| 112 | 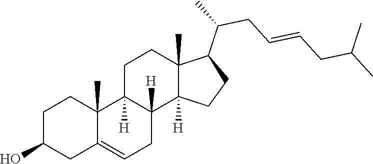 |
| 113 | 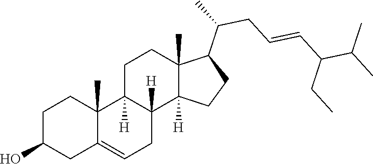 |
| 114 | 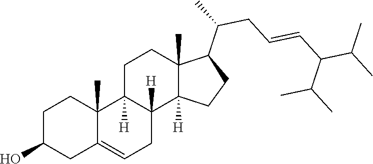 |
| 115 | 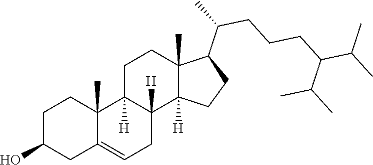 |
| 116 | 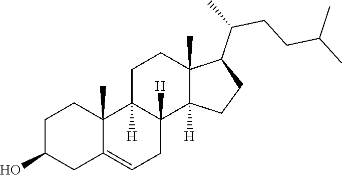 |
| 117 | 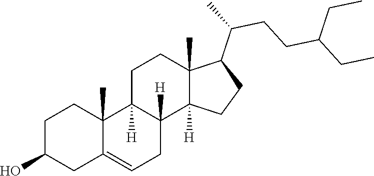 |
| 118 | 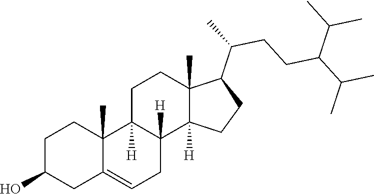 |
| 119 | 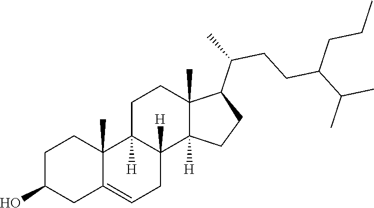 |
| 120 | 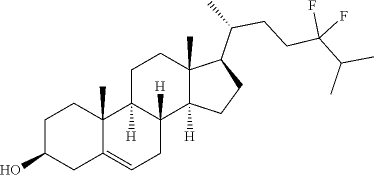 |
| 121 | 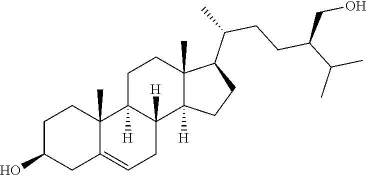 |
| 122 | 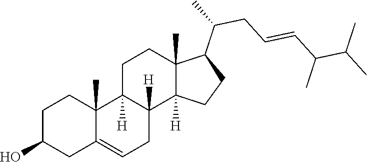 |
| 123 | 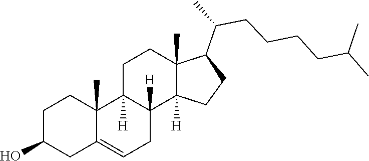 |
| 124 | 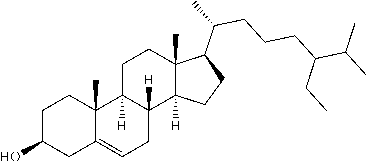 |
| 125 | 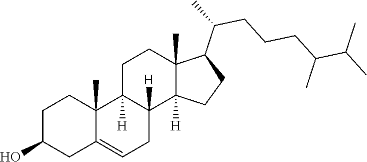 |
| 126 | 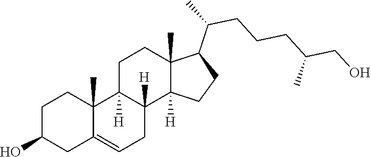 |
| 127 | 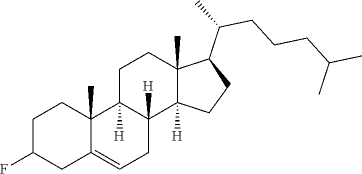 |
| 128 | 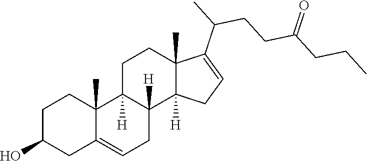 |
| 129 | 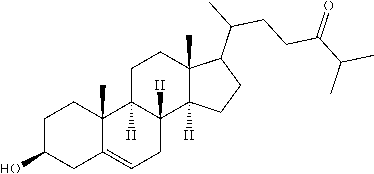 |
| 130 | 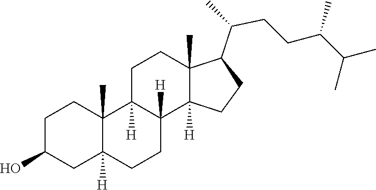 |
| 155 | 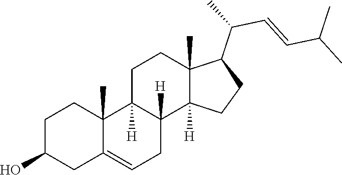 |
| 156 | 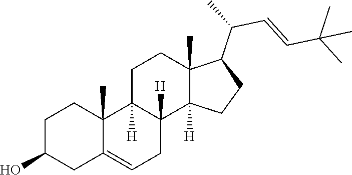 |
| 158 | 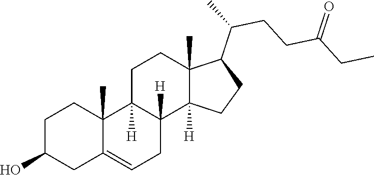 |
| 160 | 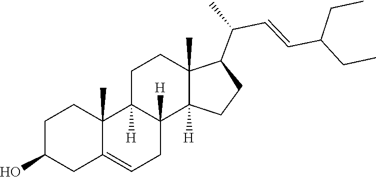 |
| 161 | 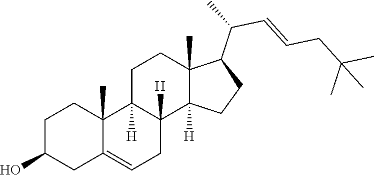 |
| 166 | 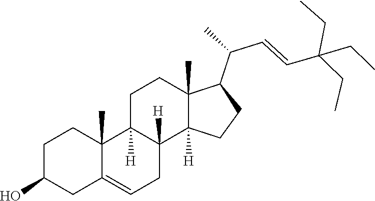 |
| 167 | 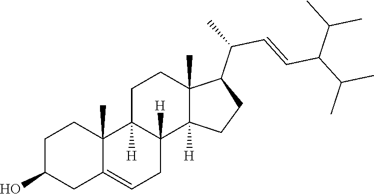 |
| 168 | 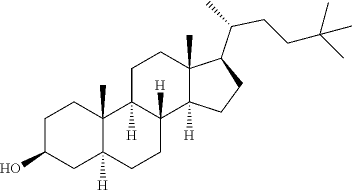 |
| 173 | 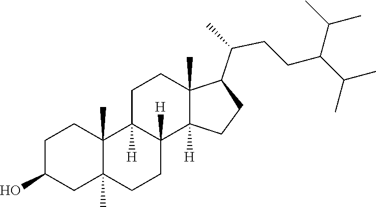 |
| 174 | 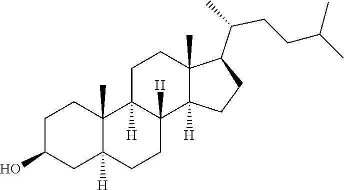 |
| 179 | 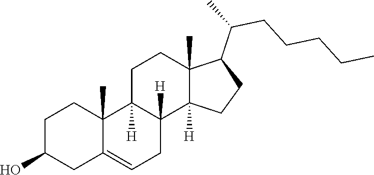 |

In an aspect, the invention features a compound having the structure of any one of compounds S-131-133 in Table 14, or any pharmaceutically acceptable salt thereof.

TABLE 14
Compounds of the Invention

| CMPD | |
|---|---|
| No. S- | Structure |
| 131 | 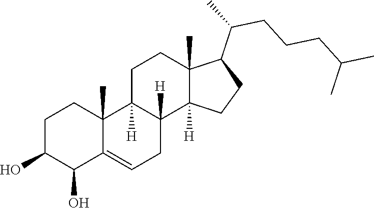 |
| 132 | 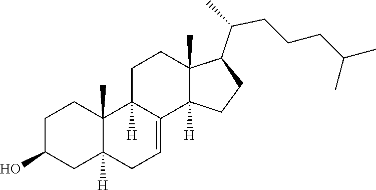 |
| 133 | 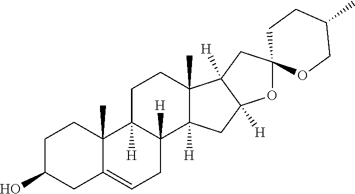 |

In an aspect, the invention features a compound having the structure of any one of compounds S-134-148, S-151 and S-159 in Table 15, or any pharmaceutically acceptable salt thereof.

TABLE 15
Compounds of the Invention

| CMPD | |
|---|---|
| No. S- | Structure |
| 134 | 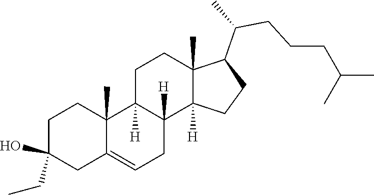 |
| 135 | 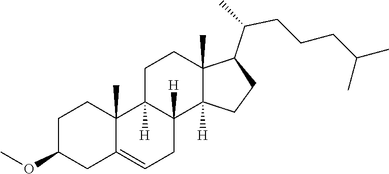 |
| 136 | 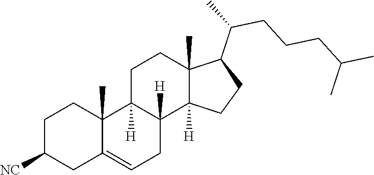 |
| 137 | 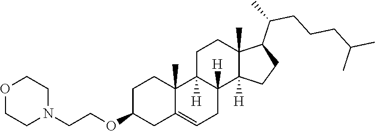 |
| 138 | 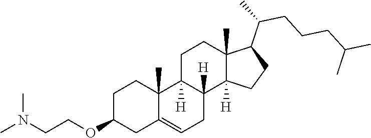 |
| 139 | 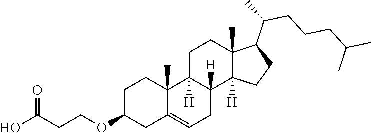 |
| 140 | 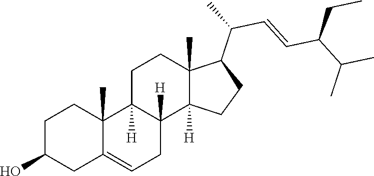 |
| 141 | 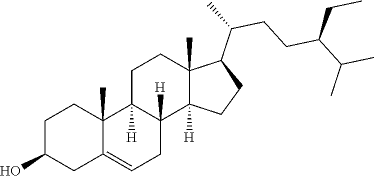 |
| 142 | 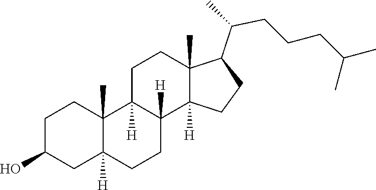 |
| 143 | 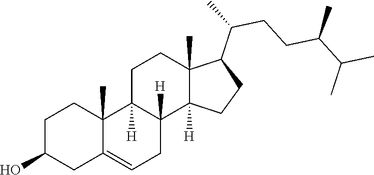 |
| 144 | 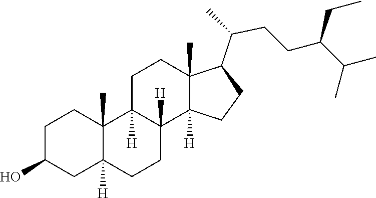 |
| 145 | 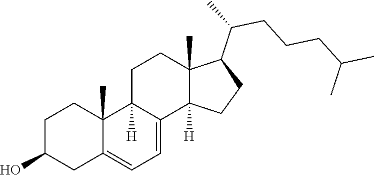 |
| 146 | 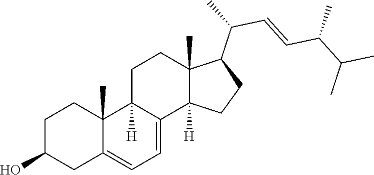 |
| 147 | 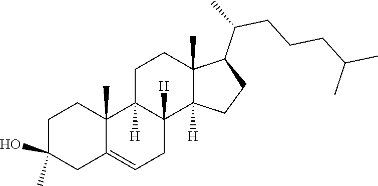 |
| 148 | 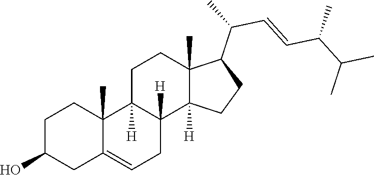 |
| 151 | 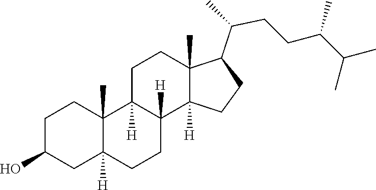 |
| 159 | 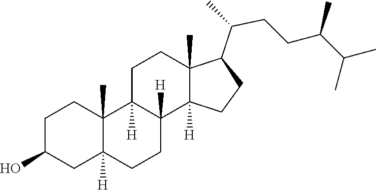 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one or more structural lipids of the lipid nanoparticles of the invention can be composition 183 in Table 16.

TABLE 16
Structural Lipid Compositions

| Composition | |
|---|---|
| S-No. | Structure |
| 183 | 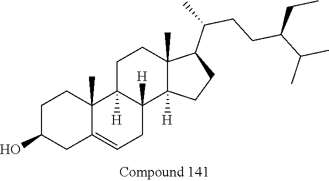 |
| | 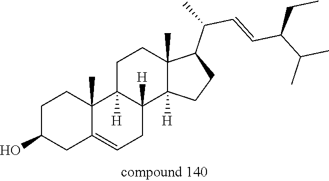 |
| | 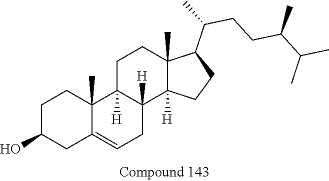 |
| | 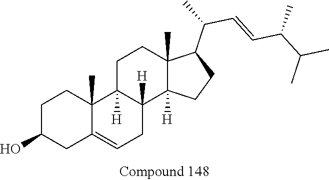 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds 5-1-148 combined with a cholesterol and/or a phytosterol.

For example, the structural component of the lipid nanoparticle can be a mixture of one or more structural compounds (e.g. any of Compounds 5-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(iii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

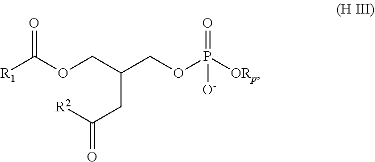

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanolamine (PE 18:3 (9Z,12Z, 15Z), 1,2-diarachidonoyl-sn-glycero-3-phosphoethanolamine (DAPE 18:3 (9Z,12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin. Each possibility represents a separate embodiment of the invention.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in a target cell target cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

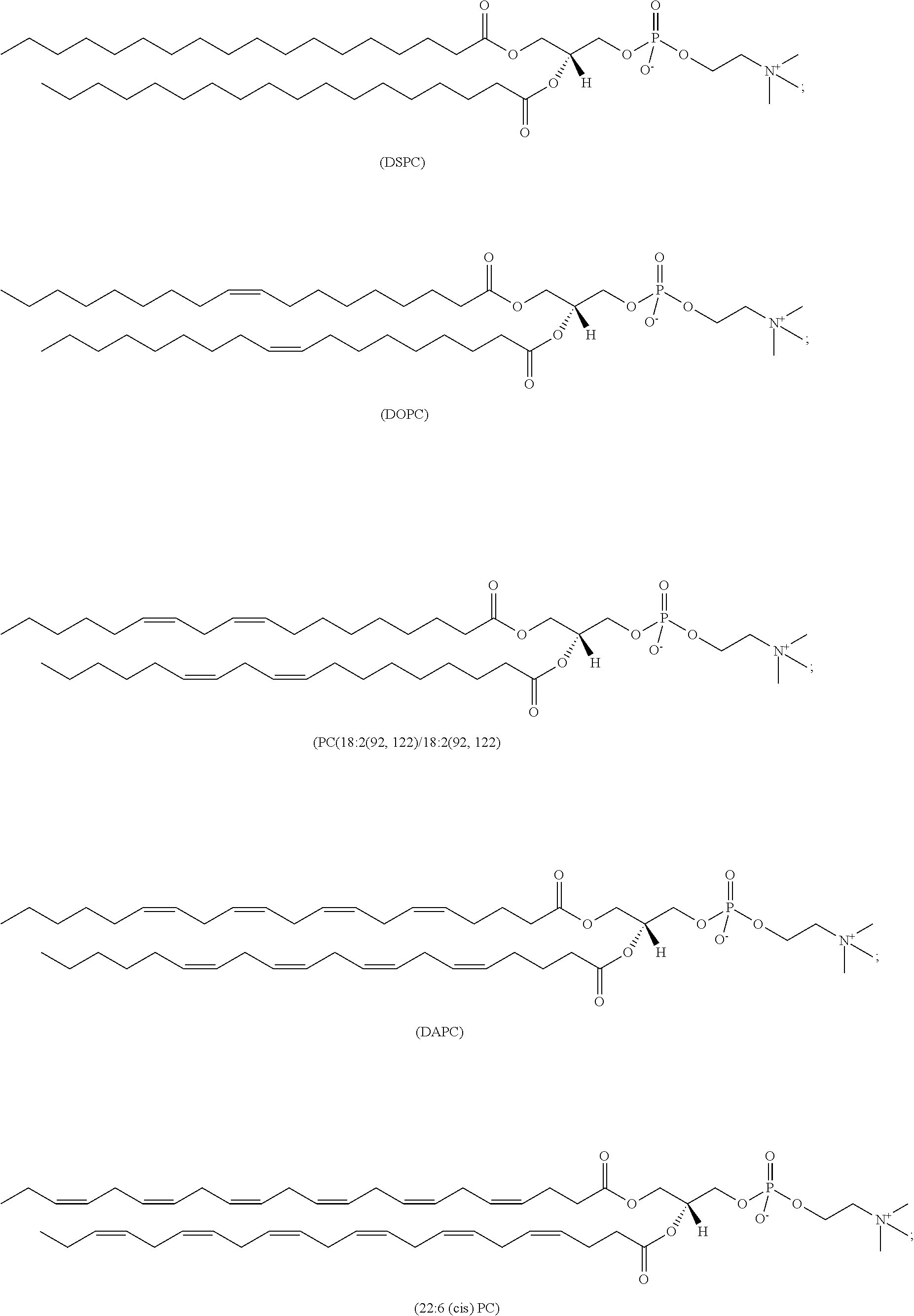

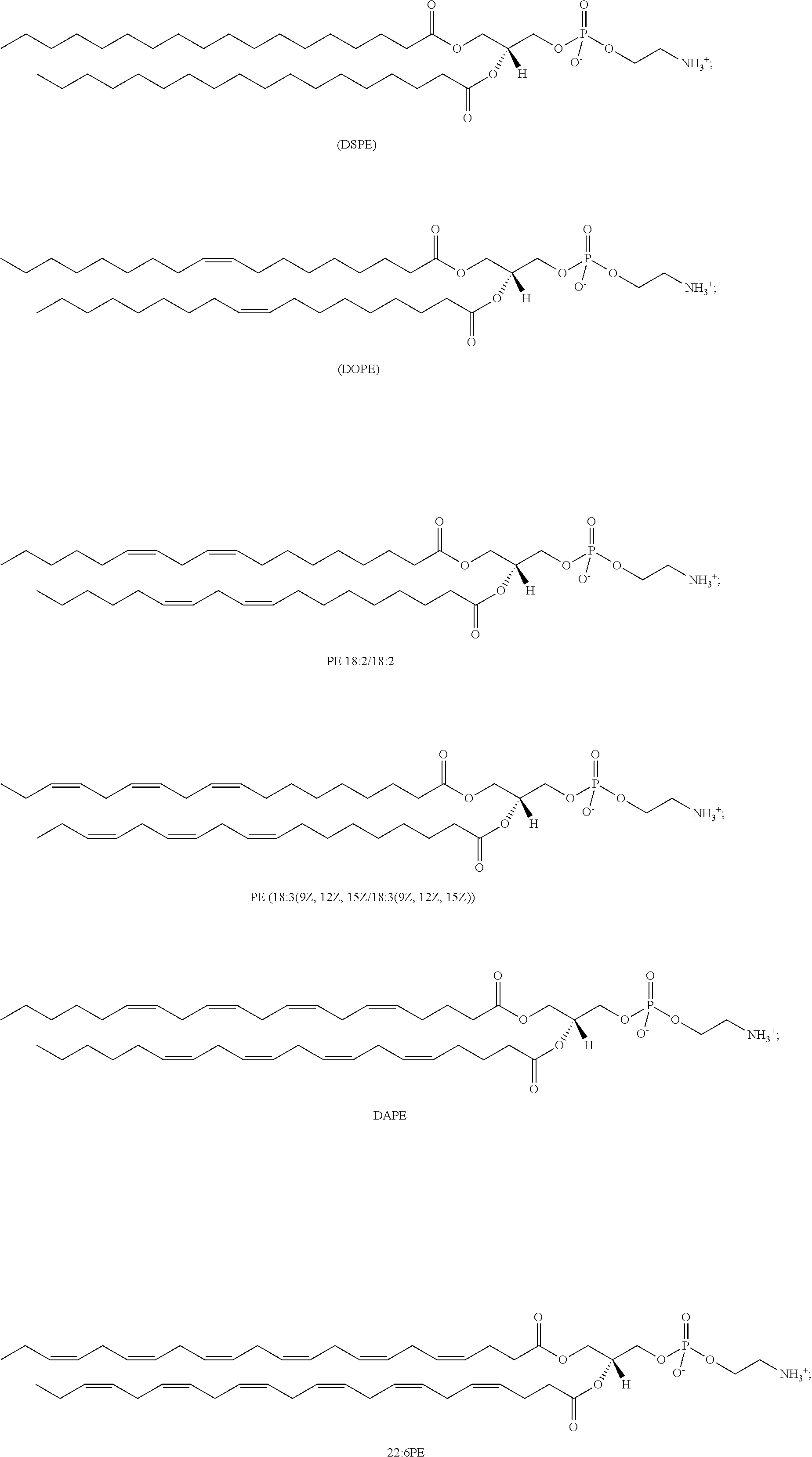

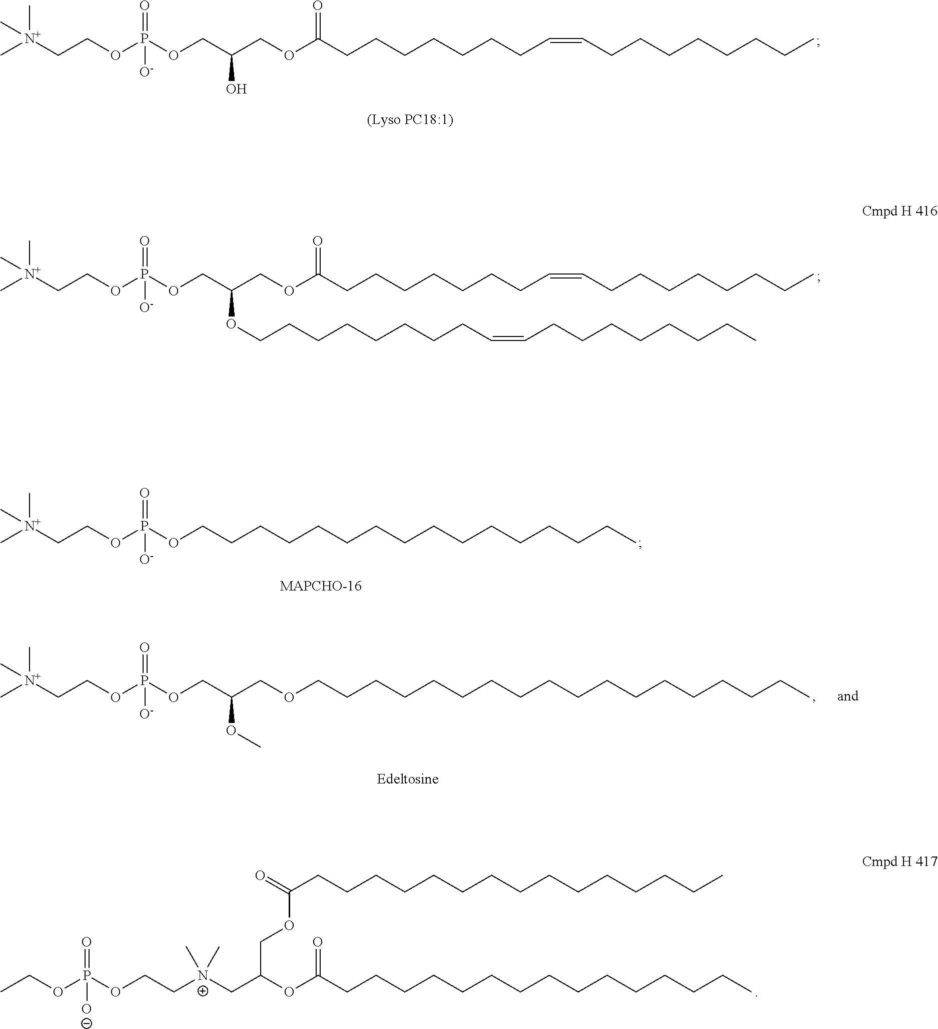

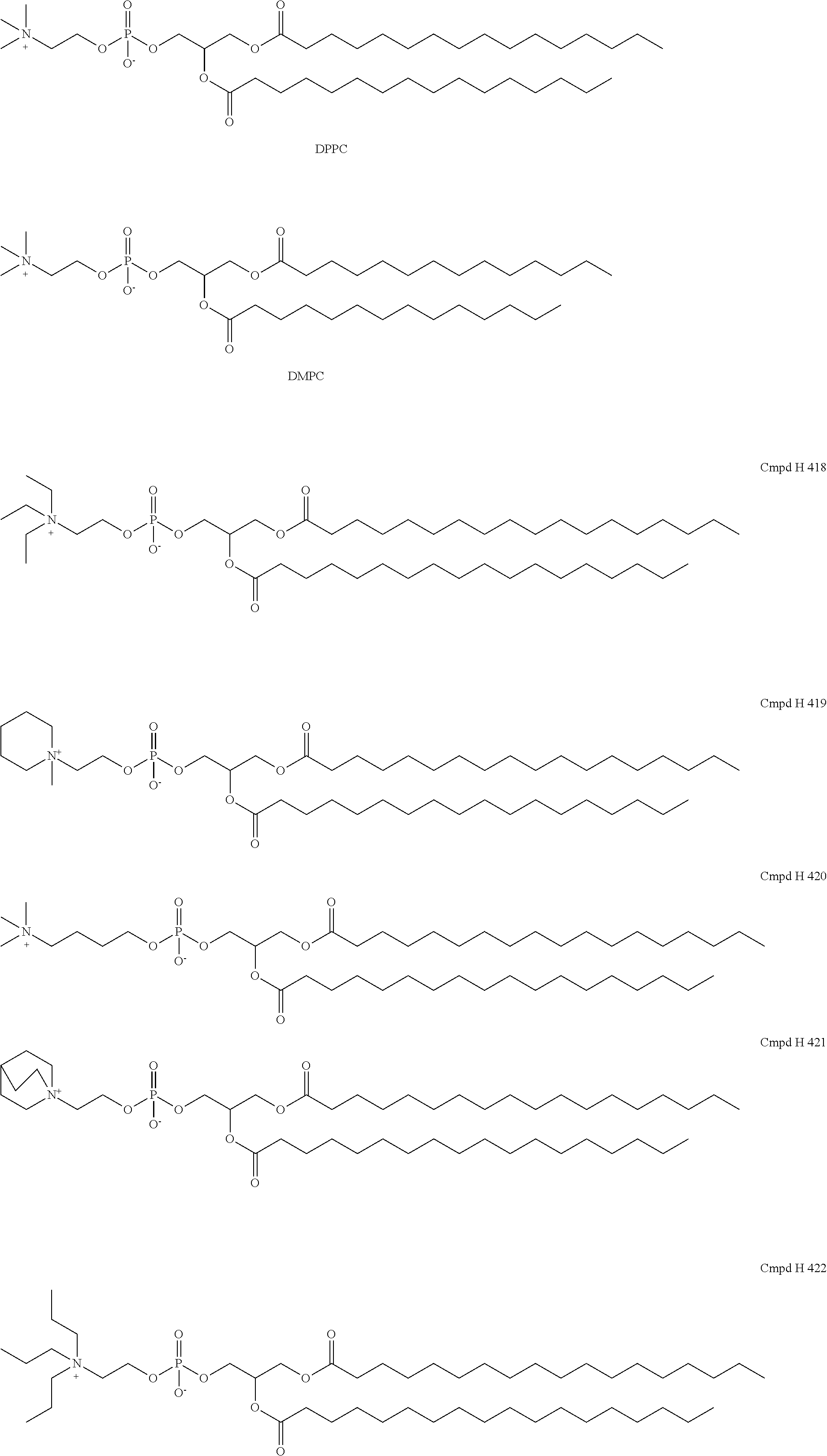

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

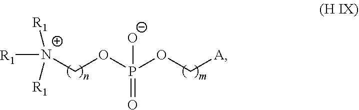

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

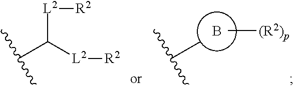

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O, —OC(O)—, —OC(O)O—, —OC(O)N(R^{N})—, —NR^{N}C(O)O—, or —NR^{N}C(O)N(R^{N})—;

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

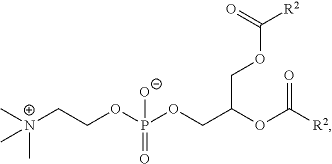

wherein each instance of R² is independently unsubstituted alkyl, unsubstituted alkinyl or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

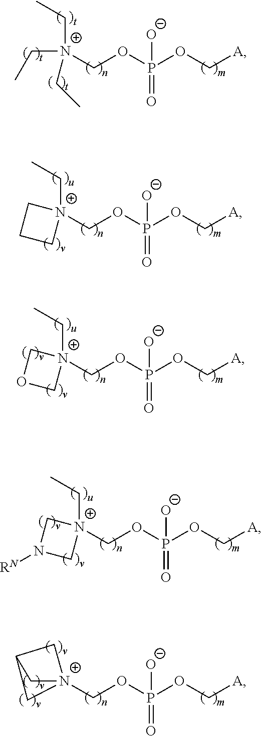

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, the compound of Formula (H IX) is of one of the following formulae:

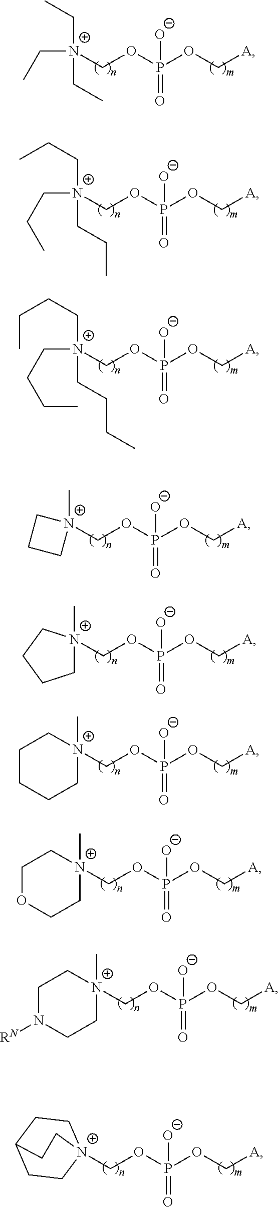

or a salt thereof.

In certain embodiments, a compound of Formula (H IX) is one of the following:

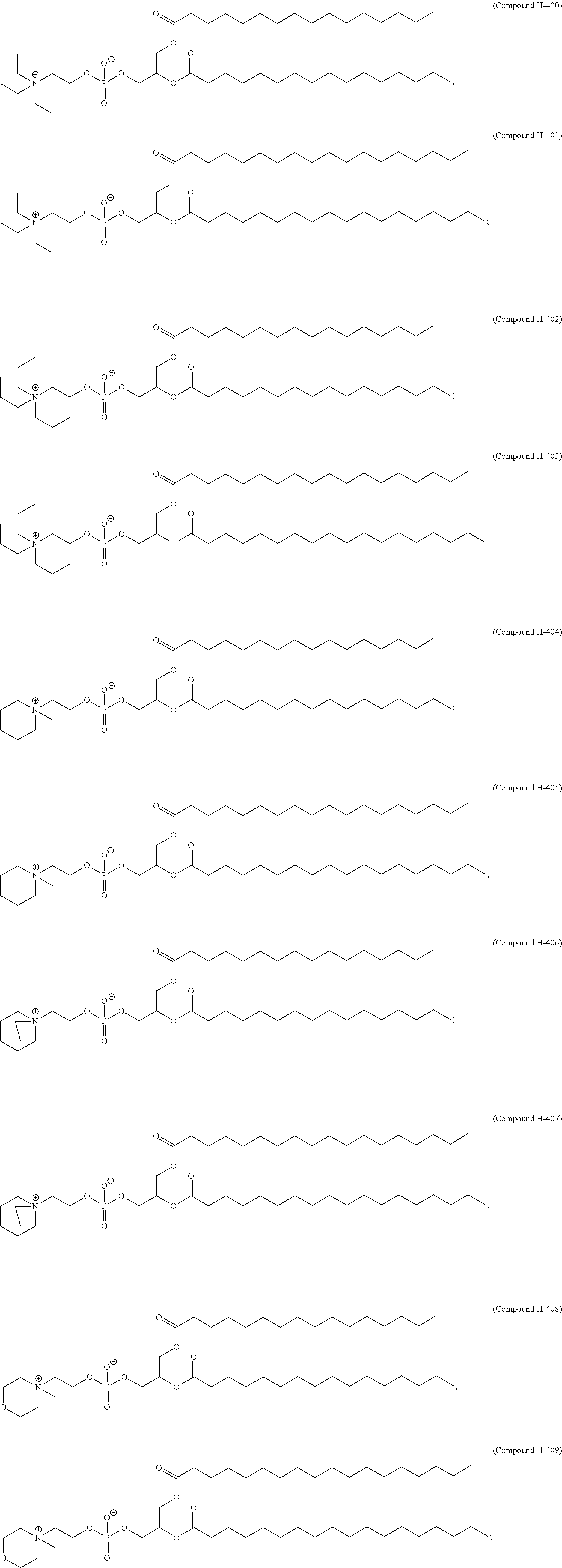

or a salt thereof.

In one embodiment, a target cell target cell delivery LNP comprises Compound 4 as a non-cationic helper lipid.

(ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})SO—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c).

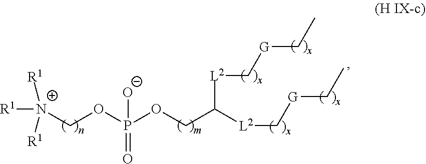

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OSO₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

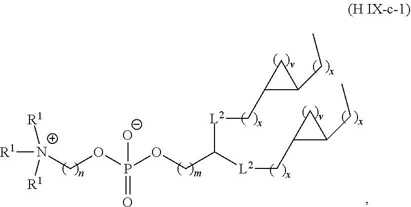

or salt thereof, wherein:
each instance of v is independently 1, 2, or 3.

In certain embodiments, the compound of Formula (H IX-c) is of Formula (H IX-c-2):

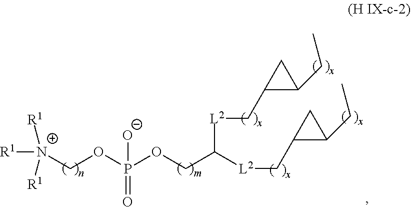

or a salt thereof.

In certain embodiments, the compound of Formula (IX-c) is of the following formula:

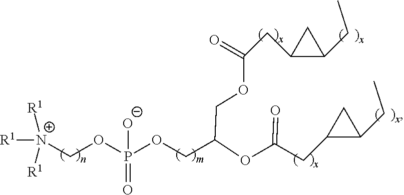

or a salt thereof.

In certain embodiments, the compound of Formula (H IX-c) is the following:

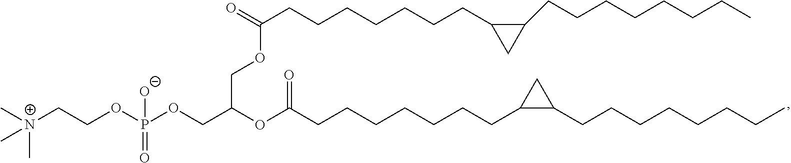 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

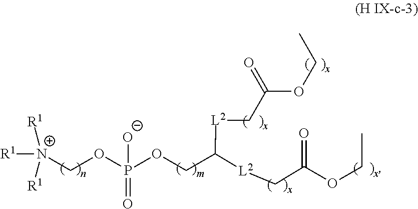

or a salt thereof.

In certain embodiments, the compound of Formula (H IX-c) is of the following formulae:

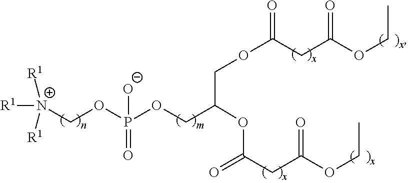

or a salt thereof.

In certain embodiments, the compound of Formula (H IX-c) is the following:

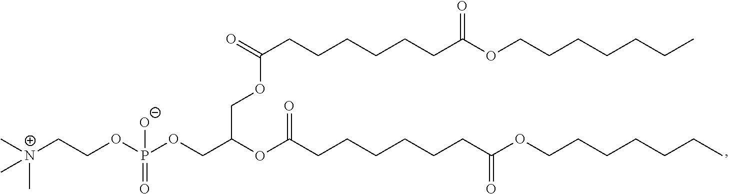 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

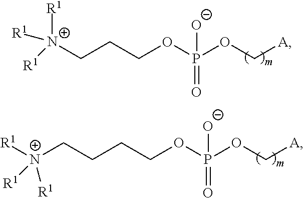

or a salt thereof.

In certain embodiments, a compound of Formula (H IX) is one of the following:

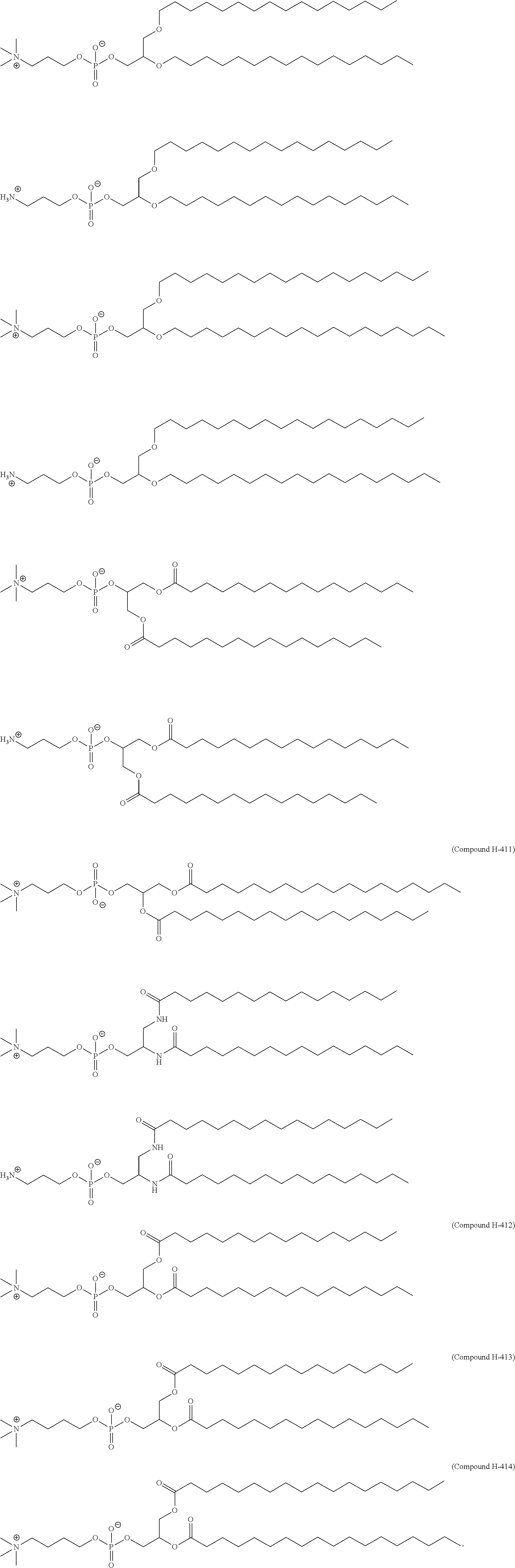

or salts thereof.

In certain embodiments, an alternative lipid is used in place of a phospholipid of the invention. Non-limiting examples of such alternative lipids include the following:

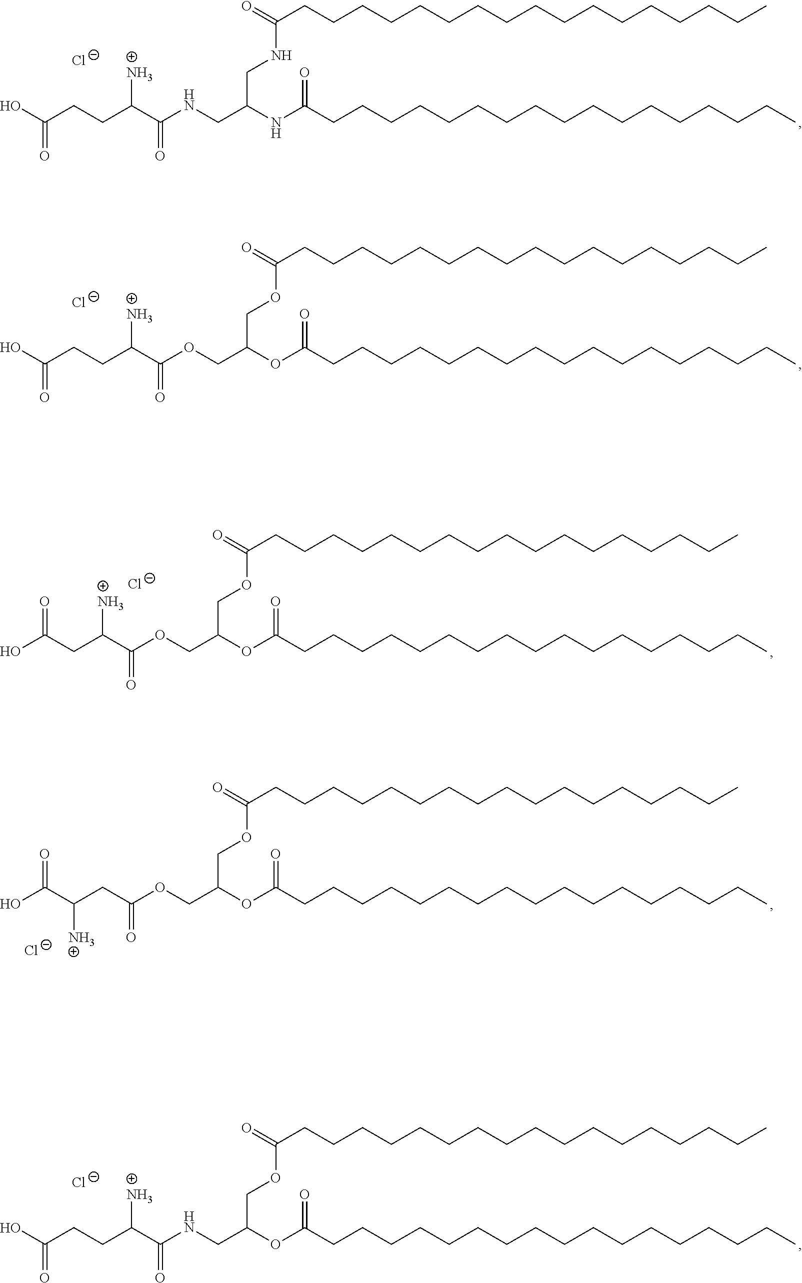

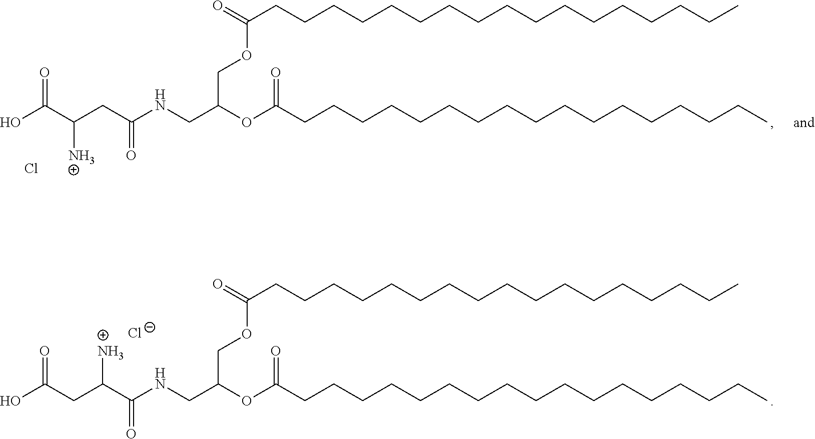 [SMILES: CCCCCCCCCCCCCCCCCC(=O)OCC(CNC(=O)CC([NH3+])C(=O)O)OC(=O)CCCCCCCCCCCCCCCCC ; [Cl-] ; CCCCCCCCCCCCCCCCCC(=O)OCC(CNC(=O)C([NH3+])CC(=O)O)OC(=O)CCCCCCCCCCCCCCCCC ; [Cl-]]

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

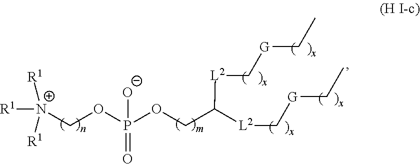

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

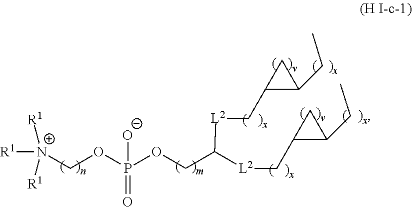

or salt thereof, wherein:

each instance of v is independently 1, 2, or 3.

In certain embodiments, the compound of Formula (H I-c) is of Formula (H I-c-2):

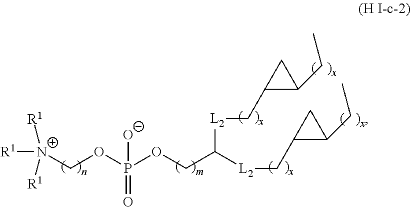

or a salt thereof.

In certain embodiments, the compound of Formula (I-c) is of the following formula:

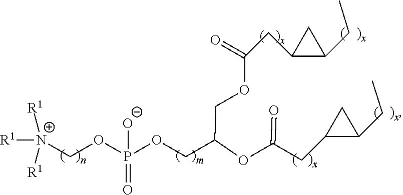

or a salt thereof.

In certain embodiments, the compound of Formula (H I-c) is the following:

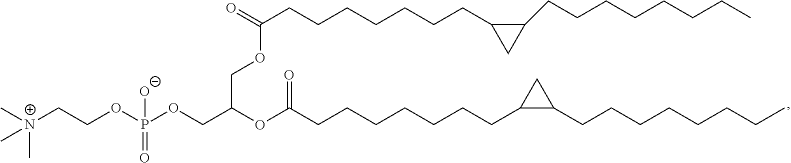 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

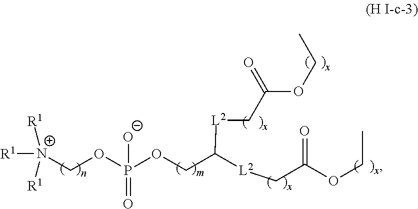

or a salt thereof.

In certain embodiments, the compound of Formula (H I-c) is of the following formulae:

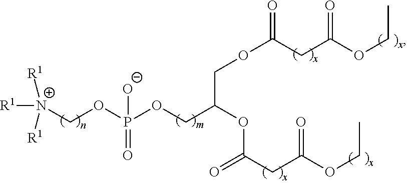

or a salt thereof.

In certain embodiments, the compound of Formula (H I-c) is the following:

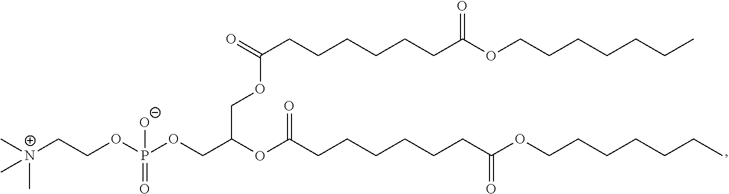 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

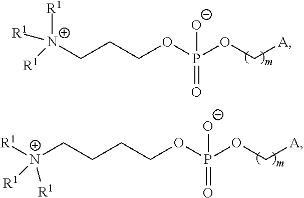

or a salt thereof.

In certain embodiments, a compound of Formula (H I) is one of the following:

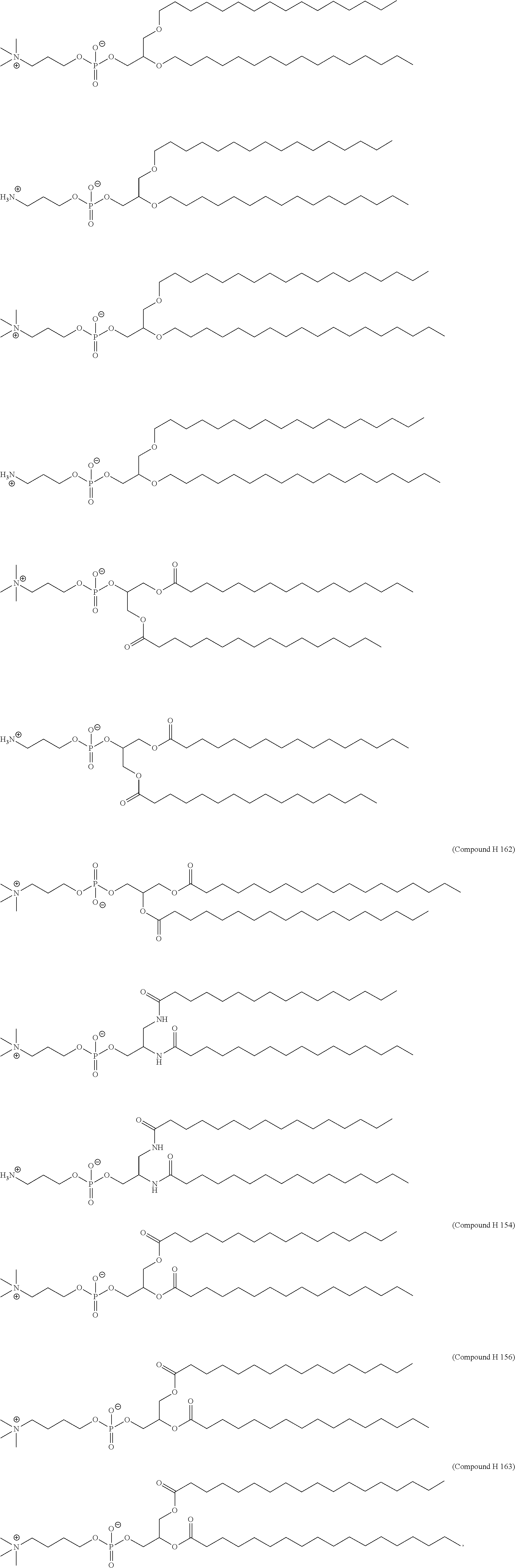

or salts thereof.

Numerous LNP formulations having phospholipids other than DSPC were prepared and tested for activity, as demonstrated in the examples below.

Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iv) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

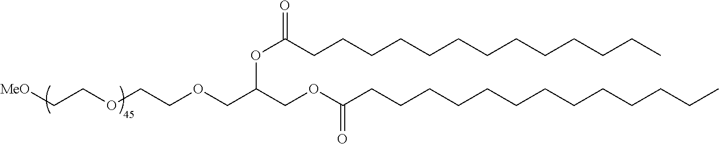

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

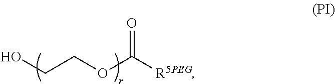

or a salt or isomer thereof, wherein:

r is an integer between 1 and 100;

R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

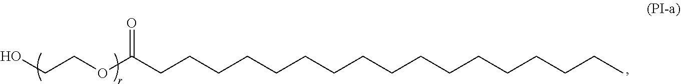

or a salt or isomer thereof, wherein r is an integer between 1 and 100.

For example, the PEG lipid is a compound of the following formula:

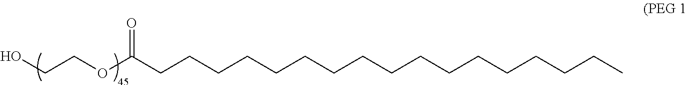

or a salt or isomer thereof.

The PEG lipid may be a compound of Formula (PII):

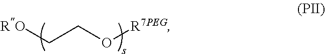

or a salt or isomer thereof, wherein:

s is an integer between 1 and 100;

R″ is a hydrogen, C₁₋₁₀ alkyl, or an oxygen protecting group;

R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

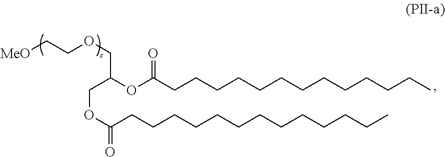

or a salt or isomer thereof, wherein s is an integer between 1 and 100.

For example, the PEG lipid is a compound of the following formula:

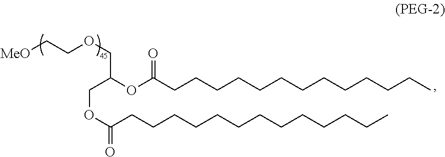

or a salt or isomer thereof.

In certain embodiments, a PEG lipid useful in the present invention is a compound of Formula (PIII). Provided herein are compounds of Formula (PIII):

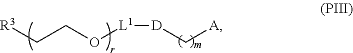

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

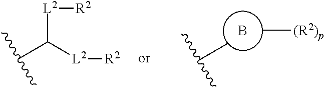

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N}); each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

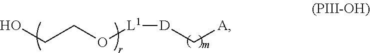

or a salt thereof.

In certain embodiments, D is a moiety obtained by click chemistry (e.g., triazole). In certain embodiments, the compound of Formula (PIII) is of Formula (PIII-a-1) or (PIII-a-2):

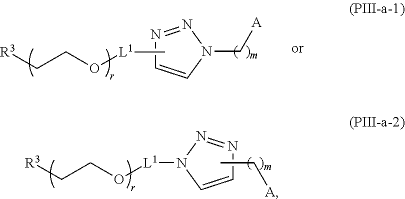

or a salt thereof.

In certain embodiments, the compound of Formula (PIII) is of one of the following formulae:

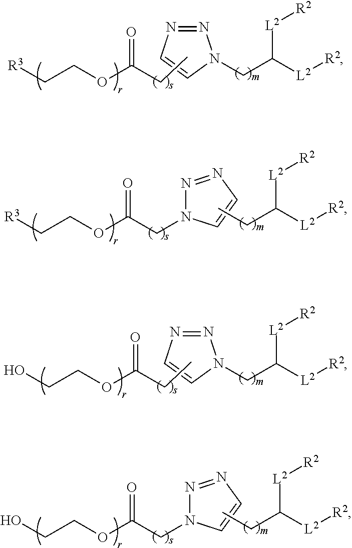

or a salt thereof, wherein

s is 0, 1, 2, 3, 4, 5, 6, 7, 8, 9, or 10.

In certain embodiments, the compound of Formula (PIII) is of one of the following formulae:

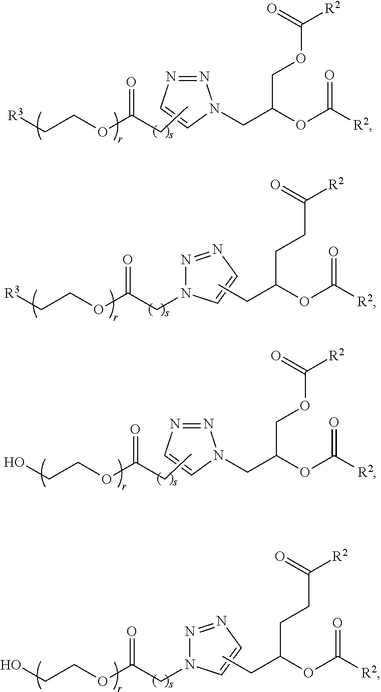

or a salt thereof.

In certain embodiments, a compound of Formula (PIII) is of one of the following formulae:

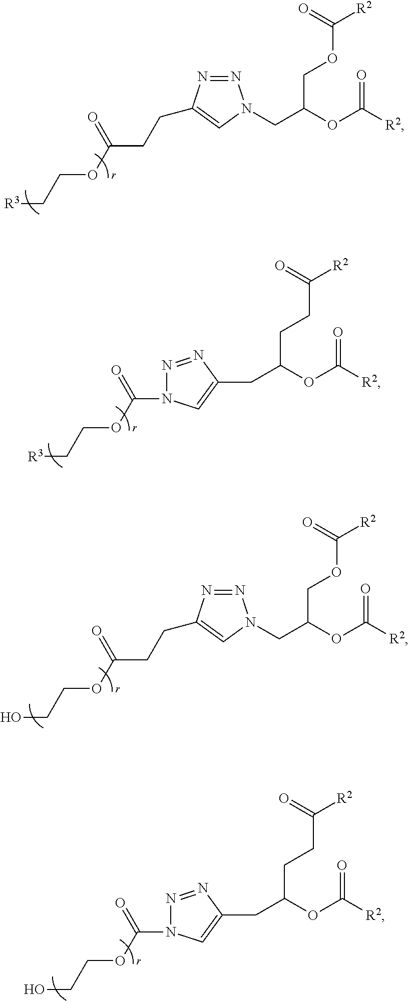

or a salt thereof.

In certain embodiments, a compound of Formula (PIII) is of one of the following formulae:

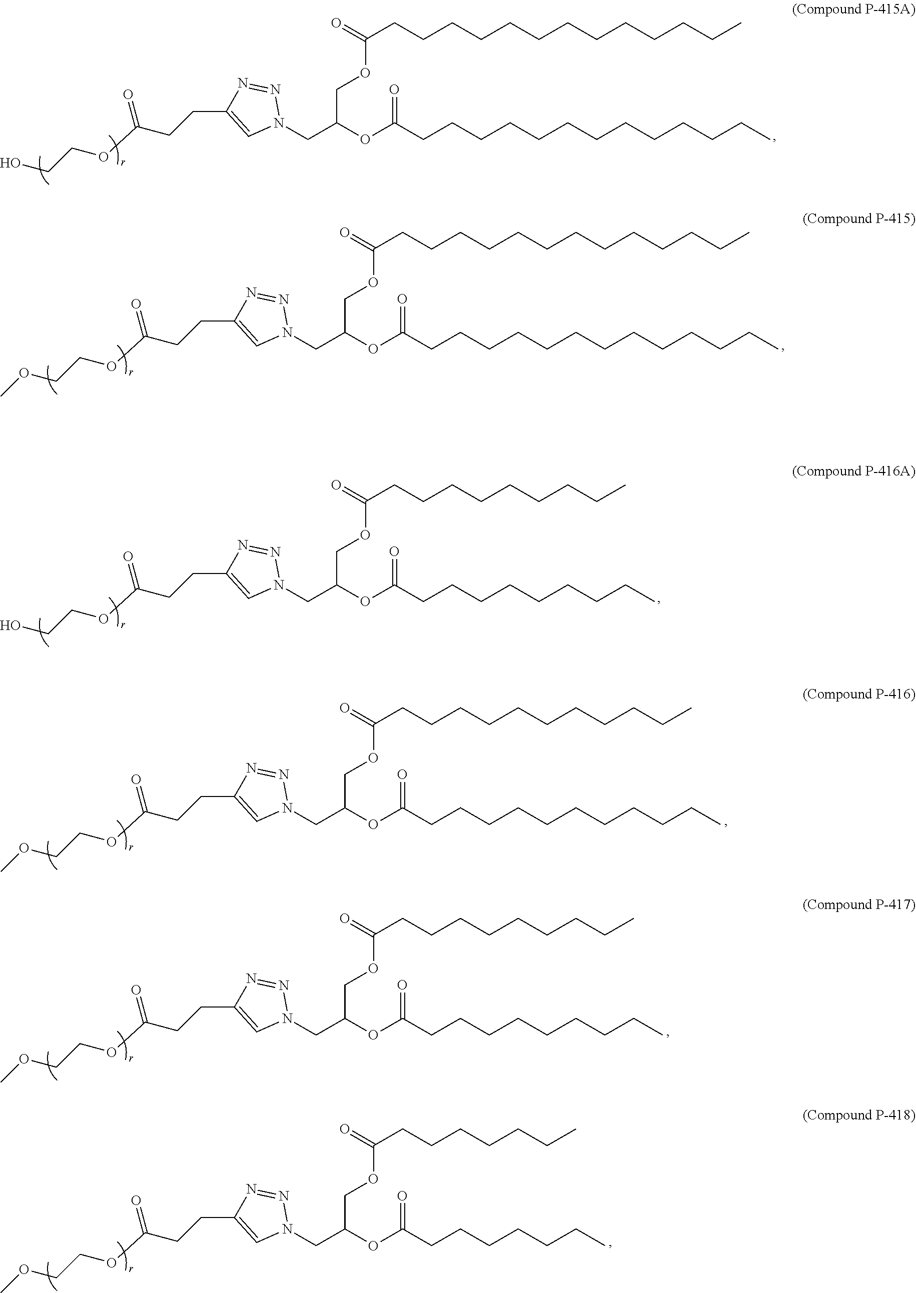

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

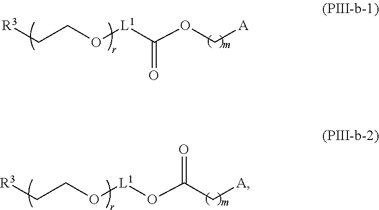

or a salt thereof.

In certain embodiments, a compound of Formula (PIII) is of Formula (PIII-b-1-OH) or (PIII-b-2-OH):

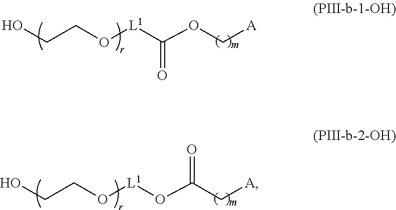

or a salt thereof.

In certain embodiments, the compound of Formula (PIII) is of one of the following formulae:

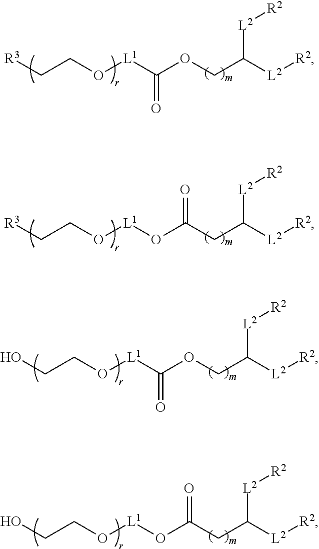

or a salt thereof.

In certain embodiments, a compound of Formula (PIII) is of one of the following formulae:

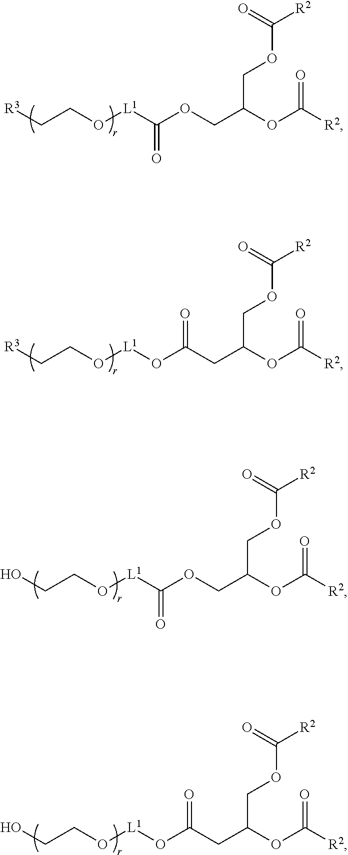

or a salt thereof.

In certain embodiments, a compound of Formula (PIII) is of one of the following formulae:

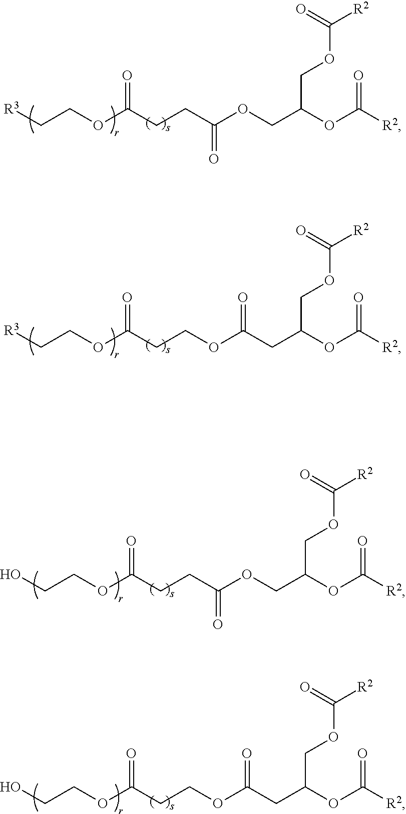

or a salt thereof.

In certain embodiments, a compound of Formula (PIII) is of one of the following formulae:

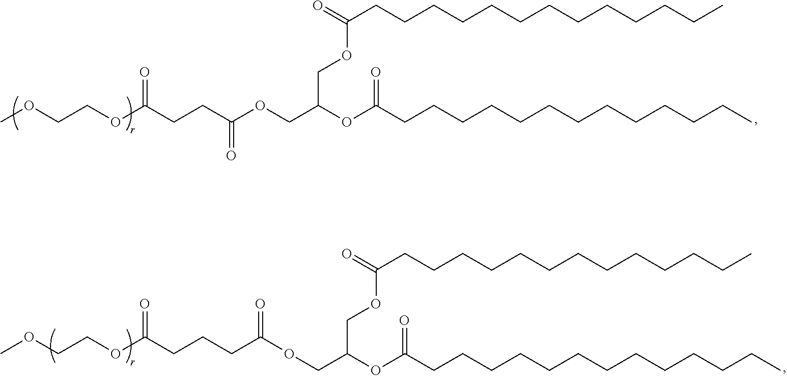

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

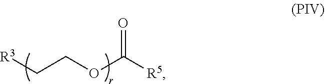

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and each instance of R^{N} is independently hydrogen, optionally substituted alkyl, or a nitrogen protecting group.

In certain embodiments, the compound of Formula (PIV is of Formula (PIV-OH):

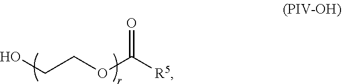

or a salt thereof. In some embodiments, r is 40-50. In some embodiments, r is 45.

In certain embodiments, a compound of Formula (PIV) is of one of the following formulae:

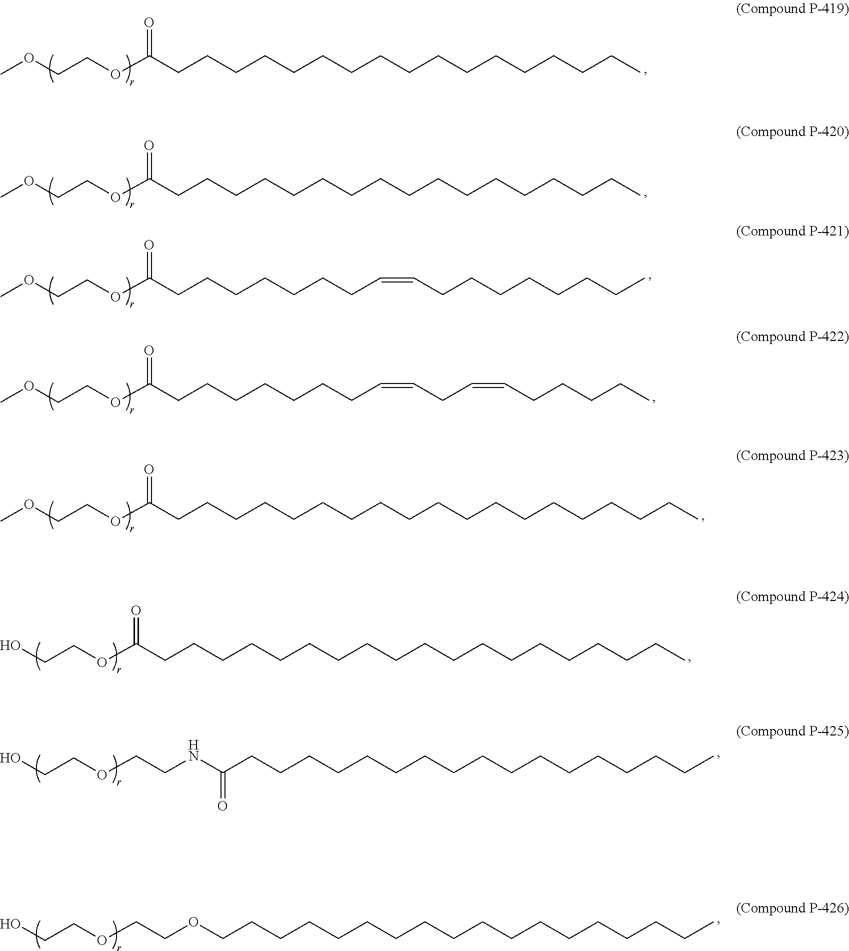

or a salt thereof. In some embodiments, r is 40-50. In some embodiments, r is 45.

In yet other embodiments the compound of Formula (PIV) is:

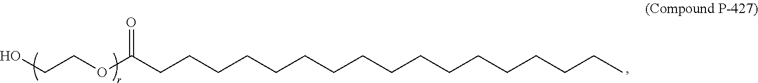

or a salt thereof.

In one embodiment, the compound of Formula (PIV) is

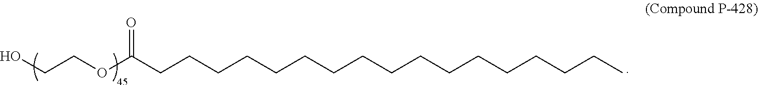

In one aspect, provided herein are lipid nanoparticles (LNPs) comprising PEG lipids of Formula (PV):

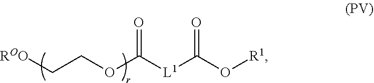

or pharmaceutically acceptable salts thereof; wherein

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

R¹ is optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group; and

r is an integer from 2 to 100, inclusive.

In certain embodiments, the PEG lipid of Formula (PV) is of the following formula:

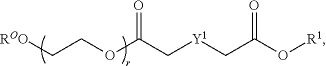

or a pharmaceutically acceptable salt thereof; wherein:

Y¹ is a bond, —CR₂—, —O—, —NR^{N}—, or —S—;

each instance of R is independently hydrogen, halogen, or optionally substituted alkyl; and

R^{N} is hydrogen, optionally substituted alkyl, optionally substituted acyl, or a nitrogen protecting group.

In certain embodiments, the PEG lipid of Formula (PV) is of one of the following formulae:

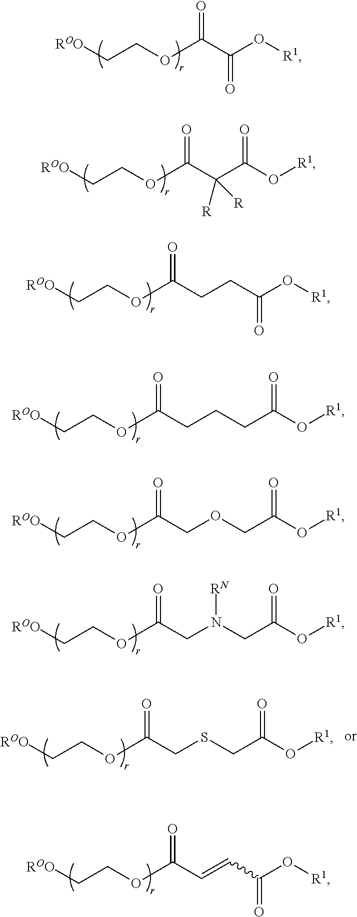

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

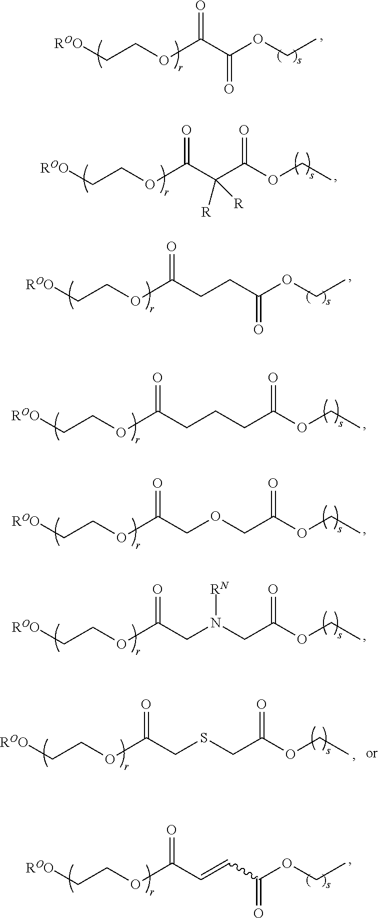

or a pharmaceutically acceptable salt thereof, wherein:

s is an integer from 5-25, inclusive.

In certain embodiments, the PEG lipid of Formula (PV) is of one of the following formulae:

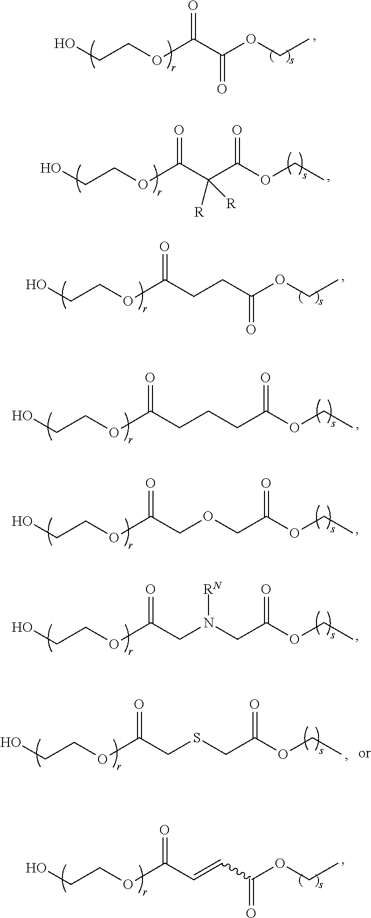

or a pharmaceutically acceptable salt thereof.

In certain embodiments, the PEG lipid of Formula (PV) is selected from the group consisting of:

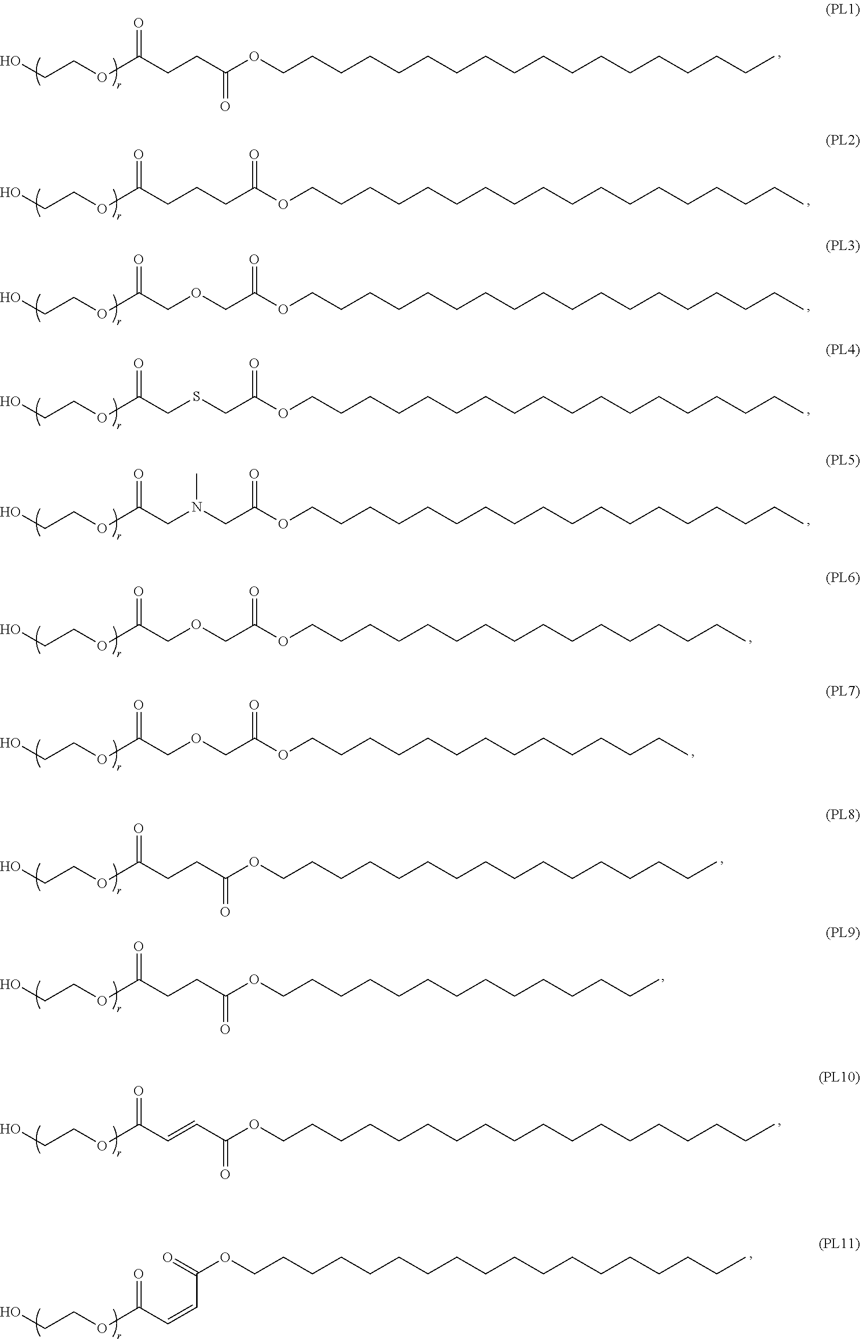

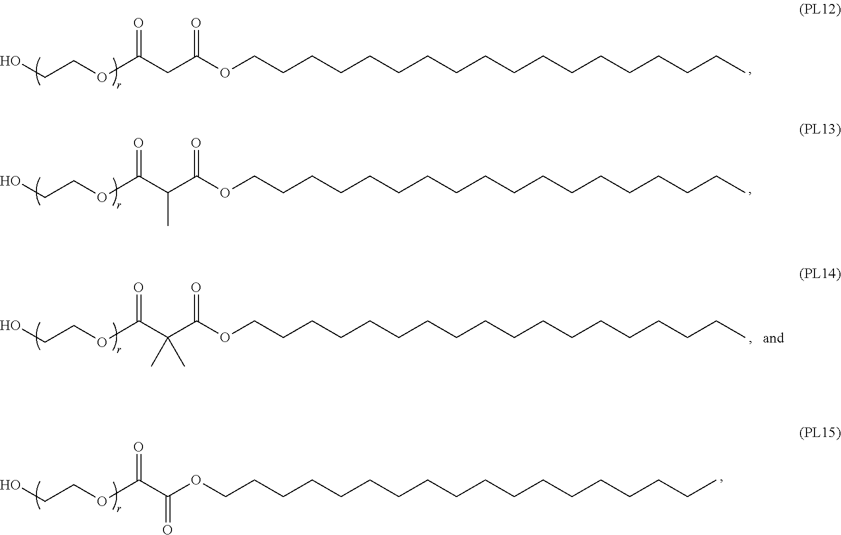

and pharmaceutically acceptable salts thereof.

In another aspect, provided herein are lipid nanoparticles (LNPs) comprising PEG lipids of Formula (PVI):

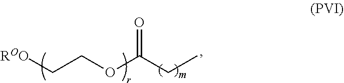

or pharmaceutically acceptable salts thereof; wherein:

R^{O} is hydrogen, optionally substituted alkyl, optionally substituted acyl, or an oxygen protecting group;

r is an integer from 2 to 100, inclusive; and

m is an integer from 5-15, inclusive, or an integer from 19-30, inclusive.

In certain embodiments, the PEG lipid of Formula (PVI) is of one of the following formulae:

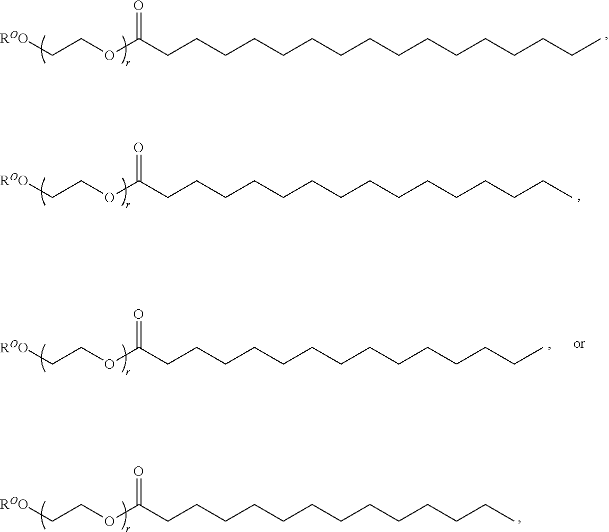

or a pharmaceutically acceptable salt thereof.

In certain embodiments, the PEG lipid of Formula (PVI) is of one of the following formulae:

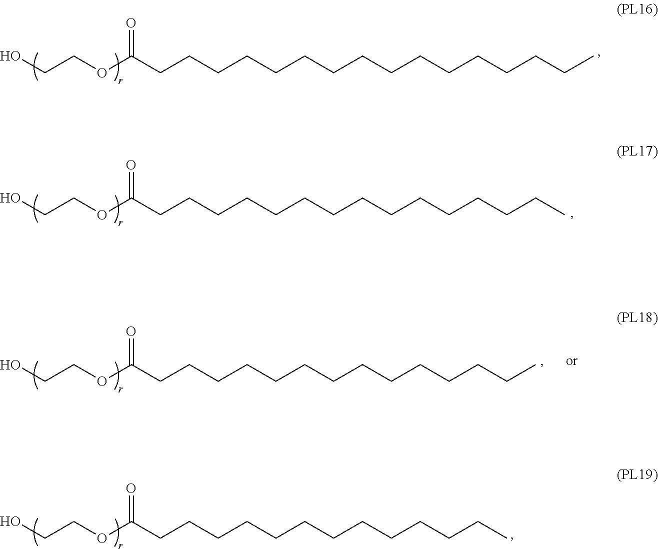

or a pharmaceutically acceptable salt thereof.

In another aspect, provided herein are lipid nanoparticles (LNPs) comprising PEG lipids of Formula (PVII):

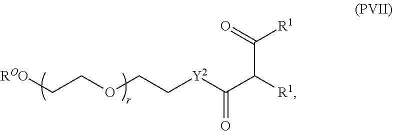

or pharmaceutically acceptable salts thereof, wherein:

Y² is —O—, —NR^{N}—, or —S—

each instance of R¹ is independently optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group;

R^{N} is hydrogen, optionally substituted alkyl, optionally substituted acyl, or a nitrogen protecting group; and

r is an integer from 2 to 100, inclusive.

In certain embodiments, the PEG lipid of Formula (PVII) is of one of the following formulae:

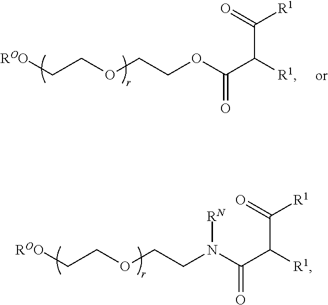

or a pharmaceutically acceptable salt thereof.

In certain embodiments, the PEG lipid of Formula (PVII) is of one of the following formulae:

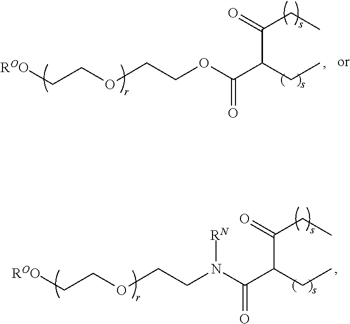

or a pharmaceutically acceptable salt thereof; wherein:

each instance of s is independently an integer from 5-25, inclusive.

In certain embodiments, the PEG lipid of Formula (PVII) is of one of the following

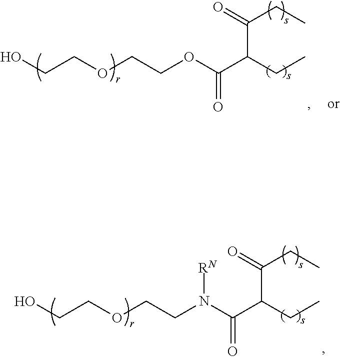

or a pharmaceutically acceptable salt thereof.

In certain embodiments, the PEG lipid of Formula (PVII) is selected from the group consisting of:

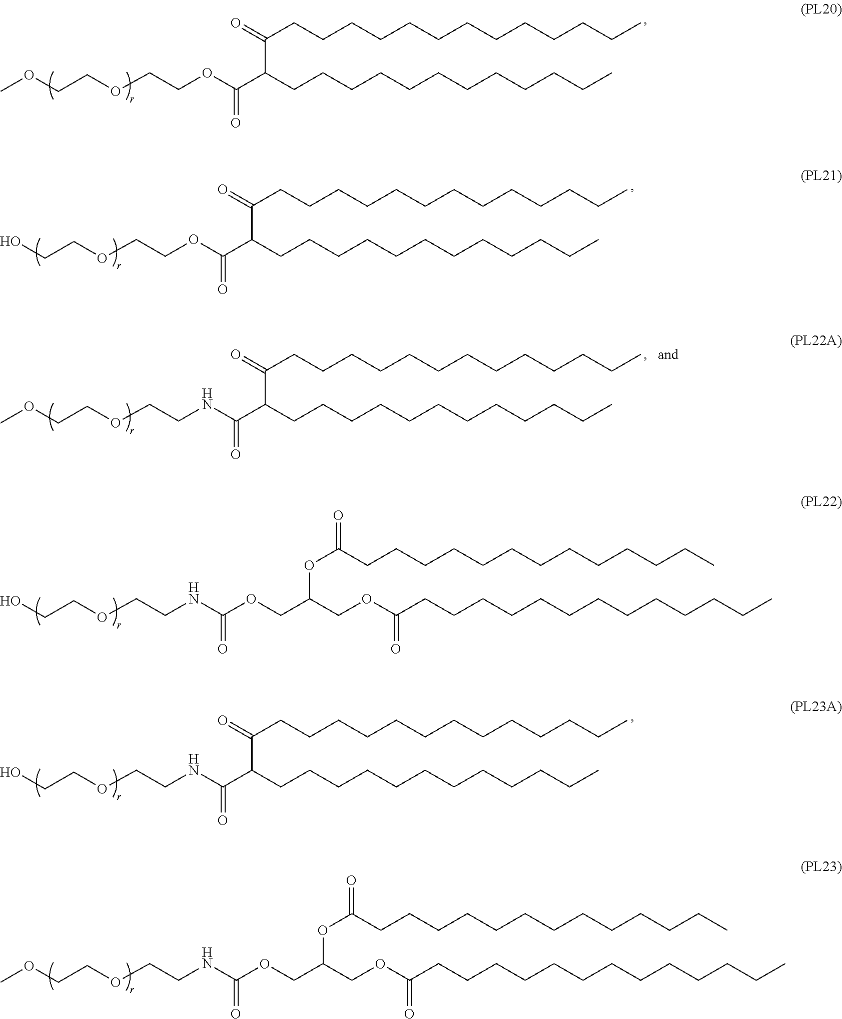

and pharmaceutically acceptable salts thereof.

In another aspect, provided herein are lipid nanoparticles (LNPs) comprising PEG lipids of Formula (PVIII):

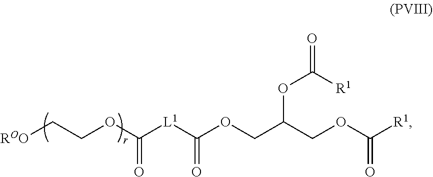

or pharmaceutically acceptable salts thereof, wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

each instance of R¹ is independently optionally substituted C₅₋₃₀ alkyl, optionally substituted C₃₋₃₀ alkenyl, or optionally substituted C₅₋₃₀ alkynyl; R^{O} is hydrogen, optionally substituted alkyl, optionally substituted acyl, or an oxygen protecting group;

r is an integer from 2 to 100, inclusive;

provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

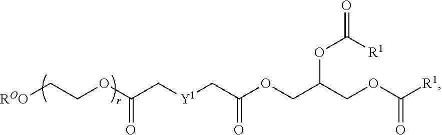

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl;

R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

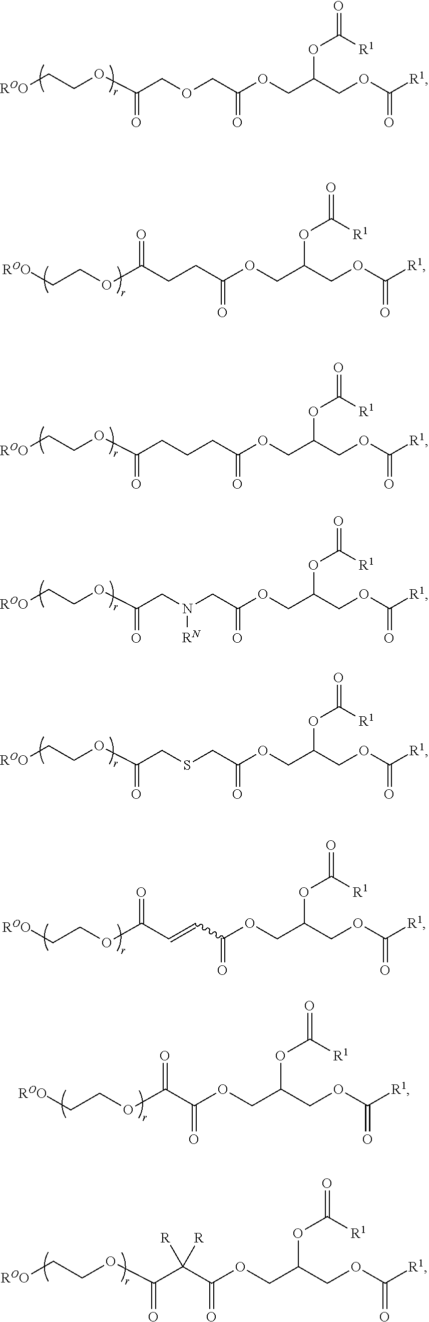

or a pharmaceutically acceptable salt thereof, wherein:

each instance of R is independently hydrogen, halogen, or optionally substituted alkyl.

In certain embodiments, the PEG lipid of Formula (PVIII) is of one of the following formulae:

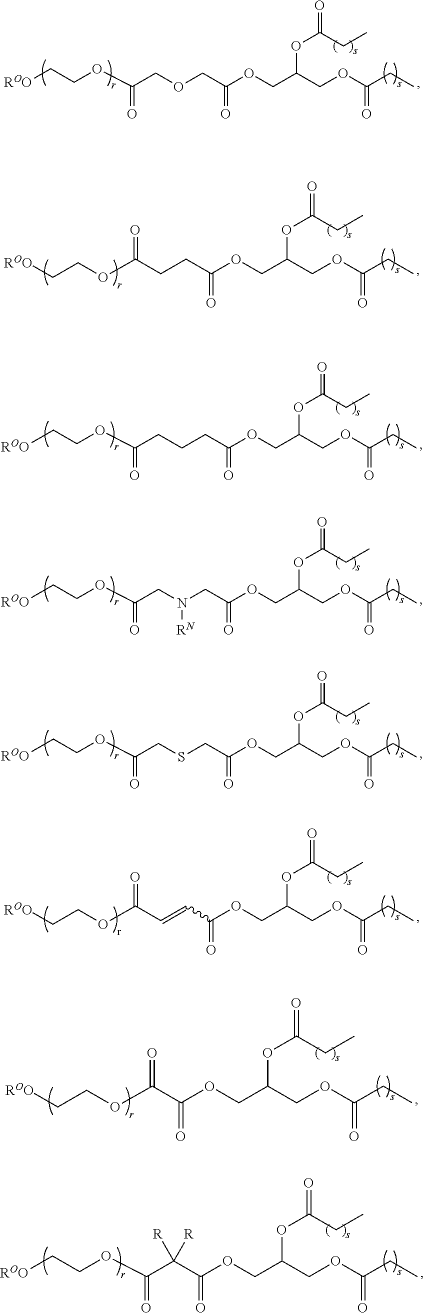

or a pharmaceutically acceptable salt thereof; wherein:

each instance of R is independently hydrogen, halogen, or optionally substituted alkyl; and

each s is independently an integer from 5-25, inclusive.

In certain embodiments, the PEG lipid of Formula (PVIII) is of one of the following formulae:

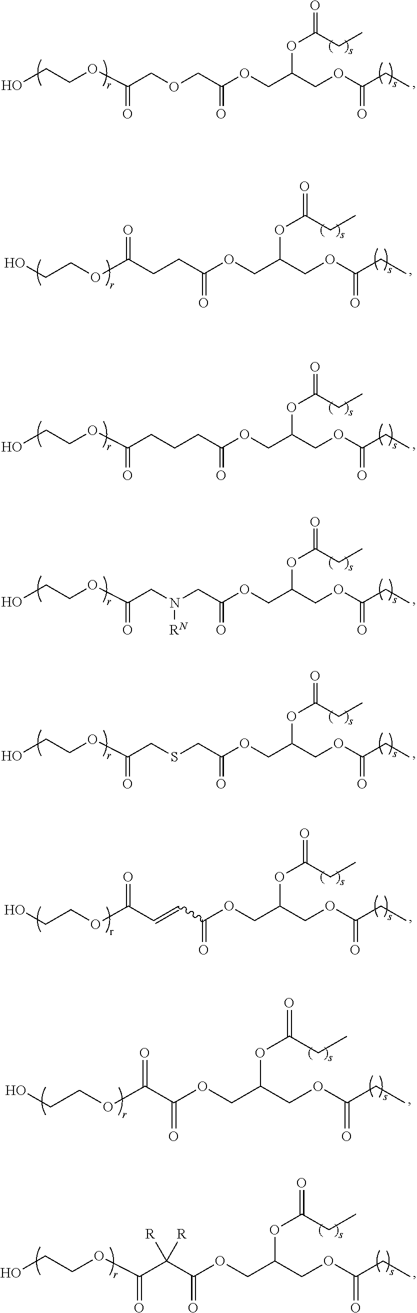

or a pharmaceutically acceptable salt thereof.

In certain embodiments, the PEG lipid of Formula (PVIII) is selected from the group consisting of:

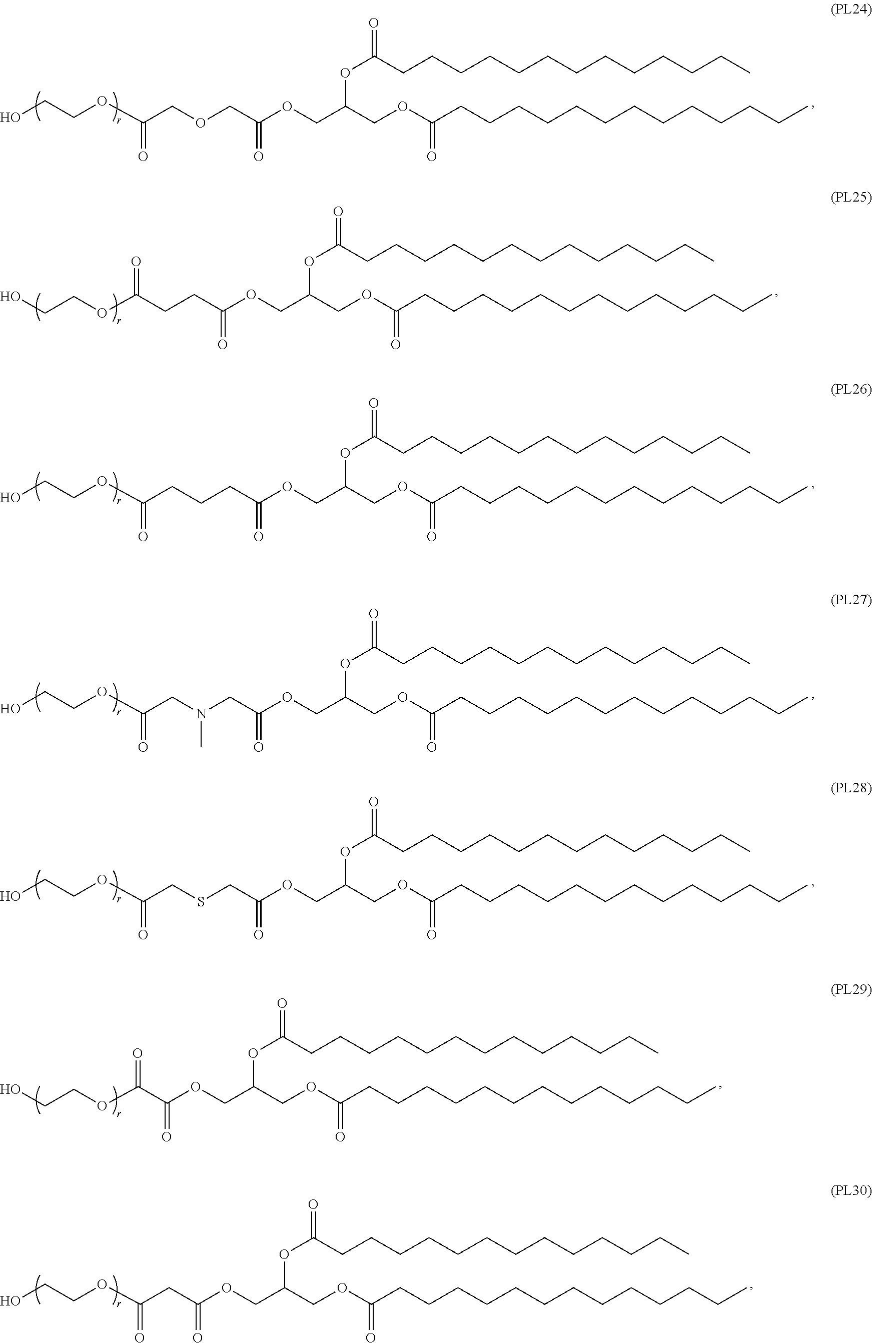

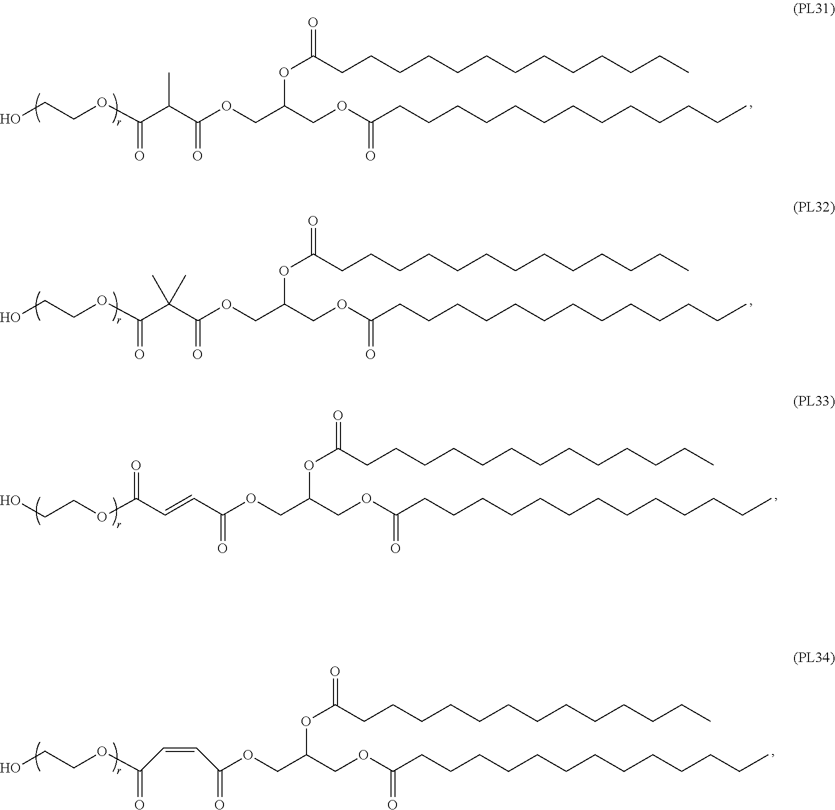

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

Compound: HO-PEG₂₀-ester-C18

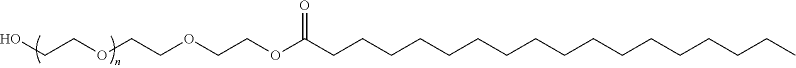

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated and backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, a target cell delivery lipid of the pharmaceutical compositions disclosed herein does not comprise a PEG-lipid.

In one embodiment, a target cell target cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, a target cell target cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of. Cmpd 428, PL16, PL17, PL 18, PL19, PL 1, and PL 2.

Target cell Delivery Potentiating Lipids

An effective amount of the target cell delivery potentiating lipid in an LNP enhances delivery of the agent to a target cell (e.g., a human or primate target cell, e.g., liver cell or splenic cells) relative to an LNP lacking the target cell delivery potentiating lipid, thereby creating a target cell target cell delivery LNP. Target cell delivery potentiating lipids can be characterized in that, when present in an LNP, they promote delivery of the agent present in the LNP to target cells as compared to a reference LNP lacking the target cell delivery potentiating lipid.

In one embodiment, the presence of at least one target cell delivery potentiating lipid in an LNP results in an increase in the percentage of LNPs associated with target cells as compared to a reference LNP lacking at least one target cell delivery potentiating lipid. In another embodiment, the presence of at least one target cell delivery potentiating lipid in an LNP results in an increase in the delivery of a nucleic acid molecule agent to target cells as compared to a reference LNP lacking the target cell delivery potentiating lipid. In one embodiment, the presence of at least one target cell delivery potentiating lipid in an LNP results in an increase in the delivery of a nucleic acid molecule agent to liver cells as compared to a reference LNP lacking the target cell delivery potentiating lipid. In particular, in one embodiment, the presence of at least one target cell delivery potentiating lipid in an LNP results in an increase in the delivery of a nucleic acid molecule agent to hepatocyte cells as compared to a reference LNP lacking the target cell delivery potentiating lipid. In one embodiment, the presence of at least one target cell delivery potentiating lipid in an LNP results in an increase in the delivery of a nucleic acid molecule agent to Kupffer cells as compared to a reference LNP lacking the target cell delivery potentiating lipid. In one embodiment, the presence of at least one target cell delivery potentiating lipid in an LNP results in an increase in the delivery of a nucleic acid molecule agent to liver sinusoidal cells as compared to a reference LNP lacking the target cell delivery potentiating lipid. In one embodiment, the presence of at least one target cell delivery potentiating lipid in an LNP results in an increase in the delivery of a nucleic acid molecule agent to hepatic stellate cells as compared to a reference LNP lacking the target cell delivery potentiating lipid.

In one embodiment, the presence of at least one target cell delivery potentiating lipid in an LNP results in preferentially uptake of the LNP in the target cell as compared to a reference LNP lacking at least one target cell delivery potentiating lipid. In one embodiment, the presence of at least one target cell delivery potentiating lipid in an LNP results in an increase in the percentage of LNPs taken up by target cells (e.g., opsonized by target cells) as compared to a reference LNP lacking at least one target cell delivery potentiating lipid.

In one embodiment, when the nucleic acid molecule is an mRNA, the presence of at least one target cell delivery potentiating lipid results in at least about 2-fold greater expression of a protein molecule encoded by the mRNA in target cells (e.g., liver cells (e.g., a hepatocyte, a hepatic stellate cell, a Kupffer cell, or a liver sinusoidal cell) or splenic cells) as compared to a reference LNP lacking the target cell delivery potentiating lipid.

In one embodiment, a target cell delivery potentiating lipid is an ionizable lipid. In any of the foregoing or related aspects, the ionizable lipid (denoted by I) of the LNP of the disclosure comprises a compound included in any e.g. a compound having any of Formula (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), (I VIIId), (I XI), (I XI-a), or (I XI-b), (I IX), (I IXa1), (I IXa2), (I IXa3), (I IXa4), (I IXa5), (I IXa6), (I IXa7), or (I IXa8) and/or any of Compounds X, Y, I 48, I 49, I 50, I 109, I 111, I 113, I 181, I 182, I 244, I 292, I 301, I 321, I 322, I 326, I 328, I 330, I 331, I 332 or I M.

In one embodiment, a target cell delivery potentiating lipid is an ionizable lipid. In any of the foregoing or related aspects, the ionizable lipid of the LNP of the disclosure comprises a compound described herein as Compound Y, Compound I-321, Compound I-292, Compound I-326, Compound I-182, Compound I-301, Compound I-48, Compound I-49, Compound I-50, Compound I-328, Compound I-330, Compound I-109, Compound I-111 or Compound I-181.

In any of the foregoing or related aspects, the ionizable lipid of the LNP of the disclosure comprises at least one compound selected from the group consisting of: I 25 (also referred to as Compound Y), I 48, I 49, I 50, I 109, I 111, I 113, I 181, I 182, I 244, I 292, I 301, I 309, I 317, I 321, I 322, I 326, I 328, I 330, I 331, I 332, I 347, I 348, I 349, I 350, I 351 and I 352. In another embodiment, the ionizable lipid of the LNP of the disclosure comprises a compound selected from the group consisting of: I 25 (also referred to as Compound Y), I 48, I 49, I 50, I 109, I111, I 181, I 182, I 292, I 301, I 321, I 326, I 328, and I 330. In another embodiment, the ionizable lipid of the LNP of the disclosure comprises a compound selected from the group consisting of: Compound Nos. I 49, I 182, I301, I 321, and I 326.

It will be understood that in embodiments where the target cell delivery potentiating lipid comprises an ionizable lipid, it may be the only ionizable lipid present in the LNP or it may be present as a blend with at least one additional ionizable lipid. That is to say that a blend of ionizable lipids (e.g., more than one that have target cell delivery potentiating effects or one that has a target cell delivery potentiating effect and at least one that does not) may be employed.

In one embodiment, a target cell delivery potentiating lipid comprises a sterol. In another embodiment, a target cell delivery potentiating lipid comprises a naturally occurring sterol. In another embodiment, a target cell delivery potentiating lipid comprises a modified sterol. In one embodiment, a target cell delivery potentiating lipid comprises one or more phytosterols. In one embodiment, the target cell delivery potentiating lipid comprises a phytosterol/cholesterol blend.

In one embodiment, the target cell delivery potentiating lipid comprises an effective amount of a phytosterol.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

The term “effective amount of phytosterol” is intended to mean an amount of one or more phytosterols in a lipid-based composition, including an LNP, that will elicit a desired activity (e.g., enhanced delivery, enhanced target cell uptake, enhanced nucleic acid activity). In some embodiments, an effective amount of phytosterol is all or substantially all (i.e., about 99-100%) of the sterol in a lipid nanoparticle. In some embodiments, an effective amount of phytosterol is less than all or substantially all of the sterol in a lipid nanoparticle (less than about 99-100%), but greater than the amount of non-phytosterol sterol in the lipid nanoparticle. In some embodiments, an effective amount of phytosterol is greater than 50%, greater than 60%, greater than 70%, greater than 75%, greater than 80%, greater than 85%, greater than 90% or greater than 95% the total amount of sterol in a lipid nanoparticle. In some embodiments, an effective amount of phytosterol is 95-100%, 75-100%, or 50-100% of the total amount of sterol in a lipid nanoparticle.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

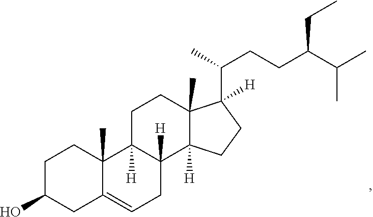

including analogs, salts or esters thereof.
In some embodiments, the sitosterol is a stigmasterol.

In some embodiments, the stigmasterol has the formula:

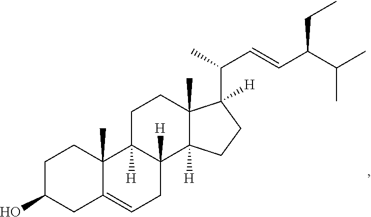

including analogs, salts or esters thereof.

In some embodiments, the sitosterol is a campesterol.

In some embodiments, the campesterol has the formula:

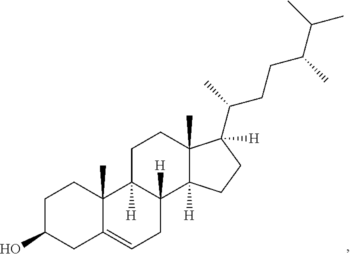

including analogs, salts or esters thereof.

In some embodiments, the sitosterol is a sitostanol.

In some embodiments, the sitostanol has the formula:

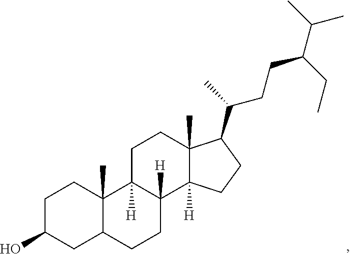

including analogs, salts or esters thereof.

In some embodiments, the sitosterol is a campestanol.

In some embodiments, the campestanol has the formula:

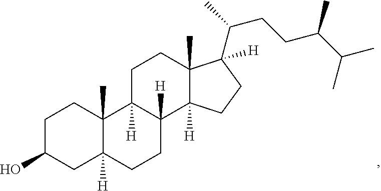

including analogs, salts or esters thereof.

In some embodiments, the sitosterol is a brassicasterol.

In some embodiments the brassicasterol has the formula:

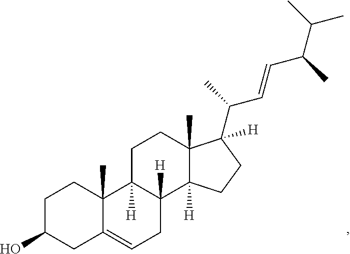

including analogs, salts or esters thereof.

In some embodiments, the sitosterol is a fucosterol.

In some embodiments, the fucosterol has the formula:

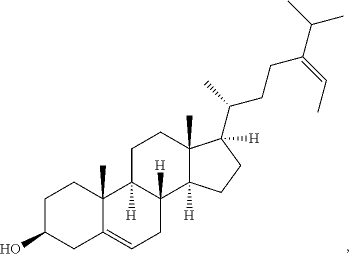

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, a target cell delivery enhancing LNP comprises more than one type of structural lipid.

For example, in one embodiment, the target cell delivery enhancing LNP comprises at least one target cell delivery potentiating lipid which is a phytosterol. In one embodiment, the phytosterol is the only structural lipid present in the LNP. In another embodiment, the target cell target cell delivery LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g, phospholipid), 38.5% structural lipid, and 1.5% PEG lipid. In certain embodiments, the LNP comprises 50% ionizable lipid, 10% helper lipid (e.g, phospholipid), 38% structural lipid, and 2% PEG lipid. In certain embodiments, the LNP comprises 50% ionizable lipid, 20% helper lipid (e.g, phospholipid), 28.5% structural lipid, and 1.5% PEG lipid. In certain embodiments, the LNP comprises 50% ionizable lipid, 20% helper lipid (e.g, phospholipid), 28% structural lipid, and 2% PEG lipid. In certain embodiments, the LNP comprises 40% ionizable lipid, 30% helper lipid (e.g, phospholipid), 28.5% structural lipid, and 1.5% PEG lipid. In certain embodiments, the LNP comprises 40% ionizable lipid, 30% helper lipid (e.g, phospholipid), 28% structural lipid, and 2% PEG lipid. In certain embodiments, the LNP comprises 45% ionizable lipid, 20% helper lipid (e.g, phospholipid), 33.5% structural lipid, and 1.5% PEG lipid. In certain embodiments, the LNP comprises 45% ionizable lipid, 20% helper lipid (e.g, phospholipid), 33% structural lipid, and 2% PEG lipid.

In one aspect, the target cell delivery enhancing LNP comprises phytosterol and the LNP does not comprise an additional structural lipid. Accordingly, the structural lipid (sterol) component of the LNP consists of phytosterol. In another aspect, the target cell delivery enhancing LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition 5-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, a target cell delivery enhancing LNP comprises a non-cationic helper lipid, e.g., phospholipid. In any of the foregoing or related aspects, the non-cationic helper lipid (e.g, phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L1, P L2, P L16, P L17, P L18, P L19, P L22, P L23, DMG, DPG and DSG. In another embodiment, the PEG lipid is selected from the group consisting of Cmpd 428, PL16, PL17, PL 18, PL19, P L5, PL 1, and PL 2.

In one embodiment, a target cell delivery potentiating lipid comprises an effective amount of a combination of an ionizable lipid and a phytosterol.

In other embodiments, the disclosure provides lipid nanoparticles comprising one or more target cell delivery potentiating lipids, wherein the LNP comprises Compound Y as the ionizable lipid, DSPC as the phospholipid, cholesterol or a cholesterol/β-sitosterol blend as the structural lipid and Compound 428 as the PEG lipid. In various embodiments of these Compound Y-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-182 as the ionizable lipid, DSPC as the phospholipid, cholesterol or a cholesterol/β-sitosterol blend as the structural lipid and Compound 428 as the PEG lipid. In various embodiments of these Compound I-182-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-321 as the ionizable lipid, DSPC as the phospholipid, cholesterol or a cholesterol/β-sitosterol blend as the structural lipid and Compound 428 as the PEG lipid. In various embodiments of these Compound I-321-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-292 as the ionizable lipid, DSPC as the phospholipid, cholesterol or a cholesterol/β-sitosterol blend as the structural lipid and Compound 428 as the PEG lipid. In various embodiments of these Compound I-292-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-326 as the ionizable lipid, DSPC as the phospholipid, cholesterol or a cholesterol/β-sitosterol blend as the structural lipid and Compound 428 as the PEG lipid. In various embodiments of these Compound I-326-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-301 as the ionizable lipid, DSPC as the phospholipid, cholesterol or a cholesterol/β-sitosterol blend as the structural lipid and Compound 428 as the PEG lipid. In various embodiments of these Compound I-301-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-48 as the ionizable lipid, DSPC as the phospholipid, cholesterol or a cholesterol/β-sitosterol blend as the structural lipid and Compound 428 as the PEG lipid. In various embodiments of these Compound I-48-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-49 as the ionizable lipid, DSPC as the phospholipid, cholesterol or a cholesterol/β-sitosterol blend as the structural lipid and Compound 428 as the PEG lipid. In various embodiments of these Compound I-49-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-50 as the ionizable lipid, DSPC as the phospholipid, cholesterol or a cholesterol/β-sitosterol blend as the structural lipid and Compound 428 as the PEG lipid. In various embodiments of these Compound I-50-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-328 as the ionizable lipid, DSPC as the phospholipid, cholesterol or a cholesterol/β-sitosterol blend as the structural lipid and Compound 428 as the PEG lipid. In various embodiments of these Compound I-328-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-330 as the ionizable lipid, DSPC as the phospholipid, cholesterol or a cholesterol/β-sitosterol blend as the structural lipid and Compound 428 as the PEG lipid. In various embodiments of these Compound I-330-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-109 as the ionizable lipid, DSPC as the phospholipid, cholesterol or a cholesterol/β-sitosterol blend as the structural lipid and Compound 428 as the PEG lipid. In various embodiments of these Compound I-109-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-111 as the ionizable lipid, DSPC as the phospholipid, cholesterol or a cholesterol/β-sitosterol blend as the structural lipid and Compound 428 as the PEG lipid. In various embodiments of these Compound I-111-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises Compound I-181 as the ionizable lipid, DSPC as the phospholipid, cholesterol or a cholesterol/β-sitosterol blend as the structural lipid and Compound 428 as the PEG lipid. In various embodiments of these Compound I-181-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more target cell delivery potentiating lipids, wherein the LNP comprises any of Compounds X, Y, I-321, I-292, I-326, I-182, I-301, I-48, I-49, I-50, I-328, I-330, I-109, I-111 or I-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesterol/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% β-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% β-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% β-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiments, the disclosure provides lipid nanoparticles comprising one or more target cell delivery potentiating lipids, wherein the LNP comprises Compound I-49, Compound I-301, Compound I-321 or Compound I-326 as the ionizable lipid; DSPC as the phospholipid; cholesterol or a cholesterol/β-sitosterol blend as the structural lipid; and Compound 428 as the PEG lipid. In one embodiment, the LNP enhances delivery to target cells, e.g., liver cells or splenic cells.

In other embodiment, the disclosure provides lipid nanoparticles comprising one or more target cell delivery potentiating lipids, wherein the LNP comprises Compound I-109, Compound I-111, Compound I-181, Compound I-182 or Compound I-244, wherein the LNP enhances delivery to monocytes. The other components of the LNP can be selected from those disclosed herein, for example DSPC as the phospholipid; cholesterol or a cholesterol/β-sitosterol blend as the structural lipid; and Compound 428 as the PEG lipid.

In other embodiment, the disclosure provides lipid nanoparticles comprising one or more target cell delivery potentiating lipids, wherein the LNP comprises camposterol, β-sitostanol or stigmasterol as the structural lipid, wherein the LNP enhances delivery to monocytes. The other components of the LNP can be selected from those disclosed herein, for example Compound I-109, Compound I-111, Compound I-181, Compound I-182 or Compound I-244 as the ionizable lipid; DSPC as the phospholipid; and Compound 428 as the PEG lipid.

In other embodiment, the disclosure provides lipid nanoparticles comprising one or more target cell delivery potentiating lipids, wherein the LNP comprises DOPC, DMPE or H-409 as the helper lipid (e.g., phospholipid), wherein the LNP enhances delivery to monocytes. The other components of the LNP can be selected from those disclosed herein, for example Compound I-109, Compound I-111, Compound I-181, Compound I-182 or Compound I-244 as the ionizable lipid; cholesterol, a cholesterol/β-sitosterol blend, camposterol, β-sitostanol or stigmasterol as the structural lipid; and Compound 428 as the PEG lipid.

### Exemplary Additional LNP Components

Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

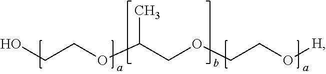

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

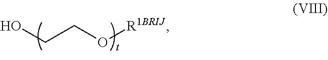

or a salt or isomer thereof, wherein:

t is an integer between 1 and 100;

R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

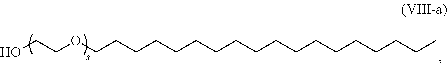

or a salt or isomer thereof.

In some embodiments, R^{1BRIJ} is C₁₈ alkenyl. For example, the polyethylene glycol ether is a compound of Formula (VIII-b):

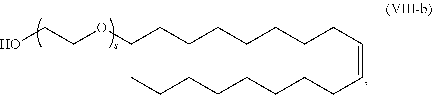

or a salt or isomer thereof.

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 04, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer© solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

LNP Compositions

A lipid nanoparticle (LNP) described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The LNPs of the invention comprise at least one target cell delivery potentiating lipid. The subject LNPs comprise: an effective amount of a target cell delivery potentiating lipid as a component of an LNP, wherein the LNP comprises an (i) ionizable lipid; (ii) cholesterol or other structural lipid; (iii) a non-cationic helper lipid or phospholipid; a (iv) PEG lipid and (v) an agent (e.g, an nucleic acid molecule) encapsulated in and/or associated with the LNP, wherein the effective amount of the target cell delivery potentiating lipid enhances delivery of the agent to a target cell (e.g., a human or primate target cell, e.g., liver cell or splenic cell) relative to an LNP lacking the target cell delivery potentiating lipid.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol % ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol % ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol % ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol % ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

Lipid nanoparticles of the disclosure may be designed for one or more specific applications or targets. For example, the subject lipid nanoparticles may optionally be designed to further enhance delivery of a nucleic acid molecule, such as an RNA, to a particular target cell (e.g., liver cell or splenic cell), tissue, organ, or system or group thereof in a mammal's, e.g., a human's body. Physiochemical properties of lipid nanoparticles may be altered in order to increase selectivity for particular bodily targets. For instance, particle sizes may be adjusted to promote target cell uptake. As set forth above, the nucleic acid molecule included in a lipid nanoparticle may also be selected based on the desired delivery to target cells. For example, a nucleic acid molecule may be selected for a particular indication, condition, disease, or disorder and/or for delivery to a particular cell, tissue, organ, or system or group thereof (e.g., localized or specific delivery).

In certain embodiments, a lipid nanoparticle may include an mRNA encoding a polypeptide of interest capable of being translated within a cell to produce a polypeptide of interest. In other embodiments, the lipid nanoparticle can include other types of agents, such as other nucleic acid agents, including DNA and/or RNA agents, as described herein, e.g., siRNAs, miRNAs, antisense nucleic acid and the like as described in further detail below.

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of a nucleic acid molecule and other elements (e.g., lipids) in a lipid nanoparticle may also vary. In some embodiments, the wt/wt ratio of the ionizable lipid component to a nucleic acid molecule, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to a nucleic acid molecule may be from about 10:1 to about 40:1. In certain embodiments, the wt/wt ratio is about 20:1. The amount of a nucleic acid molecule in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In an embodiment, the N:P ratio may be about 3:1. In an embodiment, the N:P ratio may be about 4:1. In an embodiment, the N:P ratio may be about 5:1. In an embodiment, the N:P ratio may be about 6:1. In an embodiment, the N:P ratio may be about 7:1. In an embodiment, the N:P ratio may be about 8:1.

In an embodiment, the N:P ratio may be about 3-8:1. In an embodiment, the N:P ratio may be about 3-7:1. In an embodiment, the N:P ratio may be about 3-6:1. In an embodiment, the N:P ratio may be about 3-5:1. In an embodiment, the N:P ratio may be about 3-4:1. In an embodiment, the N:P ratio may be about 4-8:1. In an embodiment, the N:P ratio may be about 5-8:1. In an embodiment, the N:P ratio may be about 6-8:1. In an embodiment, the N:P ratio may be about 7-8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Exemplary Agents

Agents to be Delivered

The target cell delivery lipids, and LNPs containing them, of the disclosure can be used to deliver a wide variety of different agents to target cells (e.g., liver cells (e.g., a hepatocyte, a hepatic stellate cell, a Kupffer cell, or a liver sinusoidal cell, or a combination thereof) or splenic cells (e.g., splenocytes)) through association with, e.g., encapsulation of the agent. Typically the agent delivered by the LNP is a nucleic acid, although non-nucleic acid agents, such as small molecules, chemotherapy drugs, peptides, proteins and other biological molecules are also encompassed by the disclosure. Nucleic acids that can be delivered include DNA-based molecules (i.e., comprising deoxyribonucleotides) and RNA-based molecules (i.e., comprising ribonucleotides). Furthermore, the nucleic acid can be a naturally occurring form of the molecule or a chemically-modified form of the molecule (i.e., comprising one or more modified nucleotides).

Agents for Enhancing Protein Expression

In one embodiment, the agent associated with/encapsulated by the lipid-based composition (e.g., LNP) is an agent that enhances (i.e., increases, stimulates, upregulates) protein expression. In one embodiment, the agent increases protein expression in the target cells (e.g., liver cells (e.g., a hepatocyte, a hepatic stellate cell, a Kupffer cell, or a liver sinusoidal cell, or a combination thereof) or splenic cells (e.g., splenocytes)) to which the lipid-based composition is delivered. Additionally or alternatively, in another embodiment, the agent results in increased protein expression in other cells, e.g., bystander cells, other than the target cell to which the lipid-based composition is delivered. Non-limiting examples of types of agents that can be used for enhancing protein expression include RNAs, mRNAs, dsRNAs, CRISPR/Cas9 technology, ssDNAs and DNAs (e.g., expression vectors).

DNA Agents

In one embodiment, the agent associated with/encapsulated by the LNP is a DNA agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA agent associated with/encapsulated by the LNP can be a DNA molecule that is capable of transferring a gene into a cell, e.g., that encodes and can express a transcript. For example, the DNA agent can encode a protein of interest, to thereby increase expression of the protein of interest in a target cell upon delivery into the target cell by the LNP. In some embodiments, the DNA molecule can be naturally-derived, e.g., isolated from a natural source. In other embodiments, the DNA molecule is a synthetic molecule, e.g., a synthetic DNA molecule produced in vitro. In some embodiments, the DNA molecule is a recombinant molecule. Non-limiting exemplary DNA agents include plasmid expression vectors and viral expression vectors.

The DNA agents described herein, e.g., DNA vectors, can include a variety of different features. The DNA agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

RNA Agents

In one embodiment, the agent associated with/encapsulated by the LNP is an RNA agent. The RNA molecule can be a single-stranded RNA, a double-stranded RNA (dsRNA) or a molecule that is a partially double-stranded RNA, i.e., has a portion that is double-stranded and a portion that is single-stranded. The RNA molecule can be a circular RNA molecule or a linear RNA molecule.

An RNA agent associated with/encapsulated by the LNP can be an RNA agent that is capable of transferring a gene into a cell, e.g., encodes a protein of interest, to thereby increase expression of the protein of interest in a target cell upon delivery into the target cell by the LNP. In some embodiments, the RNA molecule can be naturally-derived, e.g., isolated from a natural source. In other embodiments, the RNA molecule is a synthetic molecule, e.g., a synthetic RNA molecule produced in vitro.

Non-limiting examples of RNA agents include messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and CRISPR/Cas9 technology, each of which is described further in subsections below.

Messenger RNA (mRNA)

In some embodiments, the disclosure provides a lipid composition (e.g., lipid nanoparticle) comprising at least one mRNA, for use in the methods described herein.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 60. An exemplary 3′ UTR for use in the constructs is shown in SEQ ID NO: 61. An exemplary 3′ UTR comprising miR-122 and/or miR-142-3p binding sites for use in the constructs is shown in SEQ ID NO: 62. In one embodiment, hepatocyte expression is reduced by including miR122 binding sites. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

(5′ UTR)
SEQ ID NO: 60
TCAAGCTTTTGGACCCTCGTACAGAAGCTAATACGACTCACTATAGGGAA
ATAAGAGAGAAAAGAAGAGTAAGAAGAAATATAAGAGCCACC
(3′ UTR)
SEQ ID NO: 61
TGATAATAGGCTGGAGCCTCGGTGGCCATGCTTCTTGCCCCTTGGGCCTC
CCCCCAGCCCCTCCTCCCCTTCCTGCACCCGTACCCCCGTGGTCTTTGAA
TAAAGTCTGAGTGGGCGGC
(3′ UTR with miR-122 and miR-142-3p sites)
SEQ ID NO: 62
TGATAATAGGCTGGAGCCTCGGTGGCCATGCTTCTTGCCCCTTGGGCCCA
AACACCATTGTCACACTCCATCCCCCCAGCCCCTCCTCCCCTTCCTCCAT
AAAGTAGGAAACACTACATGCACCCGTACCCCCGTGGTCTTTGAATAAAG
TCTGAGTGGGCGGC

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3 [symbol] deoxyuridine, 3 [symbol] deoxycytosine, 3 [symbol] deoxyguanosine, 3 [symbol] deoxythymine, and 2 [symbol] 3 [symbol] dideoxynucleosides, such as 2′,3′-dideoxyadenosine, 2 [symbol] 3 [symbol] dideoxyuridine, 2 [symbol] 3 [symbol] dideoxycytosine, 2 [symbol] 3 [symbol] dideoxyguanosine, and 2 [symbol] 3 [symbol] dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a therapeutic payload or prophylactic payload, an effector molecule and/or a tether molecule) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N, pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length.

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

The invention also includes a polynucleotide that comprises both a start codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of RES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 63), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 63) fragments or variants thereof. One example of a polynucleotide sequence encoding the 2A peptide is: GGAAGCGGAGCTACTAACTTCAGCCTGCTGAAGCAGGCTGGAGACGTGGAGGAGAA CCCTGGACCT (SEQ ID NO: 64). In one illustrative embodiment, a 2A peptide is encoded by the following sequence: 5′-TCCGGACTCAGATCCGGGGATCTCAAAATTGTCGCTCCTGTCAAACAAACTCTTAAC TTTGATTTACTCAAACTGGCTGGGGATGTAGAAAGCAATCCAGGTCCACTC-3′(SEQ ID NO: 65). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP (SEQ ID NO: 179) is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ϕ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τ m5U), 1-taurinomethyl-pseudouridine, 5-taurinomethyl-2-thio-uridine (τ m5s2U), 1-taurinomethyl-4-thio-pseudouridine, 5-methyl-uridine (m5U, i.e., having the nucleobase deoxythymine), 1-methyl-pseudouridine (m1 ϕ), 5-methyl-2-thio-uridine (m5s2U), 1-methyl-4-thio-pseudouridine (m1s4 ϕ), 4-thio-1-methyl-pseudouridine, 3-methyl-pseudouridine (m3 ϕ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ϕ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ϕ m), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (fSCm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2 g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ϕ), N1-methylpseudouridine (m1 ϕ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1 ϕ) and the mRNA of the disclosure is fully modified with N1-methylpseudouridine (m1 ϕ). In some embodiments, N1-methylpseudouridine (m1 ϕ) represents from 75-100% of the uracils in the mRNA. In some embodiments, N1-methylpseudouridine (m1 ϕ)) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.) In some embodiments, the modified nucleobase is 1-methyl-pseudouridine (m1 ϕ), 5-methoxy-uridine (mo5U), 5-methyl-cytidine (m5C), pseudouridine (ϕ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ϕ). In some embodiments, the mRNA comprises pseudouridine (ϕ) and 5-methyl-cytidine (m5C). In some embodiments, the mRNA comprises 1-methyl-pseudouridine (m1 ϕ). In some embodiments, the mRNA comprises 1-methyl-pseudouridine (m1 ϕ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1 ϕ) or 5-methyl-cytidine (m5C), meaning that all uridines or all cytosine nucleosides in the mRNA sequence are replaced with N1-methylpseudouridine (m1 ϕ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 17 and Table 18. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 17
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 18
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75%
UTP
25% 5-Aminoallyl-CTP + 75% CTP/75% 5-Methoxy-UTP + 25%
UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP + 75%
UTP
25% 5-Hydroxymethyl-CTP + 75% CTP/75% 5-Methoxy-UTP + 25%
UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-
pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-
Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-
Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75%
UTP
25% 5-Trifluoromethyl-CTP + 75% CTP/75% 5-Methoxy-UTP + 25%
UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-
Methyl-pseudo-UTP
50% 5-Methyl-CTP + 50% CTP/25% 5-Methoxy-UTP + 75%
UTP
50% 5-Methyl-CTP + 50% CTP/50% 5-Methoxy-UTP + 50% 1-
Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-
Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxymethyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoromethyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75%
UTP
75% 5-Aminoallyl-CTP + 25% CTP/75% 5-Methoxy-UTP + 25%
UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75%
UTP
75% 5-Hydroxymethyl-CTP + 25% CTP/75% 5-Methoxy-UTP + 25%
UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-
Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-
Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-
Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75%
UTP
75% 5-Trifluoromethyl-CTP + 25% CTP/75% 5-Methoxy-UTP + 25%
UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 17 or Table 18.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.” Non-limiting examples of sensor sequences are described in U.S. Publication 2014/0200261, the contents of which are incorporated herein by reference in their entirety.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5 iUTR and/or 3 iUTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5 □ terminus, the 3 □terminus, or both. In still other embodiments, the microRNA binding site is two nucleotides shorter than the corresponding microRNA at the 5 □terminus, the 3 □terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR can be included in a nucleic acid molecule (e.g., an RNA, e.g., mRNA) to minimize expression in cell types other than lymphoid cells. In one embodiment, miR122 can be used. In another embodiment, miR126 can be used. In still another embodiment, multiple copies of these miRs or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

In one embodiment, a nucleic acid molecule (e.g., RNA, e.g., mRNA) of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a nucleic acid molecule (e.g., RNA, e.g., mRNA) of the disclosure can include two, three, four, five, six, seven, eight, nine, ten, or more miRNA-binding sites in the 5 [symbol] UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in target cells (e.g., liver cells (e.g., a hepatocyte, a hepatic stellate cell, a Kupffer cell, or a liver sinusoidal cell, or a combination thereof) or splenic cells (e.g., splenocytes)). Target cell specific miRNAs are involved in immunogenicity, autoimmunity, the immune response to infection, inflammation, as well as unwanted immune response after gene therapy and tissue/organ transplantation. Target cell specific miRNAs also regulate many aspects of development, proliferation, differentiation and apoptosis of hematopoietic cells (target cells).

In one embodiment, binding sites for miRNAs that are known to be expressed in target cells, in particular, can be engineered into a nucleic acid molecule (e.g., RNA, e.g., mRNA) of the disclosure to suppress the expression of the nucleic acid molecule (e.g., RNA, e.g., mRNA) in target cells through miRNA mediated RNA degradation. Expression of the nucleic acid molecule (e.g., RNA, e.g., mRNA) is maintained in non-target cells where the target cell specific miRNAs are not expressed. For example, in some embodiments, to prevent an immunogenic reaction against a liver specific protein, any miR-122 binding site can be removed and a miR-142 (and/or mirR-146) binding site can be engineered into the 5 UTR and/or 3 UTR of a nucleic acid molecule of the disclosure.

To further drive the selective degradation and suppression in target cells, a nucleic acid molecule (e.g., RNA, e.g., mRNA) of the disclosure can include a further negative regulatory element in the 5 UTR and/or 3 UTR, either alone or in combination with a miR binding site. As a non-limiting example, the further negative regulatory element is a Constitutive Decay Element (CDE).

Liver target cell specific miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a nucleic acid molecule (e.g., RNA, e.g., mRNA) of the disclosure to regulate expression of the nucleic acid molecule (e.g., RNA, e.g., mRNA) in the liver. In one embodiment, miRNA binding sites that promote degradation of mRNAs by hepatocytes are present in an mRNA molecule agent.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a nucleic acid molecule (e.g., RNA, e.g., mRNA) of the disclosure to regulate expression of the nucleic acid molecule (e.g., RNA, e.g., mRNA) in the lung. Lung specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a nucleic acid molecule (e.g., RNA, e.g., mRNA) of the disclosure to regulate expression of the nucleic acid molecule (e.g., RNA, e.g., mRNA) in the heart. Heart specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a nucleic acid molecule (e.g., RNA, e.g., mRNA) of the disclosure to regulate expression of the nucleic acid molecule (e.g., RNA, e.g., mRNA) in the nervous system. Nervous system specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a nucleic acid molecule (e.g., RNA, e.g., mRNA) of the disclosure to regulate expression of the nucleic acid molecule (e.g., RNA, e.g., mRNA) in the pancreas. Pancreas specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a nucleic acid molecule (e.g., RNA, e.g., mRNA) of the disclosure to regulate expression of the nucleic acid molecule (e.g., RNA, e.g., mRNA) in the kidney. Kidney specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs that are known to be expressed in the muscle include, but are not limited to, let-7 g-3p, let-7 g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. miRNA binding sites from any muscle specific miRNA can be introduced to or removed from a nucleic acid molecule (e.g., RNA, e.g., mRNA) of the disclosure to regulate expression of the nucleic acid molecule (e.g., RNA, e.g., mRNA) in the muscle. Muscle specific miRNA binding sites can be engineered alone or further in combination with target cell (e.g., liver cells or splenic cells) miRNA binding sites in a nucleic acid molecule (e.g., RNA, e.g., mRNA) of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548 g-3p, miR-548 g-5p, miR-548i, miR-548k, miR-548l, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, the binding sites of embryonic stem cell specific miRNAs can be included in or removed from the 3 UTR of a nucleic acid molecule (e.g., RNA, e.g., mRNA) of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3 UTR of a nucleic acid molecule (e.g., RNA, e.g., mRNA) of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the nucleic acid molecules (e.g., RNA, e.g., mRNA) of the disclosure, miRNA binding sites that are involved in such processes can be removed or introduced, in order to tailor the expression of the nucleic acid molecules (e.g., RNA, e.g., mRNA) to biologically relevant cell types or relevant biological processes. In this context, the nucleic acid molecules (e.g., RNA, e.g., mRNA) of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into a nucleic acid molecule (e.g., RNA, e.g., mRNA) encoding the polypeptide. In one example, a nucleic acid molecule (e.g., RNA, e.g., mRNA) may include one or more miRNA binding sites that are bound by miRNAs that have higher expression in one tissue type as compared to another. In another example, a nucleic acid molecule (e.g., RNA, e.g., mRNA) may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the nucleic acid molecule (e.g., RNA, e.g., mRNA) typically will show increased expression.

Liver cancer cells (e.g., hepatocellular carcinoma cells) typically express low levels of miR-122 as compared to normal liver cells. Therefore, a nucleic acid molecule (e.g., RNA, e.g., mRNA) encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the nucleic acid molecule (e.g., RNA, e.g., mRNA) includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 75, wherein the miRNA binding site binds to miR-122. In another particular aspect, the miRNA binding site comprises a nucleotide sequence at least 80%, at least 85%, at least 90%, at least 95%, or 100% identical to SEQ ID NO: 73, wherein the miRNA binding site binds to miR-122. These sequences are shown below in Table 19.

In some embodiments, a nucleic acid molecule (e.g., RNA, e.g., mRNA) of the disclosure comprises a miRNA binding site, wherein the miRNA binding site comprises one or more nucleotide sequences selected from Table 19, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 19, including any combination thereof. In some embodiments, the miRNA binding site binds to miR-142 or is complementary to miR-142. In some embodiments, the miR-142 comprises SEQ ID NO: 66. In some embodiments, the miRNA binding site binds to miR-142-3p or miR-142-5p. In some embodiments, the miR-142-3p binding site comprises SEQ ID NO: 68. In some embodiments, the miR-142-5p binding site comprises SEQ ID NO: 70. In some embodiments, the miRNA binding site comprises a nucleotide sequence at least 80%, at least 85%, at least 90%, at least 95%, or 100% identical to SEQ ID NO: 68 or SEQ ID NO: 70.

TABLE 19
Representative microRNAs and
microRNA binding sites

| SEQ | | |
|---|---|---|
| ID NO. | Description | Sequence |
| 66 | mmiR-142 | GACAGUGCAGUCACCCAUAAAGUAGAAAGC |
| | | ACUACUAACAGCACUGGAGGGUGUAGUGUU |
| | | UCCUACUUUAUGGAUGAGUGUACUGUG |
| 67 | mmiR-142-3p | UGUAGUGUUUCCUACUUUAUGGA |
| 68 | mmiR-142-3p | UCCAUAAAGUAGGAAACACUACA |
| | binding | |
| | site | |
| 69 | mmiR-142-5p | CAUAAAGUAGAAAGCACUACU |
| 70 | mmiR-142-5p | AGUAGUGCUUUCUACUUUAUG |
| | binding | |
| | site | |
| 71 | miR-122 | CCUUAGCAGAGCUGUGGAGUGUGACAAUGG |
| | | UGUUUGUGUCUAAACUAUCAAACGCCAUUA |
| | | UCACACUAAAUAGCUACUGCUAGGC |
| 72 | miR-122-3p | AACGCCAUUAUCACACUAAAUA |
| 73 | miR-122-3p | UAUUUAGUGUGAUAAUGGCGUU |
| | binding | |
| | site | |
| 74 | miR-122-5p | UGGAGUGUAGACAAUGGUGUUUG |
| 75 | miR-122-5p | CAAACACCAUUGUCACACUCCA |
| | binding | |
| | site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5 [symbol] and/or 3 [symbol] TR). In some embodiments, the 5 [symbol] TR comprises a miRNA binding site. In some embodiments, the 3 [symbol] TR comprises a miRNA binding site. In GC some embodiments, the 5 [symbol] TR and the 3 [symbol] TR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure. miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a nucleic acid molecule (e.g., RNA, e.g., mRNA) of the disclosure can be engineered to include more than one miRNA site expressed in different tissues or different cell types of a subject. As a non-limiting example, a nucleic acid molecule (e.g., RNA, e.g., mRNA) of the disclosure can be engineered to include miR-192 and miR-122 to regulate expression of the nucleic acid molecule (e.g., RNA, e.g., mRNA) in the liver and kidneys of a subject. In another embodiment, a nucleic acid molecule (e.g., RNA, e.g., mRNA) of the disclosure can be engineered to include more than one miRNA site for the same tissue. In some embodiments, the therapeutic window and or differential expression associated with the polypeptide encoded by a nucleic acid molecule (e.g., RNA, e.g., mRNA) of the disclosure can be altered with a miRNA binding site. For example, a nucleic acid molecule (e.g., RNA, e.g., mRNA) encoding a polypeptide that provides a death signal can be designed to be more highly expressed in cancer cells by virtue of the miRNA signature of those cells. Where a cancer cell expresses a lower level of a particular miRNA, the nucleic acid molecule (e.g., RNA, e.g., mRNA) encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple nucleic acid molecule (e.g., RNA, e.g., mRNA) can be designed and administered having different signals based on the use of miRNA binding sites as described herein.

In some embodiments, the expression of a nucleic acid molecule (e.g., RNA, e.g., mRNA) of the disclosure can be controlled by incorporating at least one sensor sequence in the polynucleotide and formulating the nucleic acid molecule (e.g., RNA, e.g., mRNA) for administration. As a non-limiting example, a nucleic acid molecule (e.g., RNA, e.g., mRNA) of the disclosure can be targeted to a tissue or cell by incorporating a miRNA binding site and formulating the nucleic acid molecule (e.g., RNA, e.g., mRNA) in a lipid nanoparticle comprising a cationic lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the nucleic acid molecule (e.g., RNA, e.g., mRNA). In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop. In another embodiment, a miRNA seed sequence can be incorporated in the loop of a stem loop and a miRNA binding site can be incorporated into the 5′ or 3′ stem of the stem loop. In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a nucleic acid molecule (e.g., RNA, e.g., mRNA) of the disclosure can comprise at least one miRNA sequence. The miRNA sequence can be, but is not limited to, a 19 or 22 nucleotide sequence and/or a miRNA sequence without the seed. In one embodiment the miRNA sequence in the 5′UTR can be used to stabilize a nucleic acid molecule (e.g., RNA, e.g., mRNA) of the disclosure described herein.

In another embodiment, a miRNA sequence in the 5′UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site. In some embodiments, a nucleic acid molecule (e.g., RNA, e.g., mRNA) of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure to dampen antigen presentation is miR-142-3p.

In some embodiments, a nucleic acid molecule (e.g., RNA, e.g., mRNA) of the disclosure can include at least one miRNA in order to dampen expression of the encoded polypeptide in a tissue or cell of interest. As a non-limiting example, a nucleic acid molecule (e.g., RNA, e.g., mRNA) of the disclosure can include at least one miR-122 binding site in order to dampen expression of an encoded polypeptide of interest in the liver. As another non-limiting example a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the target cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule (e.g., RNA, e.g., mRNA) that can interact with an RNA binding protein.

In some embodiments, the nucleic acid molecule (e.g., RNA, e.g., mRNA) of the disclosure comprising (i) a sequence-optimized nucleotide sequence (e.g., an ORF) and (ii) a miRNA binding site (e.g., a miRNA binding site that binds to miR-142).

In some embodiments, the nucleic acid molecule (e.g., RNA, e.g., mRNA) of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uricil in a uracil-modified sequence encoding a polypeptide is 5-methoxyuridine. In some embodiments, the nucleic acid molecule (e.g., RNA, e.g., mRNA) comprising a nucleotide sequence encoding a polypeptide disclosed herein and a miRNA binding site is formulated with a delivery agent, e.g., a compound having the Formula (I), e.g., any of Compounds 1-147.

Modified RNA Molecules Comprising Functional RNA Elements

The present disclosure provides synthetic nucleic acid molecules (e.g., RNA, e.g., mRNA) comprising a modification (e.g., an RNA element), wherein the modification provides a desired translational regulatory activity. In some embodiments, the disclosure provides a nucleic acid molecule (e.g., RNA, e.g., mRNA) comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a nucleic acid molecule (e.g., RNA, e.g., mRNA), comprising an RNA element that comprises a sequence and/or an RNA secondary structure(s) that provides a desired translational regulatory activity as described herein. In some aspects, the nucleic acid molecule (e.g., RNA, e.g., mRNA) comprises an RNA element that comprises a sequence and/or an RNA secondary structure(s) that promotes and/or enhances the translational fidelity of translation. In some aspects, the nucleic acid molecule (e.g., RNA, e.g., mRNA) comprises an RNA element that comprises a sequence and/or an RNA secondary structure(s) that provides a desired translational regulatory activity, such as inhibiting and/or reducing leaky scanning. In some aspects, the disclosure provides a nucleic acid molecule (e.g., RNA, e.g., mRNA) that comprises an RNA element that comprises a sequence and/or an RNA secondary structure(s) that inhibits and/or reduces leaky scanning thereby promoting the translational fidelity of the nucleic acid molecule (e.g., RNA, e.g., mRNA).

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides a nucleic acid molecule (e.g., RNA, e.g., mRNA) having one or more structural modifications that inhibits leaky scanning and/or promotes the translational fidelity of translation, wherein at least one of the structural modifications is a GC-rich RNA element. In some aspects, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA). In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA). In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the nucleic acid molecule (e.g., RNA, e.g., mRNA).

In some embodiments, the GC-rich RNA element comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In some embodiments, the GC-rich RNA element comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, a GC-rich RNA element comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In some embodiments, a GC-rich RNA element comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA), and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA), and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5 (SEQ ID NO: 180). In some embodiments, the sequence comprises a repeating GC-motif [CCG]n, wherein n=1, 2, 3, 4 or 5 (SEQ ID NO: 181). In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 177). In some embodiments, the sequence comprises a repeating GC-motif [CCG]n, wherein n=5 (SEQ ID NO: 178).

In some embodiments, the disclosure provides a modified nucleic acid molecule (e.g., RNA, e.g., mRNA) comprising at least one modification, wherein at least one modification is a GC-rich RNA element comprising a sequence of linked nucleotides, or derivatives or analogs thereof, preceding a Kozak consensus sequence in a 5′ UTR of the nucleic acid molecule (e.g., RNA, e.g., mRNA), wherein the GC-rich RNA element comprises any one of the sequences set forth in Table 20. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the nucleic acid molecule (e.g., RNA, e.g., mRNA). In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V1 [CCCCGGCGCC] (SEQ ID NO: 80) as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence V1 as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence V1 as set forth in Table 5 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence V1 as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V2 [CCCCGGC] as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence V2 as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence V2 as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence V2 as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence EK [GCCGCC] as set forth in Table 20, or derivatives or analogs thereof, preceding a Kozak consensus sequence in the 5′ UTR of the nucleic acid molecule (e.g., RNA, e.g., mRNA). In some embodiments, the GC-rich element comprises the sequence EK as set forth in Table 20 located immediately adjacent to and upstream of the Kozak consensus sequence in the 5′ UTR of the nucleic acid molecule (e.g., RNA, e.g., mRNA). In some embodiments, the GC-rich element comprises the sequence EK as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA). In other embodiments, the GC-rich element comprises the sequence EK as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA).

In some embodiments, the disclosure provides a modified nucleic acid molecule (e.g., RNA, e.g., mRNA) comprising at least one modification, wherein at least one modification is a GC-rich RNA element comprising the sequence V1 [CCCCGGCGCC] (SEQ ID NO: 80) as set forth in Table 20, or derivatives or analogs thereof, preceding a Kozak consensus sequence in the 5′ UTR of the nucleic acid molecule (e.g., RNA, e.g., mRNA), wherein the 5′ UTR comprises the following sequence shown in Table 20:

(SEQ ID NO: 77)
GGGAAATAAGAGAGAAAAGAAGAGTAAGAAGAAATATAAGA.

In some embodiments, the GC-rich element comprises the sequence V1 as set forth in Table 20 located immediately adjacent to and upstream of the Kozak consensus sequence in the 5′ UTR sequence shown in Table 20. In some embodiments, the GC-rich element comprises the sequence VI as set forth in Table 20 located 1, 2, 3, 4, 5, 6, 7, 8, 9 or 10 bases upstream of the Kozak consensus sequence in the 5′ UTR of the nucleic acid molecule (e.g., RNA, e.g., mRNA) wherein the 5′ UTR comprises the following sequence shown in Table 20:

(SEQ ID NO: 77)
GGGAAATAAGAGAGAAAAGAAGAGTAAGAAGAAATATAAGA.

In other embodiments, the GC-rich element comprises the sequence V1 as set forth in Table 20 located 1-3, 3-5, 5-7, 7-9, 9-12, or 12-15 bases upstream of the Kozak consensus sequence in the 5′ UTR of the nucleic acid molecule (e.g., RNA, e.g., mRNA), wherein the 5′ UTR comprises the following sequence shown in Table 20:

(SEQ ID NO: 77)
GGGAAATAAGAGAGAAAAGAAGAGTAAGAAGAAATATAAGA.

In some embodiments, the 5′ UTR comprises the following sequence set forth in Table 20:

(SEQ ID NO: 78)
GGGAAATAAGAGAGAAAAGAAGAGTAAGAAGAAATATAAGACCCCGGCGC
CGCCACC

In some embodiments, the 5′ UTR comprises the following sequence set forth in Table 20:

(SEQ ID NO: 79)
GGGAAATAAGAGAGAAAAGAAGAGTAAGAAGAAATATAAGACCCCGGCGC
CACC

TABLE 20

| SEQ | | |
|---|---|---|
| ID NO: | 5 UTRs | 5 UTR Sequence |
| 76 | Standard | GGGAAATAAGAGAGAAAAGAAGAGTAAGAAGAA |
| | | ATATAAGAGCCACC |
| 77 | UTR | GGGAAATAAGAGAGAAAAGAAGAGTAAGAAGAA |
| | | ATATAAGA |
| 78 | V1-UTR | GGGAAATAAGAGAGAAAAGAAGAGTAAGAAGAA |
| | | ATATAAGACCCCGGCGCCGCCACC |
| 79 | V2-UTR | GGGAAATAAGAGAGAAAAGAAGAGTAAGAAGAA |
| | | ATATAAGACCCCGGCGCCACC |

In some embodiments, the disclosure provides a modified nucleic acid molecule (e.g., RNA, e.g., mRNA) comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In some embodiments, the stable RNA secondary structure is upstream of the Kozak consensus sequence. In some embodiments, the stable RNA secondary structure is located about 30, about 25, about 20, about 15, about 10, or about 5 nucleotides upstream of the Kozak consensus sequence. In some embodiments, the stable RNA secondary structure is located about 20, about 15, about 10 or about 5 nucleotides upstream of the Kozak consensus sequence. In some embodiments, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In some embodiments, the modification is operably linked to an open reading frame encoding a polypeptide and wherein the modification and the open reading frame are heterologous.

In some embodiments, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of nucleic acid molecule (e.g., RNA, e.g., mRNA), by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along a nucleic acid molecule (e.g., RNA, e.g., mRNA), footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

Agents for Reducing Protein Expression

In one embodiment, the agent associated with/encapsulated by the lipid-based composition (e.g., LNP) is an agent that reduces (i.e., decreases, inhibits, downregulates) protein expression. In one embodiment, the agent reduces protein expression in the target cell (e.g., liver cells (e.g., a hepatocyte, a hepatic stellate cell, a Kupffer cell, or a liver sinusoidal cell, or a combination thereof) or splenic cells (e.g., splenocytes)) to which the lipid-based composition is delivered. Additionally or alternatively, in another embodiment, the agent results in reduced protein expression in other cells, e.g., bystander cells, than the target cell to which the lipid-based composition is delivered. Non-limiting examples of types of agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

RNA Interference Molecules

RNA interference (RNAi) refers to a biological process in which RNA molecules inhibit gene expression or translation by neutralizing targeted mRNA molecules. RNAi is a gene silencing process that is controlled by the RNA-induced silencing complex (RISC) and is initiated by short double-stranded RNA molecules (dsRNA) in a cell's cytoplasm. Two types of small ribonucleic acid molecules, small interfering RNAs (siRNAs) and microRNAs (miRNAs), are central to RNA interference. While RNAi is a natural cellular process, the components of RNAi also have been synthesized and exploited for inhibiting expression of target genes/mRNAs of interest in vitro and in vivo.

As a natural process, dsRNA initiates RNAi by activating the ribonuclease protein Dicer, which binds and cleaves dsRNA and short hairpin RNAs (shRNAs) to produce double-stranded fragments of 20-25 base pairs. These short double-stranded fragments are called small interfering RNAs (siRNAs). These siRNAs are then separated into single strands and integrated into an active RISC, by the RISC-Loading Complex (RLC). After integration into the RISC, siRNAs base-pair to their target mRNA and cleave it, thereby preventing it from being used as a translation template.

The phenomenon of RNAi, broadly defined, also includes the gene silencing effects of miRNAs. MicroRNAs are genetically-encoded non-coding RNAs that help regulate gene expression, for example during development. Naturally-occurring mature miRNAs are structurally similar to siRNAs produced from exogenous dsRNA, but before reaching maturity, miRNAs undergo extensive post-transcriptional modification, including a dsRNA portion of pre-miRNA being cleaved by Dicer to produce the mature miRNA molecule that can be integrated into the RISC complex.

Accordingly, in one embodiment, the agent associated with/encapsulated by the lipid-based composition, e.g., LNP, is an RNAi molecule (i.e., a molecule that mediates or is involved in RNA interference), including siRNAs and miRNAs, each of which is described in further detail below.

Small Interfering RNAs

Small interfering RNAs (siRNAs), also referred to as short interfering RNAs or silencing RNAs, are a class of double-stranded RNA molecules, typically 20-25 base pairs in length, that operate within the RNAi pathway to interfere with the expression of specific target sequences with complementary nucleotide sequences. siRNAs inhibit gene expression by degrading mRNA after transcription, thereby preventing translation. As used herein, the term “siRNA” encompasses all forms of siRNAs known in the art, including, but not limited to, shortmers, longmers, 2′5′-isomers and Dicer-substrate RNAs. Naturally-occurring and artificially synthesized siRNAs, and their use in therapy (e.g., delivered by nanoparticles), have been described in the art (see e.g., Hamilton and Balcombe (1999) Science 286:950-952; Elbashir et al. (2001) Nature 411:494-498; Shen et al. (2012) Cancer Gene Therap. 19:367-373; Wittrup et al. (2015) Nat. Rev. Genet. 16:543-552).

Accordingly, in one embodiment, the agent associated with/encapsulated by the lipid-based composition, e.g., LNP, is an siRNA. In one embodiment, the siRNA inhibits expression of a target sequence expressed in target cells. In one embodiment, the siRNA inhibits expression of a target sequence expressed in liver cells (e.g., a hepatocyte, a hepatic stellate cell, a Kupffer cell, or a liver sinusoidal cell, or a combination thereof). In one embodiment, the siRNA inhibits expression of a target sequence expressed in splenic cells (e.g., splenocytes)).

In another embodiment, the siRNA inhibits the expression of a transcription factor in the target cell (e.g., liver cells (e.g., a hepatocyte, a hepatic stellate cell, a Kupffer cell, or a liver sinusoidal cell, or a combination thereof) or splenic cells (e.g., splenocytes)) In one embodiment, the siRNA inhibits the expression of a cytoplasmic protein in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a transmembrane protein (e.g., cell surface receptors) in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a secreted protein) in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an intracellular signaling protein in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an enzyme (e.g., AMPKa1, AMPKa2, HDAC10, or CAMKK2,) in the target cell ((e.g., liver cells (e.g., a hepatocyte, a hepatic stellate cell, a Kupffer cell, or a liver sinusoidal cell, or a combination thereof) or splenic cells (e.g., splenocytes)).

MicroRNAs

MicroRNAs (miRNAs) are small non-coding RNA molecules (typically containing about 22 nucleotides) that function in RNA silencing and post-transcriptional regulation of gene expression. miRNAs inhibit gene expression via base-pairing with complementary sequences within mRNA molecules, leading to cleavage of the mRNA, destabilization of the mRNA through shortening of its polyA tail and/or less efficient translation of the mRNA into protein by ribosomes. With respect to mRNA cleavage, it has been demonstrated that given complete complementarity between the miRNA and the target mRNA sequence, the protein Ago2 can cleave the mRNA, leading to direct mRNA degradation. miRNAs and their function have been described in the art (see e.g., Ambros (2004) Nature 431:350-355; Bartel (2004) Cell 116:281-297; Bartel (2009) Cell 136:215-233; Fabian et al. (2010) Ann. Rev. Biochem. 79:351-379).

Accordingly, in one embodiment, the agent associated with/encapsulated by the lipid-based composition, e.g., LNP, is a miRNA. In one embodiment, the miRNA inhibits expression of a target sequence expressed in target cells. In one embodiment, the miRNA inhibits expression of a target sequence expressed in liver cells (e.g., a hepatocyte, a hepatic stellate cell, a Kupffer cell, or a liver sinusoidal cell, or a combination thereof). In one embodiment, the miRNA inhibits expression of a target sequence expressed in splenic cells (e.g., splenocytes)).

In another embodiment, the miRNA inhibits the expression of a transcription factor in the target cell (e.g., liver cells (e.g., a hepatocyte, a hepatic stellate cell, a Kupffer cell, or a liver sinusoidal cell, or a combination thereof) or splenic cells (e.g., splenocytes)) In one embodiment, the siRNA inhibits the expression of a cytoplasmic protein in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a transmembrane protein (e.g., cell surface receptors) in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a secreted protein) in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an intracellular signaling protein in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an enzyme (e.g., AMPKa1, AMPKa2, HDAC10, or CAMKK2,) in the target cell ((e.g., liver cells (e.g., a hepatocyte, a hepatic stellate cell, a Kupffer cell, or a liver sinusoidal cell, or a combination thereof) or splenic cells (e.g., splenocytes)).

For modulation of target cell activity and/or modulation of target cell responses, non-limiting examples of suitable miRNAs include Let-7d-5p, miR-7, miR-10a, miR-10b, miR-15, miR-18a, miR-20a, miR-20b, miR-21, miR-26a, miR-34a, miR-96, miR-99a, miR-100, miR-124, miR-125a, miR-126, miR-142-3p, miR-146, miR-150, miR-155, miR-181a and miR-210.

Antagomirs

Antagomirs, also known in the art as anti-miRs or blockmirs, are a class of chemically engineered oligonucleotides that prevent other molecules from binding to a desired site on an mRNA molecule. Antagomirs are used to silence endogenous miRNAs. An antagomir is a small synthetic RNA that is perfectly complementary to the specific miRNA target, with either mispairing at the cleavage site of Ago2 or some sort of base modification to inhibit Ago2 cleavage. Typically, antagomirs have one or more modifications, such as 2′-methoxy groups and/or phosphorothioates, to make them more resistant to degradation. Antagomirs and their function have been described in the art (see e.g., Krutzfeldt et al. (2005) Nature 438:685-689; Czech (2006) New Eng. J. Med. 354:1194-1195).

Accordingly, in one embodiment, the agent associated with/encapsulated by the lipid-based composition, e.g., LNP, is an antagomir. Since antagomirs block (inhibit) the activity of endogenous miRNAs that downregulate gene expression, the effect of an antagomir can be to enhance (i.e., increase, stimulate, upregulate) expression of a gene of interest. Accordingly, in one embodiment, the antagomir enhances expression of a target sequence expressed in target cells. In one embodiment, the antagomir enhances expression of a target sequence expressed in liver cells (e.g., a hepatocyte, a hepatic stellate cell, a Kupffer cell, or a liver sinusoidal cell, or a combination thereof). In one embodiment, the antagomir enhances expression of a target sequence expressed in splenic cells (e.g., splenocytes)).

In another embodiment, the antagomir enhances the expression of a transcription factor in the target cell (e.g., liver cells (e.g., a hepatocyte, a hepatic stellate cell, a Kupffer cell, or a liver sinusoidal cell, or a combination thereof) or splenic cells (e.g., splenocytes)) In one embodiment, the siRNA inhibits the expression of a cytoplasmic protein in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a transmembrane protein (e.g., cell surface receptors) in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of a secreted protein) in the target (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an intracellular signaling protein in the target cell (e.g., liver cells (e.g., a hepatocyte, a hepatic stellate cell, a Kupffer cell, or a liver sinusoidal cell, or a combination thereof) or splenic cells (e.g., splenocytes)). In another embodiment, the siRNA inhibits the expression of an enzyme (e.g., AMPKa1, AMPKa2, HDAC10, or CAMKK2,) in the target cell ((e.g., liver cells (e.g., a hepatocyte, a hepatic stellate cell, a Kupffer cell, or a liver sinusoidal cell, or a combination thereof) or splenic cells (e.g., splenocytes)).

For modulation of target cell activity and/or modulation of target cell responses, non-limiting examples of suitable antagomirs include those that specifically target miRNAs selected from miR-7, miR-15a, miR-16, miR-17, miR-21, miR-22, miR-23, miR-24, miR-25, miR-27, miR-31, miR-92, miR-106b, miR-146b, miR-148a, miR-155 and miR-210.

Antisense RNAs

Antisense RNAs (asRNAs), also referred to in the art as antisense transcripts, are naturally-occurring or synthetically produced single-stranded RNA molecules that are complementary to a protein-coding messenger RNA (mRNA) with which it hybridizes and thereby blocks the translation of the mRNA into a protein. Antisense transcript are classified into short (less than 200 nucleotides) and long (greater than 200 nucleotides) non-coding RNAs (ncRNAs). The primary natural function of asRNAs is in regulating gene expression and synthetic versions have been used widely as research tools for gene knockdown and for therapeutic applications. Antisense RNAs and their functions have been described in the art (see e.g., Weiss et al. (1999) Cell. Molec. Life Sci. 55:334-358; Wahlstedt (2013) Nat. Rev. Drug Disc. 12:433-446; Pelechano and Steinmetz (2013) Nat. Rev. Genet. 14:880-893). Accordingly, in one embodiment, the agent associated with/encapsulated by the lipid-based composition, e.g., LNP, is a nucleic acid (e.g., RNA or DNA) that encodes or that is an antisense RNA.

Ribozymes

Ribozymes (ribonucleic acid enzymes) are RNA molecules that are capable of catalyzing biochemical reactions, similar to the action of protein enzymes. The most common activities of natural or in vitro-evolved ribozymes are the cleavage or ligation of RNA and DNA and peptide bond formation. Moreover, self-cleaving RNAs that have good enzymatic activity have been described in the art. Therapeutic use of ribozymes, in particular for the cleavage of RNA-based viruses, is under development. Ribozymes and their functions have been described in the art (see e.g., Kruger et al. (1982) Cell 31:147-157; Tang and Baker (2000) Proc. Natl. Acad. Sci. USA 97:84-89; Fedor and Williamson (2005) Nat. Rev. Mol. Cell. Biol. 6:399-412). Accordingly, in one embodiment, the agent associated with/encapsulated by the lipid-based composition, e.g., LNP, is a nucleic acid (e.g., RNA or DNA) that encodes or that is a ribozyme.

Small Hairpin RNAs

Small (or short) hairpin RNA (shRNA) is a type of synthetic RNA molecule with a tight hairpin turn that can be used to silence target gene expression via RNA interference. shRNA is an advantageous mediator of RNA interference in that it has a relatively low rate of degradation and turnover. Expression of shRNA in cells typically is accomplished by delivery of plasmids or through viral vectors (e.g., adeno-associated virus, adenovirus or lentivirus vectors) or bacterial vectors encoding the shRNA. shRNAs and their use in gene therapy has been described in the art (see e.g., Paddison et al. (2002) Genes Dev. 16:948-958; Xiang et al. (2006) Nat. Biotech. 24:697-702; Burnett et al. (2012) Biotech. Journal 6:1130-1146). Accordingly, in one embodiment, the agent associated with/encapsulated by the lipid-based composition, e.g., LNP, is a nucleic acid (e.g., RNA or DNA) that encodes or that is an shRNA.

Locked Nucleic Acids

Locked nucleic acids, also referred to as inaccessible RNA, are modified RNA nucleotide molecules in which the ribose moiety of the LNA is modified with an extra bridge connecting the 2′ oxygen and the 4′ carbon. This bridge “locks” the ribose in the 3′-endo (North) conformation. LNA nucleotides can be mixed with DNA or RNA residues in an oligonucleotide whenever desired and hybridize with DNA or RNA according to Watson-Crick base-pairing rules. The locked ribose conformation enhances base stacking and backbone pre-organization. This significantly increases the hybridization properties (e.g., melting temperature) of oligonucleotides containing LNA nucleotides. LNA molecules, and their properties, have been described in the art (see e.g., Obika et al. (1997) Tetrahedron Lett. 38:8735-8738; Koshkin et al. (1998) Tetrahedron 54:3607-3630; Elmen et al. (2005) Nucl. Acids Res. 33:439-447). Accordingly, in one embodiment, the agent associated with/encapsulated by the lipid-based composition, e.g., LNP, is a nucleic acid (e.g., RNA or DNA) comprising one or more locked nucleic acid (LNA) nucleotides.

CRISPR/Cas9 Agents

In some embodiments, the lipid-based compositions (e.g., lipid nanoparticle) described herein are useful in methods involving the CRISPR (Clustered Regularly Interspaced Short Palindromic Repeats)-Cas9 system. CRISPR/Cas9 is used to edit the genome, wherein the enzyme Cas9 makes cuts in the DNA and allows new genetic sequences to be inserted. Single-guide RNAs are used to direct Cas9 to the specific spot in DNA where cuts are desired.

There remains a need to introduce the CRISPR/Cas9 into target cells (e.g., liver cells and/or splenic cells) in vivo. Accordingly, the present disclosure provides methods of editing the genome of target cells (e.g., liver cells (e.g., a hepatocyte, a hepatic stellate cell, a Kupffer cell, or a liver sinusoidal cell, or a combination thereof) or splenic cells (e.g., splenocytes)) with the CRISPR/Cas9 system by using the lipid-based compositions comprising delivery lipids described herein. Accordingly, in some embodiments, the agent(s) that is associated with/encapsulated by the lipids (e.g., LNP) is one or more components of the CRISPR/Cas9 system. For example, the Cas9 enzyme and single-guide RNA can be associated with/encapsulated in the lipid-based compositions described herein. Optionally, genetic material of interest to be modified (e.g., DNA) can also be encapsulated in the lipid-based composition or, alternatively, the CRISPR/Cas9 system delivered by the lipid-based composition can act on endogenous genetic material of interest in the target cells (e.g., liver cells (e.g., a hepatocyte, a hepatic stellate cell, a Kupffer cell, or a liver sinusoidal cell, or a combination thereof) or splenic cells (e.g., splenocytes)).

### Exemplary Target Proteins

The molecule targeted (e.g., encoded by the nucleic acid in the LNP or targeted for knock down) can be chosen based on the desired outcome. Given that the LNPs of the invention have now been found to be preferentially taken up by target cells, one of ordinary skill in the art can deliver numerous art recognized proteins to target cells Exemplary proteins that can be delivered (e.g., nucleic acid molecules such as DNA, RNA, mRNA, RNAi) are well known in the art and exemplary targets for such molecules are also well known in the art and exemplary such molecules are disclosed herein. When expressing proteins (e.g., using mRNA), such proteins can be a full-length protein or, alternatively, a functional fragment thereof (e.g., a fragment of the full-length protein that includes one or more functional domains such that the functional activity of the full-length protein is retained). Furthermore, in certain embodiments, the protein encoded by a nucleic acid in the LNP can be a modified protein, e.g., can comprise one or more heterologous domains, e.g., the protein can be a fusion protein that contains one more domains that do not naturally occur in the protein such that the function of the protein is altered. An example of a protein comprising a heterologous domain is a chimeric antigen receptor (described further below).

Induction or reduction of a protein of interest in or on a target cell can be measured by standard methods known in the art, such as by immunofluorescence, ELISA, immunohistochemistry, or flow cytometry.

Naturally Occurring Targets

In one embodiment, the agent associated with/encapsulated by the lipid-based composition, e.g., LNP, modulates a naturally-occurring target (e.g., up- or down-regulates the activity of a naturally-occurring target) of a target cell (e.g., liver cell (e.g., a hepatocyte, a hepatic stellate cell, a Kupffer cell, or a liver sinusoidal cell, or a combination thereof) or splenic cell (e.g., splenocyte)). The agent may itself encode the naturally-occurring target, or may function to modulate a naturally-occurring target (e.g., in a cell in vivo, such as in a subject). The naturally-occurring target can be a full-length target, such as a full-length protein, or can be a fragment or portion of a naturally-occurring target, such as a fragment or portion of a protein. The agent that modulates a naturally-occurring target (e.g., by encoding the target itself or by functioning to modulate the activity of the target) can act in an autocrine fashion, i.e., the agent exerts an effect directly on the cell into which the agent is delivered. Additionally or alternatively, the agent that modulates a naturally-occurring target can function in a paracrine fashion, i.e., the agent exerts an effect indirectly on a cell other than the cell into which the agent is delivered (e.g., delivery of the agent into one type of cell results in secretion of a molecule that exerts effects on another type of cell, such as bystander cells). Agents that modulate naturally-occurring targets include nucleic acid molecules that induce (e.g., enhance, stimulate, upregulate) protein expression, such as mRNAs and DNA. Agents that modulate naturally-occurring targets also include nucleic acid molecules that reduce (e.g., inhibit, decrease, downregulate) protein expression, such as siRNAs, miRNAs and antagomirs. Non-limiting examples of naturally-occurring targets include soluble proteins (e.g., secreted proteins), intracellular proteins (e.g., intracellular signaling proteins, transcription factors) and membrane-bound or transmembrane proteins (e.g., receptors).

### Soluble Targets

In one embodiment, the agent associated with/encapsulated by the lipid-based composition, e.g., LNP, modulates the activity of a naturally-occurring soluble target, for example by encoding the soluble target itself or by modulating the expression (e.g., transcription or translation) of the soluble target in a target cell (e.g., liver cells (e.g., a hepatocyte, a hepatic stellate cell, a Kupffer cell, or a liver sinusoidal cell, or a combination thereof) or splenic cells (e.g., splenocytes)). In one embodiment, the cell is a hepatocyte. Non-limiting examples of naturally-occurring soluble targets include secreted proteins. As demonstrated in Example 6, the lipid-based compositions of the disclosure are effective at delivering mRNA encoding a soluble target into target cells such that the soluble target is expressed by the target cells. In an embodiment, the soluble target can be secreted by the target cell and detected in the plasma.

Additional examples of soluble targets include antibody molecules, e.g., naturally-occurring antibodies, engineered antibodies and antigen binding portions thereof. An antibody molecule can include, e.g., an antibody or an antigen-binding fragment thereof (e.g., Fab, Fab [symbol] F(ab [symbol] 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Exemplary antibody molecules include, but are not limited to, humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In one embodiment, a target cell delivery LNP disclosed herein is effective at delivering an mRNA encoding an antibody molecule into target cells such that the antibody molecule is expressed by the target cells. In an embodiment, the antibody molecule can be secreted by the target cell and detected in the plasma.

In an embodiment, a target cell delivery LNP disclosed herein results in about a 10-90 fold increase in antibody molecule production compared to a reference LNP. In an embodiment, a target cell delivery LNP disclosed herein results in about 10-80 fold, 10-70 fold, 10-60 fold, 10-50 fold, 10-40 fold, 10-30 fold, 10-20 fold, 20-80 fold, 20-70 fold, 20-60 fold, 20-50 fold, 20-40 fold, or 20-30 fold more antibody molecule production compared to a reference LNP. In an embodiment, a target cell delivery LNP disclosed herein results in about 30-50 fold more antibody molecule production compared to a reference LNP.

In one embodiment, the method of using the lipid-based composition, e.g. LNP, is used to stimulate (upregulate, enhance) the activation or activity of a target cell. In another embodiment, the method of using the lipid-based composition, e.g. LNP, is used to inhibit (downregulate, reduce) the activation or activity of a target cell.

In one embodiment of stimulating the activation or activity of a target cell, the protein is a recruitment factor. As used herein a “recruitment factor” refers to any protein that promotes recruitment of a target cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8). Non-limiting examples of recruitment factors include CXCR3, CXCR5, CCR5, CCL5, CXCL10, CXCL12, and CXCL16.

Intracellular Targets

In one embodiment, the agent associated with/encapsulated by the lipid-based composition, e.g., LNP, modulates the activity of a naturally-occurring intracellular target, for example by encoding the intracellular target itself or by modulating the expression (e.g., transcription or translation) of the intracellular target in a target cell (e.g., liver cells (e.g., a hepatocyte, a hepatic stellate cell, a Kupffer cell, or a liver sinusoidal cell, or a combination thereof) or splenic cells (e.g., splenocytes)). In one embodiment, the cell is a hepatocyte. Non-limiting examples of naturally-occurring intracellular targets include transcription factors and cell signaling cascade molecules, including enzymes.

In one embodiment of stimulating the activation or activity of a target cell, the protein target is a transcription factor. As used herein, a “transcription factor” refers to a DNA-binding protein that regulates the transcription of a gene.

Membrane Bound/Transmembrane Targets

In one embodiment, the agent associated with/encapsulated by the lipid-based composition, e.g., LNP, modulates the activity of a naturally-occurring membrane-bound/transmembrane target, for example by encoding the membrane-bound/transmembrane target itself or by modulating the expression (e.g., transcription or translation) of the membrane-bound/transmembrane target in a target cell (e.g., liver cells (e.g., a hepatocyte, a hepatic stellate cell, a Kupffer cell, or a liver sinusoidal cell, or a combination thereof) or splenic cells (e.g., splenocytes)).

Modified Targets

In one embodiment, the agent associated with/encapsulated by the lipid-based composition, e.g., LNP, modulates a modified target (e.g., up- or down-regulates the activity of a non-naturally-occurring target) of a target cell (e.g., liver cells (e.g., a hepatocyte, a hepatic stellate cell, a Kupffer cell, or a liver sinusoidal cell, or a combination thereof) or splenic cells (e.g., splenocytes)). Typically, the agent itself either is or encodes the modified target. Alternatively, if a cell expresses a modified target the agent can function to modulate the activity of this modified target in the cell. The non-naturally-occurring target can be a full-length target, such as a full-length modified protein, or can be a fragment or portion of a non-naturally-occurring target, such as a fragment or portion of a modified protein. The agent that modulates a modified target can act in an autocrine fashion, i.e., the agent exerts an effect directly on the cell into which the agent is delivered. Additionally or alternatively, the agent that modulates a modified target can function in a paracrine fashion, i.e., the agent exerts an effect indirectly on a cell other than the cell into which the agent is delivered (e.g., delivery of the agent into one type of cell results in secretion of a molecule that exerts effects on another type of cell, such as bystander cells). Agents that are themselves modified targets include nucleic acid molecules, such as mRNAs or DNA, that encodie modified proteins. Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

Modified Soluble Targets

In one embodiment, the agent associated with/encapsulated by the lipid-based composition, e.g., LNP, modulates a modified soluble target (e.g., up- or down-regulates the activity of a non-naturally-occurring soluble target) of a target cell (e.g., liver cells (e.g., a hepatocyte, a hepatic stellate cell, a Kupffer cell, or a liver sinusoidal cell, or a combination thereof) or splenic cells (e.g., splenocytes)). In one embodiment, the agent (e.g., mRNA) encodes a modified soluble target. In one embodiment, the modified soluble target is a soluble protein that has been modified to alter (e.g., increase or decrease) the half-life (e.g., serum half-life) of the protein. Modified soluble proteins with altered half-lifes include modified cytokines and chemokines. In another embodiment, the modified soluble target is a soluble protein that has been modified to incorporate a tether such that the soluble protein becomes tethered to a cell surface. Modified soluble proteins incorporating a tether include tethered cytokines and chemokines.

In one embodiment, the agent (e.g., mRNA) encodes a modified soluble target, e.g., an antibody molecule as described herein. In an embodiment, the antibody molecule can be a naturally-occurring antibody molecule, an engineered antibody molecule or a antigen binding portions thereof.

Modified Intracellular Targets

In one embodiment, the agent associated with/encapsulated by the lipid-based composition, e.g., LNP, modulates a modified intracellular target (e.g., up- or down-regulates the activity of a non-naturally-occurring intracellular target) of a target cell (e.g., liver cells (e.g., a hepatocyte, a hepatic stellate cell, a Kupffer cell, or a liver sinusoidal cell, or a combination thereof) or splenic cells (e.g., splenocytes)). In one embodiment, the cell is a lymphoid cell. In one embodiment, the agent (e.g., mRNA) encodes a modified intracellular target. In one embodiment, the modified intracellular target is a constitutively active mutant of an intracellular protein, such as a constitutively active transcription factor or intracellular signaling molecule. In another embodiment, the modified intracellular target is a dominant negative mutant of an intracellular protein, such as a dominant negative mutant of a transcription factor or intracellular signaling molecule. In another embodiment, the modified intracellular target is an altered (e.g., mutated) enzyme, such as a mutant enzyme with increased or decreased activity within an intracellular signaling cascade.

Modified Membrane Bound/Transmembrane Targets

In one embodiment, the agent associated with/encapsulated by the lipid-based composition, e.g., LNP, modulates a modified membrane-bound/transmembrane target (e.g., up- or down-regulates the activity of a non-naturally-occurring membrane-bound/transmembrane target) of a target cell (e.g., liver cells (e.g., a hepatocyte, a hepatic stellate cell, a Kupffer cell, or a liver sinusoidal cell, or a combination thereof) or splenic cells (e.g., splenocytes)). In one embodiment, the agent (e.g., mRNA) encodes a modified membrane-bound/transmembrane target. In one embodiment, the modified membrane-bound/transmembrane target is a constitutively active mutant of a membrane-bound/transmembrane protein, such as a constitutively active cell surface receptor (i.e., activates intracellular signaling through the receptor without the need for ligand binding). In another embodiment, the modified membrane-bound/transmembrane target is a dominant negative mutant of a membrane-bound/transmembrane protein, such as a dominant negative mutant of a cell surface receptor

### Uses of Lipid-Based Compositions

The present disclosure provides improved lipid-based compositions, in particular LNP compositions, with enhanced delivery of nucleic acids to target cells. The present disclosure is based, at least in part, on the discovery that components of LNPs, act as target cell delivery potentiating lipids that enhance delivery of an encapsulated nucleic acid molecule (e.g., an mRNA) to target cells, such as liver cells and splenic cells.

The improved lipid-based compositions of the disclosure, in particular LNPs, are useful for a variety of purposes, both in vitro and in vivo, such as for nucleic acid delivery to target cells, protein expression in or on target cells, and/or modulating target cell (e.g., liver cells (e.g., a hepatocyte, a hepatic stellate cell, a Kupffer cell, or a liver sinusoidal cell, or a combination thereof) or splenic cells (e.g., splenocytes)) activation or activity.

For in vitro protein expression, the target cell is contacted with the LNP by incubating the LNP and the target cell ex vivo. Such target cells may subsequently be introduced in vivo.

For in vivo protein expression, the target cell is contacted with the LNP by administering the LNP to a subject to thereby increase or induce protein expression in or on target cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the target cell is contacted with the LNP by incubating the LNP and the target cell ex vivo. In one embodiment, the target cell is a human target cell. In another embodiment, the target cell is a primate target cell. In another embodiment, the target cell is a human or non-human primate target cell. Various types of target cells have been demonstrated to be transfectable by the LNP.

In one embodiment the target cell is a liver cell. In one embodiment the target cell is a hepatocyte. In one embodiment the target cell is a Kupffer cell. In one embodiment the target cell is a hepatic stellate cells. In one embodiment the target cell is a liver sinusoidal cell.

In one embodiment the target cell is a spleen cell. In one embodiment the target cell is a splenocyte.

In another embodiment, the target cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the target cell is contacted with the LNP for a single treatment/transfection. In another embodiment, the target cell is contacted with the LNP for multiple treatments/transfections (e.g., two, three, four or more treatments/transfections of the same cells).

In another embodiment, for in vivo delivery, the target cell is contacted with the LNP by administering the LNP to a subject to thereby deliver the nucleic acid to target cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, an intracellular concentration of the nucleic acid molecule in the target cell is enhanced. In one embodiment, an activity of the nucleic acid molecule in the target cell is enhanced. In one embodiment, expression of the nucleic acid molecule in the target cell is enhanced. In on embodiment, the nucleic acid molecule modulates the activation or activity of the target cell. In one embodiment, the nucleic acid molecule increases the activation or activity of the target cell. In one embodiment, the nucleic acid molecule decreases the activation or activity of the target cell.

In certain embodiments, delivery of a nucleic acid to a target cell by the target cell delivery potentiating lipid-containing LNP results in delivery to a detectable amount of target cells (e.g., delivery to a certain percentage of target cells), e.g., in vivo following administration to a subject. In some embodiments, the target cell delivery potentiating lipid containing LNP does not include a targeting moiety for target cells (e.g., does not include an antibody with specificity for a target cell marker, or a receptor ligand which targets the LNP to target cells). For example, in one embodiment, administration of the target cell delivery potentiating lipid-containing LNP results in delivery of the nucleic acid to at least about 30% liver cells in vivo after a single intravenous injection (e.g., in a non-human primate such as described in Example 5). In another embodiment, administration of the target cell delivery potentiating lipid-containing LNP results in delivery of the nucleic acid to at least about 20% of splenic cells in vivo after a single intravenous injection (e.g., in a non-human primate such as described in Example 5). The levels of delivery demonstrated herein make in vivo therapy possible.

In one embodiment, uptake of the nucleic acid molecule by the target cell is enhanced. Uptake can be determined by methods known to one of skill in the art. For example, association/binding and/or uptake/internalization may be assessed using a detectably labeled, such as fluorescently labeled, LNP and tracking the location of such LNP in or on target cells following various periods of incubation. In addition, mathematical models, such as the ordinary differential equation (ODE)-based model described by Radu Mihaila, et al., (Molecular Therapy: Nucleic Acids, Vol. 7: 246-255, 2017; herein incorporated by reference), allow for quantitation of delivery and uptake.

In another embodiment, function or activity of a nucleic acid molecule can be used as an indication of the delivery of the nucleic acid molecule. For example, in the case of siRNA, reduction in protein expression in a certain proportion of target cells can be measured to indicate delivery of the siRNA to that proportion of cells. Similarly, in the case of mRNA, increase in protein expression in a certain proportion of target cells can be measured to indicate delivery of the siRNA to that proportion of cells. One of skill in the art will recognize various ways to measure delivery of other nucleic acid molecules to target cells.

In certain embodiments, the nucleic acid delivered to the target cell encodes a protein of interest. Accordingly, in one embodiment, an activity of a protein of interest encoded by the nucleic acid molecule in the target cell is enhanced. In one embodiment, expression of a protein encoded by the nucleic acid molecule in the target cell is enhanced. In one embodiment, the protein modulates the activation or activity of the target cell. In one embodiment, the protein increases the activation or activity of the target cell. In one embodiment, the protein decreases the activation or activity of the target cell.

In one embodiment, various agents can be used to label cells to measure delivery to that specific target cell population. For example, the LNP can encapsulate a reporter nucleic acid (e.g., an mRNA encoding a detectable reporter protein), wherein expression of the reporter nucleic acid results in labeling of the cell population to which the reporter nucleic acid is delivered. Non-limiting examples of detectable reporter proteins include enhanced green fluorescent protein (EGFP) and luciferase.

Delivery of the nucleic acid to the target cell by the target cell delivery potentiating lipid-containing LNP can be measured in vitro or in vivo by, for example, detecting expression of a protein encoded by the nucleic acid associated with/encapsulated by the LNP or by detecting an effect (e.g., a biological effect) mediated by the nucleic acid associated with/encapsulated by the LNP. For protein detection, the protein can be, for example, a cell surface protein that is detectable, for example, by immunofluorescence or flow cytometery using an antibody that specifically binds the cell surface protein. Alternatively, a reporter nucleic acid encoding a detectable reporter protein can be used and expression of the reporter protein can be measured by standard methods known in the art.

Methods of the disclosure are useful to deliver nucleic acid molecules to a variety of target cell types, including normal target cells and malignant target cells.

The methods can be used to deliver nucleic acid to target cells located, for example, in the liver or in the spleen.

In one embodiment, the target cell is a malignant cell, a cancer cell, e.g., as demonstrated by deregulated control of G1 progression. In one embodiment, the target cell is a liver cell that is malignant, cancerous or that exhibits deregulated control of G1 progression. In one embodiment, the target cell is a leukemia cell or lymphoma cell. In one embodiment, the target cell is a hepatic cancer cell. In one embodiment, the target cell is a hepatocellular carcinoma cell. In one embodiment, the target cell is a cholangiocarcinoma cell. In one embodiment, the target cell is a liver angiosarcoma cell. In one embodiment, the target cell is a hepatoblastoma cell.

The improved lipid-based compositions, including LNPs of the disclosure are useful to deliver more than one nucleic acid molecules to a target cell or different populations of target cells, by for example, administration of two or more different LNPs. In one embodiment, the method of the disclosure comprises contacting the target cell (or administering to a subject), concurrently or consecutively, a first LNP and a second LNP, wherein the first and second LNP encapsulate the same or different nucleic acid molecules, wherein the first and second LNP include a phytosterol as a component. In other embodiments, the method of the disclosure comprises contacting the target cell (or administering to a subject), concurrently or consecutively, a first LNP and a second LNP, wherein the first and second LNP encapsulate the same or different nucleic acid molecules, wherein the first LNP includes a phytosterol as a component and the second LNP lacks a phytosterol.

### (i) Enzyme Replacement Therapy

In another embodiment, the LNPs of the disclosure provide a nucleic acid that encodes for an enzyme associated with a disease or disorder. In an embodiment, the enzyme associated with the disease or disorder is not expressed at sufficient levels in a subject having the disease or disorder. In an embodiment, the LNP of the disclosure encoding for the enzyme associated with the disease or disorder, can be administered to a subject to increase (e.g., enhance) and/or restore expression and/or activity of the enzyme in the subject, e.g., as enzyme replacement therapy. In an embodiment, the LNP of the disclosure encoding for the enzyme associated with the disease or disorder, results in increased expression and/or activity of the enzyme, e.g., in the subject. In an embodiment, administration of the LNP encoding the enzyme associated with the disease or disorder results in amelioration of one or more symptoms associated with the disease or disorder.

In an embodiment, the disease or disorder is a rare disease (e.g., a lysosomal storage disease), or a metabolic disorder (e.g., as described herein).

In an embodiment, the disease is a metabolic disorder. In an embodiment, the enzyme is a urea cycle enzyme.

### Pharmaceutical Compositions

Formulations comprising lipid nanoparticles of the invention may be formulated in whole or in part as pharmaceutical compositions. Pharmaceutical compositions may include one or more lipid nanoparticles. For example, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

A lipid nanoparticle of the disclosure formulated into a pharmaceutical composition can encapsulate a single nucleic acid or multiple nucleic acids. When encapsulating multiple nucleic acids, the nucleic acids can be of the same type (e.g., all mRNA) or can be of different types (e.g., mRNA and DNA). Furthermore, multiple LNPs can be formulated into the same or separate pharmaceutical compositions. For example, the same or separate pharmaceutical compositions can comprise a first LNP and a second LNP, wherein the first and second LNP encapsulate the same or different nucleic acid molecules, wherein the first and second LNP include na target cell delivery potentiating lipid as a component. In other embodiments, the same or separate pharmaceutical compositions can comprise a first LNP and a second LNP, wherein the first and second LNP encapsulate the same or different nucleic acid molecules, wherein the first LNP includes a target cell delivery potentiating lipid as a component and the second LNP lacks a target cell delivery potentiating lipid.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In one embodiment, such compositions are prepared in liquid form or are lyophylized (e.g., and stored at 4° C. or below freezing). For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer [symbol] solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a LNP, “about” may mean +/−5% of the recited value. For instance, a LNP including a lipid component having about 40% of a given compound may include 30-50% of the compound. In another example, delivery to at least about 30% liver cells may include delivery to 25-35% of liver cells.

Cancer: As used herein, “cancer” is a condition involving abnormal and/or unregulated cell growth, e.g., a cell having deregulated control of G1 progression. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Enhanced delivery: As used herein, the term “enhanced delivery” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a nucleic acid (e.g., a therapeutic and/or prophylactic mRNA) by a nanoparticle to a target cell of interest compared to the level of delivery of the nucleic acid (e.g., a therapeutic and/or prophylactic mRNA) by a control nanoparticle to a target cell of interest (e.g., target cell). For example, “enhanced delivery” by a target cell delivery potentiating lipid-containing LNP of the disclosure can be evaluated by comparison to the same LNP lacking a target cell delivery potentiating lipid. The level of delivery of a target cell delivery potentiating lipid-containing LNP to a particular cell (e.g., target cell) may be measured by comparing the amount of protein produced in target cells using the phytoserol-containing LNP versus the same LNP lacking the target cell delivery potentiating lipid (e.g., by mean fluorescence intensity using flow cytometry), comparing the % of target cells transfected using the target cell delivery potentiating lipid-containing LNP versus the same LNP lacking the target cell delivery potentiating lipid (e.g., by quantitative flow cytometry), or comparing the amount of therapeutic and/or prophylactic in target cells in vivo using the target cell delivery potentiating lipid-containing LNP versus the same LNP lacking the target cell delivery potentiating lipid. It will be understood that the enhanced delivery of a nanoparticle to a target cell need not be determined in a subject being treated, it may be determined in a surrogate such as an animal model (e.g., a mouse or non-human primate model). For example, for determining enhanced delivery to target cells, a mouse or NHP model (e.g., as described in the Examples) can be used and delivery of an mRNA encoding a protein of interest by a target cell delivery potentiating lipid-containing LNP can be evaluated in target cells (e.g., from liver and/or spleen) (e.g., flow cytometry, fluorescence microscopy and the like) as compared to the same LNP lacking the target cell delivery potentiating lipid.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of liver cells (e.g., as described in Example 5) after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5 [symbol] untranslated region (5′-UTR), a 3 [symbol] TR, a 5 [symbol] ap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2 [symbol] amino-LNA having a 2 [symbol] amino functionalization, and 2 [symbol] amino-α-LNA having a 2 [symbol] amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell, e.g., liver cells or splenic cells) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model). For example, for determining specific delivery to target cells, a mouse or NHP model (e.g., as described in the Examples) can be used and delivery of an mRNA encoding a protein of interest can be evaluated in target cells (e.g., from liver and/or spleen) as compared to non-target cells by standard methods (e.g., flow cytometry, fluorescence microscopy and the like).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Target cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient. Target cells include, for example, liver cells (e.g., a hepatocyte, a hepatic stellate cell, a Kupffer cell, or a liver sinusoidal cell, or a combination thereof) or splenic cells (e.g., splenocytes)).

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning. Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

The Examples demonstrate the physiological effect of target cell target cell delivery LNPs and were designed to further test the uptake of the subject LNPs by target cells. These experiments support development of LNPs for delivery of therapeutic molecules for expression in target cells in patients in vivo or in target cells from patients ex vivo.

### EXAMPLES

Table of Contents

| Example | Title |
|---|---|
| 1 | Syntheses of compounds |
| 2 | Production of Nanoparticle Compositions |
| 3 | Biodistribution and pharmacological |
| | profile of Compound 301 containing LNP |
| 4 | Additional pharmacological profile |
| | of Compound 301 containing LNP |
| 5 | Enhanced delivery of Compound 301 |
| | containing LNPs in the liver |
| 6 | Human EPO Protein Plasma Pharmacokinetics |
| | in non-human primates (NHP) |
| 7 | Effect of molar composition of Compound |
| | 301 containing LNP on mRNA expression |
| 8 | Effect of molar composition of Compound |
| | 301 containing LNP on physical properties of LNP |
| 9 | Effect of molar composition of Compound |
| | 301 containing LNP on mRNA expression |

### Example 1: Syntheses of Compounds

Syntheses of representative ionizable lipids of the invention, e.g. Compounds having any of Formulae (I I), (I IA), (I IB), (I II), (I IIa), (I IIb), (I IIc), (I IId), (I IIe), (I IIf), (I IIg), (I IIh), (I IIj), (I IIk), (I III), (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), (I VIIId), (I XI), (I XI-a), or (I XI-b). are described in co-pending applications PCT/US2016/052352, and PCT/US2018/022717, the contents of each of which are incorporated herein by reference in their entireties.

### Example 2: Production of Nanoparticle Compositions

### A. Production of Nanoparticle Compositions

In order to investigate safe and efficacious nanoparticle compositions for use in the delivery of therapeutic and/or prophylactics to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the therapeutic and/or prophylactic and the other has the lipid components.

Lipid compositions are prepared by combining a lipid according to Formulae (I), (IA), (II), (IIa), (IIb), (IIc), (IId), (IIe), (III), and (IIIa1-8) and/or any of Compounds X, Y, Z, Q or M or a non-cationic helper lipid (such as DOPE, DSPC, or oleic acid obtainable from Avanti Polar Lipids, Alabaster, Ala.), a PEG lipid (such as 1,2-dimyristoyl-sn-glycerol methoxypolyethylene glycol, also known as PEG-DMG, obtainable from Avanti Polar Lipids, Alabaster, Ala.), and a phytosterol (optionally including a structural lipid such as cholesterol) at concentrations of about, e.g., 50 mM in a solvent, e.g., ethanol. Solutions should be refrigeration for storage at, for example, −20° C. Lipids are combined to yield desired molar ratios (see, for example, Table 21 below) and diluted with water and ethanol to a final lipid concentration of e.g., between about 5.5 mM and about 25 mM. Phytosterol* in Table 21 refers to phytosterol or optionally a combination of phytosterol and structural lipid such as beta-phytosterol and cholesterol. Table 21. Exemplary formulations including Compounds according to Formulae (I), (IA), (II), (IIa), (IIb), (IIc), (IId), (IIe), (III), and (IIIa1-8) and/or any of Compounds X, Y, Z, Q or M.

TABLE 21

| Composition (mol %) | Components |
|---|---|
| 40:20:38.5:1.5 | Compound:Phospholipid:Phytosterol*:PEG-DMG |
| 45:15:38.5:1.5 | Compound:Phospholipid:Phytosterol*:PEG-DMG |
| 50:10:38.5:1.5 | Compound:Phospholipid:Phytosterol*:PEG-DMG |
| 55:5:38.5:1.5 | Compound:Phospholipid:Phytosterol*:PEG-DMG |
| 60:5:33.5:1.5 | Compound:Phospholipid:Phytosterol*:PEG-DMG |
| 45:20:33.5:1.5 | Compound:Phospholipid:Phytosterol*:PEG-DMG |
| 50:20:28.5:1.5 | Compound:Phospholipid:Phytosterol*:PEG-DMG |
| 55:20:23.5:1.5 | Compound:Phospholipid:Phytosterol*:PEG-DMG |
| 60:20:18.5:1.5 | Compound:Phospholipid:Phytosterol*:PEG-DMG |
| 40:15:43.5:1.5 | Compound:Phospholipid:Phytosterol*:PEG-DMG |
| 50:15:33.5:1.5 | Compound:Phospholipid:Phytosterol*:PEG-DMG |
| 55:15:28.5:1.5 | Compound:Phospholipid:Phytosterol*:PEG-DMG |
| 60:15:23.5:1.5 | Compound:Phospholipid:Phytosterol*:PEG-DMG |
| 40:10:48.5:1.5 | Compound:Phospholipid:Phytosterol*:PEG-DMG |
| 45:10:43.5:1.5 | Compound:Phospholipid:Phytosterol*:PEG-DMG |
| 55:10:33.5:1.5 | Compound:Phospholipid:Phytosterol*:PEG-DMG |
| 60:10:28.5:1.5 | Compound:Phospholipid:Phytosterol*:PEG-DMG |
| 40:5:53.5:1.5 | Compound:Phospholipid:Phytosterol*:PEG-DMG |
| 45:5:48.5:1.5 | Compound:Phospholipid:Phytosterol*:PEG-DMG |
| 50:5:43.5:1.5 | Compound:Phospholipid:Phytosterol*:PEG-DMG |
| 40:20:40:0 | Compound:Phospholipid:Phytosterol*:PEG-DMG |
| 45:20:35:0 | Compound:Phospholipid:Phytosterol*:PEG-DMG |
| 50:20:30:0 | Compound:Phospholipid:Phytosterol*:PEG-DMG |
| 55:20:25:0 | Compound:Phospholipid:Phytosterol*:PEG-DMG |
| 60:20:20:0 | Compound:Phospholipid:Phytosterol*:PEG-DMG |
| 40:15:45:0 | Compound:Phospholipid:Phytosterol*:PEG-DMG |
| 45:15:40:0 | Compound:Phospholipid:Phytosterol*:PEG-DMG |
| 50:15:35:0 | Compound:Phospholipid:Phytosterol*:PEG-DMG |
| 55:15:30:0 | Compound:Phospholipid:Phytosterol*:PEG-DMG |
| 60:15:25:0 | Compound:Phospholipid:Phytosterol*:PEG-DMG |
| 40:10:50:0 | Compound:Phospholipid:Phytosterol*:PEG-DMG |
| 45:10:45:0 | Compound:Phospholipid:Phytosterol*:PEG-DMG |
| 50:0:48.5:1.5 | Compound:Phospholipid:Phytosterol*:PEG-DMG |
| 50:10:40:0 | Compound:Phospholipid:Phytosterol*:PEG-DMG |
| 55:10:35:0 | Compound:Phospholipid:Phytosterol*:PEG-DMG |
| 60:10:30:0 | Compound:Phospholipid:Phytosterol*:PEG-DMG |

Nanoparticle compositions including a therapeutic and/or prophylactic and a lipid component are prepared by combining the lipid solution with a solution including the therapeutic and/or prophylactic at lipid component to therapeutic and/or prophylactic wt:wt ratios between about 5:1 and about 50:1. The lipid solution is rapidly injected using a NanoAssemblr microfluidic based system at flow rates between about 10 ml/min and about 18 ml/min into the therapeutic and/or prophylactic solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kDa. The first dialysis is carried out at room temperature for 3 hours. The formulations are then dialyzed overnight at 4° C. The resulting nanoparticle suspension is filtered through 0.2 m sterile filters (Sarstedt, Numbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a therapeutic and/or prophylactic (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the nanoparticle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 g/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver therapeutic and/or prophylactics to targeted cells, different nanoparticle compositions including a particular therapeutic and/or prophylactic (for example, a modified or naturally occurring RNA such as an mRNA) are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a therapeutic and/or prophylactic in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

### Example 3: Biodistribution and Pharmacological Profile of Compound 301 Containing LNP

In this example, a Compound 301 containing LNP was used to deliver a Luciferase-encoding mRNA (NPI-Luc) to rats in vivo and the biodistribution of the LNP and its pharmacological profile at various time points was assessed in the plasma and in various tissues.

Rats were dosed intravenously with an NPI-Luc mRNA-encapsulated LNP at 2 mg/kg on Day 1 (Groups 2-8), left untreated (Group 1) or dosed on Day 1, Day 8 and Day 15 (Groups 9-16). Table 22 summarizes the treatment and dosing parameters.

TABLE 22
Study design

| | IV | Dose | Dose | Total Number | | | Tissue Collection | Blood Collection |
|---|---|---|---|---|---|---|---|---|
| | Infusion | Level | Volume | of Animals | | Dose | Intervals | Intervals |
| Group | Treatment | (mg/kg) | (mL/kg) | M | F | Days | (Hours Postdose) | (Hours Postdose) |
| 1 | NPI-Luc | 0 | 0 | 3 | 3 | 1 | Day 1: Predose | Day 1: Predoseₐ |
| | mRNA | | | | | | | |
| 2 | NPI-Luc | 2.0 | 5 | 3 | 3 | 1 | Day 1: 2 hr | Day 1: 2 hrₐ |
| | mRNA | | | | | | | |
| 3 | NPI-Luc | 2.0 | 5 | 3 | 3 | 1 | Day 1: 6 hr | Day 1: 6 hrₐ |
| | mRNA | | | | | | | |
| 4 | NPI-Luc | 2.0 | 5 | 3 | 3 | 1 | Day 1: 24 hr | Day 1: 0.25 |
| | mRNA | | | | | | | (15 min), 24 hrₐ |
| 5 | NPI-Luc | 2.0 | 5 | 3 | 3 | 1 | Day 1: 48 hr | Day 1: 0.5 |
| | mRNA | | | | | | | (30 min), 48 hrₐ |
| 6 | NPI-Luc | 2.0 | 5 | 3 | 3 | 1 | Day 1: 72 hr | Day 1: 1, 72 hrₐ |
| | mRNA | | | | | | | |
| 7 | NPI-Luc | 2.0 | 5 | 3 | 3 | 1 | Day 1: 96 hr | Day 1: 4, 96 hrₐ |
| | mRNA | | | | | | | |
| 8 | NPI-Luc | 2.0 | 5 | 3 | 3 | 1 | Day 1: 168 hr | Day 1: 10, 168 hrₐ |
| | mRNA | | | | | | | |
| 9 | NPI-Luc | 2.0 | 5 | 3 | 3 | 1, 8, 15 | Day 15: Predose | Day 15: Predoseₐ |
| | mRNA | | | | | | | |
| 10 | NPI-Luc | 2.0 | 5 | 3 | 3 | 1, 8, 15 | Day 15: 2 hr | Day 15: 2 hrₐ |
| | mRNA | | | | | | | |
| 11 | NPI-Luc | 2.0 | 5 | 3 | 3 | 1, 8, 15 | Day 15: 6 hr | Day 15: 6 hrₐ |
| | mRNA | | | | | | | |
| 12 | NPI-Luc | 2.0 | 5 | 3 | 3 | 1, 8, 15 | Day 15: 24 hr | Day 15: 24 hrₐ |
| | mRNA | | | | | | | |
| 13 | NPI-Luc | 2.0 | 5 | 3 | 3 | 1, 8, 15 | Day 15: 0.25, 48 hr | Day 15: 48 hrₐ |
| | mRNA | | | | | | | |
| 14 | NPI-Luc | 2.0 | 5 | 3 | 3 | 1, 8, 15 | Day 15: 0.5, 72 hr | Day 15: 72 hrₐ |
| | mRNA | | | | | | | |
| 15 | NPI-Luc | 2.0 | 5 | 3 | 3 | 1, 8, 15 | Day 15: 1, 96 hr | Day 15: 96 hrₐ |
| | mRNA | | | | | | | |
| 16 | NPI-Luc | 2.0 | 5 | 3 | 3 | 1, 8, 15 | Day 15: 4, 168 hr | Day 15: 168 hrₐ |
| | mRNA | | | | | | | |

Plasma sample and tissues were collected from the dosed animals at 0 hours, 24 hours, 48 hours, 72 hours, 96 hours, 120 hours, 144 hours and 168 hours. LNP level in the plasma and in various tissues was determined by LC-MS/MS. mRNA level in the plasma was determined by Atlas and mRNA level in various tissues was determined by methods known in the art.

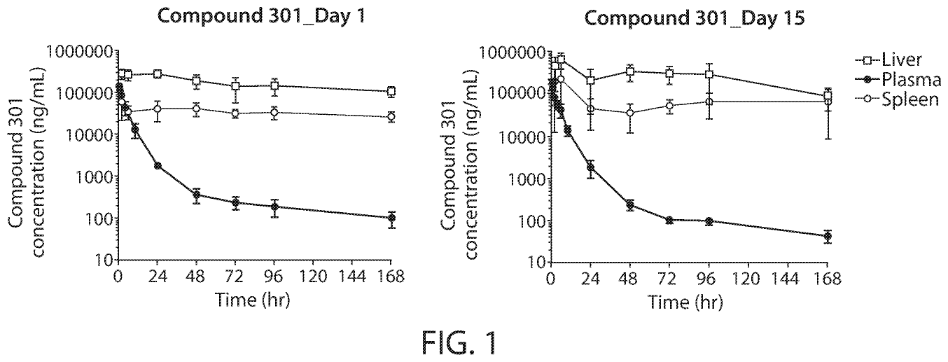

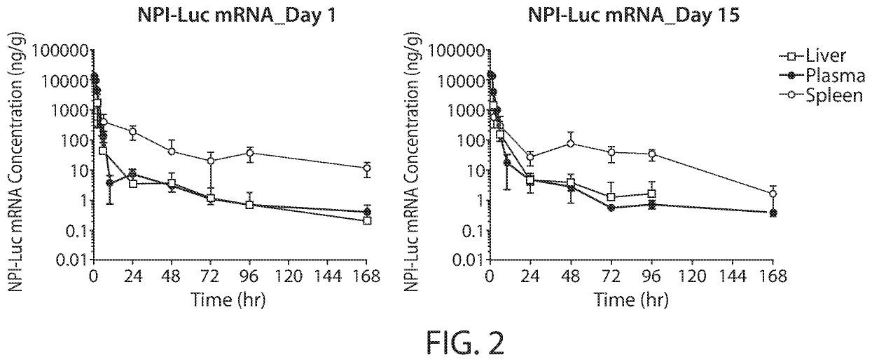

The results of the analysis of LNP levels are shown in FIG. 1, which demonstrates the concentration of Compound 301 containing lipid in various tissues and plasma at the indicated time points on Day 1 and Day 15. Samples obtained from the liver and ovaries of dosed rats showed the highest concentration of Compound 301 containing lipid. The results of the analysis of mRNA levels are shown in FIG. 2, which demonstrates the NPI Luc mRNA concentration in various tissues and plasma at the indicated time points on Day 1 and Day 15. Highest NPI Luc mRNA concentration was observed in the plasma, followed by spleen, liver and ovaries on Day 1. On Day 15, expression of NPI Luc mRNA was highest in the plasma, followed by spleen liver and lung.

Pharmacokinetic properties of the LNPs was also assessed in the plasma and in various tissues. The results are shown in Table 23. C max and area under the curve (AUC) was highest in samples obtained from the plasma.

TABLE 23
Pharmacokinetic properties of LNP

| | tₘₐₓ | Cₘₐₓ | Cₗₐₛₜ | t_{1/2} | AUCₗₐₛₜ | AUC_{inf} | AUC_{% Extrap} | |
|---|---|---|---|---|---|---|---|---|
| Matrix | (h) | (ng/mL) | (ng/mL) | (h) | (h*ng/mL) | (h*ng/mL) | (%) | R² |
| Liver | 2 | 279000 | 105000 | 210 | 28100000 | 59700000 | 53.0 | 0.884 |
| Plasma | 0.5 | 142000 | 100 | 77.9 | 678000 | 689000 | 1.63 | 1 |
| Spleen | 2 | 68100 | 26500 | 240 | 5690000 | 14800000 | 61.7 | 0.834 |

### Example 4: Additional Pharmacological Profile of Compound 301 Containing LNP

In this example, Compound 301, Compound 18 or Compound 50 containing LNPs were used to deliver an mRNA which encodes for human EPO and a microRNA 126 (miR 126) and miR 142 to mice and the lipid metabolism was evaluated.

Mice were dosed intravenously with human EPO and miR mRNA-encapsulated LNP at 0.5 mg/kg. The metabolism of human EPO in the liver and spleen was assessed at 2 hours, 6 hours, 24 hours, 48 hours, 72 hours, and 96 hours and 192 hours.

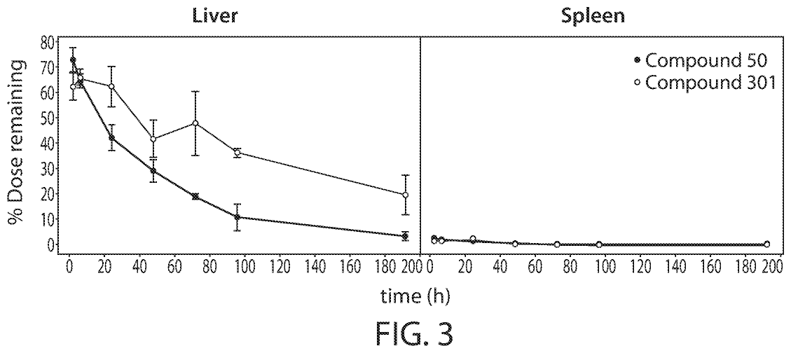

The results are shown in FIG. 3, which demonstrates that Compound 301 containing LNPs show a slower liver metabolism compared to Compound 50 containing LNP and Compound 18 containing LNP. Compound 18 containing LNPs were undetectable within hours of administration.

### Example 5: Enhanced Delivery of Compound 301 Containing LNPs in the Liver

In this Example, a compound 301 containing LNP or a compound 18 containing LNP was used to deliver a Luciferase-encoding mRNA (NPI-Luc) to non-human primates (NHPs) and the cellular biodistribution of the LNPs were assessed in the plasma and in various tissues. NHPs were dosed once intravenously with either LNP at 2 mg/kg. Table 24 summarizes the treatment and dosing parameters and LNP formulations. PEG lipids used in this Example correspond to Compound 428 (also referred to as PEG 1).

TABLE 24
Study design

| | | | Dose level | Dose volume | Dose conc. | Number |
|---|---|---|---|---|---|---|
| LNP | Lipid Ratio | N:P | (mg/kg/day) | mL/kg | (mg/mL) | of males |
| Compound | 48:11:38.2.7 | 5.8 | 2 | 5 | 0.4 | 3 |
| 18/DSPC/ | | | | | | |
| Cholesterol/ | | | | | | |
| PEG lipid | | | | | | |
| Compound | 48:11:38.3 | 4 | 2 | 5 | 0.4 | 6 |
| 301/DSPC/ | | | | | | |
| Cholesterol/ | | | | | | |
| PEG lipid | | | | | | |

Liver samples were collected from the dosed animals and processed for NPI-luc protein quantitation and NPI-luc immunohistochemistry (IHC). NPI-luc protein quantitation was performed using an ELISA from Meso Scale Discovery (MSD) according to the manufacturer's protocol. Briefly, MSD plates were pre-coated overnight with a capture antibody. The residual antibody was then removed, and the plate was blocked with the Super Block reagent. The homogenized sample was then added to the plate and incubated for 1 hour at room temperature. The plate was then washed, and a secondary Ab was added. The washing step was repeated and an anti-rabbit Sulfotag antibody was then added to the samples. After a final washing step, MSD read buffer as added and the samples were analyzed on the MSD reader.

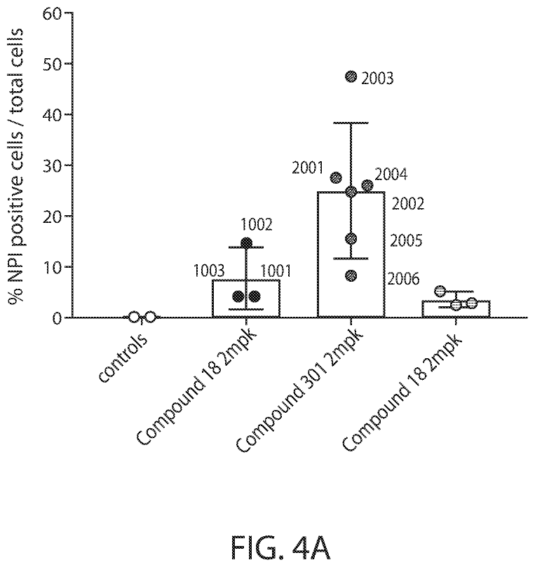

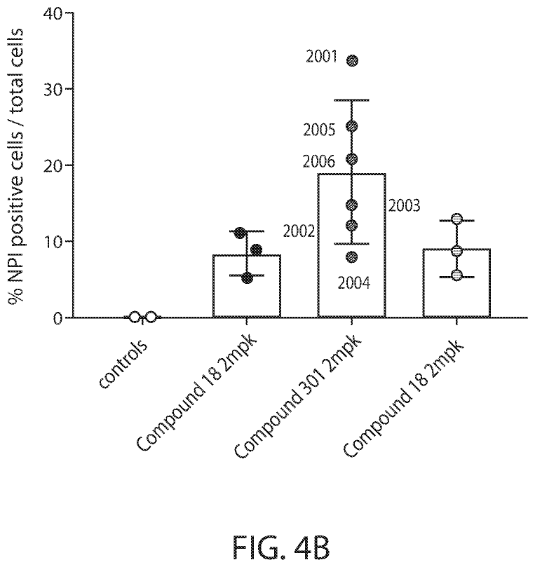

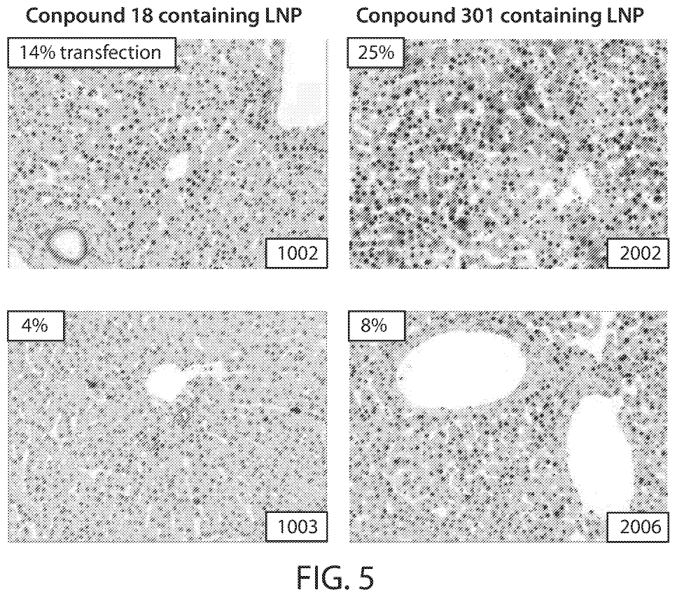

The results are shown in FIGS. 4A-4B and FIG. 5. FIG. 4A shows an average of about a three-fold increase in liver cell (e.g., hepatocyte) expression of NPI Luc in animals dosed with NPI-Luc mRNA-encapsulated Compound 301 LNP as compared to animals dosed with NPI-Luc mRNA-encapsulated Compound 18 LNP. FIG. 4B shows an average of about a two-fold increase in NPI-Luc expression in spleen cells of animals dosed with NPI-Luc mRNA-encapsulated Compound 301 LNP as compared to animals dosed with NPI-Luc mRNA-encapsulated Compound 18 LNP. Non-hepatocyte expression of NPI-Luc mRNA was estimated to be less than 10%. Exemplary IHC stains from the liver samples of the dosed animals is shown in FIG. 5.

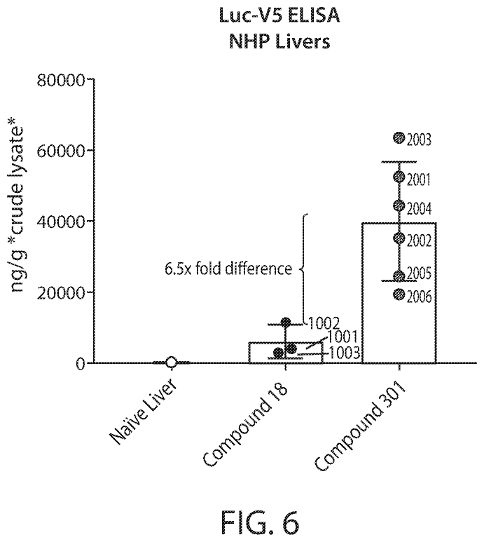

NPI-luc protein levels in the liver samples is shown in FIG. 6, which demonstrates an approximately 6.5-fold higher NPI-luc protein expression in samples from animals dosed with the NPI-Luc mRNA-encapsulated Compound 301 LNP as compared to animals dosed with NPI-Luc mRNA-encapsulated Compound 18 LNP.

### Example 6: Human EPO Protein Plasma Pharmacokinetics in Non-Human Primates (NHP)

In this example, Compound 301 or Compound I-18 containing LNPs were used to deliver an mRNA which encodes for human EPO and a micro RNA 126 (miR 126) and miR 142 to NHPs. The pharmacokinetics of human EPO delivered by the various LNPs was assessed.

NHPs were dosed intravenously with the indicated LNP at 0.1 mg/kg on Day 1, 15 and 29. Table 25 summarizes the treatment and dosing parameters. At the indicated time points, samples were collected for ELISA based protein analysis. The PEG lipids used in this Example correspond to Compound 428 (also referred to as PEG 1).

TABLE 25
Dosing parameters

| | | | Dose | Dose | |
|---|---|---|---|---|---|
| | | Dose | volume | concentration | Number of |
| Group No. | Test material | level | (mL/kg) | (mg/mL) | males |
| 1 | hEPO mRNA with | 0.1 | 5 | 0.02 | 6 |
| | miR126/142 in Compound | | | | |
| | 18/PEG lipid containing | | | | |
| | LNP | | | | |
| 2 | hEPO mRNA with | 0.1 | 5 | 0.02 | 6 |
| | miR126/142 in Compound | | | | |
| | 301/PEG lipid containing | | | | |
| | LNP | | | | |

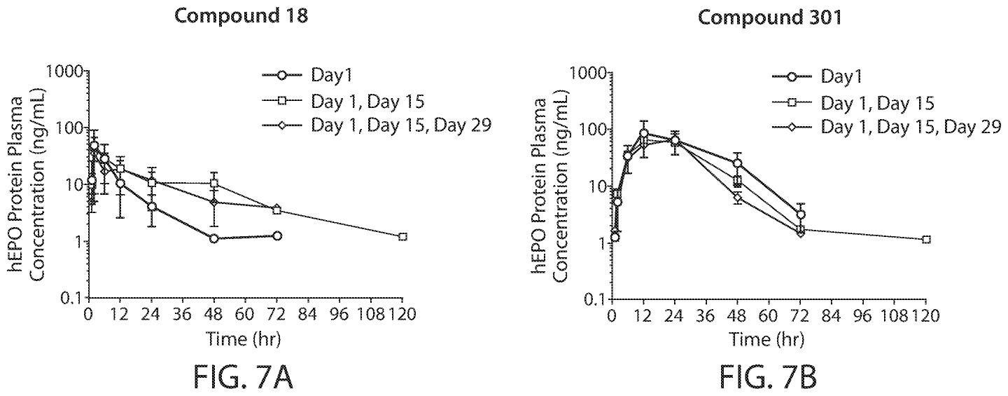

The results are shown in FIGS. 7A-7B, which demonstrate higher human EPO protein level on Days 1, 15 and 29 in the plasma of NHPs dosed with Compound 301 containing LNP compared to NHPs dosed with Compound 18 containing LNPs. The C max and the area under the curve (AUC) was higher in Compound 301 containing LNP compared to Compound 18 containing LNPs, as shown in Table 26. The half-life of human EPO was comparable in all groups. The PEG lipid used in this Example corresponds to Compound 428 (also referred to as PEG 1).

The results from the Day 15 and Day 29 dosing with Compound 301 containing LNPs shows similar levels of human EPO in the plasma compared to the Day 1 dosings. This demonstrates that repeat dosing with Compound 301 containing LNP does not result in reduced plasma level (expression) of the payload and suggests that Compound 301 containing LNPs do not promote accelerated blood clearance.

TABLE 26
PK/PD properties of LNPs

| | | tₘₐₓ * | | | Cₘₐₓ | | | t_{1/2} | | | AUCₗₐₛₜ | | |
|---|---|---|---|---|---|---|---|---|---|---|---|---|---|
| | | (h) | | | (ng/mL) | | | (h) | | | (h*ng/mL) | | |
| | | Day | | | | | | | | | | | |
| Formulation | | 1 | 15 | 29 | 1 | 15 | 29 | 1 | 15 | 29 | 1 | 15 | 29 |
| Compound | Mean | 2 | 2 | 2 | 48 | 54 | 39 | 10.9 | 13.5 | 10.4 | 408 | 670 | 554 |
| 18/PEG lipid | SD | | | | 43 | 46 | 31 | 7.8 | 14.7 | 5.7 | 310 | 571 | 758 |
| Compound | Mean | 12 | 12 | 24 | 87 | 70 | 72 | 11.0 | 12.9 | 9.8 | 2570 | 2000 | 1734 |
| 301/PEG lipid | SD | | | | 52 | 26 | 34 | 0.7 | 7.3 | 2.2 | 1290 | 767 | 784 |

### Example 7: Effect of Molar Composition of Compound 301 Containing LNP on mRNA Expression in Hepatocytes

In this example, Compound 301 containing LNPs were used to deliver an mRNA which encodes for human EPO and a micro RNA 126 (miR 126) and miR 142 to human EPO levels in the plasma was measured.

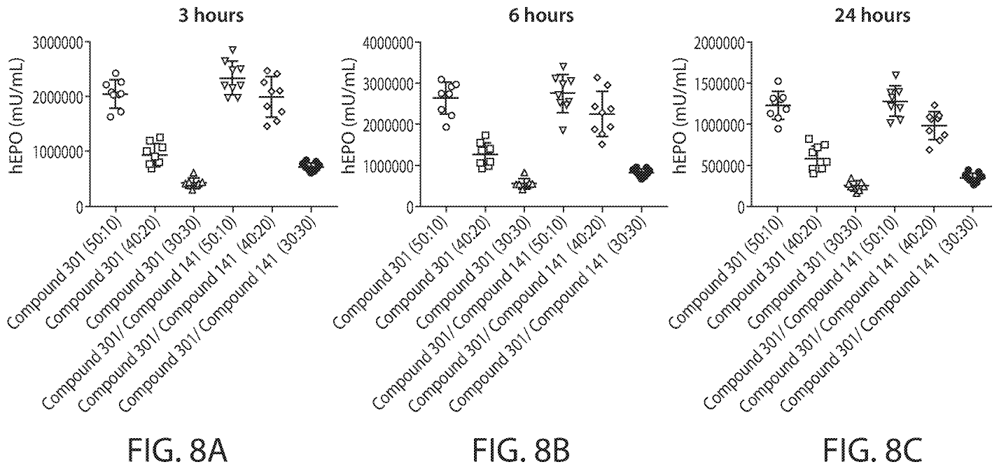

Compound 301 containing LNPs were formulated at the following ionizable lipid to phospholipid (DSPC) ratios: 50:10, 40:20 or 30:30. The results are shown in FIGS. 8A-8C, which demonstrate increased hepatocyte delivery of human EPO from Compound 301 containing LNPs formulated at a 50:10 ionizable lipid:DSPC ratio. Addition of a Compound 141 containing LNP did not restore the ability to transfect hepatocytes.

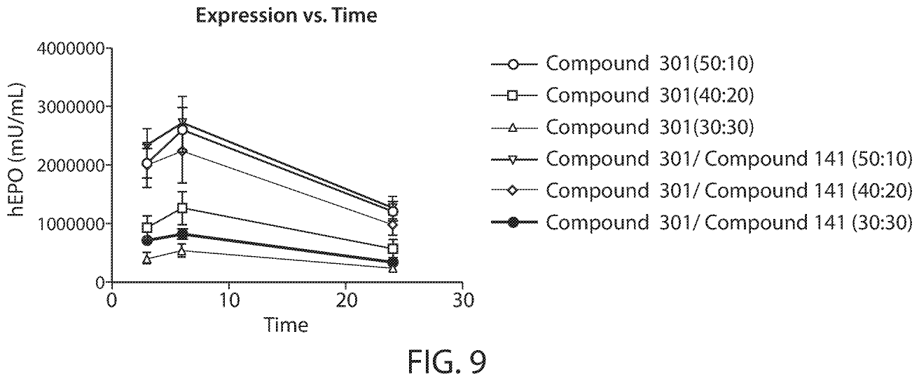

FIG. 9 shows the expression of human EPO over time in mice administered Compound 301 containing LNPs with the indicated ionizable lipid:DSPC ratios. Compound 301 containing LNPs formulated at a 50:10 ionizable lipid:DSPC ratio demonstrated increased AUC ratio as compared to other formulations and co-administration of a Compound 141 containing LNP. Table 27 summarizes the observed AUC ratios for the various LNP formulations.

TABLE 27
AUC ratios for the various LNP formulations

| LNP | AUC Ratio |
|---|---|
| Compound 301 (50:10) | 1.00 |
| Compound 301 (40:20) | 0.48 |
| Compound 301 (30:30) | 0.21 |
| Compound 301/Compound | 1.05 |
| 141 (50:10) | |
| Compound 301/Compound | 0.85 |
| 141 (40:20) | |
| Compound 301/Compound | 0.31 |
| 141 (30:30) | |

### Example 8: Effect of Molar Composition of Compound 301 Containing LNP on Physical Properties of LNP

In this example, a Compound 301 containing LNP was used to deliver a Luciferase-encoding mRNA (NPI-Luc) to rats in vivo and the biodistribution and stability of the LNPs were assessed.

Rats were dosed intravenously with an NPI-Luc mRNA-encapsulated LNP at 0.5 mg/kg. The LNPs were formulated with different ionizable lipid:DSPC ratios as shown in Tables 28 and 29. The PEG lipids used in this Example correspond to Compound 428 (also referred to as PEG 1).

TABLE 28
Molar composition of LNP formulations

| | | Mol % | | Mol % | Final |
|---|---|---|---|---|---|
| | | Compound | Mol % | Choles- | Mol % |
| Group | Formulation | 301 | DSPC | terol | OL-56 |
| 1 | Compound 301/ | 50 | 5 | 42 | 3 |
| 2 | DSPC/Chol/PEG | 50 | 10 | 37 | 3 |
| 3 | lipid (0.25/2/3) | 50 | 15 | 32 | 3 |
| 4 | N:P 4 | 50 | 20 | 27 | 3 |
| 5 | | 55 | 5 | 37 | 3 |
| 6 | | 55 | 10 | 32 | 3 |
| 7 | | 55 | 15 | 27 | 3 |

TABLE 29
Molar composition of LNP formulations

| | | Mol % | | Mol % | Final |
|---|---|---|---|---|---|
| | | Compound | Mol % | Choles- | Mol % |
| Group | Formulation | 301 | DSPC | terol | OL-56 |
| 1 | Compound 301/ | 40 | 15 | 42 | 3 |
| 2 | DSPC/Chol/PEG | 40 | 20 | 37 | 3 |
| 3 | lipid (0.25/2/3) | 45 | 10 | 42 | 3 |
| 4 | N:P 4 | 45 | 15 | 37 | 3 |
| 5 | | 60 | 10 | 27 | 3 |
| 6 | | 50 | 10 | 37 | 3 |

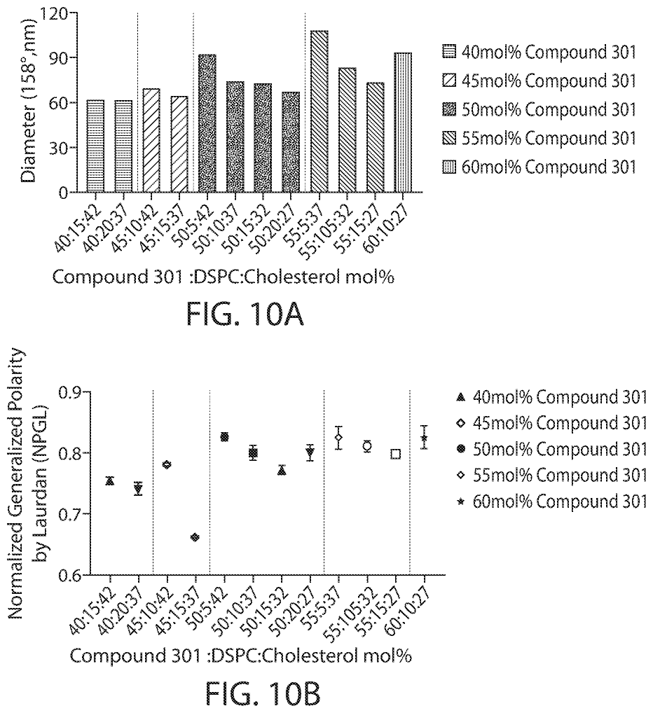

The results are shown in FIGS. 10A-10B, which demonstrate that a 50:10:37 ionizable lipid:DSPC:cholesterol molar ratio had an effect on the particle diameter and surface polarity of the LNP. The tested LNP formulations did not affect processability. Table 30 provides a summary of the physical properties of the LNP formulations. A higher mol 00 of Compound 301 typically resulted in a larger particle diameter. A lower mol 0% DSPC and a higher mol 0% cholesterol typically resulted in larger particle diameter.

TABLE 30
Properties of LNPs

| Compound | | | |
|---|---|---|---|
| 301:DSPC:Chol | Diameter | | |
| mol % | (nm) | PDI¹ | % EE² |
| 40:15:42 | 62.1 | 0.06 | 99 |
| 40:20:37 | 61.7 | 0.12 | 99 |
| 45:10:42 | 70.0 | 0.06 | 99 |
| 45:15:37 | 64.8 | 0.07 | 99 |
| 50:5:42 | 92.7 | 0.05 | 99 |
| 50:10:37 | 74.6 | 0.06 | 99 |
| 50:15:32 | 73.2 | 0.06 | 99 |
| 50:20:27 | 67.7 | 0.07 | 99 |
| 55:5:37 | 108.7 | 0.06 | 99 |
| 55:10:32 | 83.7 | 0.05 | 99 |
| 55:15:27 | 74.0 | 0.06 | 99 |
| 60:10:27 | 93.7 | 0.05 | 99 |

PDI¹: polydispersity index
EE²: encapsulation efficiency

### Example 9: Effect of Molar Composition of Compound 301 Containing LNP on mRNA Expression

In this example, a Compound 301 containing LNP was used to deliver a Luciferase-encoding mRNA (NPI-Luc) to rats in vivo and the expression of NPI-Luc in the animals was assessed.

Rats were dosed intravenously with an NPI-Luc mRNA-encapsulated LNP at 0.5 mg/kg. The LNPs were formulated with different ionizable lipid:DSPC ratios as shown in Tables 28 and 29. Whole body imaging of the animals was performed 6 hours after administration of the compounds.

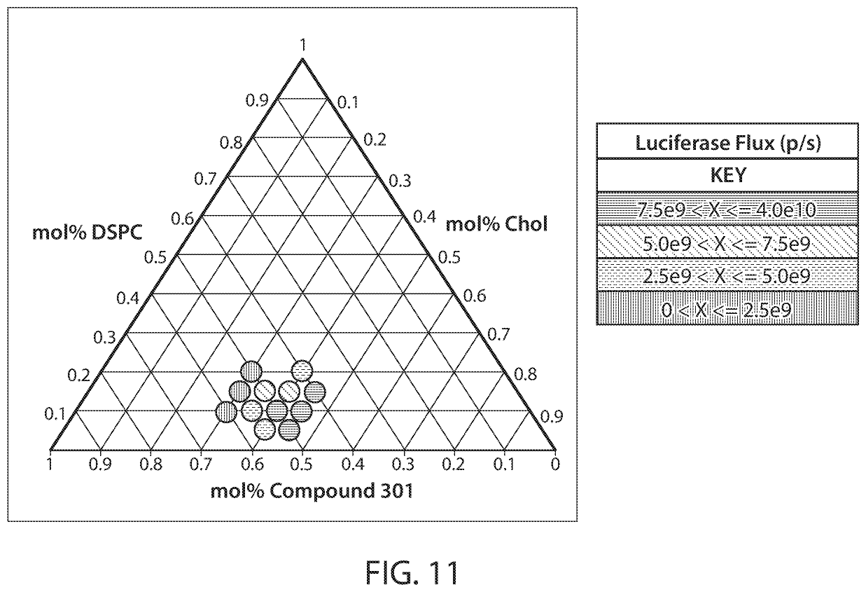

A summary of the results are shown in FIG. 11, which demonstrates the optimal composition ratio of ionizable lipid:DSPC:cholesterol for in vivo expression.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A target cell delivery lipid nanoparticle (LNP) comprising:
(i) an ionizable lipid, e.g., an amino lipid;
(ii) a sterol or other structural lipid;
(iii) a non-cationic helper lipid or phospholipid;
(iv) a payload; and
(v) optionally, a PEG-lipid,
wherein the target cell delivery LNP results in one, two, three or all of:
(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;
(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;
(c) expression and/or activity of payload in greater than 30%, 40%, 50%, 60%, 65%, 70%, 75% or more total liver cells, e.g., in about 60% of total liver cells; or
(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

2. The delivery LNP of claim 1, wherein the target cell is a liver cell, e.g., a hepatocyte.

3. The delivery LNP of claim 1 or 2, which results in expression and/or activity of payload in greater than 30%, 40%, 50%, 55%, 60%, 65%, 70%, 75% or more total liver cells.

4. The delivery LNP of claim 3, which results in expression and/or activity of payload in about 60% of total liver cells.

5. The delivery LNP of any of the preceding claims, which results in enhanced payload level (e.g., expression) in liver cells, e.g., hepatocytes, relative to a reference LNP.

6. The delivery LNP of any of the preceding claims, which results in about 1.5-fold, 2-fold, 3-fold, 4-fold, 5-fold, or 6-fold increase in liver cell expression, e.g., hepatocyte expression, relative to a reference LNP.

7. The delivery LNP of any of the preceding claims, which has an increased efficiency of cytosolic delivery, e.g., as compared to a reference LNP, e.g., as described herein.

8. The delivery LNP of any of the preceding claims, which results in one, two or all of:
a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;
b) greater half-life (t ½) in the liver relative to plasma, e.g., a t ½ that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or
c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.

9. The delivery LNP of any of the preceding claims, which has an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced endosomal escape; or
8) slower lipid metabolism in the liver relative to the spleen, e.g., at least 10%, 20%, 30%, 40%, 50%, 60%, 70%, 80%, 90% or more lipid remaining in the liver 24 hours post-administration.

10. The delivery LNP of any one of the preceding claims, which results in one, two, three or all of:
13) an increased response rate, e.g., a defined by at specified threshold of liver cell transfection;
14) at least 5%, 10%, 15%, 20%, 25%, 30%, 34%, 35%, 36%, 37%, 38%, 39%, 40% or more liver cell transfection;
15) an increased responder rate, e.g., a defined by at specified threshold of liver cell transfection; or
16) an increased response rate greater than a reference LNP, e.g., at least 1-fold, 1.5-fold, 2-fold, 2.5-fold, or 3-fold or greater response rate.

11. The delivery LNP of any one of the preceding claims, wherein the target cell delivery LNP is formulated for systemic delivery.

12. The delivery LNP of any one of the preceding claims, wherein the target cell delivery LNP is administered systemically, e.g., parenterally (e.g., intravenously, intramuscularly, subcutaneously, intrathecally, or intradermally) or enterally (e.g., orally, rectally or sublingually).

13. The delivery LNP of any one of the preceding claims, which delivers the payload to a cell capable of protein synthesis and/or a cell having a high engulfing capacity.

14. The delivery LNP of any one of the preceding claims, which delivers the payload to a liver cell, e.g., a hepatocyte, a hepatic stellate cell, a Kupffer cell, or a liver sinusoidal cell, or a combination thereof.

15. The delivery LNP of any one of the preceding claims, which delivers the payload to a hepatocyte.

16. The delivery LNP of any one of the preceding claims, which delivers the payload to a non-immune cell.

17. The delivery LNP of any one of the preceding claims, which delivers the payload to a splenic cell, e.g., a non-immune splenic cell (e.g., a splenocyte).

18. The delivery LNP of any one of the preceding claims, which delivers the payload to a cell chosen from an ovarian cell, a lung cell, an intestinal cell, a heart cell, a skin cell, an eye cell or a brain cell, or a skeletal muscle cell.

19. The delivery LNP of any one of the preceding claims, wherein an intracellular concentration of the nucleic acid molecule in the target cell is enhanced.

20. The delivery LNP of any one of the preceding claims, wherein uptake of the nucleic acid molecule by the target cell is enhanced.

21. The delivery LNP of any one of the preceding claims, wherein an activity of the nucleic acid molecule in the target cell is enhanced.

22. The delivery LNP of any one of the preceding claims, wherein expression of the nucleic acid molecule in the target cell is enhanced.

23. The delivery LNP of any one of the preceding claims, wherein an activity of a protein encoded by the nucleic acid molecule in the target cell is enhanced.

24. The delivery LNP of any one of the preceding claims, wherein expression of a protein encoded by the nucleic acid molecule in the target cell is enhanced.

25. The delivery LNP of any one of the preceding claims, wherein delivery is enhanced in vivo.

26. The delivery LNP of any one of the preceding claims, wherein the payload is a peptide, polypeptide, protein or a nucleic acid.

27. The delivery LNP of any one of the preceding claims, wherein the payload is a nucleic acid molecule chosen from RNA, mRNA, dsRNA, siRNA, antisense RNA, ribozyme, CRISPR/Cas9, ssDNA and DNA.

28. The delivery LNP of any one of the preceding claims, wherein the payload is chosen from a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), or a combination thereof.

29. The delivery LNP of any one of the preceding claims, wherein the payload is an mRNA, a siRNA, a miR, or a CRISPR.

30. The delivery LNP of any one of the preceding claims, wherein the payload is an mRNA.

31. The delivery LNP of any one of the preceding claims, wherein the payload is an mRNA encoding a protein of interest other than an immune cell payload.

32. The delivery LNP of any one of the preceding claims, wherein the payload is chosen from an mRNA encoding secreted protein, a membrane-bound protein, an intracellular protein, an antibody molecule or an enzyme.

33. The delivery LNP of any one of the preceding claims, wherein the payload is an mRNA encoding an antibody molecule.

34. The delivery LNP of any one of the preceding claims, wherein the payload is an mRNA encoding an enzyme.

35. The delivery LNP of claim 34, wherein the enzyme is associated with a rare disease (e.g., a lysosomal storage disease).

36. The delivery LNP of claim 34, wherein the enzyme is associated with a metabolic disorder (e.g., as described herein).

37. The delivery LNP of claim 34, wherein the payload is an mRNA encoding a urea cycle enzyme.

38. The delivery LNP of any one of the preceding claims, wherein the target cell delivery LNP can be administered at a lower dose compared to a reference LNP, e.g., as described herein.

39. The delivery LNP of claim 38, wherein the target cell delivery LNP administered at a dose that is at least 10%, 20%, 30%, 40%, 50%, 60%, 70%, 80%, or 90% lower compared to the dose of a reference LNP.

40. A method of enhancing a payload level (e.g., payload expression) in a subject, comprising:
administering to the subject a delivery lipid nanoparticle (LNP) comprising:
(i) an ionizable lipid, e.g., an amino lipid;
(ii) a sterol or other structural lipid;
(iii) a non-cationic helper lipid or phospholipid;
(iv) a payload; and
(v) optionally, a PEG-lipid,
wherein the target cell delivery LNP is administered in an amount sufficient to result in one, two or all of:
(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;
(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;
(c) expression and/or activity of payload in greater than 30%, 40%, 50%, 60%, 65%, 70%, 75% or more total liver cells, e.g., in about 60% of total liver cells; or
(d) enhanced payload level (e.g., expression) and/or lipid level, e.g., about 1.5-fold, 2-fold, 3-fold, 4-fold, 5-fold, or 6-fold (e.g., about 3-fold), in liver cell expression, e.g., hepatocyte expression, relative to a reference LNP.

41. A method of treating or ameliorating a symptom of a disorder or disease, e.g., a rare disease, in a subject, the method comprising:
administering to the subject a delivery lipid nanoparticle (LNP) comprising:
(i) an ionizable lipid, e.g., an amino lipid;
(ii) a sterol or other structural lipid;
(iii) a non-cationic helper lipid or phospholipid;
(iv) a payload; and
(v) optionally, a PEG-lipid,
wherein the target cell delivery LNP is administered in an amount sufficient to result in one, two or all of:
(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;
(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;
(c) expression and/or activity of payload in greater than 30%, 40%, 50%, 60%, 65%, 70%, 75% or more total liver cells, e.g., in about 60% of total liver cells; or
(d) enhanced payload level (e.g., expression) and/or lipid level, e.g., about 1.5-fold, 2-fold, 3-fold, 4-fold, 5-fold, or 6-fold (e.g., about 3-fold), in liver cell expression, e.g., hepatocyte expression, relative to a reference LNP,
thereby treating or ameliorating a symptom of the disorder or disease.

42. The method of claim 40 or 41, wherein the target cell delivery LNP is administered in an amount that results in one, two or all of:
a) greater Maximum Concentration Observed (Cmax) in the liver relative to plasma, e.g., a Cmax that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5-fold or more in the liver relative to plasma;
b) greater half-life (t ½) in the liver relative to plasma, e.g., a t ½ that is at least 1-, 1.1-, 1.2-, 1.3-, 1.4-, 1.5-, 1.6-, 1.7-, 1.8-, 1.9-, 2-, 2.1-, 2.2-, 2.3-, 2.4-, 2.5, 2.6-, 2.7-, 2.8-, 2.9, 3-fold or more in the liver relative to plasma; or
c) greater % Extrapolated Area under the Concentration Time Curve (AUC % Extrap) in the liver relative to plasma, e.g., AUC % Extrap that is at least 5-, 10-, 15-, 20-, 25, 30-, 35-, 40-fold or more in the liver relative to plasma.

43. The method of any one of claims 40-42, wherein the target cell delivery LNP is administered in an amount that results in an improved parameter in vivo relative to a reference LNP, wherein said improved parameter is chosen from one, two, three, four, five, six, seven or more (e.g., all), or any combination of the following:
1) enhanced payload level in the liver, e.g., increased the level of payload mRNA or payload protein in the liver, e.g., increased delivery, transfection and/or expression, by at least 1-, 2-, 3-, 4-, 5-, 6-, 7-, 8- or more post-administration to a subject, e.g., IV administration to a non-human primate;
2) enhanced serum stability by at least 20%, 30%, 40%, 50%, 60%, 70%, 80% or more lipid remaining after 24 hours of administration, e.g., IV administration to a subject, e.g., mouse;
3) reduced immunogenicity, e.g., reduced levels of IgM or IgG which recognize the LNP, e.g., reduced IgM clearance by at least 1.2 to 5-fold;
4) increased bioavailability post-administration to a subject, e.g., IV administration to a non-human primate, e.g., at least 1.2-fold, 2-fold, 3-fold, 4-fold, 5-fold, 6-fold, 7-fold, 8-fold or more, e.g., as observed by increased AUC post-administration to a subject, e.g., a non-human primate;
5) enhanced liver distribution, e.g., enhanced liver cell positivity relative to a reference LNP, e.g., by at least 1-fold, 2-fold, 3-fold, 4-fold, 5-fold, 6-fold, 7-fold, 8-fold, 9-fold or more, post-administration to a subject, e.g., a non-human primate;
6) enhanced tissue concentration of lipid and/or payload in the liver, e.g., at least 6 hours, at least 12 hours, at least 24 hours post-administration to a subject;
7) enhanced endosomal escape; or
8) slower lipid metabolism in the liver relative to the spleen, e.g., at least 10%, 20%, 30%, 40%, 50%, 60%, 70%, 80%, 90% or more lipid remaining in the liver 24 hours post-administration.

44. The method of any one of claims 40-43, wherein the target cell delivery LNP is administered in an amount that results in one, two, three or all of:
1) an increased response rate, e.g., a defined by at specified threshold of liver cell transfection;
2) at least 5%, 10%, 15%, 20%, 25%, 30%, 34%, 35%, 36%, 37%, 38%, 39%, 40% or more liver cell transfection;
3) an increased responder rate, e.g., a defined by at specified threshold of liver cell transfection; or
4) an increased response rate greater than a reference LNP, e.g., at least 1-fold, 1.5-fold, 2-fold, 2.5-fold, or 3-fold or greater response rate.

45. The method of any one of claims 40-44, wherein the target cell delivery LNP is formulated for systemic delivery.

46. The method of any one of claims 40-45, wherein the target cell delivery LNP is administered systemically, e.g., parenterally (e.g., intravenously, intramuscularly, subcutaneously, intrathecally, or intradermally) or enterally (e.g., orally, rectally or sublingually).

47. The method of any one of claims 40-46, wherein the target cell delivery LNP delivers the payload to a cell capable of protein synthesis and/or a cell having a high engulfing capacity.

48. The method of any one of claims 40-47, wherein the target cell delivery LNP delivers the payload to a liver cell, e.g., a hepatocyte, a hepatic stellate cell, a Kupffer cell, or a liver sinusoidal cell, or a combination thereof.

49. The method of any one of claims 40-48, wherein the target cell delivery LNP delivers the payload to a hepatocyte.

50. The method of any one of claims 40-49, wherein the target cell delivery LNP delivers the payload to a splenic cell, e.g., a non-immune splenic cell (e.g., a splenocyte).

51. The method of any one of claims 40-50, wherein the target cell delivery LNP delivers the payload to a cell chosen from an ovarian cell, a lung cell, an intestinal cell, a heart cell, a skin cell, an eye cell or a brain cell, or a skeletal muscle cell.

52. The method of any one of claims 40-51, wherein the target cell delivery LNP delivers the payload to a non-immune cell.

53. The method of any one of claims 40-52, wherein an intracellular concentration of the nucleic acid molecule in the target cell is enhanced.

54. The method of any one of claims 40-53, wherein uptake of the nucleic acid molecule by the target cell is enhanced.

55. The method of any one of claims 40-54, wherein an activity of the nucleic acid molecule in the target cell is enhanced.

56. The method of any one of claims 40-55, wherein expression of the nucleic acid molecule in the target cell is enhanced.

57. The method of any one of claims 40-56, wherein an activity of a protein encoded by the nucleic acid molecule in the target cell is enhanced.

58. The method of any one of claims 40-57, wherein expression of a protein encoded by the nucleic acid molecule in the target cell is enhanced.

59. The method of any one of claims 40-58, wherein delivery is enhanced in vivo.

60. The method of any one of claims 40-59, wherein the payload is a peptide, polypeptide, protein or a nucleic acid.

61. The method of any one of claims 40-60, wherein the is a nucleic acid molecule chosen from RNA, mRNA, dsRNA, siRNA, antisense RNA, ribozyme, CRISPR/Cas9, ssDNA and DNA.

62. The method of any one of claims 40-61, wherein the payload is chosen from a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), or a combination thereof.

63. The method of any one of claims 40-62, wherein the payload is an mRNA, a siRNA, a miR, or a CRISPR.

64. The method of any one of claims 40-63, wherein the payload is an mRNA encoding a protein of interest other than an immune cell payload.

65. The method of any one of claims 40-64, wherein the payload is chosen from an mRNA encoding secreted protein, a membrane-bound protein, an intracellular protein, an enzyme.

66. The method of any one of claims 40-65, wherein the payload is an mRNA encoding an antibody molecule.

67. The method of any one of claims 40-66, wherein the payload is an mRNA encoding an enzyme.

68. The method of any one of claims 40-67, wherein the enzyme is associated with a rare disease (e.g., a lysosomal storage disease), or a metabolic disorder (e.g., as described herein).

69. The method of claim 68, wherein the payload is an mRNA encoding a urea cycle enzyme.

70. The method of claim 68, wherein the disease is a metabolic disorder.

71. The method of any one of claims 40-70, wherein the target cell delivery LNP can be administered at a lower dose compared to a reference LNP, e.g., as described herein.

72. The method of any one of claims 40-71, wherein the target cell delivery LNP administered at a dose that is at least 10%, 20%, 30%, 40%, 50%, 60%, 70%, 80%, or 90% lower compared to the dose of a reference LNP.

73. The method of claim 72, wherein the target cell delivery LNP delivered at a lower dose results in similar or enhanced lipid and/or payload level in a target cell, organ or cellular compartment.

74. The method of claim 71 or 72, wherein the target cell delivery LNP can be administered at a reduced frequency compared to a reference LNP, e.g., as described herein.

75. The delivery LNP or the method of any of the preceding claims, wherein the ionizable lipid comprises an amino lipid.

76. The delivery LNP or the method of any of the preceding claims, wherein the ionizable lipid comprises a compound of any of Formulae (I VI), (I VI-a), (I VII), (I VIII), (I VIIa), (I VIIIa), (I VIIIb), (I VIIb-1), (I VIIb-2), (I VIIb-3), (I VIIb-4), (I VIIb-5), (I VIIc), (I VIId), (I VIIIc), or (I VIIId).

77. The delivery LNP or the method of any of the preceding claims, wherein the ionizable lipid comprises an amino lipid having a squaramide head group.

78. The delivery LNP or the method of any of the preceding claims, wherein the ionizable lipid comprises a compound selected from the group consisting of Compound I-301, Compound (R)-I-301, Compound (S)-I-301, Compound I-49, Compound (R)-I-49, Compound (S)-I-49, Compound I-292, Compound I-309, Compound I-317, Compound I-326, Compound I-347, Compound I-348, Compound I-349, Compound I-350, and Compound I-352.

79. The delivery LNP or the method of any of the preceding claims, wherein the ionizable lipid comprises a compound selected from Compound I-301 and Compound I-49.

80. The delivery LNP or the method of any of the preceding claims, wherein the ionizable lipid comprises Compound I-301.

81. The delivery LNP or the method of any one of claims 1-79, wherein the ionizable lipid comprises Compound I-49.

82. The delivery LNP or the method of any of the preceding claims, wherein the cell is a liver cell, e.g., a hepatocyte, and the ionizable lipid comprises a compound selected from the group consisting of Compound I-301 and Compound I-49.

83. The delivery LNP or the method of any of the preceding claims, wherein the cell is a splenic cell, e.g., a splenocyte, and the ionizable lipid comprises a compound selected from the group consisting of Compound I-301 and Compound I-49.

84. The delivery LNP or the method of any of the preceding claims, wherein the ionizable lipid comprises is a racemic mixture of the amino lipid, e.g., a mixture comprising a (R)-enantiomer and an (S)-enantiomer of an amino lipid.

85. The delivery LNP or the method of any of the preceding claims, wherein the reference LNP comprises an ionizable lipid having Formula I-XII.

86. The delivery LNP or the method of claim 85, wherein the reference LNP does not comprises an ionizable lipid having a chiral center.

87. The delivery LNP or the method of claim 85, wherein the reference LNP does not comprises an ionizable lipid comprising more than one branched alkyl chains.

88. The delivery LNP or the method of claim 85, wherein the reference LNP does not comprises a cyclic-substituted amino lipid.

89. The target cell delivery LNP or the method of claim 85, wherein the reference LNP does not comprise a carbocyclic-substituted amino lipid.

90. The target cell delivery LNP or the method of claim 85, wherein the reference LNP does not comprise a cycloalkenyl-substituted amino lipid.

91. The delivery LNP or the method of any of the preceding claims, wherein the target cell delivery LNP comprises an amino lipid having a chiral center.

92. The delivery LNP or the method of any of the preceding claims, wherein the target cell delivery LNP comprises an amino lipid comprising more than one branched alkyl chains.

93. The delivery LNP or the method of any of the preceding claims, wherein the target cell delivery LNP comprises a cyclic-substituted amino lipid.

94. The delivery LNP or the method of any of claims 1-92, wherein the target cell delivery LNP comprises a carbocyclic-substituted amino lipid.

95. The delivery LNP or the method of any of claims 1-92, wherein the target cell delivery LNP comprises a cycloalkenyl-substituted amino lipid.

96. The delivery LNP or the method of any of the preceding claims, wherein the target cell delivery LNP comprises a cyclobutenyl-substituted amino lipid.

97. The delivery LNP or the method of any of the preceding claims, wherein the target cell delivery LNP comprises a cyclobutene-1,2-dione-substituted amino lipid.

98. The delivery LNP or the method of any of the preceding claims, wherein the target cell delivery LNP comprises a squaramide-substituted amino lipid, e.g., an amino lipid comprising a squaramide group.

99. The delivery LNP or the method of any of the preceding claims, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.

100. The delivery LNP or the method of claim 99, wherein the cell is a liver cell, e.g., a hepatocyte, and the non-cationic helper lipid or phospholipid comprises a compound selected from the group consisting of DSPC, DMPE, and Compound H-409.

101. The delivery LNP or the method of claim 99, wherein the phospholipid is DSPC.

102. The delivery LNP or the method of claim 99, wherein the phospholipid is DMPE.

103. The delivery LNP or the method of claim 99, wherein the phospholipid is Compound H-409.

104. The delivery LNP or the method of any of the preceding claims, which comprises a PEG-lipid.

105. The delivery LNP or the method of claim 104, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

106. The delivery LNP or the method of claim 104, wherein the PEG lipid is selected from the group consisting of PEG-c-DOMG, PEG-DMG, PEG-DLPE, PEG-DMPE, PEG-DPPC and PEG-DSPE lipid.

107. The delivery LNP or the method of any one of claims 104-106, wherein the PEG-lipid is PEG-DMG.

108. The delivery LNP or the method of claim 104, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

109. The target cell delivery LNP or the method of claim 104 or 108, wherein the PEG lipid comprises a compound selected from the group consisting of Compound P-428, Compound PL-16, Compound PL-17, Compound PL-18, Compound PL-19, Compound PL-1, and Compound PL-2.

110. The delivery LNP or the method of any of the preceding claims, wherein the LNP comprises a molar ratio of (i) ionizable lipid: (iii) a non-cationic helper lipid or phospholipid, of about 50:10, 49:11, 48:12, 47:13, 46:14, 45:15, 44:16, 43:17, 42:18 or 41:19.

111. The delivery LNP or the method of any of the preceding claims, wherein the LNP comprises about 41 mol % to about 50 mol % of ionizable lipid and about 10 mol % to about 19 mol % of non-cationic helper lipid or phospholipid.

112. The delivery LNP or the method of any of the preceding claims, wherein the LNP comprises about 50 mol % of ionizable lipid and about 10 mol % of non-cationic helper lipid or phospholipid.

113. The delivery LNP or the method of any of the preceding claims, wherein the molar ratio of (i) ionizable lipid: (iii) a non-cationic helper lipid or phospholipid, is about 50:10.

114. The delivery LNP or the method of any of the preceding claims, wherein the lipid nanoparticle comprises Compound I-301 as the ionizable lipid, DSPC as the phospholipid, cholesterol or a cholesterol/β-sitosterol blend as the structural lipid and Compound 428 as the PEG lipid.

115. The delivery LNP or the method of any of the preceding claims, wherein the ionizable lipid:phospholipid:structural lipid:PEG lipid are in a ratio chosen from: (i) 50:10:38:2; (ii) 50:20:28:2; (iii) 40:20:38:2; or (iv) 40:30:28:2.

116. The delivery LNP, or method of claim 115, wherein the LNP comprises:
i) about 50 mol % ionizable lipid, wherein the ionizable lipid is a compound selected from the group consisting of Compound I-301, Compound I-321, Compound I-182 or Compound I-49;
(ii) about 10 mol % phospholipid, wherein the phospholipid is DSPC;
(iii) about 38.5 mol % structural lipid, wherein the structural lipid is selected from β-sitosterol and cholesterol; and
(iv) about 1.5 mol % PEG lipid, wherein the PEG lipid is Compound P-428.

117. A pharmaceutical composition comprising the delivery lipid nanoparticle of any of claim 1-40 or 75-116, and a pharmaceutically acceptable carrier.

118. A GMP-grade pharmaceutical composition comprising the delivery lipid nanoparticle of any of claim 1-40 or 75-116, and a pharmaceutically acceptable carrier.

119. The pharmaceutical composition of claim 117 or 118, which has greater than 95%, 96%, 97%, 98%, or 99% purity, e.g., at least 1%, 2%, 3%, 4%, 5%, or more contaminants removed.


SEQUENCE LISTING (181 sequences)

SEQ ID NO: 1-59 - skipped (59 entries)

SEQ ID NO: 60 - DNA, 92 bp, Artificial Sequence
  source: /note=“Description of Artificial Sequence: Synthetic oligonucleotide”
tcaagcttttggaccctcgtacagaagctaatacgactcactatagggaaataagagagaaaagaagagtaagaagaaatataagagccacc

SEQ ID NO: 61 - DNA, 119 bp, Artificial Sequence
  source: /note=“Description of Artificial Sequence: Synthetic polynucleotide”
tgataataggctggagcctcggtggccatgcttcttgccccttgggcctccccccagcccctcctccccttcctgcacccgtacccccgtggtctttgaataaagtctgagtgggcggc

SEQ ID NO: 62 - DNA, 164 bp, Artificial Sequence
  source: /note=“Description of Artificial Sequence: Synthetic polynucleotide”
tgataataggctggagcctcggtggccatgcttcttgccccttgggcccaaacaccattgtcacactccatccccccagcccctcctccccttcctccataaagtaggaaacactacatgcacccgtacccccgtggtctttgaataaagtctgagtgggcggc

SEQ ID NO: 63 - protein, 22 aa, Artificial Sequence
  source: /note=“Description of Artificial Sequence: Synthetic peptide”
GSGATNFSLLKQAGDVEENPGP

SEQ ID NO: 64 - DNA, 66 bp, Artificial Sequence
  source: /note=“Description of Artificial Sequence: Synthetic oligonucleotide”
ggaagcggagctactaacttcagcctgctgaagcaggctggagacgtggaggagaaccctggacct

SEQ ID NO: 65 - DNA, 108 bp, Artificial Sequence
  source: /note=“Description of Artificial Sequence: Synthetic polynucleotide”
tccggactcagatccggggatctcaaaattgtcgctcctgtcaaacaaactcttaactttgatttactcaaactggctggggatgtagaaagcaatccaggtccactc

SEQ ID NO: 66 - RNA, 87 nt, Unknown
  source: /note=“Description of Unknown: mmiR-142 sequence”
gacagugcagucacccauaaaguagaaagcacuacuaacagcacuggaggguguaguguuuccuacuuuauggaugaguguacugug

SEQ ID NO: 67 - RNA, 23 nt, Unknown
  source: /note=“Description of Unknown: mmiR-142-3p sequence”
uguaguguuuccuacuuuaugga

SEQ ID NO: 68 - RNA, 23 nt, Unknown
  source: /note=“Description of Unknown: mmiR-142-3p binding site sequence”
uccauaaaguaggaaacacuaca

SEQ ID NO: 69 - RNA, 21 nt, Unknown
  source: /note=“Description of Unknown: mmiR-142-5p sequence”
cauaaaguagaaagcacuacu

SEQ ID NO: 70 - RNA, 21 nt, Unknown
  source: /note=“Description of Unknown: mmiR-142-5p binding site sequence”
aguagugcuuucuacuuuaug

SEQ ID NO: 71 - RNA, 85 nt, Unknown
  source: /note=“Description of Unknown: miR-122 sequence”
ccuuagcagagcuguggagugugacaaugguguuugugucuaaacuaucaaacgccauuaucacacuaaauagcuacugcuaggc

SEQ ID NO: 72 - RNA, 22 nt, Unknown
  source: /note=“Description of Unknown: miR-122-3p sequence”
aacgccauuaucacacuaaaua

SEQ ID NO: 73 - RNA, 22 nt, Unknown
  source: /note=“Description of Unknown: miR-122-3p binding site sequence”
uauuuagugugauaauggcguu

SEQ ID NO: 74 - RNA, 22 nt, Unknown
  source: /note=“Description of Unknown: miR-122-5p sequence”
uggagugugacaaugguguuug

SEQ ID NO: 75 - RNA, 22 nt, Unknown
  source: /note=“Description of Unknown: miR-122-5p binding site sequence”
caaacaccauugucacacucca

SEQ ID NO: 76 - DNA, 47 bp, Artificial Sequence
  source: /note=“Description of Artificial Sequence: Synthetic oligonucleotide”
gggaaataagagagaaaagaagagtaagaagaaatataagagccacc

SEQ ID NO: 77 - DNA, 41 bp, Artificial Sequence
  source: /note=“Description of Artificial Sequence: Synthetic oligonucleotide”
gggaaataagagagaaaagaagagtaagaagaaatataaga

SEQ ID NO: 78 - DNA, 57 bp, Artificial Sequence
  source: /note=“Description of Artificial Sequence: Synthetic oligonucleotide”
gggaaataagagagaaaagaagagtaagaagaaatataagaccccggcgccgccacc

SEQ ID NO: 79 - DNA, 54 bp, Artificial Sequence
  source: /note=“Description of Artificial Sequence: Synthetic oligonucleotide”
gggaaataagagagaaaagaagagtaagaagaaatataagaccccggcgccacc

SEQ ID NO: 80 - RNA, 10 nt, Artificial Sequence
  source: /note=“Description of Artificial Sequence: Synthetic oligonucleotide”
ccccggcgcc

SEQ ID NO: 81-176 - skipped (96 entries)

SEQ ID NO: 177 - RNA, 12 nt, Artificial Sequence
  source: /note=“Description of Artificial Sequence: Synthetic oligonucleotide”
ccgccgccgccg

SEQ ID NO: 178 - RNA, 15 nt, Artificial Sequence
  source: /note=“Description of Artificial Sequence: Synthetic oligonucleotide”
ccgccgccgccgccg

SEQ ID NO: 179 - protein, 4 aa, Artificial Sequence
  source: /note=“Description of Artificial Sequence: Synthetic peptide”
NPGP

SEQ ID NO: 180 - RNA, 30 nt, Artificial Sequence
  source: /note=“Description of Artificial Sequence: Synthetic oligonucleotide”
  misc_feature (1)..(30): /note=“This sequence may encompass 1 to 10, 2 to 8, 3 to 6, or 4 to 5 ′ccg′ repeating units”
ccgccgccgccgccgccgccgccgccgccg

SEQ ID NO: 181 - RNA, 15 nt, Artificial Sequence
  source: /note=“Description of Artificial Sequence: Synthetic oligonucleotide”
  misc_feature (1)..(15): /note=“This sequence may encompass 1-5 ′ccg′ repeating units”
ccgccgccgccgccg